(19) United States
(12) Patent Application Publication
(10) Pub. No.: US 2021/0046160 A1
(43) Pub. Date: Feb. 18, 2021

(54) Immuno Oncology Combination Therapies With IL-2 Conjugates

(71) Applicant: Synthorx, Inc., La Jolla, CA (US)

(72) Inventors: Jerod PTACIN, La Jolla, CA (US); Carolina E. CAFFARO, La Jolla, CA (US); Marcos MILLA, La Jolla, CA (US)

(73) Assignee: Synthorx, Inc., La Jolla, CA (US)

(21) Appl. No.: 16/993,967

(22) Filed: Aug. 14, 2020

Related U.S. Application Data
(60) Provisional application No. 62/962,668, filed on Jan. 17, 2020.
(60) Provisional application No. 62/903,187, filed on Sep. 20, 2019.
(60) Provisional application No. 62/887,400, filed on Aug. 15, 2019.

(51) Int. Cl. A61K 38/20; A61K 47/68

(52) U.S. Cl. CPC A61K 38/2013; A61K 47/6849

(57) ABSTRACT

Disclosed herein are compositions, kits, and methods comprising interleukin (IL) conjugates (e.g., IL-2 conjugates) in combination with other agents or methods useful for the treatment of one or more indications, such as the treatment of proliferative diseases. Also described herein are pharmaceutical compositions and kits comprising one or more of the interleukin conjugates (e.g., IL-2 conjugates).

### CROSS-REFERENCE TO RELATED APPLICATIONS

This application claims priority to U.S. Provisional Application No. 62/887,400, filed on Aug. 15, 2019, U.S. Provisional Application No. 62/903,187, filed on Sep. 20, 2019, and U.S. Provisional Application No. 62/962,668, filed on Jan. 17, 2020, the disclosures of each of which are hereby incorporated by reference in their entireties.

### SEQUENCE LISTING

This application contains a Sequence Listing which has been submitted electronically in ASCII format and is hereby incorporated by reference in its entirety. Said ASCII copy, created on Aug. 12, 2020, is named 2020-08-12_01183-0073-00PCT_seq_listing.txt and is 128,000 bytes in size.

### BACKGROUND OF THE DISCLOSURE

Distinct populations of T cells modulate the immune system to maintain immune homeostasis and tolerance. For example, regulatory T (Treg) cells prevent inappropriate responses by the immune system by preventing pathological self-reactivity while cytotoxic T cells target and destroy infected cells and/or cancerous cells. In some instances, modulation of the different populations of T cells provides an option for treatment of a disease or indication. In some instances, this is benefited by the presence of additional agents or methods in combination therapy.

Accordingly, in one aspect, provided herein are methods of treating cancer in a subject, comprising administering to a subject an IL-2 conjugate in combination with one or more immune checkpoint inhibitors.

### SUMMARY OF THE DISCLOSURE

Described herein, in certain embodiments, are methods for treating cancer. The following embodiments are encompassed.

Embodiment A1 is a method of treating cancer in a subject in need thereof, comprising administering to the subject a therapeutically effective amount of (a) an IL-2 conjugate, and (b) one or more immune checkpoint inhibitors, wherein the IL-2 conjugate comprises the amino acid sequence of SEQ ID NO: 3 in which at least one amino acid residue in the IL-2 conjugate is replaced by the structure of Formula (I):

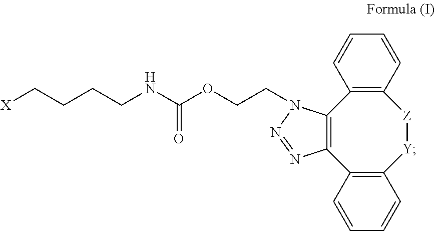

wherein:

### Z is CH₂ and Y is

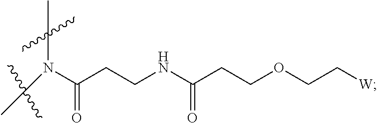

### Y is CH₂ and Z is

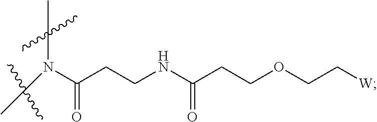

### Z is CH₂ and Y is

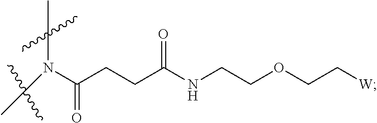

### Y is CH₂ and Z is

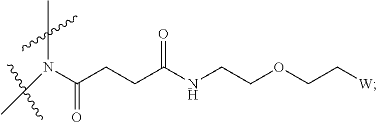

W is a PEG group having an average molecular weight selected from 5 kDa, 10 kDa, 15 kDa, 20
kDa, 25 kDa, 30 kDa, 35 kDa, 40 kDa, 45 kDa, 50 kDa, and 60 kDa;
X has the structure:

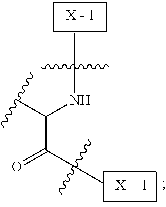 [SMILES: *NC(*)C(*)=O]

X−1 indicates the point of attachment to the preceding amino acid residue; and
X+1 indicates the point of attachment to the following amino acid residue;
wherein the position of the structure of Formula (I) in SEQ ID NO: 3 is selected from K34, F41, F43, K42, E61, P64, R37, 140, E67, Y44, V68, and L71.

Embodiment A2 is the method according to embodiment A1, wherein in the IL-2 conjugate Z is CH₂ and Y is

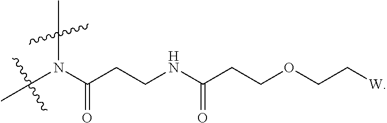

Embodiment A3 is the method according to embodiment A1, wherein in the IL-2 conjugate Y is CH₂ and Z is

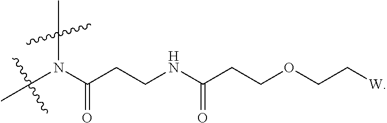

Embodiment A4 is the method according to embodiment A1, wherein in the IL-2 conjugate Z is CH₂ and Y is

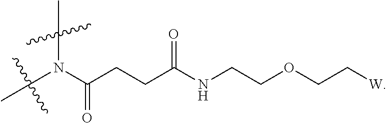

Embodiment A5 is the method according to embodiment A1, wherein in the IL-2 conjugate Y is CH₂ and Z is

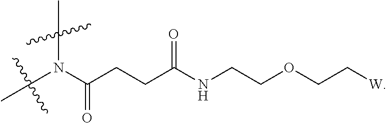

Embodiment A6 is the method according to any one of embodiments A1-A5, wherein in the IL-2 conjugate the PEG group has an average molecular weight of 25 kDa, 30 kDa, or 35 kDa.

Embodiment A7 is the method according to embodiment A6, wherein in the IL-2 conjugate the PEG group has an average molecular weight of 30 kDa.

Embodiment A8 is the method according to any one of embodiments A1-A7, wherein in the IL-2 conjugate the position of the structure of Formula (I) in SEQ ID NO: 3 is P64.

Embodiment A9 is the method of embodiment A1, wherein the structure of Formula (I) has the structure of Formula (X) or Formula (XI), or is a mixture of Formula (X) and Formula (XI):

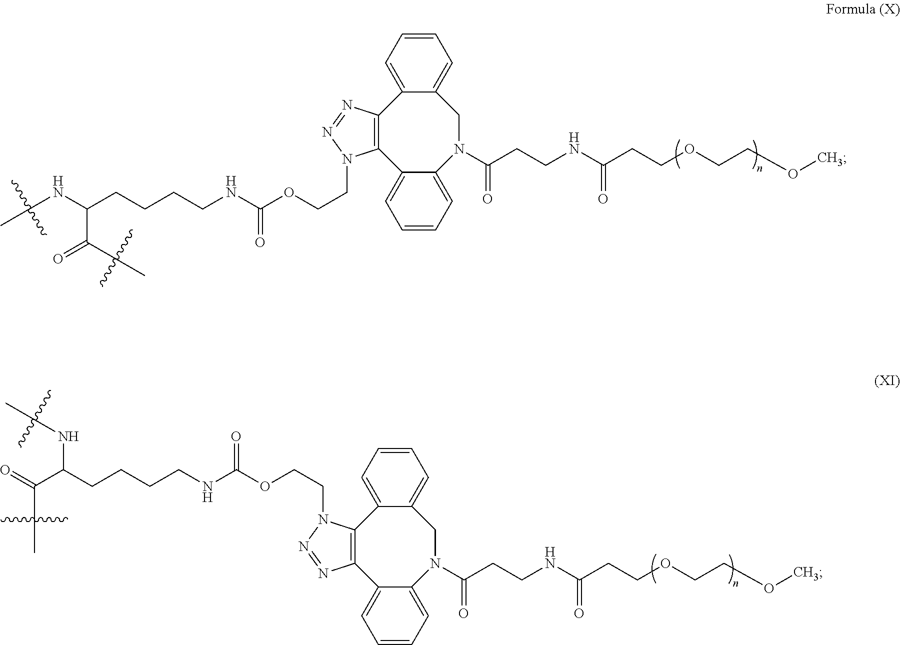

wherein:
n is an integer in the range from about 2 to about 5000; and
the wavy lines indicate covalent bonds to amino acid residues within SEQ ID NO: 3 that are not replaced.

Embodiment A10 is the method of embodiment A9, wherein in the IL-2 conjugate the position of the structure of Formula (X) or Formula (XI) in SEQ ID NO: 3 is P64.

Embodiment A11 is the method of embodiment A9 or A10, wherein in the IL-2 conjugate n is an integer such that —(OCH₂CH₂)ₙ—OCH₃ has a molecular weight of about 25 kDa, 30 kDa, or 35 kDa.

Embodiment A12 is the method of embodiment A11, wherein in the IL-2. conjugate n is an integer such that —(OCH₂CH₂)ₙ—OCH₃ has a molecular weight of about 30 kDa.

Embodiment A13 is the method of embodiment A1, wherein the structure of Formula (I) has the structure of Formula (XII) or Formula (XIII), or is a mixture of Formula (XII) and Formula (XIII):

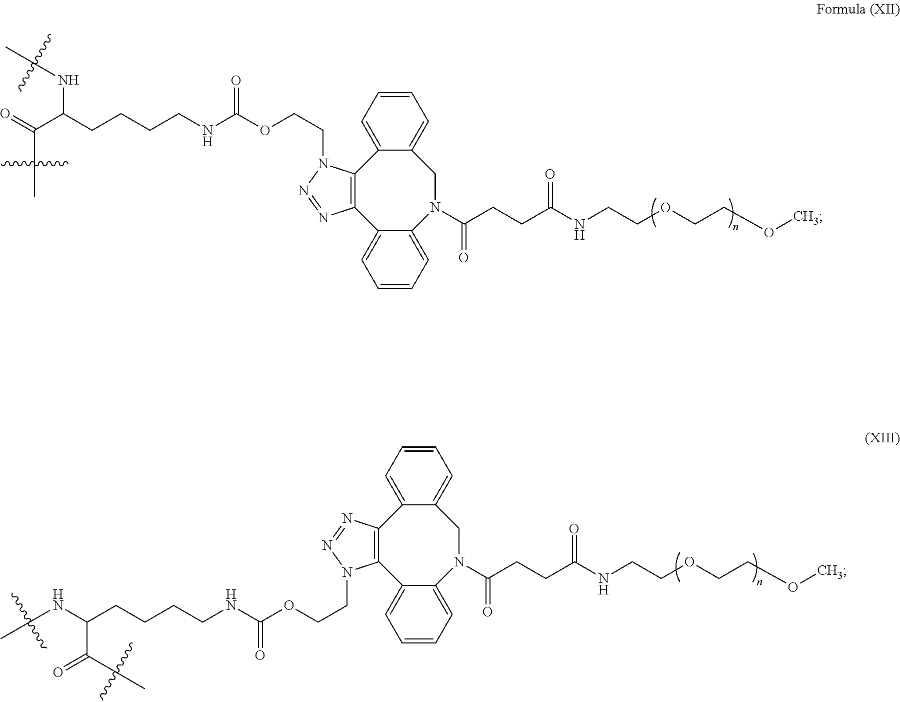

wherein:
n is an integer in the range from about 2 to about 5000; and
the wavy lines indicate covalent bonds to amino acid residues within SEQ ID NO: 3 that are not replaced.

Embodiment A14 is the method of embodiment A13, wherein in the IL-2 conjugate the position of the structure of Formula (XII) or Formula (XIII) in SEQ ID NO: 3 is P64.

Embodiment A15 is the method of embodiment A13 or A14, wherein in the IL-2 conjugate n is an integer such that —(OCH₂CH₂)ₙ—OCH₃ has a molecular weight of about 25 kDa, 30 kDa, or 35 kDa.

Embodiment A16 is the method of embodiment A15, wherein in the IL-2. conjugate n is an integer such that —(OCH₂CH₂)ₙ—OCH₃ has a molecular weight of about 30 kDa.

Embodiment A17 is a method of treating a cancer in a subject in need thereof, comprising administering to the subject a therapeutically effective amount of (a) an IL-2 conjugate; and (b) one or more immune checkpoint inhibitors, wherein the IL-2 conjugate comprises the amino acid sequence of SEQ ID NO: 50, wherein [AzK_L1_PEG30kD] has the structure of Formula (IV) or Formula (V), or is a mixture of the structures of Formula (IV) and Formula (V):

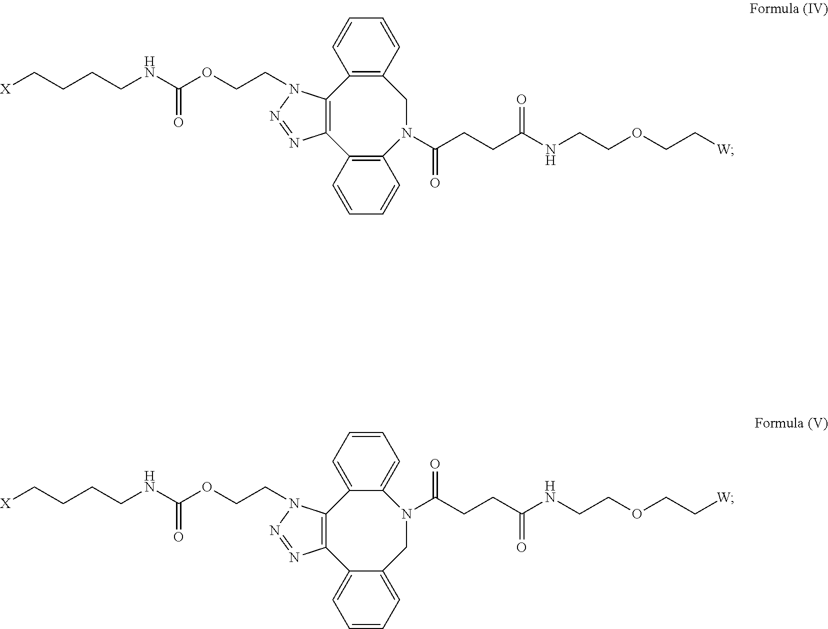

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
X has the structure:

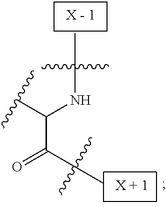 [SMILES: *NC(*)C(*)=O]

X−1 indicates the point of attachment to the preceding amino acid residue; and
X+1 indicates the point of attachment to the following amino acid residue.

Embodiment A18 is the method according to embodiment A17, wherein W is a PEG group having an average molecular weight selected from 25 kDa, 30 kDa, or 35 kDa.

Embodiment A19 is the method according to embodiment A18, wherein W is a PEG group having an average molecular weight of 30 kDa.

Embodiment A20 is a method of treating a cancer in a subject in need thereof, comprising administering to the subject a therapeutically effective amount of (a) an IL-2 conjugate, and (b) one or more immune checkpoint inhibitors, wherein the IL-2 conjugate comprises the amino acid sequence of SEQ ID NO: 50, wherein [AzK_L1_PEG30kD] has the structure of Formula (XII) or Formula (XIII), or is a mixture of the structures of Formula (XII) and Formula (XIII):

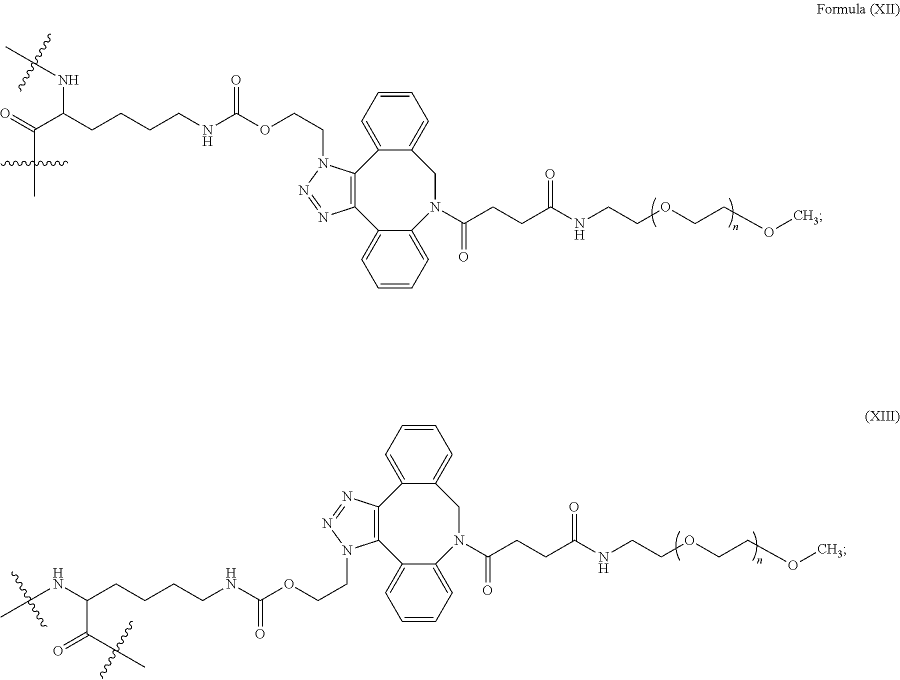

wherein:
n is an integer such that —(OCH₂CH₂)ₙ—OCH₃ has a molecular weight of about 30 kDa; and the wavy lines indicate covalent bonds to amino acid residues within SEQ ID NO: 50 that are not replaced.

Embodiment A21 is the method according to anyone of embodiments A1-A20, wherein the one or more immune checkpoint inhibitors is one or more PD-1 inhibitors.

Embodiment A22 is the method according to embodiment A21, wherein the one or more PD-1 inhibitors is selected from pembrolizumab, nivolumab, and cemiplimab.

Embodiment A23 is the method according to embodiment A22, wherein the one or more PD-1 inhibitors is pembrolizumab.

Embodiment A24 is the method according to embodiment A22, wherein the one or more PD-1 inhibitors is nivolumab.

Embodiment A25 is the method according to anyone of embodiments A1-A24, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

Embodiment A26 is the method according to anyone of embodiments A1-A25, wherein the IL-2 conjugate is administered to the subject once per week, once every two weeks, once every three weeks, once every 4 weeks, once every 5 weeks, once every 6 weeks, once every 7 weeks, or once every 8 weeks.

Embodiment A27 is the method according to anyone of embodiments A1-A26, wherein the IL-2 conjugate is administered to a subject by intravenous administration.

Embodiment A28 is the method according to anyone of embodiments A1-A27, wherein the IL-2 conjugate is a pharmaceutically acceptable salt, solvate, or hydrate.

### BRIEF DESCRIPTION OF THE DRAWINGS

A better understanding of the features and advantages of the present invention will be obtained by reference to the following detailed description that sets forth illustrative embodiments, in which the principles of the invention are utilized, and the accompanying drawings of which:

FIG. 1 shows a graph of anti-tumor activity of Compound A dosed IV on a QWx3 Schedule from Study 1 in Example 11. Black arrows denote days of dosing with Compound A.

FIG. 2 shows a graph of tumor volumes with Compound A dosed IV on a QWx3 Schedule from Study 1 in Example 11.

FIG. 3 shows tumor volumes on Day 15 post treatment for each animal treated QWx3 dosing with Compound A from Study 1 in Example 11. Black arrows denote days of dosing with Compound A.

FIG. 4 shows tumor volumes on Day 15 post treatment for each animal with Q2Wx2 dosing with Compound A from Study 1 in Example 11.

FIG. 5 shows mean tumor growth curves from treatment of mice with vehicle, 6 mg/kg Compound A as a single agent, anti-PD-1 antibody as a single agent, and the combination of 6 mg/kg Compound A and anti-PD-1 antibody from Study 2 of Example 11. Black arrows denote days of dosing with Compound A.

FIG. 6 shows a graph of % TGI data on Day 15 post treatment in the group treated with the combination of Compound A and anti-PD-1 antibody, compared to the groups treated with vehicle, Compound A alone or the anti-PD-1 antibody alone from Study 2 of Example 11. *p<0.05, **p<0.01, and ***p<0.01; vs. vehicle control. ^{⊥}p<0.05 vs. anti-PD-1 antibody. #p<0.05 vs. Compound A. Data represent mean tumor volume±SEM (14 mice/group).

FIG. 7 shows a graph of Kaplan-Meier survival curves for treatment groups from Study 2 of Example 11. *p<0.05 vs. vehicle control. p<0.05 vs. anti-PD-1 antibody. #p<0.05 vs. Compound A.

FIG. 8 represents mean tumor growth curves when Compound A was dosed a single agent at 1 mg/kg, 3 mg/kg, 6 mg/kg, and 9 mg/kg in Study 3 of Example 11. Data represent mean tumor volume±SEM (14 mice/group; except 12 mice/group for 9 mg/kg Compound A). Black arrows denote days of Compound A dosing.

FIG. 9 represent individual tumor volumes on Day 15 post-treatment from Study 3 of Example 11. Data represent individual tumor volumes; the mean SEM and % TGI compared to the vehicle control are also displayed. ***p<0.01 vs. vehicle control.

FIG. 10 shows a graph of Kaplan-Meier survival curves for treatment groups treated with vehicle (control), anti-PD-1 antibody alone, Compound A alone, and the combination of Compound A and anti-PD-1 antibody. *p<0.05 vs. vehicle control from Study 3 of Example 11. p<0.05 vs. anti-PD-1 antibody. #p<0.05 vs. Compound A.

FIG. 11A and FIG. 11B show graphs of representative cytokine levels for IL-2 and IL-2_P65[AzK_L1_PEG30kD]-1 alone and in combination with Nivolumab (Nivo) or Pembrolizumab (Pem) for a single donor of Example 12. FIG. 11A shows a graph of IFN-gamma, IL-8, IL-6, TNF-alpha, IL-4, and IL-5 levels. FIG. 11B shows a graph of IL-6, TNF-alpha, and IL-5 levels.

FIG. 12 shows the release of interferon-gamma in a mixed lymphocyte reaction (MLR) assay of a combination of Compound B (IL-2_P65[AzK_L1_PEG30kD]-1) and pembrolizumab according to Example 13.

FIG. 13 and FIG. 14 show the release of interferon-gamma in a mixed lymphocyte reaction (MLR) assay of a combination of Compound B (IL-2_P65[AzK_L1_PEG30kD]-1) and nivolumab according to Example 13.

FIG. 15 shows the pharmacokinetic properties of Compound B from Example 14.

FIGS. 16A-16D show the amount of pSTAT5+ cells in peripheral blood CD8+ T cells, CD8+ memory T cells, NK cells, and Treg cells, respectively, following administration of Compound B according to Example 14.

FIGS. 17A-17G show activation of Ki67 in CD8+T, NK, and Treg cell populations by Compound B according to Example 14.

FIGS. 18A-18D show analyses of tumor samples (CD8+ T cell, NK cell, and Treg cell levels and CD8+/Treg ratio) after treatment with Compound B according to Example 14.

FIG. 19 shows TCR diversity following treatment with Compound B and mouse anti-PD-1 antibody according to Example 15.

FIG. 20 shows TIL clonality versus T cell fraction following the indicated treatments (e.g., Compound B and/or mouse anti-PD-1 antibody) according to Example 15.

FIG. 21 shows T cell clonality following treatment with Compound B compared to vehicle control according to Example 15.

FIG. 22 shows an expression heatmap from Day 8 CT26 tumor samples following treatment with control (vehicle), Compound B (6 mg/kg), mouse anti-PD-1 (10 mg/kg), or combination of Compound B and mouse anti-PD-1 (N=10 mice per group) from Example 16.

FIG. 23A-23C show the key expression reporters of the state of the tumor microenvironment following Compound B treatment according to Example 16: analysis of infiltration of activated CD8+ effector and effector memory T cells, and cytolytic NK cells. CTL=control (vehicle); Cmpd B=Compound B; aPD1=mouse anti-PD-1 antibody; Cmpd B aPD1=combination of Compound B and mouse anti-PD-1 antibody.

FIG. 24A-24B show the profiler analysis interferon 7 gene expression signature levels in response to therapy according to Example 16. CTL=control (vehicle); Cmpd B=Compound B: aPD1=mouse anti-PD-1 antibody; Cmpd B aPD1=combination of Compound B and mouse anti-PD-1 antibody.

FIG. 25 and FIG. 26 show the survival and tumor growth assessment in re-challenged tumor-free animals according to Example 17.

FIG. 27A and FIG. 27B show that Compound B promotes an overall increase in peripheral memory T cells (CD3+), including memory CD8+ T cells, in re-challenged mice according to Example 17.

### DETAILED DESCRIPTION OF THE DISCLOSURE

It is to be understood that the foregoing general description and the following detailed description are exemplary and explanatory only and are not restrictive of any subject matter claimed. To the extent any material incorporated herein by reference is inconsistent with the express content of this disclosure, the express content controls.

### Definitions

Unless defined otherwise, all technical and scientific terms used herein have the same meaning as is commonly understood by one of skill in the art to which the claimed subject matter belongs. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as for example, within 15%, 10%, or 5%.

The section headings used herein are for organizational purposes only and are not to be construed as limiting the subject matter described.

The term “or” is used in the inclusive sense, equivalent to “and/or,” unless the context clearly dictates otherwise.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “significant” or “significantly” in reference to binding affinity means a change in the binding affinity of the cytokine (e.g., IL-2 polypeptide) sufficient to impact binding of the cytokine (e.g., IL-2 polypeptide) to a target receptor. In some instances, the term refers to a change of at least 10%, 20%, 30%, 40%, 50%, 60%, 70%, 80%, 90%, 95%, or more. In some instances, the term means a change of at least 2-fold, 3-fold, 4-fold, 5-fold, 6-fold, 7-fold, 8-fold, 9-fold, 10-fold, 50-fold, 100-fold, 500-fold, 1000-fold, or more.

In some instances, the term “significant” or“significantly” in reference to activation of one or more cell populations via a cytokine signaling complex means a change sufficient to activate the cell population. In some cases, the change to activate the cell population is measured as a receptor signaling potency. In such cases, an EC50 value may be provided. In other cases, an ED50 value may be provided. In additional cases, a concentration or dosage of the cytokine may be provided.

As used herein, the term “potency” refers to the amount of a cytokine (e.g., IL-2 polypeptide) required to produce a target effect. In some instances, the term “potency” refers to the amount of cytokine (e.g., IL-2 polypeptide) required to activate a target cytokine receptor (e.g., IL-2 receptor). In other instances, the term “potency” refers to the amount of cytokine (e.g., IL-2 polypeptide) required to activate a target cell population. In some cases, potency is measured as ED50 (Effective Dose 50), or the dose required to produce 50% of a maximal effect. In other cases, potency is measured as EC50 (Effective Concentration 50), or the dose required to produce the target effect in 50% of the population.

As used herein, the term “unnatural amino acid” refers to an amino acid other than one of the 20 naturally occurring amino acids. Exemplary unnatural amino acids are described in Young et al., “Beyond the canonical 20 amino acids: expanding the genetic lexicon,” J. of Biological Chemistry 285(15): 11039-11044 (2010), the disclosure of which is incorporated herein by reference.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. In some embodiments, the antigen is EGFR.

The term “monoclonal antibody(ies)” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

As used herein, “nucleotide” refers to a compound comprising a nucleoside moiety and a phosphate moiety. Exemplary natural nucleotides include, without limitation, adenosine triphosphate (ATP), uridine triphosphate (UTP), cytidine triphosphate (CTP), guanosine triphosphate (GTP), adenosine diphosphate (ADP), uridine diphosphate (UDP), cytidine diphosphate (CDP), guanosine diphosphate (GDP), adenosine monophosphate (AMP), uridine monophosphate (UMP), cytidine monophosphate (CMP), and guanosine monophosphate (GMP), deoxyadenosine triphosphate (dATP), deoxythymidine triphosphate (dTTP), deoxycytidine triphosphate (dCTP), deoxyguanosine triphosphate (dGTP), deoxyadenosine diphosphate (dADP), thymidine diphosphate (dTDP), deoxycytidine diphosphate (dCDP), deoxyguanosine diphosphate (dGDP), deoxyadenosine monophosphate (dAMP), deoxythymidine monophosphate (dTMP), deoxycytidine monophosphate (dCMP), and deoxyguanosine monophosphate (dGMP). Exemplary natural deoxyribonucleotides, which comprise a deoxyribose as the sugar moiety, include dATP, dTTP, dCTP, dGTP, dADP, dTDP, dCDP, dGDP, dAMP, dTMP, dCMP, and dGMP. Exemplary natural ribonucleotides, which comprise a ribose as the sugar moiety, include ATP, UTP, CTP, GTP, ADP, UDP, CDP, GDP, AMP, UMP, CMP, and GMP.

As used herein, “base” or “nucleobase” refers to at least the nucleobase portion of a nucleoside or nucleotide (nucleoside and nucleotide encompass the ribo or deoxyribo variants), which may in some cases contain further modifications to the sugar portion of the nucleoside or nucleotide. In some cases, “base” is also used to represent the entire nucleoside or nucleotide (for example, a “base” may be incorporated by a DNA polymerase into DNA, or by an RNA polymerase into RNA). However, the term “base” should not be interpreted as necessarily representing the entire nucleoside or nucleotide unless required by the context. In the chemical structures provided herein of a base or nucleobase, only the base of the nucleoside or nucleotide is shown, with the sugar moiety and, optionally, any phosphate residues omitted for clarity. As used in the chemical structures provided herein of a base or nucleobase, the wavy line represents connection to a nucleoside or nucleotide, in which the sugar portion of the nucleoside or nucleotide may be further modified. In some embodiments, the wavy line represents attachment of the base or nucleobase to the sugar portion, such as a pentose, of the nucleoside or nucleotide. In some embodiments, the pentose is a ribose or a deoxyribose.

In some embodiments, a nucleobase is generally the heterocyclic base portion of a nucleoside. Nucleobases may be naturally occurring, may be modified, may bear no similarity to natural bases, and/or may be synthesized, e.g., by organic synthesis. In certain embodiments, a nucleobase comprises any atom or group of atoms in a nucleoside or nucleotide, where the atom or group of atoms is capable of interacting with a base of another nucleic acid with or without the use of hydrogen bonds. In certain embodiments, an unnatural nucleobase is not derived from a natural nucleobase. It should be noted that unnatural nucleobases do not necessarily possess basic properties, however, they are referred to as nucleobases for simplicity. In some embodiments, when referring to a nucleobase, a “(d)” indicates that the nucleobase can be attached to a deoxyribose or a ribose, while “d” without parentheses indicates that the nucleobase is attached to deoxyribose.

As used herein, a “nucleoside” is a compound comprising a nucleobase moiety and a sugar moiety. Nucleosides include, but are not limited to, naturally occurring nucleosides (as found in DNA and RNA), abasic nucleosides, modified nucleosides, and nucleosides having mimetic bases and/or sugar groups. Nucleosides include nucleosides comprising any variety of substituents. A nucleoside can be a glycoside compound formed through glycosidic linking between a nucleic acid base and a reducing group of a sugar.

An “analog” of a chemical structure, as the term is used herein, refers to a chemical structure that preserves substantial similarity with the parent structure, although it may not be readily derived synthetically from the parent structure. In some embodiments, a nucleotide analog is an unnatural nucleotide. In some embodiments, a nucleoside analog is an unnatural nucleoside. A related chemical structure that is readily derived synthetically from a parent chemical structure is referred to as a “derivative.”

Although various features of the invention may be described in the context of a single embodiment, the features may also be provided separately or in any suitable combination. Conversely, although the invention may be described herein in the context of separate embodiments for clarity, the invention may also be implemented in a single embodiment.

### IL-2 Conjugates

Cytokines comprise a family of cell signaling proteins such as chemokines, interferons, interleukins, lymphokines, tumor necrosis factors, and other growth factors playing roles in innate and adaptive immune cell homeostasis. Cytokines are produced by immune cells such as macrophages, B lymphocytes, T lymphocytes and mast cells, endothelial cells, fibroblasts, and different stromal cells. In some instances, cytokines modulate the balance between humoral and cell-based immune responses.

Interleukins are signaling proteins which modulate the development and differentiation of T and B lymphocytes, cell of the monocytic lineage, neutrophils, basophils, eosinophils, megakaryocytes, and hematopoietic cells. Interleukins are produced by helper CD4 T and B lymphocytes, monocytes, macrophages, endothelial cells, and other tissue residents.

Interleukin 2 (IL-2) is a pleiotropic type-1 cytokine whose structure comprises a 15.5 kDa four α-helix bundle. The precursor form of IL-2 is 153 amino acid residues in length, with the first 20 amino acids forming a signal peptide and residues 21-153 forming the mature form. IL-2 is produced primarily by CD4+ T cells post antigen stimulation and to a lesser extent, by CD8+ cells, Natural Killer (NK) cells, and Natural killer T (NKT) cells, activated dendritic cells (DCs), and mast cells. IL-2 signaling occurs through interaction with specific combinations of IL-2 receptor (IL-2R) subunits, IL-2Rα (also known as CD25), IL-2Rβ (also known as CD122), and IL-2Ry (also known as CD132). Interaction of IL-2 with the IL-2Rα forms the “low-affinity” IL-2 receptor complex with a K_{d} of about 10⁻⁸ M. Interaction of IL-2 with IL-2Rβ and IL-2Rγ forms the “intermediate-affinity” IL-2 receptor complex with a K_{d} of about 10⁻⁹ M. Interaction of IL-2 with all three subunits, IL-2Rα, IL-2Rβ, and IL-2Rγ, forms the “high-affinity” IL-2 receptor complex with a K_{d} of about >10⁻¹¹ M.

In some instances, IL-2 signaling via the “high-affinity” IL-2Rαβγ complex modulates the activation and proliferation of regulatory T cells. Regulatory T cells, or CD4⁺CD25⁺Foxp3⁺ regulatory T (Treg) cells, mediate maintenance of immune homeostasis by suppression of effector cells such as CD4⁺ T cells, CD8⁺ T cells, B cells, NK cells, and NKT cells. In some instances, Treg cells are generated from the thymus (tTreg cells) or are induced from naïve T cells in the periphery (pTreg cells). In some cases, Treg cells are considered as the mediator of peripheral tolerance. Indeed, in one study, transfer of CD25-depleted peripheral CD4⁺ T cells produced a variety of autoimmune diseases in nude mice, whereas cotransfer of CD4⁺CD25⁺ T cells suppressed the development of autoimmunity (Sakaguchi, et al., “Immunologic self-tolerance maintained by activated T cells expressing IL-2 receptor alpha-chains (CD25),”J. Immunol. 155(3): 1151-1164 (1995), the disclosure of which is incorporated herein by reference). Augmentation of the Treg cell population down-regulates effector T cell proliferation and suppresses autoimmunity and T cell anti-tumor responses.

IL-2 signaling via the “intermediate-affinity” IL-2Rβγ complex modulates the activation and proliferation of CD8⁺ effector T (Teff) cells, NK cells, and NKT cells. CD8⁺ Teff cells (also known as cytotoxic T cells, Tc cells, cytotoxic T lymphocytes, CTLs, T-killer cells, cytolytic T cells, Tcon, or killer T cells) are T lymphocytes that recognize and kill damaged cells, cancerous cells, and pathogen-infected cells. NK and NKT cells are types of lymphocytes that, similar to CD8⁺ Teff cells, target cancerous cells and pathogen-infected cells.

In some instances, IL-2 signaling is utilized to modulate T cell responses and subsequently for treatment of a cancer. For example, IL-2 is administered in a high-dose form to induce expansion of Teff cell populations for treatment of a cancer. However, high-dose IL-2 further leads to concomitant stimulation of Treg cells that dampen anti-tumor immune responses. High-dose IL-2 also induces toxic adverse events mediated by the engagement of IL-2R alpha chain-expressing cells in the vasculature, including type 2 innate immune cells (ILC-2), eosinophils and endothelial cells. This leads to eosinophilia, capillary leak and vascular leak syndrome VLS).

Adoptive cell therapy enables physicians to effectively harness a patient's own immune cells to fight diseases such as proliferative disease (e.g., cancer) as well as infectious disease. Disclosed herein, in some embodiments, are methods of treating cancer in a subject, comprising administering to a subject in need thereof a therapeutically effective amount of (a) an IL-2 conjugate, and (b) one or more additional agents, wherein the one or more additional agents may comprise one or more immune checkpoint inhibitors.

In some embodiments, described herein are interleukin 2 (IL-2) conjugates. In some embodiments, described herein are the exemplary polypeptides shown in Table 1. In some embodiments, the IL-2 conjugates described herein are exemplified in Table 1.

TABLE 1

| | | SEQ |
|---|---|---|
| | | ID |
| Name | Sequence | NO: |
| IL-2 | APTSSSTKKTQLQLEHLLLDLQMILNGINNYKNPKLTRM | 1 |
| (homo sapiens) | LTFKFYMPKKATELKHLQCLEEELKPLEEVLNLAQSKN | |
| (mature form) | FHLRPRDLISNINVIVLELKGSETTFMCEYADETATIVEF | |
| | LNRWITFCQSIISTLT | |
| IL-2 | MYRMQLLSCIALSLALVTNSAPTSSSTKKTQLQLEHLLL | 2 |
| (homo sapiens) | DLQMILNGINNYKNPKLTRMLTFKFYVIPKKATELKHLQ | |
| (precursor) | CLEEELKPLEEVLNLAQSKNFHLRPRDLISNINVIVLELK | |
| NCBI Accession | GSETTFMCEYADETATIVEFLNRWITFCQSIISTLT | |
| No.: | | |
| AAB46883.1 | | |
| aldesleukin | PTSSSTKKTQLQLEHLLLDLQMILNGINNYKNPKLTRML | 3 |
| | TFKFYMPKKATELKHLQCLEEELKPLEEVLNLAQSKNF | |
| | HLRPRDLISNINVIVLELKGSETTFMCEYADETATIVEFL | |
| | NRWITFSQSIISTLT | |
| IL-2_C125S | APTSSSTKKTQLQLEHLLLDLQMILNGINNYKNPKLTRM | 4 |
| | LTFKFYMPKKATELKHLQCLEEELKPLEEVLNLAQSKN | |
| | FHLRPRDLISNINVIVLELKGSETTFMCEYADETATIVEF | |
| | LNRWITFSQSIISTLT | |
| IL-2_P65X | APTSSSTKKTQLQLEHLLLDLQMILNGINNYKNPKLTRM | 5 |
| | LTFKFYMPKKATELKHLQCLEEELKXLEEVLNLAQSKN | |
| | FHLRPRDLISNINVIVLELKGSETTFMCEYADETATIVEF | |
| | LNRWITFSQSIISTLT | |
| IL-2_E62X | APTSSSTKKTQLQLEHLLLDLQMILNGINNYKNPKLTRM | 6 |
| | LTFKFYMPKKATELKHLQCLEEXLKPLEEVLNLAQSKN | |
| | FHLRPRDLISNINVIVLELKGSETTFMCEYADETATIVEF | |
| | LNRWITFSQSIISTLT | |
| IL-2_F42X | APTSSSTKKTQLQLEHLLLDLQMILNGINNYKNPKLTRM | 7 |
| | LTXKFYMPKKATELKHLQCLEEELKPLEEVLNLAQSKN | |
| | FHLRPRDLISNINVIVLELKGSETTFMCEYADETATIVEF | |
| | LNRWITFSQSIISTLT | |
| IL-2_K43X | APTSSSTKKTQLQLEHLLLDLQMILNGINNYKNPKLTRM | 8 |
| | LTFXFYMPKKATELKHLQCLEEELKPLEEVLNLAQSKN | |
| | FHLRPRDLISNINVIVLELKGSETTFMCEYADETATIVEF | |
| | LNRWITFSQSIISTLT | |
| IL-2_K35X | APTSSSTKKTQLQLEHLLLDLQMILNGINNYKNPXLTRM | 9 |
| | LTFKFYMPKKATELKHLQCLEEELKPLEEVLNLAQSKN | |
| | FHLRPRDLISNINVIVLELKGSETTFMCEYADETATIVEF | |
| | LNRWITFSQSIISTLT | |
| IL-2_P65[AzK] | APTSSSTKKTQLQLEHLLLDLQMILNGINNYKNPKLTRM | 10 |
| | LTFKFYMPKKATELKHLQCLEEELK[AzK]LEEVLNLAQ | |
| | SKNFHLRPRDLISNINVIVLELKGSETTFMCEYADETATI | |
| | VEFLNRWITFSQSIISTLT | |
| IL-2_E62[AzK] | APTSSSTKKTQLQLEHLLLDLQMILNGINNYKNPKLTRM | 11 |
| | LTFKFYMPKKATELKHLQCLEE[AzK]LKPLEEVLNLAQ | |
| | SKNFHLRPRDLISNINVIVLELKGSETTFMCEYADETATI | |
| | VEFLNRWITFSQSIISTLT | |
| IL-2_F42[AzK] | APTSSSTKKTQLQLEHLLLDLQMILNGINNYKNPKLTRM | 12 |
| | LT[AzK]KFYMPKKATELKHLQCLEEELKPLEEVLNLAQ | |
| | SKNFHLRPRDLISNINVIVLELKGSETTFMCEYADETATI | |
| | VEFLNRWITFSQSIISTLT | |
| IL-2_K43[AzK] | APTSSSTKKTQLQLEHLLLDLQMILNGINNYKNPKLTRM | 13 |
| | LTF[AzK]FYMPKKATELKHLQCLEEELKPLEEVLNLAQS | |
| | KNFHLRPRDLISNINVIVLELKGSETTFMCEYADETATIV | |
| | EFLNRWITFSQSIISTLT | |
| IL-2_K35[AzK] | APTSSSTKKTQLQLEHLLLDLQMILNGINNYKNP[AzK]L | 14 |
| | TRMLTFKFYMPKKATELKHLQCLEEELKPLEEVLNLAQ | |
| | SKNFHLRPRDLISNINVIVLELKGSETTFMCEYADETATI | |
| | VEFLNRWITFSQSIISTLT | |
| IL-2P65[AzKPEG] | APTSSSTKKTQLQLEHLLLDLQMILNGINNYKNPKLTRM | 15 |
| | LTFKFYMPKKATELKHLQCLEEELK[AzK_PEG]LEEVL | |
| | NLAQSKNFHLRPRDLISNINVIVLELKGSETTFMCEYAD | |
| | ETATIVEFLNRWITFSQSIISTLT | |
| 1L-2_E62[AzK_PEG] | APTSSSTKKTQLQLEHLLLDLQMILNGINNYKNPKLTRM | 16 |
| | LTFKFYMPKKATELKHLQCLEE[AzK_PEG]LKPLEEVL | |
| | NLAQSKNFHLRPRDLISNINVIVLELKGSETTFMCEYAD | |
| | ETATlVEFLNRWITFSQSIISTLT | |
| IL-2_F42[AzK_PEG] | APTSSSTKKTQLQLEHLLLDLQMILNGINNYKNPKLTRM | 17 |
| | LT[AzK_PEG]KFYMPKKATELKHLQCLEEELKPLEEVL | |
| | NLAQSKNFHLRPRDLISNINVIVLELKGSETTFMCEYAD | |
| | ETAliVEFLNRWITFSQSIISTLT | |
| IL-2_K43[AzK_PEG] | APTSSSTKKTQLQLEHLLLDLQMILNGINNYKNPKLTRM | 18 |
| | LTF[AzK_PEG]FYMPKKATELKHLQCLEEELKPLEEVL | |
| | NLAQSKNFHLRPRDLISNINVIVLELKGSETTFMCEYAD | |
| | ATIVEFLNRWITFSQSIISTLT | |
| IL-2_K35[AzK_PEG] | APTSSSTKKTQLQLEHLLLDLQMILNGINNYKNP[AzK_P | 19 |
| | EG]LTRMLTFKFYMPKKATELKHLQCLEEELKPLEEVL | |
| | NLAQSKNFHLRPRDLISNINVIVLELKGSETTFMCEYAD | |
| | ETATIVEFLNRWITFSQSIISTLT | |
| IL- | APTSSSTKKTQLQLEHLLLDLQMILNGINNYKNPKLTRM | 20 |
| 2_P65[AzK_PEG5kD] | LTFKFYMPKKATELKHLQCLEEELK[AzK_PEG5kD]LEE | |
| | VLNLAQSKNFHLRPRDLISNINVIVLELKGSETTFMCEY | |
| | ADETATIVEFLNRWITFSQSIISTLT | |
| IL- | APTSSSTKKTQLQLEHLLLDLQMILNGINNYKNPKLTRM | 21 |
| 2_E62[AzK_PEG5kD] | LTFKFYMPKKATELKHLQCLEE[AzK_PEG5kD]LKPLEE | |
| | VLNLAQSKNFHLRPRDLISNINVIVLELKGSETTFMCEY | |
| | ADETATIVEFLNRWITFSQSIISTLT | |
| IL-2_F42 | APTSSSTKKTQLQLEHLLLDLQMILNGINNYKNPKLTRM | 22 |
| [AzK_PEG5kD] | LT[AzK_PEG5kD]KFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K43 | APTSSSTKKTQLQLEHLLLDLQMILNGINNYKNPKLTRM | 23 |
| [AzK_PEG5kD] | LTF[AzK_PEG5kD]FYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K35 | APTSSSTKKTQLQLEHLLLDLQMILNGINNYKNP[AzK_P | 24 |
| [AzK_PEG5kD] | EG5kD]LTRMLTFKFYMPKKATELKULQCLEEELKPLEE | |
| | NLAQSKNFHLRPRDLISNINVIVLELKGSETTFMCEYAD | |
| | ADETATIVEFLNRWITFSQSIISTLT | |
| IL-2_P65 | APTSSSTKKTQLQLEHLLLDLQMILNGINNYKNPKLTRM | 25 |
| [AzK_PEG30kD] | LTFKFYMPKKATELKHLQCLEEELK[AzK_PEG30kD]LE | |
| | EVLNLAQSKNFHLRPRDLISNINVIVLELKGSETTFMCEY | |
| | ADETATIVEFLNRWITFSQSIISTLT | |
| IL-2_E62 | APTSSSTKKTQLQLEHLLLDLQMILNGINNYKNPKLTRM | 26 |
| [AzK_PEG30kD] | LTFKFYMPKKATELKHLQCLEE[AzK_PEG30kD]LKPLE | |
| | EVLNLAQSKNFHLRPRDLISNINVIVLELKGSETTFMCEY | |
| | ADETATIVEFLNRWITFSQSIISTLT | |
| IL-2_F42 | APTSSSTKKTQLQLEHLLLDLQMILNGINNYKNPKLTRM | 27 |
| [AzK_PEG30kD] | LT[AzK_PEG30kD]KFYMPKKATELKHLQCLEEELKPLE | |
| | EVLNLAQSKNFHLRPRDLISNINVIVLELKGSETTFMCEY | |
| | ADETATIVEFLNRWITFSQSIISTLT | |
| IL-2_K43 | APTSSSTKKTQLQLEHLLLDLQMILNGINNYKNPKLTRM | 28 |
| [AzK_PEG30kD] | LTF[AzK_PEG30kD]FYMPKKATELKHLQCLEEELKPLE | |
| | EVLNLAQSKNFHLRPRDLISNINVIVLELKGSETTFMCEY | |
| | ADETATIVEFLNRWITFSQSIISTLT | |
| IL-2_K35 | APTSSSTKKTQLQLEHLLLDLQMILNGINNYKNP[AzK_P | 29 |
| [AzK_PEG30kD] | EG30kD]LTRMLTFKFYMPKKATELKHLQCLEEELKPLE | |
| | EVLNLAQSKNFHLRPRDLISNINVIVLELKGSETTFMCEY | |
| | ADETATIVEFLNRWITFSQSIISTLT | |
| IL-2_P65X-1 | PTSSSTKKTQLQLEHLLLDLQMILNGINNYKNPKLTRML | 30 |
| | TFKFYMPKKATELKHLQCLEEELKXLEEVLNLAQSKNF | |
| | HLRPRDLISNINVIVLELKGSETTFMCEYADETATIVEFL | |
| | NRWITFSQSIISTLT | |
| IL-2_E62X-1 | PTSSSTKKTQLQLEHLLLDLQMILNGINNYKNPKLTRML | 31 |
| | TFKFYMPKKATELKHLQCLEEXLKPLEEVLNLAQSKNF | |
| | HLRPRDLISNINVIVLELKGSETTFMCEYADETATIVEFL | |
| | NRWITFSQSIISTLT | |
| IL-2_F42X-1 | PTSSSTKKTQLQLEHLLLDLQMILNGINNYKNPKLTRML | 32 |
| | TXKFYMPKKATELKHLQCLEEELKPLEEVLNLAQSKNF | |
| | HLRPRDLISNINVIVLELKGSETTFMCEYADETATIVEFL | |
| | NRWITFSQSIISTLT | |
| IL-2_K43X-1 | PTSSSIKKTQLQLEHLLLDLQMILNGINNYKNPKLTRML | 33 |
| | TFXFYMPKKATELKHLQCLEEELKPLEEVLNLAQSKNF | |
| | HLRPRDLISNINVIVLELKGSETTFMCEYADETATIVEFL | |
| | NRWITFSQSIISTLT | |
| IL-2_K35X-1 | PTSSSTKKTQLQLEHLLLDLQMILNGINNYKNPXLTRML | 34 |
| | TFKFYMPKKATELKHLQCLEEELKPLEEVLNLAQSKNF | |
| | HLRPRDLISNINVIVLELKGSETTFMCEYADETATIVEFL | |
| | NRWITFSQSIISTLT | |
| IL-2_P65[AzK]-1 | PTSSSTKKTQLQLEHLLLDLQMILNGINNYKNPKLTRML | 35 |
| | TFKFYMPKKATELKHLQCLEEELK[AzK]LEEVLNLAQS | |
| | KNFHLRPRDLISNINVIVLELKGSETTFMCEYADETATIV | |
| | EFLNRWITFSQSIISTLT | |
| IL-2_E62[AzK]-1 | PTSSSTKKTQLQLEHLLLDLQMILNGINNYKNPKLTRML | 36 |
| | TFKFYMPKKATELKHLQCLEE[AzK]LKPLEEVLNLAQS | |
| | KNFHLRPRDLISNINVIVLELKGSETTFMCEYADETATIV | |
| | EFLNRWITFSQSIISTLT | |
| IL-2_F42[AzK]-1 | PTSSSTKKTQLQLEHLLLDLQMILNGINNYKNPKLTRML | 37 |
| | T[AzK]KFYMPKKATELKHLQCLEEELKPLEEVLNLAQS | |
| | KNFHLRPRDLISNINVIVLELKGSETTFMCEYADETATIV | |
| | EFLNRWITFSQSIISTLT | |
| IL-2_K43[AzK]-1 | PTSSSTKKTQLQLEHLLLDLQMILNGINNYKNPKLTRML | 38 |
| | TF[AzK]FYMPKKATELKHLQCLEEELKPLEEVLNLAQS | |
| | KNFHLRPRDLISNINVIVLELKGSETTFMCEYADETATIV | |
| | EFLNRWITFSQSIISTLT | |
| IL-2_K35[AzK]-1 | PTSSSTKKTQLQLEHLLLDLQMILNGINNYKNP[AzK]LT | 39 |
| | RMLTFKFYMPKKATELKHLQCLEEELKPLEEVLNLAQS | |
| | KNFHLRPRDLISNINVIVLELKGSETTFMCEYADETATIV | |
| | EFLNRWITFSQSIISTLT | |
| IL-2_P65 | PTSSSTKKTQLQLEHLLLDLQMILNGINNYKNPKLTRML | 40 |
| [AzK_L1_PEG]-1 | TFKFYMPKKATELKHLQCLEEELK[AzK_L1_PEG]LEEV | |
| | LNLAQSKNFHLRPRDLISNINVIVLELKGSETTFMCEYA | |
| | DETATIVEFLNRWITFSQSIISTLT | |
| IL-2_E62[AzK_ | PTSSSTKKTQLQLEHLLLDLQMILNGINNYKNPKLTRML | 41 |
| L1_PEG]-1 | TFKFYMPKKATELKHLQCLEE[AzK_L1_PEG]LKPLEEV | |
| | LNLAQSKNFHLRPRDLISNINVIVLELKGSETTFMCEYA | |
| | DETATIVEFLNRWITFSQSIISTLT | |
| IL-2_F42[AzK_ | PTSSSTKKTQLQLEHLLLDLQMILNGINNYKNPKLTRML | 42 |
| L1_PEG]-1 | T[AzK_L1_PEG]KFYMPKKATELKHLQCLEEELKPLEEV | |
| | LNLAQSKNFHLRPRDLISNINVIVLELKGSETTFMCEYA | |
| | DETATIVEFLNRWITFSQSIISTLT | |
| IL-2_K43[AzK_ | PTSSSTKKTQLQLEHLLLDLQMILNGINNYKNPKLTRML | 43 |
| L1_PEG]-1 | TF[AzK_L1_PEG]FYMPKKATELKMLQCLEEELKPLEEV | |
| | LNLAQSKNFHLRPRDLISNINVIVLELKGSETTFMCEYA | |
| | DETATIVEFLNRWITFSQSIISTLT | |
| IL-2_K35[AzK_ | PTSSSTKKTQLQLEHLLLDLQMILNGINNYKNP[AzK_L1_ | 44 |
| L1_PEG]-1 | PEG]LTRMLTFKFYMPKKATELKHLQCLEEELKPLEEV | |
| | LNLAQSKNFHLRPRDLISNINVIVLELKGSETTFMCEYA | |
| | DETATIVEFLNRWITFSQSIISTLT | |
| IL-2_P65[AzK_L1_ | PTSSSTKKTQLQLEHLLLDLQMILNGINNYKNPKLTRML | 45 |
| PEG5kD]-l | TFKFYMPKKATELKHLQCLEEELK[AzK_L1_PEG5kD]L | |
| | EEVLNLAQSKNFHLRPRDLISNINVIVLELKGSETTFMCE | |
| | YADETATIVEFLNRWITFSQSIISTLT | |
| IL-2_E62[AzK_L1_ | PTSSSTKKTQLQLEHLLLDLQMILNGINNYKNPKLTRML | 46 |
| PEG5kD]-l | TFKFYMPKKATELKHLQCLEE[AzK_L1_PEG5kD]LKPL | |
| | EEVLNLAQSKNFHLRPRDLISNINVIVLELKGSETTFMCE | |
| | YADETATIVEFLNRWITFSQSIISTLT | |
| IL-2_F42[AzK_L1_ | PTSSSTKKTQLQLEHLLLDLQMILNGINNYKNPKLTRML | 47 |
| PEG5kD]-l | T[AzK_L1_PEG5kD]KFYMPKKATELKHLQCLEEELKPL | |
| | EEVLNLAQSKNFHLRPRDLISNINVIVLELKGSETTFMCE | |
| | YADETATIVEFLNRWITFSQSIISTLT | |
| IL-2_K43[AzK_L1_ | PTSSSTKKTQLQLEHLLLDLQMILNGINNYKNPKLTRML | 48 |
| PEG5kD]-l | TF[AzK_L1_PEG5kD]FYMPKKATELKHLQCLEEELKPL | |
| | EEVLNLAQSKNFHLRPRDLISNINVIVLELKGSETTFMCE | |
| | YADETATIVEFLNRWITFSQSIISTLT | |
| IL-2_K35[AzK_L1_ | PTSSSTKKTQLQLEHLLLDLQMILNGINNYKNP[AzK_L1_ | 49 |
| PEG5kD]-l | PEG5kD]LTRMLTFKFYMPKKATELKHLQCLEEELKPL | |
| | EEVLNLAQSKNFHLRPRDLISNINVIVLELKGSETTFMCE | |
| | YADETATIVEFLNRWITFSQSIISTLT | |
| IL-2_P65[AzK | PTSSSTKKTQLQLEHLLLDLQMILNGINNYKNPKLTRML | 50 |
| L1_PEG30kD]-l | TFKFYMPKKATELKHLQCLEEELK[AzK_L1_PEG30kD] | |
| | LEEVLNLAQSKNFHLRPRDLISNINVIVLELKGSETTFMC | |
| | EYADETATIVEFLNRWITFSQSIISTLT | |
| IL-2_E62[AzK_L1_ | PTSSSTKKTQLQLEHLLLDLQMILNGINNYKNPKLTRML | 51 |
| PEG30kD]-l | TFKFYMPKKATELKHLQCLEE[AzK_L1_PEG30kD]LKP | |
| | LEEVLNLAQSKNFHLRPRDLISNINVIVLELKGSETTFMC | |
| | EYADETATIVEFLNRWITFSQSIISTLT | |
| IL-2_F42[AzK_L1_ | PTSSSTKKTQLQLEHLLLDLQMILNGINNYKNPKLTRML | 52 |
| PEG30kD]-l | T[AzK_L1_PEG30kD]KFYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL-2_K43[AzK_L1_ | PTSSSTKKTQLQLEHLLLDLQMILNGINNYKNPKLTRML | 53 |
| PEG30kD]-l | TF[AzK_L1_PEG30kD]FYMPKKATELKHLQCLEEELKPL | |
| | EEVLNLAQSKNFHLRPRDLISNINVIVLELKGSETTFMCE | |
| | YADETATIVEFLNRWITFSQSIISTLT | |
| IL-2_K35[AzK_L1_ | PTSSSTKKTQLQLEHLLLDLQMILNGINNYKNP[AzK_L1_ | 54 |
| PEG30kD]-l | PEG30kD]LTRMLTFKFYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL- | APTSSSTKKTQLQLEHLLLDLQMILNGINNYKNPKLTRM | 55 |
| 2_P65[AzK_ | LTFKFYMPKKATELKHLQCLEEELK[AzK_L1_PEG]LEE | |
| L1_PEG]-2 | VLNLAQSKNFHLRPRDLISNINVIVLELKGSETTFMCEY | |
| | ADETATIVEFLNRWITFSQSIISTLT | |
| IL-2_E62[AzK_ | APTSSSTKKTQLQLEHLLLDLQMILNGINNYKNPKLTRM | 56 |
| L1_PEG]-2 | LTFKFYMPKKATELKHLQCLEE[AzK_L1_PEG]LKPLEE | |
| | VLNLAQSKNFHLRPRDLISNINVIVLELKGSETTFMCEY | |
| | ADETATIVEFLNRWITFSQSIISTLT | |
| IL-2_F42[AzK_ | APTSSSTKKTQLQLEHLLLDLQMILNGINNYKNPKLTRM | 57 |
| L1_PEG]-2 | LT[AzK_L1_PEG]KFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K43[AzK_ | APTSSSTKKTQLQLEHLLLDLQMILNGINNYKNPKLTRM | 58 |
| L1_PEG]-2 | LTF[AzK_L1_PEG]FYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K35[AzK_ | APTSSSTKKTQLQLEHLLLDLQMILNGINNYKNP[AzK_ | 59 |
| L1_PEG]-2 | L1_PEG]LTRMLTFKFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_P65[AzK_L1_ | APTSSSTKKTQLQLEHLLLDLQMILNGINNYKNPKLTRM | 60 |
| PEG5kD]-2 | LTFKFYMPKKATELKHLQCLEEELK[AzK_L1_PEG5kD] | |
| | LEEVLNLAQSKNFHLRPRDLISNINVIVLELKGSETTFMC | |
| | EYADETATIVEFLNRWITFSQSIISTLT | |
| IL-2_E62[AzK_L1_ | APTSSSTKKTQLQLEHLLLDLQMILNGINNYKNPKLTRM | 61 |
| PEG5kD]-2 | LTFKFYMPKKATELKHLQCLEE[AzK_L1_PEG5kD]LKP | |
| | LEEVLNLAQSKNFHLRPRDLISNINVIVLELKGSETTFMC | |
| | EYADETATIVEFLNRWITFSQSIISTLT | |
| IL-2_F42[AzK_L1_ | APTSSSTKKTQLQLEHLLLDLQMILNGINNYKNPKLTRM | 62 |
| PEG5kD]-2 | LT[AzK_L1_PEG5kD]KFYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL-2_K43[AzK_ | APTSSSTKKTQLQLEHLLLDLQMILNGINNYKNPKLTRM | 63 |
| L1_PEG5kD]-2 | LTF[AzK_L1_PEG5kD]FYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL-2_K35[AzK_ | APTSSSTKKTQLQLEHLLLDLQMILNGINNYKNP[AzK_ | 64 |
| L1_PEG5kD]-2 | L1_PEG5kD]LTRMLTFKFYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL-2_P65[AzK_ | APTSSSTKKTQLQLEHLLLDLQMILNGINNYKNPKLTRM | 65 |
| L1_PEG30kD]-2 | LTFKFYMPKKATELKHLQCLEEELK[AzK_L1_PEG30kD] | |
| | LEEVLNLAQSKNFHLRPRDLISNINVIVLELKGSETTFM | |
| | CEYADETATIVEFLNRWITFSQSIISTLT | |
| IL-2_E62[AzK | APTSSSTKKTQLQLEHLLLDLQMILNGINNYKNPKLTRM | 66 |
| L1_PEG30kD]-2 | LTFKFYMPKKATELKHLQCLEE[AzK_L1_PEG30kD]LK | |
| | PLEEVLNLAQSKNFHLRPRDLISNINVIVLELKGSETTFM | |
| | CEYADETATIVEFLNRWITFSQSIISTLT | |
| IL-2_F42[AzK_L1_ | APTSSSTKKTQLQLEHLLLDLQMILNGINNYKNPKLTRM | 67 |
| PEG30kD]-2 | LT[AzK_L1_PEG30kD]KFYMPKKATELKHLQCLEEELK | |
| | PLEEVLNLAQSKNFHLRPRDLISNINVIVLELKGSETTFM | |
| | CEYADETATIVEFLNRWITFSQSIISTLT | |
| IL-2_K43[AzK_L1_ | APTSSSTKKTQLQLEHLLLDLQMILNGINNYKNPKLTRM | 68 |
| PEG30kD]-2 | LTF[AzK_L1_PEG30kD]FYMPKKATELKHLQCLEEELKP | |
| | LEEVLNLAQSKNFHLRPRDLISNINVIVLELKGSETTFMC | |
| | EYADETATIVEFLNRWITFSQSIISTLT | |
| IL-2_K35[AzK_L1_ | APTSSSTKKTQLQLEHLLLDLQMILNGINNYKNP[AzK_L1_ | 69 |
| PEG30kD]-2 | PEG30kD]LTRMLTFKFYMPKKATELKHLQCLEEELK | |
| | PLEEVLNLAQSKNFHLRPRDLISNINVIVLELKGSETTFM | |
| | CEYADETATIVEFLNRWITFSQSIISTLT | |
| IL-2_P65 | PTSSSTKKTQLQLEHLLLDLQMILNGINNYKNPKLTRML | 70 |
| [AzK_PEG]-1 | TFKFYMPKKATELKHLQCLEEELK[AzK_PEG]LEEVLN | |
| | LAQSKNFHLRPRDLISNINVIVLELKGSETTFMCEYADET | |
| | ATIVEFLNRWITFSQSIISTLT | |
| IL-2_E62 | PTSSSTKKTQLQLEHLLLDLQMILNGINNYKNPKLTRML | 71 |
| [AzK_PEG]-1 | TFKFYMPKKATELKHLQCLEE[AzK_PEG]LKPLEEVLN | |
| | LAQSKNFHLRPRDLISNINVIVLELKGSETTFMCEYADET | |
| | ATIVEFLNRWITFSQSIISTLT | |
| IL-2_F42 | PTSSSTKKTQLQLEHLLLDLQMILNGINNYKNPKLTRML | 72 |
| [AzK_PEG]-1 | T[AzK_PEG]KFYMPKKATELKHLQCLEEELKPLEEVLN | |
| | LAQSKNFHLRPRDLISNINVIVLELKGSETTFMCEYADET | |
| | ATIVEFLNRWITFSQSIISTLT | |
| IL-2_K43 | PTSSSTKKTQLQLEHLLLDLQMILNGINNYKNPKLTRML | 73 |
| [AzK_PEG]-1 | TF[AzK_PEG]FYMPKKATELKHLQCLEEELKPLEEVLNL | |
| | AQSKNFHLRPRDLISNINVIVLELKGSETTFMCEYADET | |
| | ATIVEFLNRWITFSQSIISTLT | |
| IL-2_K35 | PTSSSTKKTQLQLEHLLLDLQMILNGINNYKNP[AzK_PE | 74 |
| [AzK_PEG]-1 | G]LTRMLTFKFYMPKKATELKHLQCLEEELKPLEEVLNL | |
| | AQSKNFHLRPRDLISNINVIVLELKGSETTFMCEYADET | |
| | ATIVEFLNRWITFSQSIISTLT | |
| IL-2_P65[AzK_ | PTSSSTKKTQLQLEHLLLDLQMILNGINNYKNPKLTRML | 75 |
| PEG5kD]-l | TFKFYMPKKATELKHLQCLEEELK[AzK_PEG5kD]LEEV | |
| | LNLAQSKNFHLRPRDLISNINVIVLELKGSETTFMCEYA | |
| | DETATIVEFLNRWITFSQSIISTLT | |
| IL-2_E62 | PTSSSTKKTQLQLEHLLLDLQMILNGINNYKNPKLTRML | 76 |
| [AzK_PEG5kD]-1 | TFKFYMPKKATELKHLQCLEE[AzK_PEG5kD]LKPLEEV | |
| | LNLAQSKNFHLRPRDLISNINVIVLELKGSETTFMCEYA | |
| | DETATIVEFLNRWITFSQSIISTLT | |
| IL-2_F42 | PTSSSTKKTQLQLEHLLLDLQMILNGINNYKNPKLTRML | 77 |
| [AzK PEG5kD]-l | T[AzK_PEG5kD]KFYMPKKATELKHLQCLEEELKPLEEV | |
| | LNLAQSKNFHLRPRDLISNINVIVLELKGSETTFMCEYA | |
| | DETATIVEFLNRWITFSQSIISTLT | |
| IL-2_K43 | PTSSSTKKTQLQLEHLLLDLQMILNGINNYKNPKLTRML | 78 |
| [AzK_PEG5kD]-1 | T[AzK_PEG5kD]FYMPKKATELKHLQCLEEELKPLEEV | |
| | LNLAQSKNFHLRPRDLISNINVIVLELKGSETTFMCEYA | |
| | DETATIVEFLNRWITFSQSIISTLT | |
| IL-2_K35 | PTSSSTKKTQLQLEHLLLDLQMILNGINNYKNP[AzK_PE | 79 |
| [AzK_PEG5kD]-1 | G5kD]LTRMLTFKFYMPKKATELKHLQCLEEELKPLEEV | |
| | LNLAQSKNFHLRPRDLISNINVIVLELKGSETTFMCEYA | |
| | DETATIVEFLNRWITFSQSIISTLT | |
| IL-2_P65 | PTSSSTKKTQLQLEHLLLDLQMILNGINNYKNPKLTRML | 80 |
| [AzK_PEG30kD]-1 | TFKFYMPKKATELKHLQCLEEELK[AzK_PEG30kD]LEE | |
| | VLNLAQSKNFHLRPRDLISNINVIVLELKGSETTFMCEY | |
| | ADETATIVEFLNRWITFSQSIISTLT | |
| IL-2_E62 | PTSSSTKKTQLQLEHLLLDLQMILNGINNYKNPKLTRML | 81 |
| [AzK_PEG30kD]-1 | TFKFYMPKKATELKHLQCLEE[AzK_PEG30kD]LKPLEE | |
| | VLNLAQSKNFHLRPRDLISNINVIVLELKGSETTFMCEY | |
| | ADETATIVEFLNRWQITFSQSIISTLT | |
| IL-2_F42 | PTSSSTKKTQLQLEHLLLDLQMILNGINNYKNPKLTRML | 82 |
| [AzK_PEG30kD]-1 | T[AzK_PEG30kD]KFYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K43 | PTSSSTKKTQLQLEHLLLDLQMILNGINNYKNPKLTRML | 83 |
| [AzK_PEG30kD]-1 | TF[AzK_PEG30kD]FYMPKKATELKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_K35 | PTSSSTKKTQLQLEHLLLDLQMILNGINNYKNP[AzK_PE | 84 |
| [AzK_PEG30kD]-1 | G30kD]LTRMLTFKFYMPKKAT£LKHLQCLEEELKPLEE | |
| | VLNLAQSKNFHLRPRDLISNINVIVLELKGSETTFMCEY | |
| | ADETATIVEFLNRWITFSQSIISTLT | |
| IL-2_F44X | APTSSSTKKTQLQLEHLLLDLQMILNGINNYKNPKLTRM | 85 |
| | LTFKXYMPKKATELKHLQCLEEELKPLEEVLNLAQSKNF | |
| | HLRPRDLISNINVIVLELKGSETTFMCEYADDETATIVEF | |
| | LNRWITFSQSIISTLT | |
| IL-2_F44X-1 | PTSSSTKKTQLQLEHLLLDLQMILNGINNYKNPKLTRML | 86 |
| | TFKXYMPKKATELKHLQCLEEELKPLEEVLNLAQSKNF | |
| | HLRPRDLISNINVIVLELKGSETTFMCEYADETATIVEFL | |
| | NRWITFSQSIISTLT | |
| IL-2_R38X | APTSSSTKKTQLQLEHLLLDLQMILNGINNYKNPKLTXM | 87 |
| | LTFKFYMPKKATELKHLQCLEEELKPLEEVLNLAQSKN | |
| | FHLRPRDLISNINVIVLELKGSETTFMCEYADETATIVEF | |
| | LNRWITFCQSIISTLT | |
| IL-2_R38X-1 | PTSSSTKKTQLQLEHLLLDLQMILNGINNYKNPKLTXML | 88 |
| | TFKFYMPKKATELKHLQCLEEELKPLEEVLNLAQSKNF | |
| | HLRPRDLISNINVIVLELKGSETTFMCEYADETATIVEFL | |
| | NRWITFCQSIISTLT | |
| IL-2_T41X | APTSSSTKKTQLQLEHLLLDLQMILNGINNYKNPKLTRM | 89 |
| | LXFKFYMPKKATELKHLQCLEEELKPLEEVLNLAQSKN | |
| | FHLRPRDLISNINVIVLELKGSETTFMCEYADETATIVEF | |
| | LNRWITFCQSIISTLT | |
| IL-2_T41X-1 | PTSSSTKKTQLQLEHLLLDLQMILNGINNYKNPKLTRML | 90 |
| | XFKFYMPKKATELKHLQCLEEELKPLEEVLNLAQSKNF | |
| | HLRPRDLISNINVIVLELKGSETTFMCEYADETATIVEFL | |
| | NRWITFCQSIISTLT | |
| IL-2_E68X | APTSSSTKKTQLQLEHLLLDLQMILNGINNYKNPKLTRM | 91 |
| | LTFKFYMPKKATELKHLQCLEEELKPLEXVLNLAQSKN | |
| | FHLRPRDLISNINVIVLELKGSETTFMCEYADETATIVEF | |
| | LNRWITFCQSIISTLT | |
| IL-2_E68X-1 | PTSSSTKKTQLQLEHLLLDLQMILNGINNYKNPKLTRML | 92 |
| | TFKFYMPKKATELKHLQCLEEELKPLEXVLNLAQSKNF | |
| | HLRPRDLISNINVIVLELKGSETTFMCEYADETATIVEFL | |
| | NRWITFCQSIISTLT | |
| IL-2_Y45X | APTSSSTKKTQLQLEHLLLDLQMILNGINNYKNPKLTRM | 93 |
| | LTFKFXMPKKATELKHLQCLEEELKPLEEVLNLAQSKN | |
| | FHLRPRDLISNINVIVLELKGSETTFMCEYADETATIVEF | |
| | LNRWITFCQSIISTLT | |
| IL-2_Y45X-1 | PTSSSTKKTQLQLEHLLLDLQMILNGINNYKNPKLTRML | 94 |
| | TFKFXMPKKATELKHLQCLEEELKPLEEVLNLAQSKNF | |
| | HLRPRDLISNINVIVLELKGSETTFMCEYADETATIVEFL | |
| | NRWITFCQSIISTLT | |
| IL-2_V69X | APTSSSTKKTQLQLEHLLLDLQMILNGINNYKNPKLTRM | 95 |
| | LTFKFYMPKKATELKHLQCLEEELKPLEEXLNLAQSKN | |
| | FHLRPRDLISNINVIVLELKGSETTFMCEYADETATIVEF | |
| | LNRWITFCQSIISTLT | |
| IL-2_V69X-1 | PTSSSTKKTQLQLEHLLLDLQMILNGINNYKNPKLTRML | 96 |
| | TFKFYMPKKATELKHLQCLEEELKPLEEXLNLAQSKNF | |
| | HLRPRDLISNINVIVLELKGSETTFMCEYADETATIVEFL | |
| | NRWITFCQSIISTLT | |
| IL-2_L72X | APTSSSTKKTQLQLEHLLLDLQMILNGINNYKNPKLTRM | 97 |
| | LTFKFYMPKKATELKHLQCLEEELKPLEEVLNXAQSKN | |
| | FHLRPRDLISNINVIVLELKGSETTFMCEYADETATIVEF | |
| | LNRWITFCQSIISTLT | |
| IL-2_L72X-1 | PTSSSTKKTQLQLEHLLLDLQMILNGINNYKNPKLTRML | 98 |
| | TFKFYMPKKATELKHLQCLEEELKPLEEVLNXAQSKNF | |
| | HLRPRDLISNINVIVLELKGSETTFMCEYADETATIVEFL | |
| | NRWITFCQSIISTLT | |

X = site comprising an unnatural amino acid.
[AzK] = N6-((2-azidoethoxy)-carbonyl)-L-lysine, having Chemical Abstracts Registry No. 1167421-25-1.
[AzK_PEG] = N6-((2-azidoethoxy)-carbonyl)-L-lysine stably-conjugated to PEG via DBCO-mediated click chemistry, to form a compound comprising a structure of Formula (II) or Formula (III). For example, if specified, PEG5kD indicates a linear polyethylene glycol chain with an average molecular weight of 5 kiloDaltons, capped with a methoxy group The ratio of regioisomers generated from the click reaction is about 1:1 or greater than 1:1. The term “DBCO” means a chemical moiety comprising a dibenzocyclooctyne group, such as comprising the mPEG-DBCO compound illustrated in Scheme 1 of Example 2. An exemplary structure of a methoxy PEG group is illustrated in the mPEG-DBCO structure in Scheme 1 of Example 2.
[AzK_L1_PEG] = N6-((2-azidoethoxy)-carbonyl)-L-lysine stably-conjugated to PEG via DBCO-mediated click chemistry to form a compound comprising a structure of Formula (IV) or Formula (V). For example, if specified, PEG5kD indicates a linear polyethylene glycol chain with an average molecular weight of 5 kiloDaltons, capped with a methoxy group. The ratio of regioisomers generated from the click reaction is about 1:1 or greater than 1:1. The term “DBCO” means a chemical moiety comprising a dibenzocyclooctyne group, such as comprising the mPEG-DBCO compound illustrated in Scheme 1 of Example 2.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (I):

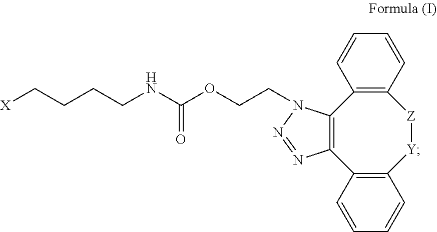

wherein:

### Z is CH₂ and Y is

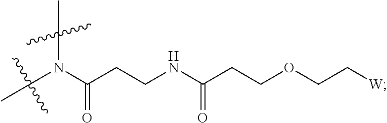

### Y is CH₂ and Z

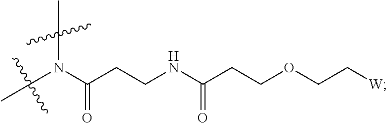

### Z is CH₂ and Y is

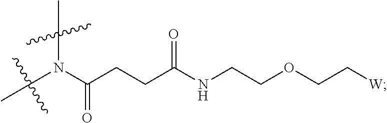

or

### Y is CH₂ and Z is

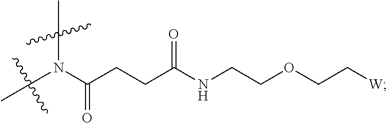

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

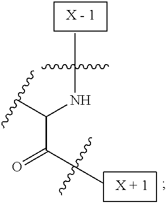 [SMILES: *NC(*)C(*)=O]

X−1 indicates the point of attachment to the preceding amino acid residue; and
X+1 indicates the point of attachment to the following amino acid residue.

Here and throughout, the term “IL-2 conjugate” encompasses pharmaceutically acceptable salts, solvates, and hydrates of the indicated structure.

Here and throughout, the structure of Formula (I) encompasses pharmaceutically acceptable salts, solvates, or hydrates thereof. In some embodiments, the structure of Formula (I), or any embodiment or variation thereof, is provided as a pharmaceutically acceptable salt thereof. In some embodiments, the structure of Formula (I), or any embodiment or variation thereof, is provided as a solvate thereof. In some embodiments, the structure of Formula (I), or any embodiment or variation thereof, is provided as a hydrate thereof. In some embodiments, the structure of Formula (I), or any embodiment or variation thereof, is provided as the free base.

In some embodiments of a method described herein, in the IL-2 conjugate Z is CH₂ and Y

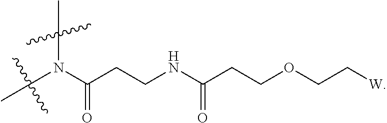

In some embodiments of a method described herein, in the IL-2 conjugate Y is CH₂ and Z is

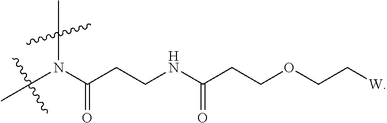

In some embodiments of a method described herein, Z is CH₂ and Y is

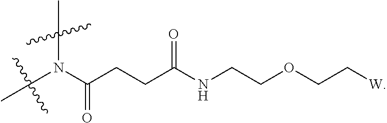

In some embodiments of a method described herein, in the IL-2 conjugate Z is CH₂ and Y is

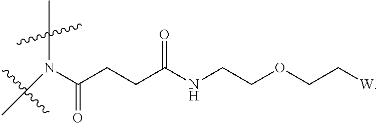

In some embodiments of a method described herein, in the IL-2 conjugate Y is CH₂ and Z is

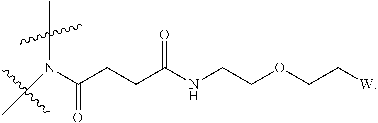

Here and throughout, embodiments of Z and Y also encompass a pharmaceutically acceptable salt, solvate, or hydrate thereof.

In some embodiments of a method described herein, in the IL-2 conjugate the PEG group has an average molecular weight selected from 5 kDa, 10 kDa, 20 kDa and 30 kDa. In some embodiments of a method described herein, in the IL-2 conjugate the PEG group has an average molecular weight of 5 kDa. In some embodiments of a method described herein, in the IL-2 conjugate the PEG group has an average molecular weight of 10 kDa. In some embodiments of a method described herein, in the IL-2 conjugate the PEG group has an average molecular weight of 15 kDa. In some embodiments, the methods use an IL-2 conjugate in which in the IL-2 conjugate the PEG group has an average molecular weight of 20 kDa. In some embodiments of a method described herein, in the IL-2 conjugate the PEG group has an average molecular weight of 25 kDa. In some embodiments of a method described herein, in the IL-2 conjugate the PEG group has an average molecular weight of 30 kDa. In some embodiments of a method described herein, in the IL-2 conjugate the PEG group has an average molecular weight of 35 kDa. In some embodiments of a method described herein, in the IL-2 conjugate the PEG group has an average molecular weight of 40 kDa. In some embodiments of a method described herein, in the IL-2 conjugate the PEG group has an average molecular weight of 45 kDa. In some embodiments of a method described herein, in the IL-2 conjugate the PEG group has an average molecular weight of 50 kDa. In some embodiments, the methods use an IL-2 conjugate in which in the IL-2 conjugate the PEG group has an average molecular weight of 60 kDa.

In some embodiments of a method described herein, in the IL-2 conjugate the position of the structure of Formula (I) in the amino acid sequence of the IL-2 conjugate is selected from K34, F41, F43, K42, E61, P64, R37, T40, E67, Y44, V68, and L71, wherein the position of the structure of Formula (I) in the amino acid sequence of the IL-2 conjugate is in reference to the positions in SEQ ID NO: 3. In some embodiments of a method described herein, in the IL-2 conjugate the position of the structure of Formula (I) in the amino acid sequence of the IL-2 conjugate is selected from F41, E61, and P64, wherein the position of the structure of Formula (I) in the amino acid sequence of the IL-2 conjugate is in reference to the positions in SEQ ID NO: 3.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] has the structure of Formula (II) or Formula (III), or a mixture of Formula (II) and Formula (III):

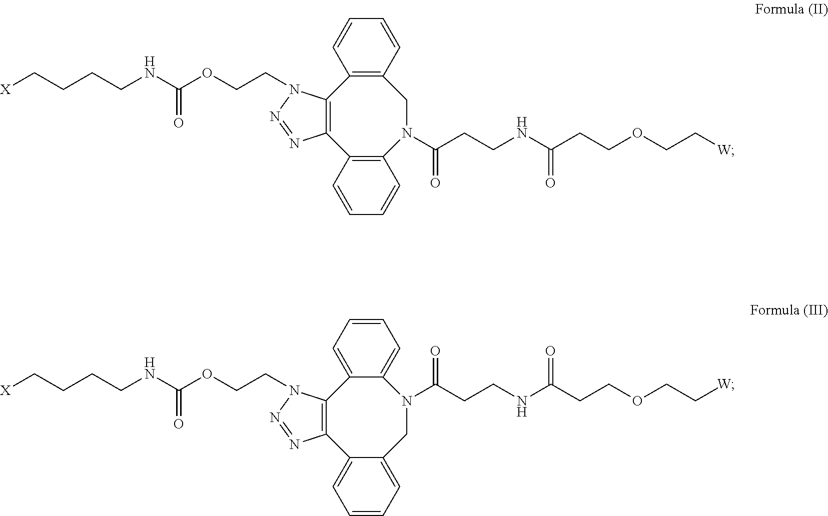

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

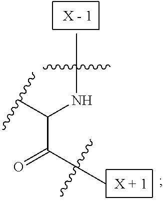

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

Here and throughout, the structure of Formula (II) encompasses pharmaceutically acceptable salts, solvates, or hydrates thereof. Here and throughout, the structure of Formula (III) encompasses pharmaceutically acceptable salts, solvates, or hydrates thereof.

In some embodiments, the [AzK_PEG] is a mixture of Formula (II) and Formula (II).

In some embodiments, the [AzK_PEG] has the structure of Formula (II):

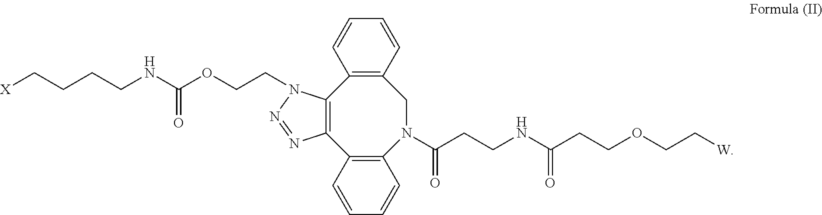

In some embodiments, the IL-2 conjugate has the amino acid sequence of SEQ ID NO: 15. In some embodiments, W in the structure of Formula (II) is a PEG group having an average molecular weight selected from 5 kDa, 10 kDa, 15 kDa, 20 kDa, 25 kDa, and 30 kDa. In some embodiments, W in the structure of Formula (II) is a PEG group having an average molecular weight selected from 5 kDa and 30 kDa. In some embodiments, W in the structure of Formula (II) is a PEG group having an average molecular weight of 5 kDa. In some embodiments, W in the structure of Formula (II) is a PEG group having an average molecular weight of 30 kDa.

In some embodiments, the IL-2 conjugate has the amino acid sequence of SEQ ID NO: 16. In some embodiments, W in the structure of Formula (II) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (II) is a PEG group having an average molecular weight selected from 5 kDa and 30 kDa. In some embodiments, W in the structure of Formula (II) is a PEG group having an average molecular weight of 5 kDa. In some embodiments, W in the structure of Formula (II) is a PEG group having an average molecular weight of 30 kDa.

In some embodiments, the IL-2 conjugate has the amino acid sequence of SEQ ID NO: 17. In some embodiments, W in the structure of Formula (II) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (II) is a PEG group having an average molecular weight selected from 5 kDa and 30 kDa. In some embodiments, W in the structure of Formula (II) is a PEG group having an average molecular weight of 5 kDa. In some embodiments, W in the structure of Formula (II) is a PEG group having an average molecular weight of 30 kDa.

In some embodiments, the IL-2 conjugate has the amino acid sequence of SEQ ID NO: 18. In some embodiments, the methods use an IL-2 conjugate in which W in the structure of Formula (II) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (II) is a PEG group having an average molecular weight selected from 5 kDa and 30 kDa. In some embodiments, W in the structure of Formula (II) is a PEG group having an average molecular weight of 5 kDa. In some embodiments, W in the structure of Formula (II) is a PEG group having an average molecular weight of 30 kDa.

In some embodiments, the IL-2 conjugate has the amino acid sequence of SEQ ID NO: 19. In some embodiments, the methods use an IL-2 conjugate in which W in the structure of Formula (II) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (II) is a PEG group having an average molecular weight selected from 5 kDa and 30 kDa. In some embodiments, W in the structure of Formula (II) is a PEG group having an average molecular weight of 5 kDa. In some embodiments, W in the structure of Formula (II) is a PEG group having an average molecular weight of 30 kDa.

In some embodiments, the [AzK_PEG] has the structure of Formula (III)

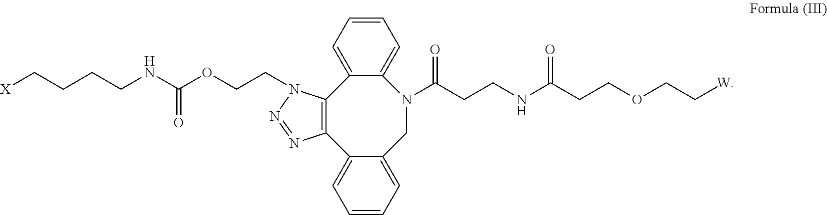

In some embodiments, the IL-2 conjugate has the amino acid sequence of SEQ ID NO: 15. In some embodiments, W in the structure of Formula (III) is a PEG group having an average molecular weight selected from 5 kDa, 10 kDa, 15 kDa, 20 kDa, 25 kDa, and 30 kDa. In some embodiments, W in the structure of Formula (III) is a PEG group having an average molecular weight selected from 5 kDa and 30 kDa. In some embodiments, W in the structure of Formula (III) is a PEG group having an average molecular weight of 5 kDa. In some embodiments, W in the structure of Formula (III) is a PEG group having an average molecular weight of 30 kDa.

In some embodiments, the IL-2 conjugate has the amino acid sequence of SEQ ID NO: 16. In some embodiments, W in the structure of Formula (III) is a PEG group having an average molecular weight selected from 5 kDa, 10 kDa, 15 kDa, 20 kDa, 25 kDa, and 30 kDa. In some embodiments, W in the structure of Formula (III) is a PEG group having an average molecular weight selected from 5 kDa and 30 kDa. In some embodiments, W in the structure of Formula (III) is a PEG group having an average molecular weight of 5 kDa. In some embodiments, W in the structure of Formula (III) is a PEG group having an average molecular weight of 30 kDa.

In some embodiments, the IL-2 conjugate has the amino acid sequence of SEQ ID NO: 17. In some embodiments, W in the structure of Formula (III) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (III) is a PEG group having an average molecular weight selected from 5 kDa and 30 kDa. In some embodiments, W in the structure of Formula (III) is a PEG group having an average molecular weight of 5 kDa. In some embodiments, W in the structure of Formula (III) is a PEG group having an average molecular weight of 30 kDa.

In some embodiments, the IL-2 conjugate has the amino acid sequence of SEQ ID NO: 18. In some embodiments, W in the structure of Formula (III) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (III) is a PEG group having an average molecular weight selected from 5 kDa and 30 kDa. In some embodiments, the methods use an IL-2 conjugate in which W in the structure of Formula (III) is a PEG group having an average molecular weight of 5 kDa. In some embodiments, the methods use an IL-2 conjugate in which W in the structure of Formula (III) is a PEG group having an average molecular weight of 30 kDa.

In some embodiments, the IL-2 conjugate has the amino acid sequence of SEQ ID NO: 19. In some embodiments, the methods use an IL-2 conjugate in which W in the structure of Formula (III) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (III) is a PEG group having an average molecular weight selected from 5 kDa and 30 kDa. In some embodiments, the methods use an IL-2 conjugate in which W in the structure of Formula (III) is a PEG group having an average molecular weight of 5 kDa. In some embodiments, the methods use an IL-2 conjugate in which W in the structure of Formula (III) is a PEG group having an average molecular weight of 30 kDa.

In some embodiments of the methods disclosed herein, an IL-2 conjugate is used having the amino acid sequence selected from any one of SEQ ID NO: 15, 16, 17, 18, and 19, wherein [AzK_PEG] contains a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 5 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 10 kDa. In some embodiments, the IL-2 conjugate has the amino acid sequence selected from any one of SEQ ID NO: 15, 16, 17, 18, and 19, wherein [AzK_PEG]contains a PEG group having an average molecular weight of 15 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 20 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 25 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 30 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 35 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 40 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 45 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 50 kDa. In some embodiments, the IL-2 conjugate has the amino acid sequence selected from any one of SEQ ID NO: 15, 16, 17, 18, and 19, wherein [AzK_PEG] contains a PEG group having an average molecular weight of 60 kDa. In some embodiments, the IL-2 conjugate has the amino acid sequence selected from any one of SEQ ID NO: 15, 16, 17, 18, and 19, wherein [AzK_PEG] contains a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa, and wherein the PEG group is a methoxy PEG group, a linear methoxy PEG group, or a branched methoxy PEG group.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20-24, wherein [AzK_PEG5kD] has the structure of Formula (II) or Formula (III), or a mixture of Formula (II) and Formula (III):

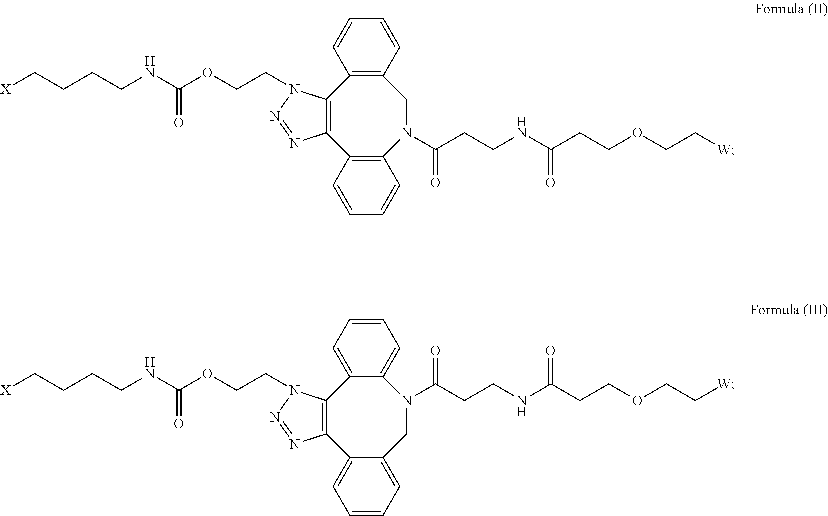

wherein:
W is a PEG group having an average molecular weight of 5 kA; and
X has the structure:

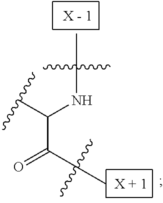

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the IL-2 conjugate has the amino acid sequence of SEQ ID NO: 20. In some embodiments, the IL-2 conjugate has the amino acid sequence of SEQ ID NO: 21. In some embodiments, the IL-2 conjugate has the amino acid sequence of SEQ ID NO: 22. In some embodiments, the IL-2 conjugate has the amino acid sequence of SEQ ID NO: 23. In some embodiments, the IL-2 conjugate has the amino acid sequence of SEQ ID NO: 24.

In some embodiments, the methods use an IL-2 conjugate in which the [AzK_PEG5kD] has the structure of Formula (II)

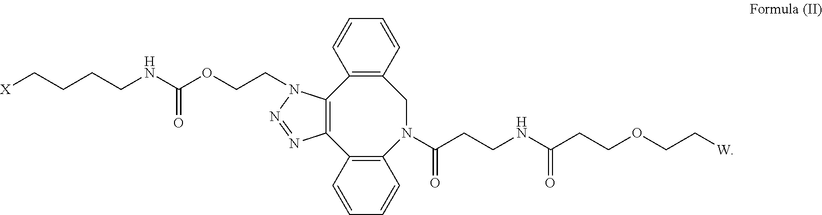

In some embodiments, the IL-2 conjugate has the amino acid sequence of SEQ ID NO: 20. In some embodiments, the IL-2 conjugate has the amino acid sequence of SEQ ID NO: 21. In some embodiments, the IL-2 conjugate has the amino acid sequence of SEQ ID NO: 22. In some embodiments, the IL-2 conjugate has the amino acid sequence of SEQ ID NO: 23. In some embodiments, the IL-2 conjugate has the amino acid sequence of SEQ ID NO: 24.

In some embodiments, the methods use an IL-2 conjugate in which the [AzK_PEG5kD] has the structure of Formula (III)

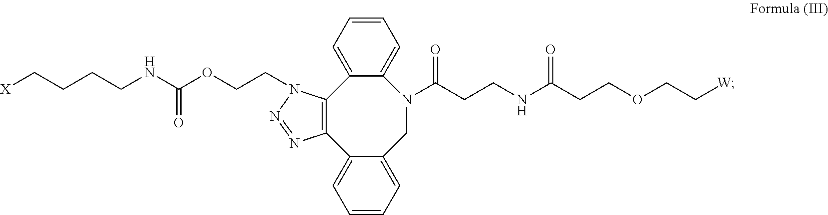

or a pharmaceutically acceptable salt, solvate, or hydrate thereof. In some embodiments, the IL-2 conjugate has the amino acid sequence of SEQ ID NO: 20. In some embodiments, the IL-2 conjugate has the amino acid sequence of SEQ ID NO: 21. In some embodiments, the IL-2 conjugate has the amino acid sequence of SEQ ID NO: 22. In some embodiments, the IL-2 conjugate has the amino acid sequence of SEQ ID NO: 23. In some embodiments, the IL-2 conjugate has the amino acid sequence of SEQ ID NO: 24.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] has the structure of Formula (II) or Formula (III), or is a mixture of the structures of Formula (II) and Formula (III):

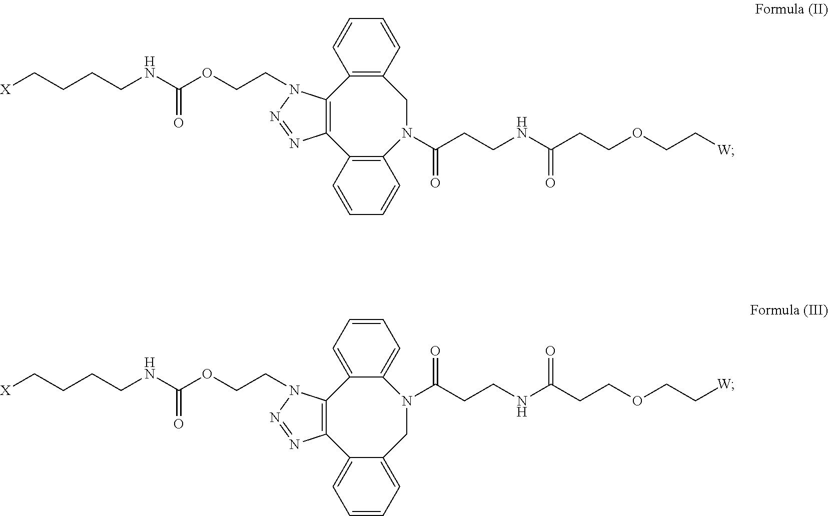

wherein:
W is a PEG group having an average molecular weight of 30 kDa; and
X has the structure:

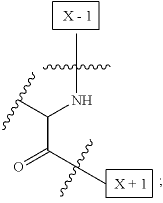

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

In some embodiments, the IL-2 conjugate has the amino acid sequence of SEQ ID NO: 25. In some embodiments, the IL-2 conjugate has the amino acid sequence of SEQ ID NO: 26. In some embodiments, the IL-2 conjugate has the amino acid sequence of SEQ ID NO: 27. In some embodiments, the IL-2 conjugate has the amino acid sequence of SEQ ID NO: 28. In some embodiments, the IL-2 conjugate has the amino acid sequence of SEQ ID NO: 29.

In some embodiments, the methods disclosed herein use an IL-2 conjugate in which the [AzK_PEG30kD] has the structure of Formula (II):

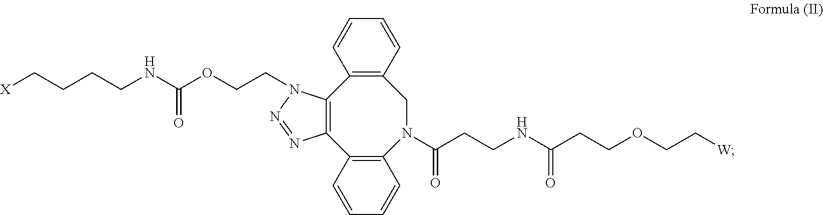

or a pharmaceutically acceptable salt, solvate, or hydrate thereof. In some embodiments, the IL-2 conjugate has the amino acid sequence of SEQ ID NO: 25. In some embodiments, the IL-2 conjugate has the amino acid sequence of SEQ ID NO: 26. In some embodiments, the IL-2 conjugate has the amino acid sequence of SEQ ID NO: 27. In some embodiments, the IL-2 conjugate has the amino acid sequence of SEQ ID NO: 28. In some embodiments, the IL-2 conjugate has the amino acid sequence of SEQ ID NO: 29.

In some embodiments, the methods use an IL-2 conjugate in which the [AzK_PEG30kD] has the structure of Formula (III)

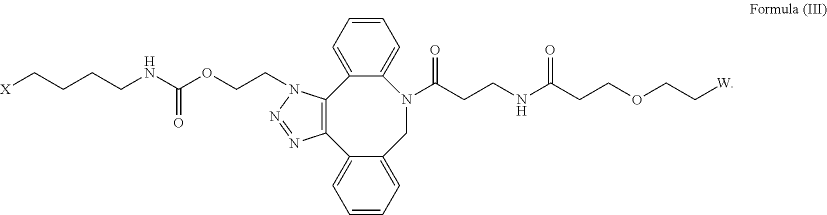

In some embodiments, the IL-2 conjugate has the amino acid sequence of SEQ ID NO: 25. In some embodiments, the IL-2 conjugate has the amino acid sequence of SEQ ID NO: 26. In some embodiments, the IL-2 conjugate has the amino acid sequence of SEQ ID NO: 27. In some embodiments, the IL-2 conjugate has the amino acid sequence of SEQ ID NO: 28. In some embodiments, the IL-2 conjugate has the amino acid sequence of SEQ ID NO: 29.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] is a mixture of the structures of Formula (II) and Formula (III):

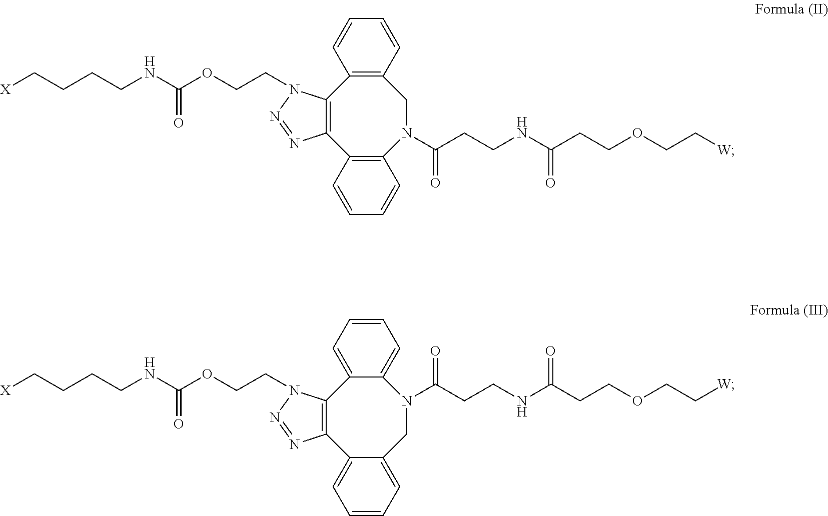

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

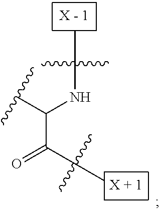

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

In some embodiments, the methods use an IL-2 conjugate in which the ratio of the amount of the structure of Formula (II) to the amount of the structure of Formula (III) comprising the total amount of [AzK_PEG] in the IL-2 conjugate is about 1:1. In some embodiments, the methods use an IL-2 conjugate in which the ratio of the amount of the structure of Formula (II) to the amount of the structure of Formula (III) comprising the total amount of [AzK_PEG] in the IL-2 conjugate is greater than 1:1. In some embodiments, the methods use an IL-2 conjugate in which the ratio of the amount of the structure of Formula (II) to the amount of the structure of Formula (III) comprising the total amount of [AzK_PEG] in the IL-2 conjugate is less than 1:1. In some embodiments, the methods use an IL-2 conjugate in which the W is a linear or branched PEG group. In some embodiments, the methods use an IL-2 conjugate in which the W is a linear PEG group. In some embodiments, the methods use an IL-2 conjugate in which W is a branched PEG group. In some embodiments, the methods use an IL-2 conjugate in which W is a methoxy PEG group. In some embodiments, the methods use an IL-2 conjugate in which the methoxy PEG group is linear or branched. In some embodiments, the methods use an IL-2 conjugate in which the methoxy PEG group is linear. In some embodiments, the methods use an IL-2 conjugate in which the methoxy PEG group is branched.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20 to 24, wherein [AzK_PEG5kD] is a mixture of the structures of Formula (II) and Formula III):

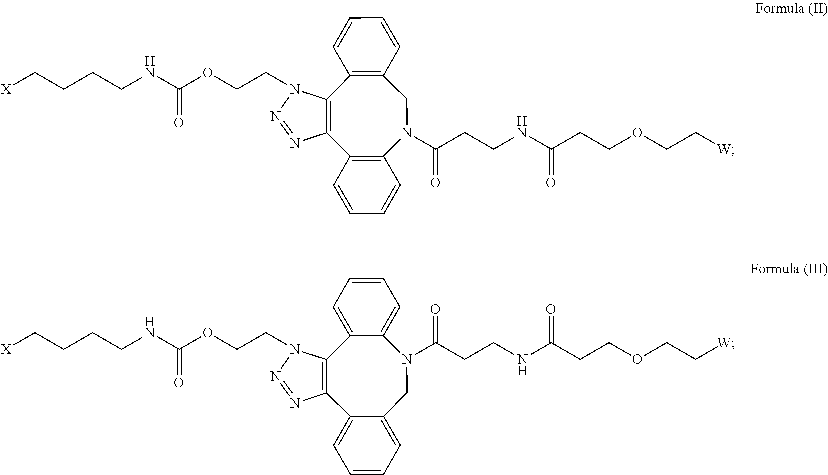

wherein:
W is a PEG group having an average molecular weight of 5 kDa; and
X has the structure:

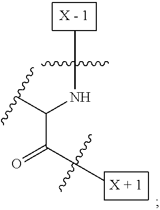

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the methods use an IL-2 conjugate in which the ratio of the amount of the structure of Formula (II) to the amount of the structure of Formula (III) comprising the total amount of [AzK_PEG5kD] in the IL-2 conjugate is about 1:1. In some embodiments, the methods use an IL-2 conjugate in which the ratio of the amount of the structure of Formula (II) to the amount of the structure of Formula (III) comprising the total amount of [AzK_PEG5kD] in the IL-2 conjugate is greater than 1:1. In some embodiments, the methods use an IL-2 conjugate in which the ratio of the amount of the structure of Formula (II) to the amount of the structure of Formula (III) comprising the total amount of [AzK_PEG5kD] in the IL-2 conjugate is less than 1:1.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] is a mixture of the structures of Formula (II) and Formula (III):

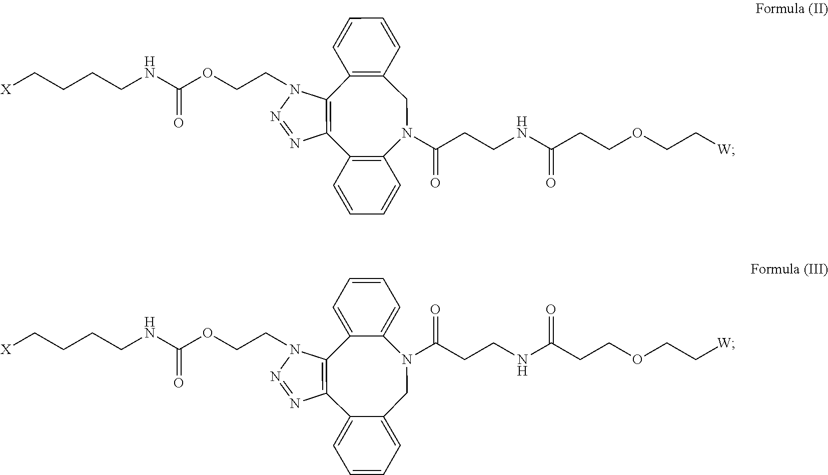

wherein:
W is a PEG group having an average molecular weight of 30 kDa; and
X has the structure:

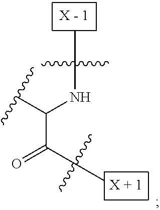

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the methods use an IL-2 conjugate in which the ratio of the amount of the structure of Formula (II) to the amount of the structure of Formula (HI) comprising the total amount of [AzK_PEG30kD] in the IL-2 conjugate is about 1:1. In some embodiments, the methods use an IL-2 conjugate in which the ratio of the amount of the structure of Formula (II) to the amount of the structure of Formula (II) comprising the total amount of [AzK_PEG30kD] in the IL-2 conjugate is greater than 1:1. In some embodiments, the methods use an IL-2 conjugate in which the ratio of the amount of the structure of Formula (II) to the amount of the structure of Formula (III) comprising the total amount of [AzK_PEG30kD] in the IL-2 conjugate is less than 1:1.

In some embodiments, the methods use an IL-2 conjugate described herein comprising the structure of Formula (II) or Formula (III), or a mixture of Formula (II) and Formula (III), wherein W is a linear or branched PEG group. In some embodiments, the methods use an IL-2 conjugate in which W in the structure of Formula (II) or Formula (III) is a linear PEG group. In some embodiments, the methods use an IL-2 conjugate in which W in the structure of Formula (II) or Formula (III) is a branched PEG group. In some embodiments, the methods use an IL-2 conjugate in which W in the structure of Formula (II) or Formula (III) is a methoxy PEG group. In some embodiments, the methods use an IL-2 conjugate in which W in the structure of Formula (II) or Formula (III) is a methoxy PEG group that is linear or branched. In some embodiments, the methods use an IL-2 conjugate in which the methoxy PEG group in the structure of Formula (II) or Formula (III) is linear. In some embodiments, the methods use an IL-2 conjugate in which the methoxy PEG group in the structure of Formula (II) or Formula (III) is branched.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [AzK_L1_PEG] has the structure of Formula (IV) or Formula (V), or a mixture of Formula (IV) and Formula (V):

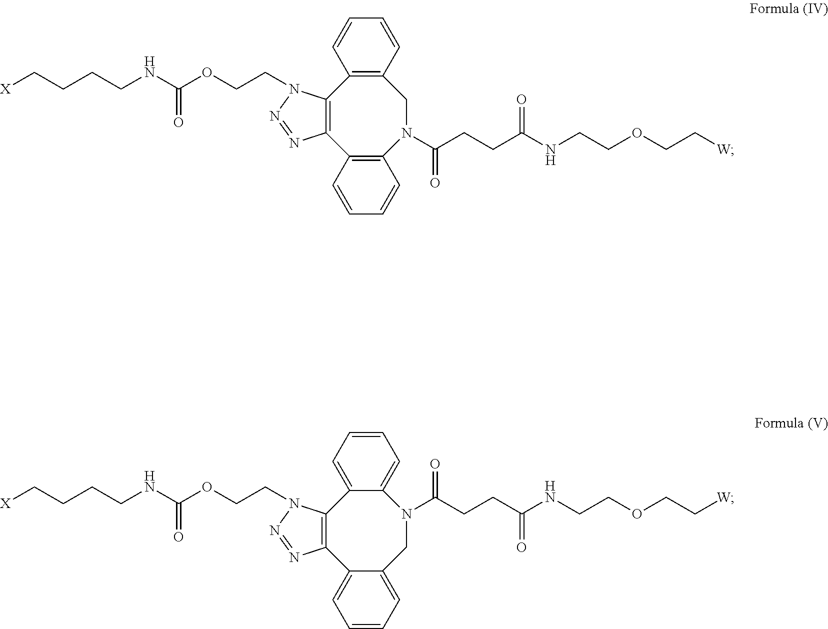

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

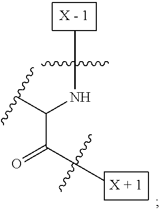

X−1 indicates the point of attachment to the preceding amino acid residue; and
X+1 indicates the point of attachment to the following amino acid residue;
or a pharmaceutically acceptable salt, solvate, or hydrate thereof.

Here and throughout, the structure of Formula (IV) encompasses pharmaceutically acceptable salts, solvates, or hydrates thereof. Here and throughout, the structure of Formula (V) encompasses pharmaceutically acceptable salts, solvates, or hydrates thereof.

In some embodiments, the methods use an IL-2 conjugate in which the [AzK_L1_PEG] is a mixture of Formula (IV) and Formula (V).

In some embodiments, the methods use an IL-2 conjugate in which the [AzK_L1_PEG] has the structure of Formula (IV):

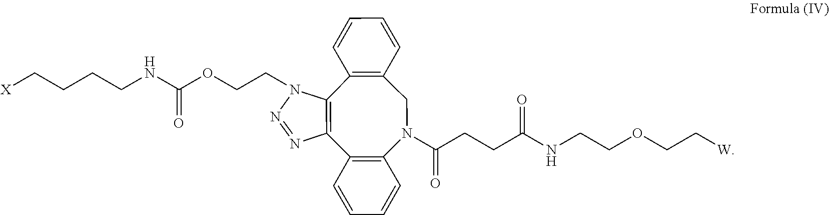

In some embodiments, the IL-2 conjugate has the amino acid sequence of SEQ ID NO: 40. In some embodiments, the methods use an IL-2 conjugate in which W in the structure of Formula (IV) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) is a PEG group having an average molecular weight selected from 5 kDa and 30 kDa. In some embodiments, the methods use an IL-2 conjugate in which W in the structure of Formula (IV) is a PEG group having an average molecular weight of 5 kDa. In some embodiments, the methods use an IL-2 conjugate in which W in the structure of Formula (IV) is a PEG group having an average molecular weight of 30 kDa.

In some embodiments, the IL-2 conjugate has the amino acid sequence of SEQ ID NO: 41. In some embodiments, the methods use an IL-2 conjugate in which W in the structure of Formula (IV) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) is a PEG group having an average molecular weight selected from 5 kDa and 30 kDa. In some embodiments, the methods use an IL-2 conjugate in which W in the structure of Formula (IV) is a PEG group having an average molecular weight of 5 kDa. In some embodiments, the methods use an IL-2 conjugate in which W in the structure of Formula (IV) is a PEG group having an average molecular weight of 30 kDa.

In some embodiments, the IL-2 conjugate has the amino acid sequence of SEQ ID NO: 42. In some embodiments, the methods use an IL-2 conjugate in which W in the structure of Formula (IV) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) is a PEG group having an average molecular weight selected from 5 kDa and 30 kDa. In some embodiments, the methods use an IL-2 conjugate in which W is a PEG group having an average molecular weight of 5 kDa. In some embodiments, the methods use an IL-2 conjugate in which W in the structure of Formula (IV) is a PEG group having an average molecular weight of 30 kDa.

In some embodiments, the IL-2 conjugate has the amino acid sequence of SEQ ID NO: 43. In some embodiments, the methods use an IL-2 conjugate in which W in the structure of Formula (IV) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) is a PEG group having an average molecular weight selected from 5 kDa and 30 kDa. In some embodiments, the methods use an IL-2 conjugate in which W in the structure of Formula (IV) is a PEG group having an average molecular weight of 5 kDa. In some embodiments, the methods use an IL-2 conjugate in which W in the structure of Formula (IV) is a PEG group having an average molecular weight of 30 kDa.

In some embodiments, the IL-2 conjugate has the amino acid sequence of SEQ ID NO: 44 In some embodiments, the methods use an IL-2 conjugate in which W in the structure of Formula (IV) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) is a PEG group having an average molecular weight selected from 5 kDa and 30 kDa. In some embodiments, the methods use an IL-2 conjugate in which W in the structure of Formula (IV) is a PEG group having an average molecular weight of 5 kDa. In some embodiments, the methods use an IL-2 conjugate in which W in the structure of Formula (IV) is a PEG group having an average molecular weight of 30 kDa.

In some embodiments, the methods use an IL-2 conjugate in which the [AzK_L1_PEG] has the structure of Formula (V)

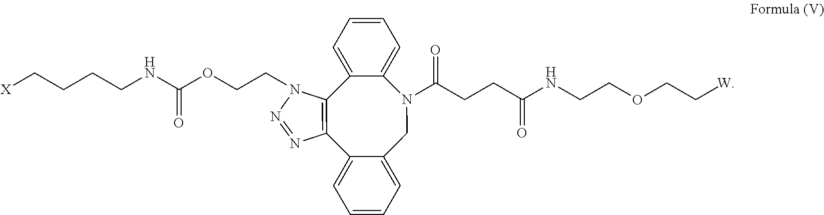

In some embodiments, the IL-2 conjugate has the amino acid sequence of SEQ ID NO: 40 In some embodiments, the methods use an IL-2 conjugate in which W in the structure of Formula (V) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (V) is a PEG group having an average molecular weight selected from 5 kDa and 30 kDa. In some embodiments, the methods use an IL-2 conjugate in which W in the structure of Formula (V) is a PEG group having an average molecular weight of 5 kDa. In some embodiments, the methods use an IL-2 conjugate in which W in the structure of Formula (V) is a PEG group having an average molecular weight of 30 kDa.

In some embodiments, the IL-2 conjugate has the amino acid sequence of SEQ ID NO: 41 In some embodiments, the methods use an IL-2 conjugate in which W in the structure of Formula (V) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (V) is a PEG group having an average molecular weight selected from 5 kDa and 30 kDa. In some embodiments, the methods use an IL-2 conjugate in which W in the structure of Formula (V) is a PEG group having an average molecular weight of 5 kDa. In some embodiments, the methods use an IL-2 conjugate in which W in the structure of Formula (V) is a PEG group having an average molecular weight of 30 kDa.

In some embodiments, the IL-2 conjugate has the amino acid sequence of SEQ ID NO: 42. In some embodiments, the methods use an IL-2 conjugate in which W in the structure of Formula (V) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (V) is a PEG group having an average molecular weight selected from 5 kDa and 30 kDa. In some embodiments, the methods use an IL-2 conjugate in which W in the structure of Formula (V) is a PEG group having an average molecular weight of 5 kDa. In some embodiments, the methods use an IL-2 conjugate in which W in the structure of Formula (V) is a PEG group having an average molecular weight of 30 kDa.

In some embodiments, the IL-2 conjugate has the amino acid sequence of SEQ ID NO: 43. In some embodiments, the methods use an IL-2 conjugate in which W in the structure of Formula (V) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (V) is a PEG group having an average molecular weight selected from 5 kDa and 30 kDa. In some embodiments, the methods use an IL-2 conjugate in which W in the structure of Formula (V) is a PEG group having an average molecular weight of 5 kDa. In some embodiments, the methods use an IL-2 conjugate in which W in the structure of Formula (V) is a PEG group having an average molecular weight of 30 kDa.

In some embodiments, the IL-2 conjugate has the amino acid sequence selected from any one of SEQ ID NO: 40, 41, 42, 43, and 44, wherein [AzK_L1_PEG] contains a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 5 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 10 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 15 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 20 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 25 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 30 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 35 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 40 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 45 kDa In some embodiments, the IL-2 conjugate has the amino acid sequence selected from any one of SEQ ID NO: 40, 41, 42, 43, and 44, wherein [AzK_L1_PEG] contains a PEG group having an average molecular weight of 50 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight of 60 kDa. In some embodiments, the IL-2 conjugate has the amino acid sequence selected from any one of SEQ ID NO: 40, 41, 42, 43, and 44, wherein [AzK_L1_PEG] contains a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa, and wherein the PEG group is a methoxy PEG group, a linear methoxy PEG group, or a branched methoxy PEG group.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45-49, wherein [AzK_L1_PEG5kD] has the structure of Formula (IV) or Formula (V), or a mixture of Formula (IV) and Formula (V):

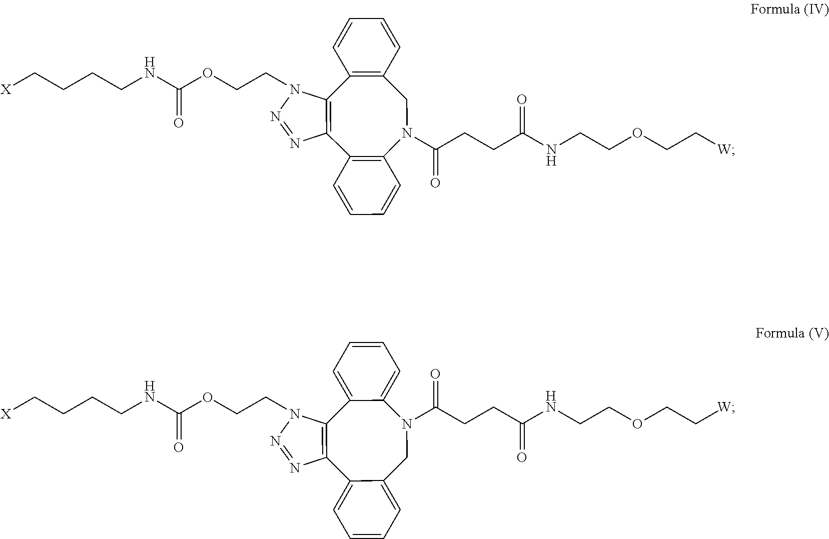

wherein:
W is a PEG group having an average molecular weight of 5 kDa; and
X has the structure:

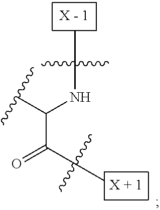

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

In some embodiments, the IL-2 conjugate has the amino acid sequence of SEQ ID NO: 45. In some embodiments, the IL-2 conjugate has the amino acid sequence of SEQ ID NO: 46. In some embodiments, the IL-2 conjugate has the amino acid sequence of SEQ ID NO: 47. In some embodiments, the IL-2 conjugate has the amino acid sequence of SEQ ID NO: 48. In some embodiments, the IL-2 conjugate has the amino acid sequence of SEQ ID NO: 49.

In some embodiments, the methods use an IL-2 conjugate in which the [AzK_L1_PEG5kD] has the structure of Formula (IV)

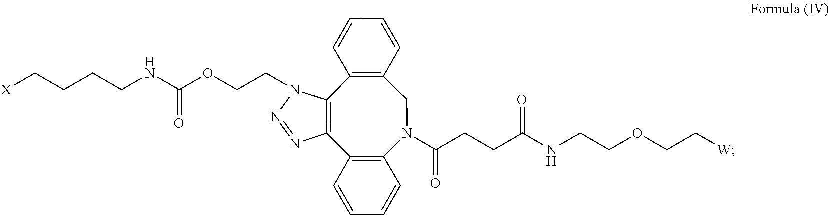

or a pharmaceutically acceptable salt, solvate, or hydrate thereof. In some embodiments, the IL-2 conjugate has the amino acid sequence of SEQ ID NO: 45. In some embodiments, the IL-2 conjugate has the amino acid sequence of SEQ ID NO: 46. In some embodiments, the IL-2 conjugate has the amino acid sequence of SEQ ID NO: 47. In some embodiments, the IL-2 conjugate has the amino acid sequence of SEQ ID NO: 48. In some embodiments, the IL-2 conjugate has the amino acid sequence of SEQ ID NO: 49.

In some embodiments, the methods use an IL-2 conjugate in which the [AzK_L1_PEG5kD] has the structure of Formula (V)

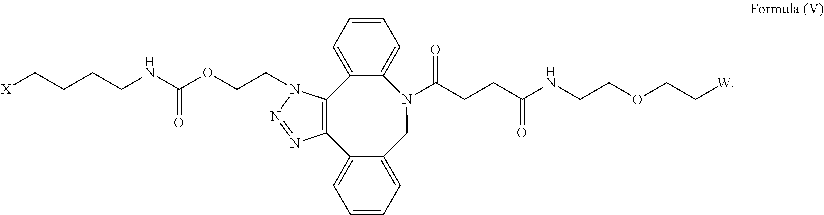

In some embodiments, the IL-2 conjugate has the amino acid sequence of SEQ ID NO: 45. In some embodiments, the IL-2 conjugate has the amino acid sequence of SEQ ID NO: 46. In some embodiments, the IL-2 conjugate has the amino acid sequence of SEQ ID NO: 47. In some embodiments, the IL-2 conjugate has the amino acid sequence of SEQ ID NO: 48. In some embodiments, the IL-2 conjugate has the amino acid sequence of SEQ ID NO: 49.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 50-54, wherein [AzK_L1_PEG30kD] has the structure of Formula (IV) or Formula (V), or is a mixture of the structures of Formula (IV) and Formula (V):

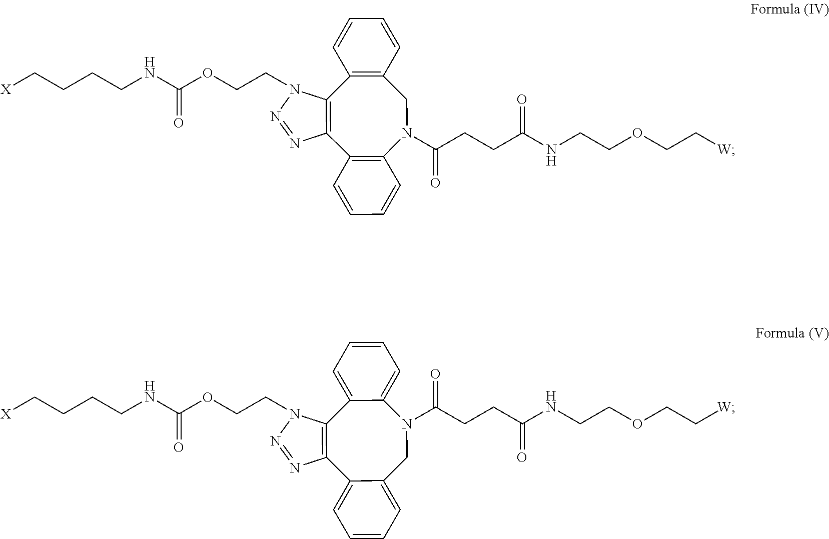

wherein:
W is a PEG group having an average molecular weight of 30 kDa; and
X has the structure:

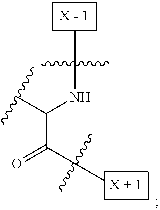

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the IL-2 conjugate has the amino acid sequence of SEQ ID NO: 50. In some embodiments, the IL-2 conjugate has the amino acid sequence of SEQ ID NO: 51. In some embodiments, the IL-2 conjugate has the amino acid sequence of SEQ ID NO: 52. In some embodiments, the IL-2 conjugate has the amino acid sequence of SEQ ID NO: 53. In some embodiments, the IL-2 conjugate has the amino acid sequence of SEQ ID NO: 54.

In some embodiments, the methods use an IL-2 conjugate in which the [AzK_L1_PEG30kD] has the structure of Formula (IV):

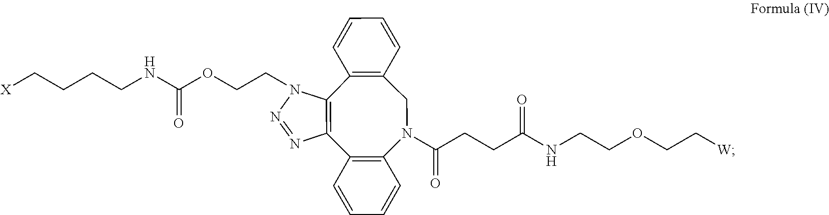

or a pharmaceutically acceptable salt, solvate, or hydrate thereof. In some embodiments, the IL-2 conjugate has the amino acid sequence of SEQ ID NO: 50. In some embodiments, the IL-2 conjugate has the amino acid sequence of SEQ ID NO: 51. In some embodiments, the IL-2 conjugate has the amino acid sequence of SEQ ID NO: 52. In some embodiments, the IL-2 conjugate has the amino acid sequence of SEQ ID NO: 53. In some embodiments, the IL-2 conjugate has the amino acid sequence of SEQ ID NO: 54.

In some embodiments, the methods use an IL-2 conjugate in which the [AzK_L1_PEG30kD] has the structure of Formula (V)

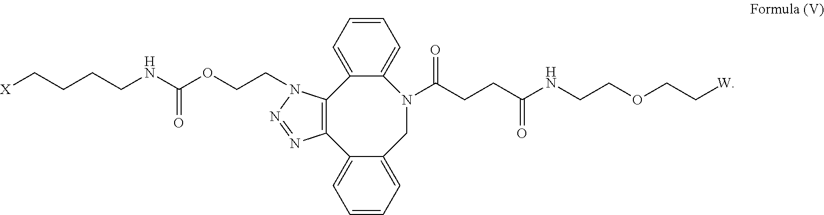

In some embodiments, the IL-2 conjugate has the amino acid sequence of SEQ ID NO: 50. In some embodiments, the IL-2 conjugate has the amino acid sequence of SEQ ID NO: 51. In some embodiments, the IL-2 conjugate has the amino acid sequence of SEQ ID NO: 52. In some embodiments, the IL-2 conjugate has the amino acid sequence of SEQ ID NO: 53. In some embodiments, the IL-2 conjugate has the amino acid sequence of SEQ ID NO: 54.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [Azk_L1_PEG] is a mixture of the structures of Formula (IV) and Formula (V):

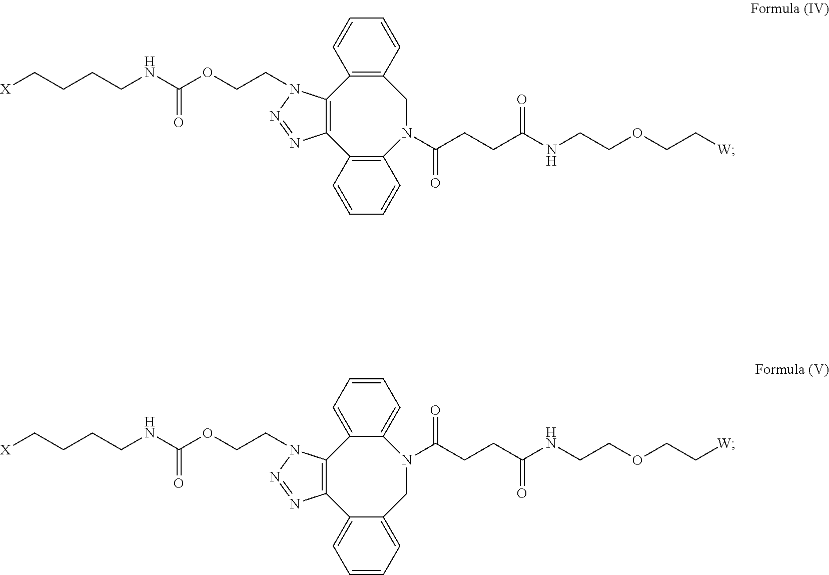

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

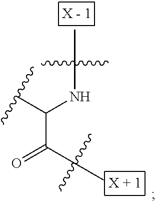

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the methods use an IL-2 conjugate in which the ratio of the amount of the structure of Formula (IV) to the amount of the structure of Formula (V) comprising the total amount of [AzK_L1_PEG] in the IL-2 conjugate is about 1:1. In some embodiments, the methods use an IL-2 conjugate in which the ratio of the amount of the structure of Formula (IV) to the amount of the structure of Formula (V) comprising the total amount of [AzK_L1_PEG] in the IL-2 conjugate is greater than 1:1. In some embodiments, the methods use an IL-2 conjugate in which the ratio of the amount of the structure of Formula (IV) to the amount of the structure of Formula (V) comprising the total amount of [AzK_L1_PEG] in the IL-2 conjugate is less than 1:1.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45 to 49, wherein [AzK_L1_PEG5kD] is a mixture of the structures of Formula (IV) and Formula (V):

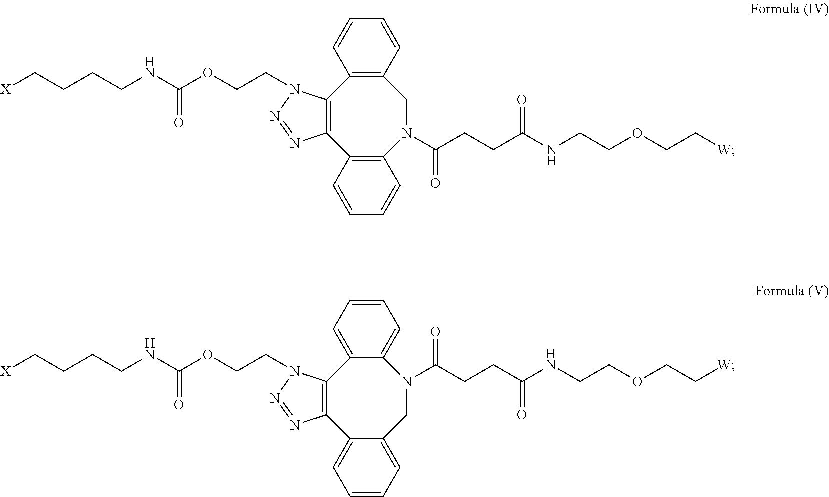

wherein:
W is a PEG group having an average molecular weight of 5 kDa; and
X has the structure:

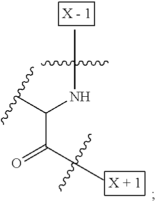

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the methods use an IL-2 conjugate in which the ratio of the amount of the structure of Formula (IV) to the amount of the structure of Formula (V) comprising the total amount of [AzK_L1_PEG5kD] in the IL-2 conjugate is about 1:1. In some embodiments, the methods use an IL-2 conjugate in which the ratio of the amount of the structure of Formula (IV) to the amount of the structure of Formula (V) comprising the total amount of [AzK_L1_PEG5kD] in the IL-2 conjugate is greater than 1:1. In some embodiments, the methods use an IL-2 conjugate in which the ratio of the amount of the structure of Formula (IV) to the amount of the structure of Formula (V) comprising the total amount of [AzK_L1_PEG5kD] in the IL-2 conjugate is less than 1:1.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 50-54, wherein [AzK_L1 PEG30kD] is a mixture of the structures of Formula (IV) and Formula (V):

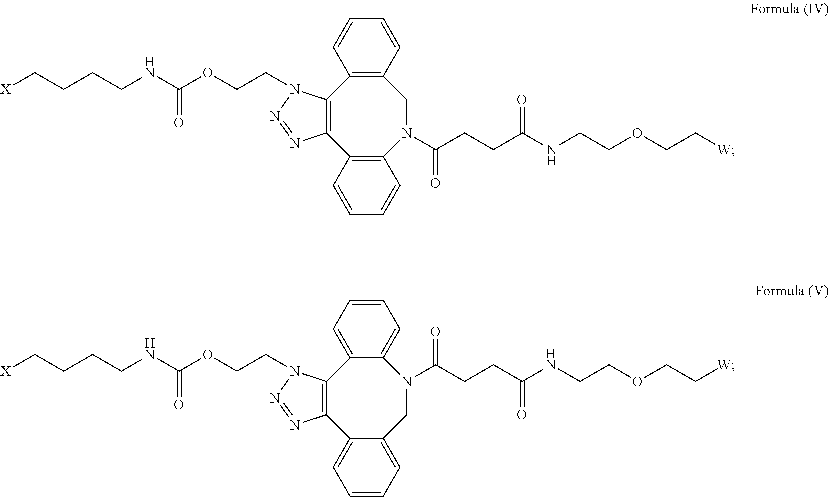

wherein:
W is a PEG group having an average molecular weight of 30 kDa; and
X has the structure:

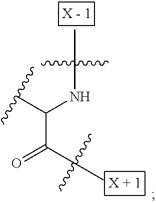

X−1 indicates the point of attachment to the preceding amino acid residue; and
X+1 indicates the point of attachment to the following amino acid residue;
or a pharmaceutically acceptable salt, solvate, or hydrate thereof.

In some embodiments, the methods use an IL-2 conjugate in which the ratio of the amount of the structure of Formula (IV) to the amount of the structure of Formula (V) comprising the total amount of [AzK_L1_PEG30kD] in the IL-2 conjugate is about 1:1. In some embodiments, the methods use an IL-2 conjugate in which the ratio of the amount of the structure of Formula (IV) to the amount of the structure of Formula (V) comprising the total amount of [AzK_L1_PEG30 kD] in the IL-2 conjugate is greater than 1:1. In some embodiments, the methods use an IL-2 conjugate in which the ratio of the amount of the structure of Formula (IV) to the amount of the structure of Formula (V) comprising the total amount of [AzK_L1_PEG30 kD] in the IL-2 conjugate is less than 1:1.

In some embodiments, the methods use an IL-2 conjugate in which W in the structure of Formula (IV) or Formula (V) is a PEG group having an average molecular weight selected from 5 kDa, 10 kDa, 15 kDa, 20 kDa, 25 kDa, and 30 kDa. In some embodiments, the methods use an IL-2 conjugate in which W in the structure of Formula (IV) or Formula (V) is a PEG group having an average molecular weight of 5 kDa. In some embodiments, the methods use an IL-2 conjugate in which W in the structure of Formula (IV) or Formula (V) is a PEG group having an average molecular weight of 30 kDa. In some embodiments, the methods use an IL-2 conjugate in which W in the structure of Formula (IV) or Formula (V) is a PEG group having an average molecular weight of 10 kDa. In some embodiments, the methods use an IL-2 conjugate in which W in the structure of Formula (IV) or Formula (V) is a PEG group having an average molecular weight of 15 kDa. In some embodiments, the methods use an IL-2 conjugate in which W in the structure of Formula (IV) or Formula (V) is a PEG group having an average molecular weight of 20 kDa. In some embodiments, the methods use an IL-2 conjugate in which W in the structure of Formula (IV) or Formula (V) is a PEG group having an average molecular weight of 25 kDa. In some embodiments, the methods use an IL-2 conjugate in which W in the structure of Formula (IV) or Formula (V) is a PEG group having an average molecular weight of 30 kDa. In some embodiments, the methods use an IL-2 conjugate in which W in the structure of Formula (IV) or Formula (V) is a PEG group having an average molecular weight of 35 kDa. In some embodiments, the methods use an IL-2 conjugate in which W in the structure of Formula (IV) or Formula (V) is a PEG group having an average molecular weight of 40 kDa. In some embodiments, the methods use an IL-2 conjugate in which W in the structure of Formula (IV) or Formula (V) is a PEG group having an average molecular weight of 45 kDa. In some embodiments, the methods use an IL-2 conjugate in which W in the structure of Formula (IV) or Formula (V) is a PEG group having an average molecular weight of 50 kDa. In some embodiments, the methods use an IL-2 conjugate in which W in the structure of Formula (IV) or Formula (V) is a PEG group having an average molecular weight of 55 kDa. In some embodiments, the methods use an IL-2 conjugate in which W in the structure of Formula (IV) or Formula (V) is a PEG group having an average molecular weight of 60 kDa.

In some embodiments, the methods use an IL-2 conjugate described herein comprising the structure of Formula (IV) or Formula (V), or a mixture of Formula (IV) and Formula (V), wherein W is a linear or branched PEG group. In some embodiments, the methods use an IL-2 conjugate in which W in the structure of Formula (IV) or Formula (V) is a linear PEG group. In some embodiments, the methods use an IL-2 conjugate in which W in the structure of Formula (IV) or Formula (V) is a branched PEG group. In some embodiments, the methods use an IL-2 conjugate in which W in the structure of Formula (IV) or Formula (V) is a methoxy PEG group. In some embodiments, the methods use an IL-2 conjugate in which W in the structure of Formula (IV) or Formula (V) is a methoxy PEG group that is linear or branched. In some embodiments, the methods use an IL-2 conjugate in which the methoxy PEG group in the structure of Formula (IV) or Formula (V) is linear. In some embodiments, the methods use an IL-2 conjugate in which the methoxy PEG group in the structure of Formula (IV) or Formula (V) is branched.

With respect to the IL-2 conjugates used in the methods described herein, an exemplary structure of a methoxy PEG group is illustrated in the mPEG-DBCO structure in Scheme 1 of Example 2. Exemplary structures of a methoxy PEG group is illustrated in the mPEG-DBCO structures below:

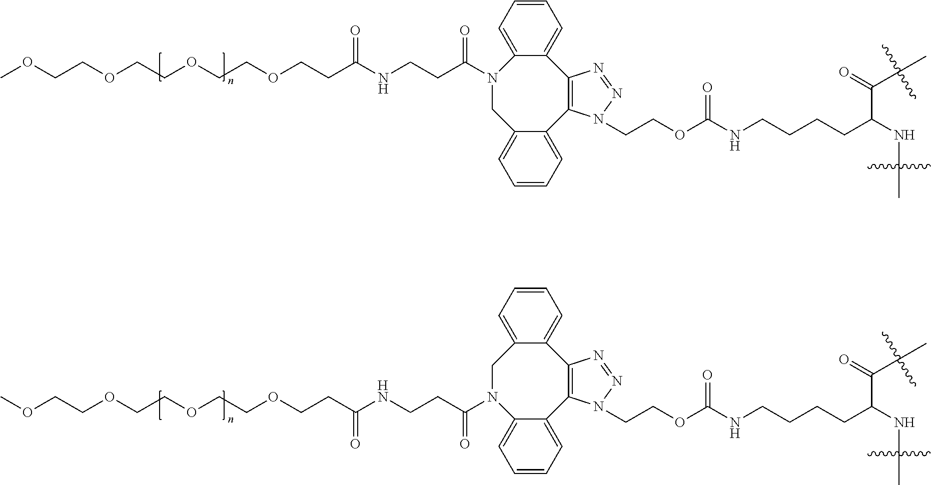

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3, in which at least one amino acid residue in the IL-2 conjugate is replaced by the structure of Formula (VI) or Formula (VII), or a mixture of Formula (VI) and Formula (VII):

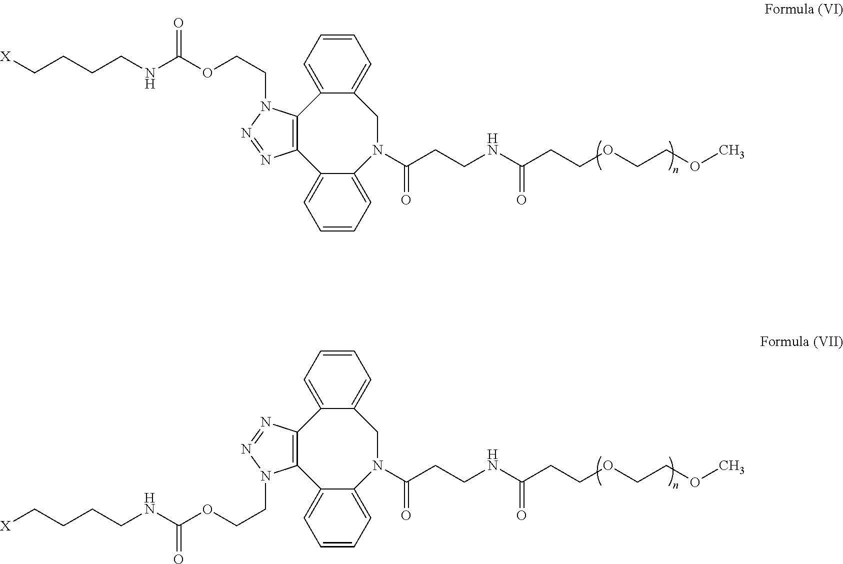

wherein:
n is an integer in the range from about 2 to about 5000; and
X has the structure:

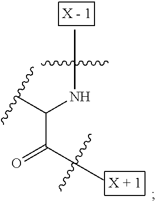

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

Here and throughout, the structure of Formula (VI) encompasses pharmaceutically acceptable salts, solvates, or hydrates thereof. Here and throughout, the structure of Formula (VII) encompasses pharmaceutically acceptable salts, solvates, or hydrates thereof.

In some embodiments, n in the compounds of Formula (VI) and Formula (VII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and Formula (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (VI), Formula (VII), or a mixture of Formula (VI) and Formula (VII) in the amino acid sequence of the IL-2 conjugate is selected from K34, F41, F43, K42, E61, P64, R37, T40, E67, Y44, V68, and L71, wherein the position of the structure of Formula (VI), Formula (VII), or a mixture of Formula (VI) and Formula (VII) in the amino acid sequence of the IL-2 conjugate is in reference to the positions in SEQ ID NO: 3. In some embodiments, the position of the structure of Formula (VI), Formula (VII), or a mixture of Formula (VI) and Formula (VII) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (VI), Formula (VII), or a mixture of Formula (VI) and Formula (VII) in the amino acid sequence of the IL-2 conjugate of SEQ ID NO: 3 is at position K34. In some embodiments, the position of the structure of Formula (VI), Formula (VII), or a mixture of Formula (VI) and Formula (VII) in the amino acid sequence of the IL-2 conjugate of SEQ ID NO: 3 is at position F41. In some embodiments, the position of the structure of Formula (VI), Formula (VII), or a mixture of Formula (VI) and Formula (VII) in the amino acid sequence of the IL-2 conjugate of SEQ ID NO: 3 is at position F43. In some embodiments, the position of the structure of Formula (VI), Formula (VII), or a mixture of Formula (VI) and Formula (VII) in the amino acid sequence of the IL-2 conjugate of SEQ ID NO: 3 is at position K42. In some embodiments, the position of the structure of Formula (VI), Formula (VII), or a mixture of Formula (VI) and Formula (VII) in the amino acid sequence of the IL-2 conjugate of SEQ ID NO: 3 is at position E61. In some embodiments, the position of the structure of Formula (VI), Formula (VII), or a mixture of Formula (VI) and Formula (VII) in the amino acid sequence of the IL-2 conjugate of SEQ ID NO: 3 is at position P64. In some embodiments, the position of the structure of Formula (VI), Formula (VII), or a mixture of Formula (VI) and Formula (VII) in the amino acid sequence of the IL-2 conjugate of SEQ ID NO: 3 is at position R37. In some embodiments, the position of the structure of Formula (VI), Formula (VII), or a mixture of Formula (VI) and Formula (VII) in the amino acid sequence of the IL-2 conjugate of SEQ ID NO: 3 is at position T40. In some embodiments, the position of the structure of Formula (VI), Formula (VII), or a mixture of Formula (VI) and Formula (VII) in the amino acid sequence of the IL-2 conjugate of SEQ ID NO: 3 is at position E67. In some embodiments, the position of the structure of Formula (VI), Formula (VII), or a mixture of Formula (VI) and Formula (VII) in the amino acid sequence of the IL-2 conjugate of SEQ ID NO: 3 is at position Y44. In some embodiments, the position of the structure of Formula (VI), Formula (VII), or a mixture of Formula (VI) and Formula (VII) in the amino acid sequence of the IL-2 conjugate of SEQ ID NO: 3 is at position V68. In some embodiments, the position of the structure of Formula (VI), Formula (VII), or a mixture of Formula (VI) and Formula (VII) in the amino acid sequence of the IL-2 conjugate of SEQ ID NO: 3 is at position L71.

In some embodiments, the methods use an IL-2 conjugate in which the ratio of the amount of the structure of Formula (VI) to the amount of the structure of Formula (VII) comprising the total amount of the IL-2 conjugate is about 1:1. In some embodiments, the methods use an IL-2 conjugate in which the ratio of the amount of the structure of Formula (VI) to the amount of the structure of Formula (VII) comprising the total amount of the IL-2 conjugate is greater than 1:1. In some embodiments, the methods use an IL-2 conjugate in which the ratio of the amount of the structure of Formula (VI) to the amount of the structure of Formula (VII) comprising the total amount of the IL-2 conjugate is less than 1:1.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and Formula (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (VI) and Formula (VII) is an integer selected from 454, 455, 568, 569, 680, 681, 682, 794, 795, 796, 908, 909, 910, 1021, 1022, 1023, 1135, 1136, 1137, and 1249.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (VI) and Formula (VII) is an integer selected from 454, 455, 568, 569, 680, 681, 682, 794, 795, 796, 908, 909, and 910.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, the methods use an IL-2 conjugate in which n in the compounds of Formula (VI) and Formula (VII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (VI) and Formula (VII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments n in the structures of Formula (VI) and Formula (VII) is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

In some embodiments, the methods use an IL-2 conjugate comprising the amino acid sequence of SEQ ID NO: 3 in which at least one amino acid residue in the IL-2 conjugate is replaced by the structure of Formula (VI) or Formula (VII), or a mixture of Formula (VI) and Formula (VII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (VIII) or Formula (IX), or a mixture of Formula (VIII) and Formula (IX):

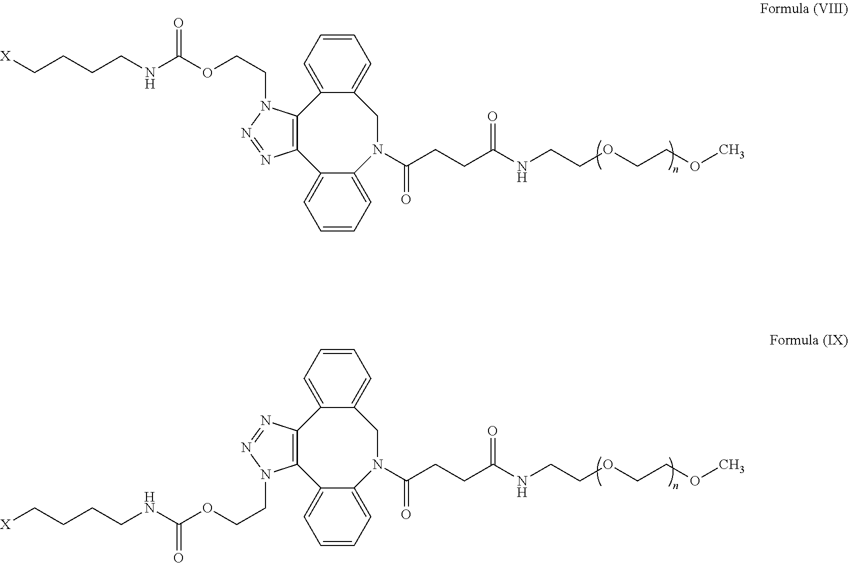

wherein:
n is an integer in the range from about 2 to about 5000; and
X has the structure:

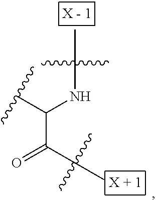

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

Here and throughout, the structure of Formula (VIII) encompasses pharmaceutically acceptable salts, solvates, or hydrates thereof. Here and throughout, the structure of Formula (IX) encompasses pharmaceutically acceptable salts, solvates, or hydrates thereof.

In some embodiments, n in the compounds of Formula (VIII) and Formula (IX) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VIII) and Formula (IX) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (VIII), Formula (IX), or a mixture of Formula (VIII) and Formula (IX) in the amino acid sequence of the IL-2 conjugate is selected from K34, F41, F43, K42, E61, P64, R37, T40, E67, Y44, V68, and L71, wherein the position of the structure of Formula (VIII), Formula (IX), or a mixture of Formula (VIII) and Formula (IX) in the amino acid sequence of the IL-2 conjugate is in reference to the positions in SEQ ID NO: 3. In some embodiments, the position of the structure of Formula (VIII), Formula (IX), or a mixture of Formula (VIII) and Formula (IX) in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (VIII), Formula (IX), or a mixture of Formula (VIII) and Formula (IX) in the amino acid sequence of the IL-2 conjugate of SEQ ID NO: 3 is at position K34. In some embodiments, the position of the structure of Formula (VIII), Formula (IX), or a mixture of Formula (VIII) and Formula (IX) in the amino acid sequence of the IL-2 conjugate of SEQ ID NO: 3 is at position F41. In some embodiments, the position of the structure of Formula (VIII), Formula (IX), or a mixture of Formula (VIII) and Formula (IX) in the amino acid sequence of the IL-2 conjugate of SEQ ID NO: 3 is at position F43. In some embodiments, the position of the structure of Formula (VIII), Formula (IX), or a mixture of Formula (VIII) and Formula (IX) in the amino acid sequence of the IL-2 conjugate of SEQ ID NO: 3 is at position K42. In some embodiments, the position of the structure of Formula (VIII), Formula (IX), or a mixture of Formula (VIII) and Formula (IX) in the amino acid sequence of the IL-2 conjugate of SEQ ID NO: 3 is at position E61. In some embodiments, the position of the structure of Formula (VIII), Formula (IX), or a mixture of Formula (VIII) and Formula (IX) in the amino acid sequence of the IL-2 conjugate of SEQ ID NO: 3 is at position P64. In some embodiments, the position of the structure of Formula (VIII), Formula (IX), or a mixture of Formula (VIII) and Formula (IX) in the amino acid sequence of the IL-2 conjugate of SEQ ID NO: 3 is at position R37. In some embodiments, the position of the structure of Formula (VIII), Formula (IX), or a mixture of Formula (VIII) and Formula (IX) in the amino acid sequence of the IL-2 conjugate of SEQ ID NO: 3 is at position T40. In some embodiments, the position of the structure of Formula (VIII), Formula (IX), or a mixture of Formula (VIII) and Formula (IX) in the amino acid sequence of the IL-2 conjugate of SEQ ID NO: 3 is at position E67. In some embodiments, the position of the structure of Formula (VIII), Formula (IX), or a mixture of Formula (VIII) and Formula (IX) in the amino acid sequence of the IL-2 conjugate of SEQ ID NO: 3 is at position Y44. In some embodiments, the position of the structure of Formula (VIII), Formula (IX), or a mixture of Formula (VIII) and Formula (IX) in the amino acid sequence of the IL-2 conjugate of SEQ ID NO: 3 is at position V68. In some embodiments, the position of the structure of Formula (VIII), Formula (IX), or a mixture of Formula (VIII) and Formula (IX) in the amino acid sequence of the IL-2 conjugate of SEQ ID NO: 3 is at position L71.

In some embodiments, the methods use an IL-2 conjugate in which the ratio of the amount of the structure of Formula (VIII) to the amount of the structure of Formula (IX) comprising the total amount of the IL-2 conjugate is about 1:1. In some embodiments, the methods use an IL-2 conjugate in which the ratio of the amount of the structure of Formula (VIII) to the amount of the structure of Formula (IX) comprising the total amount of the IL-2 conjugate is greater than 1:1. In some embodiments, the methods use an IL-2 conjugate in which the ratio of the amount of the structure of Formula (VIII) to the amount of the structure of Formula (IX) comprising the total amount of the IL-2 conjugate is less than 1:1.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VIII) and Formula (IX) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, 910, 1021, 1022, 1023, 1135, 1136, 1137, and 1249.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (VIII) and Formula (IX) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. In some embodiments, the methods use an IL-2 conjugate comprising the amino acid sequence of SEQ ID NO: 3 in which at least one amino acid residue in the IL-2 conjugate is replaced by the structure of Formula (VIII) or Formula (IX), or a mixture of Formula (VIII) and Formula (IX), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (X) or Formula (XI), or a mixture of Formula (X) and Formula (XI):

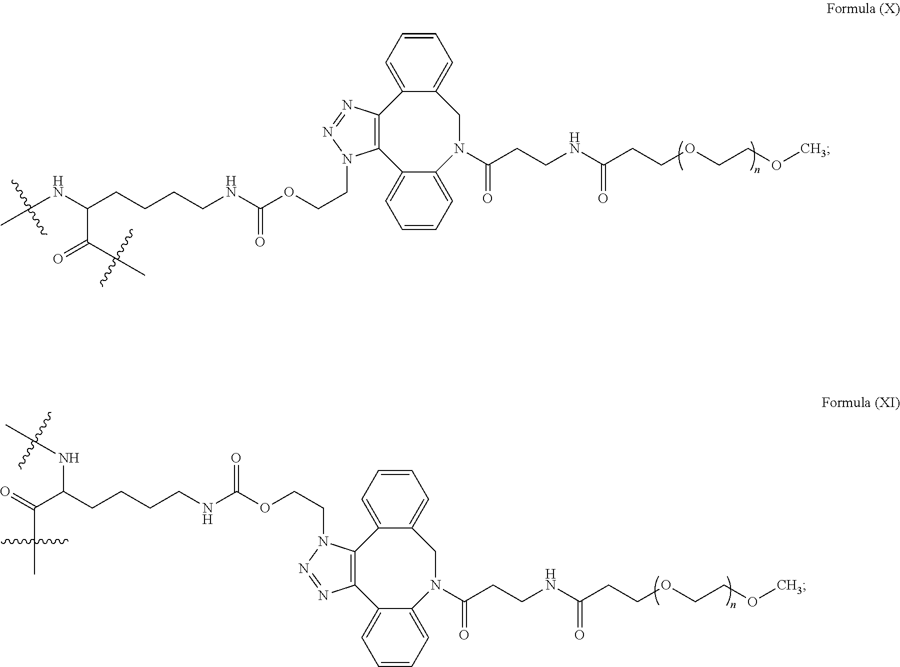

wherein:
n is an integer in the range from about 2 to about 5000; and
the wavy lines indicate covalent bonds to amino acid residues within SEQ ID NO: 3 that are not replaced.

Here and throughout, the structure of Formula (X) encompasses pharmaceutically acceptable salts, solvates, or hydrates thereof. Here and throughout, the structure of Formula (XI) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (X) and Formula (XI) is racemic, is enriched in (R), is enriched in (S), is substantially (R), is substantially (S), is (R) or is (S). In some embodiments, the stereochemistry of the chiral center within Formula (X) and Formula (XI) is racemic. In some embodiments, the stereochemistry of the chiral center within Formula (X) and Formula (XI) is enriched in (R). In some embodiments, the stereochemistry of the chiral center within Formula (X) and Formula (XI) is enriched in (S). In some embodiments, the stereochemistry of the chiral center within Formula (X) and Formula (XI) is substantially (R). In some embodiments, the stereochemistry of the chiral center within Formula (X) and Formula (XI) is substantially (S). In some embodiments, the stereochemistry of the chiral center within Formula (X) and Formula (XI) is (R). In some embodiments, the stereochemistry of the chiral center within Formula (X) and Formula (XI) is (S).

In some embodiments, n in the compounds of Formula (X) and Formula (XI) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (X) and Formula (XI) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (X) or Formula (XI), or a mixture of Formula (X) and Formula (XI), in the amino acid sequence of the IL-2 conjugate is selected from K34, F41, F43, K42, E61, P64, R37, T40, E67, Y44, V68, and L71, wherein the position of the structure of Formula (X), Formula (XI), or a mixture of Formula (X) and Formula (XI) in the amino acid sequence of the IL-2 conjugate is in reference to the positions in SEQ ID NO: 3. In some embodiments, the methods use an IL-2 conjugate in which the position of the structure of Formula (X) or Formula (XI), or a mixture of Formula (X) and Formula (XI), in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (X) or Formula (XI), or a mixture of Formula (X) and Formula (XI), in the amino acid sequence of the IL-2 conjugate of SEQ ID NO: 3 is at position K34. In some embodiments, the position of the structure of Formula (X) or Formula (XI), or a mixture of Formula (X) and Formula (XI), in the amino acid sequence of the IL-2 conjugate of SEQ ID NO: 3 is at position F41. In some embodiments, the methods use an IL-2 conjugate in which the position of the structure of Formula (X) or Formula (XI), or a mixture of Formula (X) and Formula (XI), in the amino acid sequence of the IL-2 conjugate of SEQ ID NO: 3 is at position F43. In some embodiments, the methods use an IL-2 conjugate in which the position of the structure of Formula (X) or Formula (XI), or a mixture of Formula (X) and Formula (XI), in the amino acid sequence of the IL-2 conjugate of SEQ ID NO: 3 is at position K42. In some embodiments, the methods use an IL-2 conjugate in which the position of the structure of Formula (X) or Formula (XI), or a mixture of Formula (X) and Formula (XI), in the amino acid sequence of the IL-2 conjugate of SEQ ID NO: 3 is at position E61. In some embodiments, the position of the structure of Formula (X) or Formula (XI), or a mixture of Formula (X) and Formula (XI), in the amino acid sequence of the IL-2 conjugate of SEQ ID NO: 3 is at position P64. In some embodiments, the position of the structure of Formula (X) or Formula (XI), or a mixture of Formula (X) and Formula (XI), in the amino acid sequence of the IL-2 conjugate of SEQ ID NO: 3 is at position R37. In some embodiments, the position of the structure of Formula (X) or Formula (XI), or a mixture of Formula (X) and Formula (XI), in the amino acid sequence of the IL-2 conjugate of SEQ ID NO: 3 is at position T40. In some embodiments, the position of the structure of Formula (X) or Formula (XI), or a mixture of Formula (X) and Formula (XI), in the amino acid sequence of the IL-2 conjugate of SEQ ID NO: 3 is at position E67. In some embodiments, the position of the structure of Formula (X) or Formula (XI), or a mixture of Formula (X) and Formula (XI), in the amino acid sequence of the IL-2 conjugate of SEQ ID NO: 3 is at position Y44. In some embodiments, the position of the structure of Formula (X) or Formula (XI), or a mixture of Formula (X) and Formula (XI), in the amino acid sequence of the IL-2 conjugate of SEQ ID NO: 3 is at position V68. In some embodiments, the position of the structure of Formula (X) or Formula (XI), or a mixture of Formula (X) and Formula (XI), in the amino acid sequence of the IL-2 conjugate of SEQ ID NO: 3 is at position L71.

In some embodiments, the methods use an IL-2 conjugate in which the ratio of the amount of the structure of Formula (X) to the amount of the structure of Formula (XI) comprising the total amount of the IL-2 conjugate is about 1:1. In some embodiments, the methods use an IL-2 conjugate in which the ratio of the amount of the structure of Formula (X) to the amount of the structure of Formula (XI) comprising the total amount of the IL-2 conjugate is greater than 1:1. In some embodiments, the methods use an IL-2 conjugate in which the ratio of the amount of the structure of Formula (X) to the amount of the structure of Formula (XI) comprising the total amount of the IL-2 conjugate is less than 1:1.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (VI) and Formula (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, 910, 1021, 1022, 1023, 1135, 1136, 1137, and 1249.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, and 910.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, the methods use an IL-2 conjugate in which n in the compounds of Formula (X) and Formula (XI) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (X) and Formula (XI) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, n in the structures of Formula (X) and Formula (XI) is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

In some embodiments, the methods use an IL-2 conjugate comprising the amino acid sequence of SEQ ID NO: 3 in which at least one amino acid residue in the IL-2 conjugate is replaced by the structure of Formula (X) or Formula (XI), or a mixture of Formula (X) and Formula (XI), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (XII) or Formula (XIII), or a mixture of Formula (XII) and Formula (XIII):

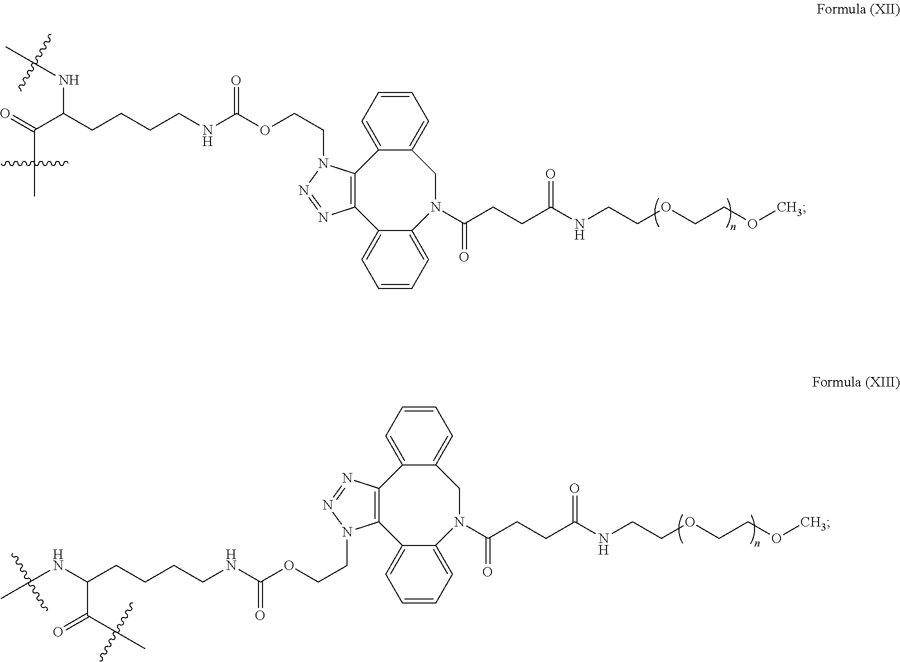

wherein:
n is an integer in the range from about 2 to about 5000; and
the wavy lines indicate covalent bonds to amino acid residues within SEQ ID NO: 3 that are not replaced.

Here and throughout, the structure of Formula (XII) encompasses pharmaceutically acceptable salts, solvates, or hydrates thereof. Here and throughout, the structure of Formula (XIII) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (XII) and Formula (XIII) is racemic, is enriched in (R), is enriched in (S), is substantially (R), is substantially (S), is (R) or is (S). In some embodiments, the stereochemistry of the chiral center within Formula (XII) and Formula (XIII) is racemic. In some embodiments, the stereochemistry of the chiral center within Formula (XII) and Formula (XIII) is enriched in (R). In some embodiments, the stereochemistry of the chiral center within Formula (XII) and Formula (XIII) is enriched in (S). In some embodiments, the stereochemistry of the chiral center within Formula (XII) and Formula (XIII) is substantially (R). In some embodiments, the stereochemistry of the chiral center within Formula (XII) and Formula (XIII) is substantially (S). In some embodiments, the stereochemistry of the chiral center within Formula (XII) and Formula (XIII) is (R). In some embodiments, the stereochemistry of the chiral center within Formula (XII) and Formula (XIII) is (S).

In some embodiments, n in the compounds of Formula (XII) and Formula (XIII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XII) and (XIII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the position of the structure of Formula (XII) or Formula (XIII), or a mixture of Formula (XII) and Formula (XIII), in the amino acid sequence of the IL-2 conjugate is selected from K34, F41, F43, K42, E61, P64, R37, T40, E67, Y44, V68, and L71, wherein the position of the structure of Formula (XII), Formula (XIII), or a mixture of Formula (XII) and Formula (XIII), in the amino acid sequence of the IL-2 conjugate is in reference to the positions in SEQ ID NO: 3. In some embodiments, the position of the structure of Formula (XII) or Formula (XIII), or a mixture of Formula (XII) and Formula (XIII), in the amino acid sequence of the IL-2 conjugate of SEQ ID NO: 3 is selected from K34, F41, F43, K42, E61, P64, R37, T40, E67, Y44, V68, and L71. In some embodiments, the position of the structure of Formula (XII) or Formula (XIII), or a mixture of Formula (XII) and Formula (XIII), in the amino acid sequence of the IL-2 conjugate of SEQ ID NO: 3 is at position K34. In some embodiments, the position of the structure of Formula (XII) or Formula (XIII), or a mixture of Formula (XII) and Formula (XIII), in the amino acid sequence of the IL-2 conjugate of SEQ ID NO: 3 is at position F41. In some embodiments, the position of the structure of Formula (XII) or Formula (XIII), or a mixture of Formula (XII) and Formula (XIII), in the amino acid sequence of the IL-2 conjugate of SEQ ID NO: 3 is at position F43. In some embodiments, the position of the structure of Formula (XII) or Formula (XIII), or a mixture of Formula (XII) and Formula (XIII), in the amino acid sequence of the IL-2 conjugate of SEQ ID NO: 3 is at position K42. In some embodiments, the position of the structure of Formula (XII) or Formula (XIII), or a mixture of Formula (XII) and Formula (XIII), in the amino acid sequence of the IL-2 conjugate of SEQ ID NO: 3 is at position E61. In some embodiments, the position of the structure of Formula (XII) or Formula (XIII), or a mixture of Formula (XII) and (XIII), in the amino acid sequence of the IL-2 conjugate of SEQ ID NO: 3 is at position P64. In some embodiments, the position of the structure of Formula (XII) or Formula (XIII), or a mixture of Formula (XII) and Formula (XIII), in the amino acid sequence of the IL-2 conjugate of SEQ ID NO: 3 is at position R37. In some embodiments, the position of the structure of Formula (XII) or Formula (XIII), or a mixture of Formula (XII) and Formula (XIII), in the amino acid sequence of the IL-2 conjugate of SEQ ID NO: 3 is at position T40. In some embodiments, the position of the structure of Formula (XII) or Formula (XIII), or a mixture of Formula (XII) and Formula (XIII), in the amino acid sequence of the IL-2 conjugate of SEQ ID NO: 3 is at position E67. In some embodiments, the position of the structure of Formula (XII) or Formula (XIII), or a mixture of Formula (XII) and Formula (XIII), in the amino acid sequence of the IL-2 conjugate of SEQ ID NO: 3 is at position Y44. In some embodiments, the position of the structure of Formula (XII) or Formula (XIII), or a mixture of Formula (XII) and Formula (XIII), in the amino acid sequence of the IL-2 conjugate of SEQ ID NO: 3 is at position V68. In some embodiments, the position of the structure of Formula (XII) or Formula (XIII), or a mixture of Formula (XII) and Formula (XIII), in the amino acid sequence of the IL-2 conjugate of SEQ ID NO: 3 is at position L71.

In some embodiments, the ratio of the amount of the structure of Formula (XII) to the amount of the structure of Formula (XIII) comprising the total amount of the IL-2 conjugate is about 1:1. In some embodiments, the ratio of the amount of the structure of Formula (XII) to the amount of the structure of Formula (XIII) comprising the total amount of the IL-2 conjugate is greater than 1:1. In some embodiments, the ratio of the amount of the structure of Formula (XII) to the amount of the structure of Formula (XIII) comprising the total amount of the IL-2 conjugate is less than 1:1.

In some embodiments described herein are IL-2 conjugates comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments, n in the compounds of Formula (XII) and Formula (XIII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments, the methods use an IL-2 conjugate comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, 910, 1021, 1022, 1023, 1135, 1136, 1137, and 1249.

In some embodiments, the methods use an IL-2 conjugate comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910.

In some embodiments, the methods use an IL-2 conjugate comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, the methods use an IL-2 conjugate comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments, n in the compounds of Formula (XII) and Formula (XIII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, n in the structures of Formula (XII) or Formula (XIII), or a mixture of Formula (XII) and Formula (XIII), is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

In some embodiments, the methods use an IL-2 conjugate comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-2 conjugates comprising the amino acid sequence of SEQ ID NO: 3 in which at least one amino acid residue in the IL-2 conjugate is replaced by the structure of Formula (XII) or Formula (XIII), or a mixture of Formula (XII) and Formula (XIII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Described herein are methods of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VIII) or Formula (IX), or a mixture of Formula (VIII) and Formula (IX):

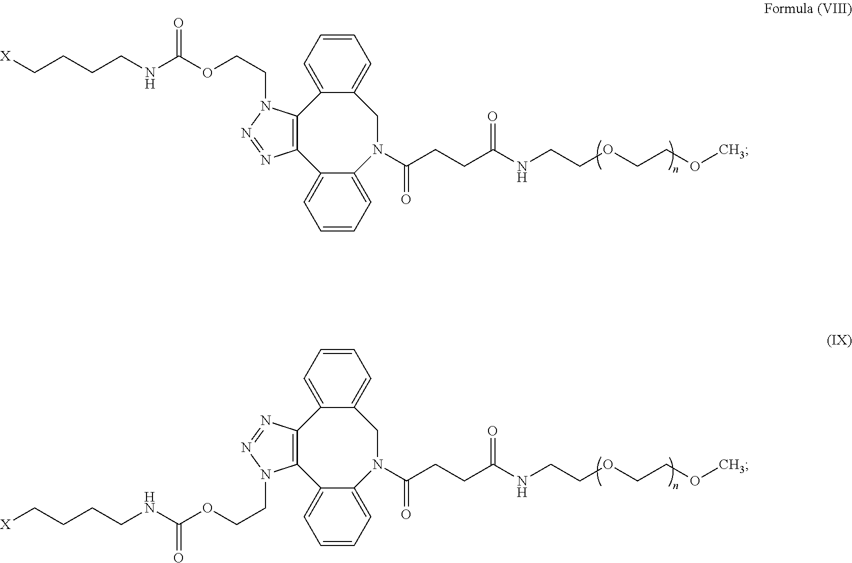

wherein:
n is an integer such that the molecular weight of the PEG group is from about 15,000 Daltons to about 60,000 Daltons; and
X has the structure:

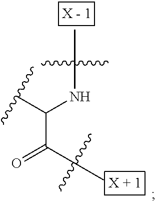

X−1 indicates the point of attachment to the preceding amino acid residue; and
X+1 indicates the point of attachment to the following amino acid residue.

In some embodiments, the IL-2 conjugate is a pharmaceutically acceptable salt, solvate, or hydrate.

In some embodiments, the amino acid residue at E61 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the one or more PD-1 inhibitors is pembrolizumab, nivolumab, or cemiplimab. In some embodiments, the one or more PD-1 inhibitors is pembrolizumab. In some embodiments, the one or more PD-1 inhibitors is nivolumab. In some embodiments, the one or more PD-1 inhibitors is cemiplimab.

In some embodiments, the amino acid residue at P64 in the IL-2 conjugate is replaced by the structure of Formula (VIII) or Formula (IX), or a mixture of Formula (VIII) and Formula (IX), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the one or more PD-1 inhibitors is pembrolizumab, nivolumab, or cemiplimab. In some embodiments, the one or more PD-1 inhibitors is pembrolizumab. In some embodiments, the one or more PD-1 inhibitors is nivolumab. In some embodiments, the one or more PD-1 inhibitors is cemiplimab.

Described herein are methods of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VI) or Formula (VII), or a mixture of Formula (VI) and Formula (VII):

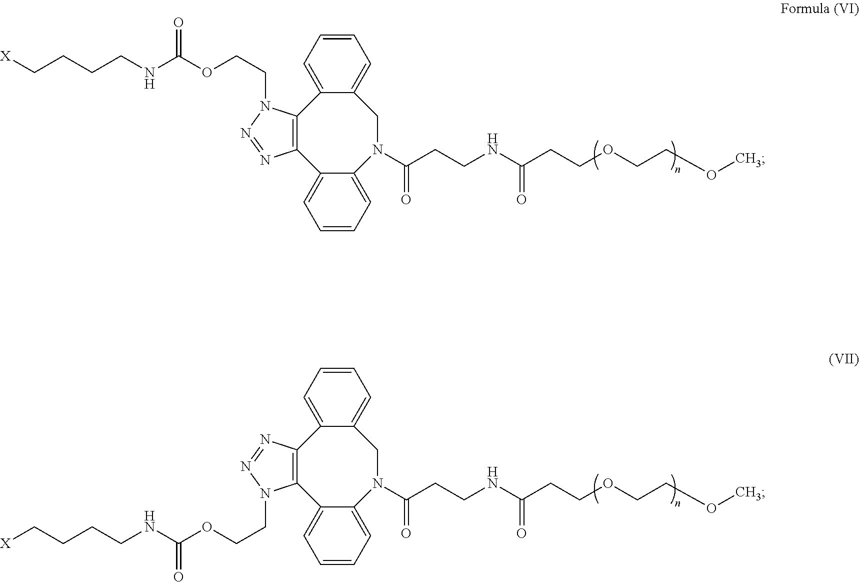

wherein:
n is an integer such that the molecular weight of the PEG group is from about 15,000 Daltons to about 60,000 Daltons; and
X has the structure:

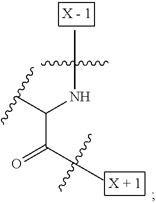

X−1 indicates the point of attachment to the preceding amino acid residue; and
X+1 indicates the point of attachment to the following amino acid residue.
In some embodiments, the IL-2 conjugate is a pharmaceutically acceptable salt, solvate, or hydrate.

In some embodiments, the amino acid residue at E61 in the IL-2 conjugate is replaced by the structure of Formula (VI) or Formula (VII), or a mixture of Formula (VI) and Formula (VII), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the one or more PD-1 inhibitors is pembrolizumab, nivolumab, or cemiplimab. In some embodiments, the one or more PD-1 inhibitors is pembrolizumab. In some embodiments, the one or more PD-1 inhibitors is nivolumab. In some embodiments, the one or more PD-1 inhibitors is cemiplimab.

In some embodiments, the amino acid residue at P64 in the IL-2 conjugate is replaced by the structure of Formula (VI) or Formula (VII), or a mixture of Formula (VI) and Formula (VII), and wherein n is an integer such that the molecular weight of the PEG group is from about 20,000 Daltons to about 40,000 Daltons. In some embodiments, n is an integer such that the molecular weight of the PEG group is from about 30,000 Daltons. In some embodiments, the one or more PD-1 inhibitors is pembrolizumab, nivolumab, or cemiplimab. In some embodiments, the one or more PD-1 inhibitors is pembrolizumab. In some embodiments, the one or more PD-1 inhibitors is nivolumab. In some embodiments, the one or more PD-1 inhibitors is cemiplimab.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprising the amino acid sequence of SEQ ID NO: 1, SEQ ID NO: 3, or SEQ ID NO: 4 in which at least one amino acid residue in the IL-2 conjugate is replaced by a cysteine covalently bonded to a PEG group. In some embodiments, the PEG group has a molecular weight selected from 5 kDa, 10 kDa, 15 kDa, 20 kDa, 25 kDa, 30 kDa, 35 kDa, 40 kDa, 45 kDa, 50 kDa, and 60 kDa. In some embodiments, the PEG group has a molecular weight of 5 kDa. In some embodiments, the PEG group has a molecular weight of 10 kDa. In some embodiments, the PEG group has a molecular weight of 15 kDa. In some embodiments, the PEG group has a molecular weight of 20 kDa. In some embodiments, the PEG group has a molecular weight of 25 kDa. In some embodiments, the PEG group has a molecular weight of 30 kDa. In some embodiments, the PEG group has a molecular weight of 35 kDa. In some embodiments, the PEG group has a molecular weight of 40 kDa. In some embodiments, the PEG group has a molecular weight of 45 kDa. In some embodiments, the PEG group has a molecular weight of 50 kDa. In some embodiments, the PEG group has a molecular weight of 60 kDa. In some embodiments, the IL-2 conjugate comprises the amino acid sequence of SEQ ID NO: 3 and the at least one amino acid residue in the IL-2 conjugate that is replaced by a cysteine is selected from K34, T36, R37, T40, F41, K42, F43, Y44, E60, E61, E67, K63, P64, V68, L71, and Y106. In some embodiments, the IL-2 conjugate comprises the amino acid sequence of SEQ ID NO: 3 and the at least one amino acid residue in the IL-2 conjugate that is replaced by a cysteine is selected from K34, T40, F41, K42, Y44, E60, E61, E67, K63, P64, V68, and L71. In some embodiments, the IL-2 conjugate comprises the amino acid sequence of SEQ ID NO: 4 and the at least one amino acid residue in the IL-2 conjugate that is replaced by a cysteine is selected from K35, T37, R38, T41, F42, K43, F44, Y45, E61, E62, E68, K64, P65, V69, L72, and Y107.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprising the amino acid sequence of SEQ ID NO: 3 in which at least one non-lysine residue is replaced by a lysine comprising a linker and a water-soluble polymer. In some embodiments, the water-soluble polymer is a PEG group.

In some embodiments, the IL-2 conjugate comprises a PEG group covalently bonded via a non-releasable linkage. In some embodiments, the IL-2 conjugate comprises a non-releasable, covalently bonded PEG group.

Described herein are methods of treating cancer in a subject, comprising administering to a subject in need thereof a therapeutically effective amount of (a) an IL-2 conjugate, and (b) one or more additional agents, wherein the IL-2 conjugate having SEQ ID NO: 3 wherein a non-lysine amino acid in the IL-2 conjugate is replaced by a lysine residue, and wherein the lysine residue comprises one or more water soluble polymers and a covalent linker. In some embodiments, the lysine residue is located in the region K34-Y106 of SEQ ID NO: 3. In some embodiments, the lysine residue is located at K34. In some embodiments, the lysine residue is located at F41. In some embodiments, the lysine residue is located at F43. In some embodiments, the lysine residue is located at K42. In some embodiments, the lysine residue is located at E61. In some embodiments, the lysine residue is located at P64. In some embodiments, the lysine residue is located at R37. In some embodiments, the lysine residue is located at T40. In some embodiments, the lysine residue is located at E67. In some embodiments, the lysine residue is located at Y44. In some embodiments, the lysine residue is located at V68. In some embodiments, the lysine residue is located at L71.

Described herein are methods of treating cancer in a subject, comprising administering to a subject in need thereof a therapeutically effective amount of (a) an IL-2 conjugate, and (b) one or more additional agents, wherein the IL-2 conjugate having SEQ ID NO: 3 wherein a non-lysine amino acid in the IL-2 conjugate is replaced by a lysine residue, and wherein the lysine residue comprises one or more water soluble polymers and a covalent linker. In some embodiments, the lysine residue is located in the region K34-Y106 of SEQ ID NO: 3. In some embodiments, the lysine residue is located at K34. In some embodiments, the lysine residue is located at F41. In some embodiments, the lysine residue is located at F43. In some embodiments, the lysine residue is located at K42. In some embodiments, the lysine residue is located at E61. In some embodiments, the lysine residue is located at P64. In some embodiments, the lysine residue is located at R37. In some embodiments, the lysine residue is located at T40. In some embodiments, the lysine residue is located at E67. In some embodiments, the lysine residue is located at Y44. In some embodiments, the lysine residue is located at V68. In some embodiments, the lysine residue is located at L71.

Described herein are methods of treating cancer in a subject, comprising administering to a subject in need thereof a therapeutically effective amount of (a) an IL-2 conjugate, and (b) one or more additional agents, wherein the IL-2 conjugate is an interleukin-2 (IL-2) variant wherein a non-lysine amino acid in the amino acid sequence of the IL-2 variant is replaced by an amino acid comprising: (a) a lysine; (b) a covalent linker; and (3) and one or more water-soluble polymers. In some embodiments, one or more water-soluble polymers comprises a PEG group.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV):

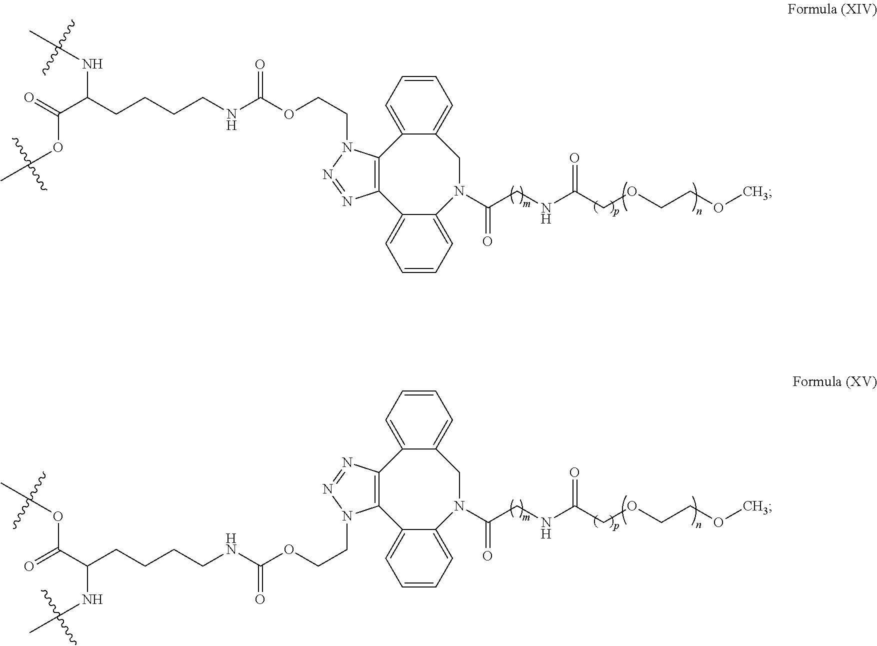

wherein:
m is an integer from 0 to 20;
p is an integer from 0 to 20;
n is an integer in the range from about 2 to about 5000; and
the wavy lines indicate covalent bonds to amino acid residues within SEQ ID NO: 3 that are not replaced.

Here and throughout, the structure of Formula (XIV) encompasses pharmaceutically acceptable salts, solvates, or hydrates thereof. Here and throughout, the structure of Formula (XV) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (XIV) and Formula (XV) is racemic, is enriched in (R), is enriched in (S), is substantially (R), is substantially (S), is (R) or is (S). In some embodiments, the stereochemistry of the chiral center within Formula (XIV) and Formula (XV) is racemic. In some embodiments, the stereochemistry of the chiral center within Formula (XIV) and Formula (XV) is enriched in (R). In some embodiments, the stereochemistry of the chiral center within Formula (XIV) and Formula (XV) is enriched in (S). In some embodiments, the stereochemistry of the chiral center within Formula (XIV) and Formula (XV) is substantially (R). In some embodiments, the stereochemistry of the chiral center within Formula (XIV) and Formula (XV) is substantially (S). In some embodiments, the stereochemistry of the chiral center within Formula (XIV) and Formula (XV) is (R). In some embodiments, the stereochemistry of the chiral center within Formula (XIV) and Formula (XV) is (S).

In some embodiments, the methods use an IL-2 conjugate comprising the amino acid sequence of SEQ ID NO: 3 in which m in the compounds of Formula (XIV) and Formula (XV) is from 0 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-2 conjugate described herein, m in the compounds of Formula (XIV) and Formula (XV) is 1. In some embodiments of an IL-2 conjugate described herein, m in the compounds of Formula (XIV) and Formula (XV) is 2. In some embodiments of an IL-2 conjugate described herein, m in the compounds of Formula (XIV) and Formula (XV) is 3. In some embodiments of an IL-2 conjugate described herein, m in the compounds of Formula (XIV) and Formula (XV) is 4. In some embodiments of an IL-2 conjugate described herein, m in the compounds of Formula (XIV) and Formula (XV) is 5. In some embodiments of an IL-2 conjugate described herein, m in the compounds of Formula (XIV) and Formula (XV) is 6. In some embodiments of an IL-2 conjugate described herein, m in the compounds of Formula (XIV) and Formula (XV) is 7. In some embodiments of an IL-2 conjugate described herein, m in the compounds of Formula (XIV) and Formula (XV) is 8. In some embodiments of an IL-2 conjugate described herein, m in the compounds of Formula (XIV) and Formula (XV) is 9. In some embodiments of an IL-2 conjugate described herein, m in the compounds of Formula (XIV) and Formula (XV) is 10. In some embodiments of an IL-2 conjugate described herein, m in the compounds of Formula (XIV) and Formula (XV) is 11. In some embodiments of an IL-2 conjugate described herein, m in the compounds of Formula (XIV) and Formula (XV) is 12. In some embodiments of an IL-2 conjugate described herein, m in the compounds of Formula (XIV) and Formula (XV) is 13. In some embodiments of an IL-2 conjugate described herein, m in the compounds of Formula (XIV) and Formula (XV) is 14. In some embodiments of an IL-2 conjugate described herein, m in the compounds of Formula (XIV) and Formula (XV) is 15. In some embodiments of an IL-2 conjugate described herein, m in the compounds of Formula (XIV) and Formula (XV) is 16. In some embodiments of an IL-2 conjugate described herein, m in the compounds of Formula (XIV) and Formula (XV) is 17. In some embodiments of an IL-2 conjugate described herein, m in the compounds of Formula (XIV) and Formula (XV) is 18. In some embodiments of an IL-2 conjugate described herein, m in the compounds of Formula (XIV) and Formula (XV) is 19. In some embodiments of an IL-2 conjugate described herein, m in the compounds of Formula (XIV) and Formula (XV) is 20.

In some embodiments, the methods use an IL-2 conjugate comprising the amino acid sequence of SEQ ID NO: 3 in which p in the compounds of Formula (XIV) and Formula (XV) is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-2 conjugate described herein, p in the compounds of Formula (XIV) and Formula (XV) is 1. In some embodiments of an IL-2 conjugate described herein, p in the compounds of Formula (XIV) and Formula (XV) is 2. In some embodiments of an IL-2 conjugate described herein, p in the compounds of Formula (XIV) and Formula (XV) is 3. In some embodiments of an IL-2 conjugate described herein, p in the compounds of Formula (XIV) and Formula (XV) is 4. In some embodiments of an IL-2 conjugate described herein, p in the compounds of Formula (XIV) and Formula (XV) is 5. In some embodiments of an IL-2 conjugate described herein, p in the compounds of Formula (XIV) and Formula (XV) is 6. In some embodiments of an IL-2 conjugate described herein, p in the compounds of Formula (XIV) and Formula (XV) is 7. In some embodiments of an IL-2 conjugate described herein, p in the compounds of Formula (XIV) and Formula (XV) is 8. In some embodiments of an IL-2 conjugate described herein, p in the compounds of Formula (XIV) and Formula (XV) is 9. In some embodiments of an IL-2 conjugate described herein, p in the compounds of Formula (XIV) and Formula (XV) is 10. In some embodiments of an IL-2 conjugate described herein, p in the compounds of Formula (XIV) and Formula (XV) is 11. In some embodiments of an IL-2 conjugate described herein, p in the compounds of Formula (XIV) and Formula (XV) is 12. In some embodiments of an IL-2 conjugate described herein, p in the compounds of Formula (XIV) and Formula (XV) is 13. In some embodiments of an IL-2 conjugate described herein, p in the compounds of Formula (XIV) and Formula (XV) is 14. In some embodiments of an IL-2 conjugate described herein, p in the compounds of Formula (XIV) and Formula (XV) is 15. In some embodiments of an IL-2 conjugate described herein, m in the compounds of Formula (XIV) and Formula (XV) is 16. In some embodiments of an IL-2 conjugate described herein, p in the compounds of Formula (XIV) and Formula (XV) is 17. In some embodiments of an IL-2 conjugate described herein, p in the compounds of Formula (XIV) and Formula (XV) is 18. In some embodiments of an IL-2 conjugate described herein, p in the compounds of Formula (XIV) and Formula (XV) is 19. In some embodiments of an IL-2 conjugate described herein, p in the compounds of Formula (XIV) and Formula (XV) is 20.

In some embodiments, the methods use an IL-2 conjugate comprising the amino acid sequence of SEQ ID NO: 3 in which n in the compounds of Formula (XIV) and Formula (XV) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments, the methods use an IL-2 conjugate comprising the amino acid sequence of SEQ ID NO: 3 in which m in the compounds of Formula (XIV) and Formula (XV) is an integer from 1 to 6, p is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is an integer from 2 to 6, p is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is an integer from 2 to 4, p is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 1, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 2, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 3, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 4, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 5, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 6, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 7, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 8, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 9, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 10, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 11, p is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XIV) and Formula (XV), m is 2, p is 2, and n is an integer selected from 680, 681, 682, 794, 795, 796, 908, 909, 910, 1021, 1022, 1023, 1135, 1136, and 1137.

In some embodiments, the methods use an IL-2 conjugate comprising the amino acid sequence of SEQ ID NO: 3 in which n in the compounds of Formula (XIV) and Formula (XV) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments of an IL-2 conjugate described herein, the position of the structure of Formula (XIV) or Formula (XV), or a mixture of Formula (XIV) and Formula (XV), in the amino acid sequence of the IL-2 conjugate is selected from K34, F41, F43, K42, E61, P64, R37, T40, E67, Y44, V68, and L71, wherein the position of the structure of Formula (XIV), Formula (XV), or a mixture of Formula (XIV) and Formula (XV) in the amino acid sequence of the IL-2 conjugate is in reference to the positions in SEQ ID NO: 3. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is selected from K34, F41, F43, K42, E61, P64, R37, T40, E67, Y44, V68, and L71. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position K34. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position F41. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position F43. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position K42. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position E61. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position P64. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position R37. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position T40. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position E67. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position Y44. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position V68. In some embodiments of an IL-2 conjugate described herein, the position of the structure of Formula (XIV), Formula (XV), or a mixture of Formula (XIV) and Formula (XV) in the amino acid sequence of the IL-2 conjugate of SEQ ID NO: 3 is at position L71.

In some embodiments of an IL-2 conjugate described herein, the ratio of the amount of the structure of Formula (XIV) to the amount of the structure of Formula (XV) comprising the total amount of the IL-2 conjugate is about 1:1. In some embodiments of an IL-2 conjugate described herein, the ratio of the amount of the structure of Formula (XIV) to the amount of the structure of Formula (XV) comprising the total amount of the IL-2 conjugate is greater than 1:1. In some embodiments of an IL-2 conjugate described herein, the ratio of the amount of the structure of Formula (XIV) to the amount of the structure of Formula (XV) comprising the total amount of the IL-2 conjugate is less than 1:1.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-2 conjugate described herein, n in the compounds of Formula (XIV) and Formula (XV) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909. In some embodiments of an IL-2 conjugate described herein, n in the compounds of Formula (XIV) and Formula (XV) is an integer selected from 454, 455, 568, 569, 680, 681, 682, 794, 795, 796, 908, 909, 910, 1021, 1022, 1023, 1135, 1136, 1137, and 1249.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909. In some embodiments of an IL-2 conjugate described herein, n in the compounds of Formula (XIV) and Formula (XV) is an integer selected from 454, 455, 568, 569, 680, 681, 682, 794, 795, 796, 908, 909, and 910.

Described herein are methods of treating cancer in a subject, comprising administering to a subject in need thereof a therapeutically effective amount of (a) an IL-2 conjugate, and (b) one or more additional agents, wherein the IL-2 conjugate is an IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is E61, and wherein n is an integer from about 450 to about 800, or from about 454 to about 796, or from about 454 to about 682, or from about 568 to about 909. In some embodiments of an IL-2 conjugate described herein, n in the compounds of Formula (XIV) and Formula (XV) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is P64, and wherein n is an integer from about 450 to about 800, or from about 454 to about 796, or from about 454 to about 682, or from about 568 to about 909. In some embodiments of an IL-2 conjugate described herein, n in the compounds of Formula (XIV) and Formula (XV) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Described herein are IL-2 conjugates comprising the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

Described herein are IL-2 conjugates comprising the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or (XV), or a mixture of (XIV) and (XV), wherein the amino acid residue in SEQ ID NO: 3 that is replaced is selected from F41, F43, K42, E61, and P64, m is an integer from 1 to 6, p is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XIV) and (XV), m is 2, p is 2, and n is an integer selected from 454, 455, 568, 569, 680, 681, 682, 794, 795, 796, 908, 909, 910, 1021, 1022, 1023, 1135, 1136, 1137, and 1249.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is selected from E61 and P64, and wherein m is an integer from 1 to 6, p is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XIV) and Formula (XV), m is 2, p is 2, and n is an integer selected from 454, 455, 568, 569, 680, 681, 682, 794, 795, 796, 908, 909, and 910.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is E61, and wherein m is an integer from 1 to 6, p is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XIV) and Formula (XV), m is 2, p is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XIV) or Formula (XV), or a mixture of Formula (XIV) and Formula (XV), wherein the amino acid residue in SEQ ID NO: 3 that is replaced is P64, and wherein m is an integer from 1 to 6, p is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XIV) and Formula (XV), m is 2, p is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

Described herein are methods of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XVI) or Formula (XVII), or a mixture of Formula (XVI) and Formula (XVII):

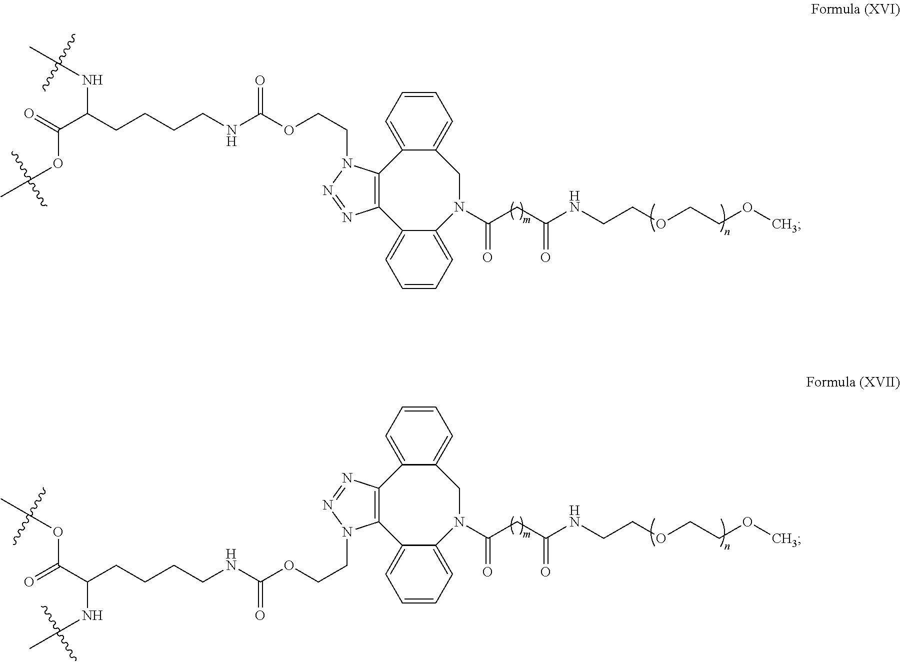

wherein:
m is an integer from 0 to 20;
n is an integer in the range from about 2 to about 5000; and
the wavy lines indicate covalent bonds to amino acid residues within SEQ ID NO: 3 that are not replaced.

Here and throughout, the structure of Formula (XVI) encompasses pharmaceutically acceptable salts, solvates, or hydrates thereof. Here and throughout, the structure of Formula (XVII) encompasses pharmaceutically acceptable salts, solvates, or hydrates thereof. In some embodiments, the IL-2 conjugate is a pharmaceutically acceptable salt, solvate, or hydrate.

In some embodiments, the stereochemistry of the chiral center within Formula (XVI) and Formula (XVII) is racemic, is enriched in (R), is enriched in (S), is substantially (R), is substantially (S), is (R) or is (S). In some embodiments, the stereochemistry of the chiral center within Formula (XVI) and Formula (XVII) is racemic. In some embodiments, the stereochemistry of the chiral center within Formula (XVI) and Formula (XVII) is enriched in (R). In some embodiments, the stereochemistry of the chiral center within Formula (XVI) and Formula (XVII) is enriched in (S). In some embodiments, the stereochemistry of the chiral center within Formula (XVI) and Formula (XVII) is substantially (R). In some embodiments, the stereochemistry of the chiral center within Formula (XVI) and Formula (XVII) is substantially (S). In some embodiments, the stereochemistry of the chiral center within Formula (XVI) and Formula (XVII) is (R). In some embodiments, the stereochemistry of the chiral center within Formula (XVI) and Formula (XVII) is (S).

In some embodiments, the methods use an IL-2 conjugate comprising the amino acid sequence of SEQ ID NO: 3 in which m in the compounds of Formula (XVI) and Formula (XVII) is from 1 to 20, or from 1 to 18, or from 1 to 16, or from 1 to 14, or from 1 to 12, or from 1 to 10, or from 1 to 9, or from 1 to 8, or from 1 to 7, or from 1 to 6, or from 1 to 5, or from 1 to 4, or from 1 to 3, or from 1 to 2. In some embodiments of an IL-2 conjugate described herein, m in the compounds of Formula (XVI) and Formula (XVII) is 1. In some embodiments of an IL-2 conjugate described herein, m in the compounds of Formula (XVI) and Formula (XVII) is 2. In some embodiments of an IL-2 conjugate described herein, m in the compounds of Formula (XVI) and Formula (XVII) is 3. In some embodiments of an IL-2 conjugate described herein, m in the compounds of Formula (XVI) and Formula (XVII) is 4. In some embodiments of an IL-2 conjugate described herein, m in the compounds of Formula (XVI) and Formula (XVII) is 5. In some embodiments of an IL-2 conjugate described herein, m in the compounds of Formula (XVI) and Formula (XVII) is 6. In some embodiments of an IL-2 conjugate described herein, m in the compounds of Formula (XVI) and Formula (XVII) is 7. In some embodiments of an IL-2 conjugate described herein, m in the compounds of Formula (XVI) and Formula (XVII) is 8. In some embodiments of an IL-2 conjugate described herein, m in the compounds of Formula (XVI) and Formula (XVII) is 9. In some embodiments of an IL-2 conjugate described herein, m in the compounds of Formula (XVI) and Formula (XVII) is 10. In some embodiments of an IL-2 conjugate described herein, m in the compounds of Formula (XVI) and Formula (XVII) is 11. In some embodiments of an IL-2 conjugate described herein, m in the compounds of Formula (XVI) and Formula (XVII) is 12. In some embodiments of an IL-2 conjugate described herein, m in the compounds of Formula (XVI) and Formula (XVII) is 13. In some embodiments of an IL-2 conjugate described herein, m in the compounds of Formula (XVI) and Formula (XVII) is 14. In some embodiments of an IL-2 conjugate described herein, m in the compounds of Formula (XVI) and Formula (XVII) is 15. In some embodiments of an IL-2 conjugate described herein, m in the compounds of Formula (XVI) and Formula (XVII) is 16. In some embodiments of an IL-2 conjugate described herein, m in the compounds of Formula (XVI) and Formula (XVII) is 17. In some embodiments of an IL-2 conjugate described herein, m in the compounds of Formula (XVI) and Formula (XVII) is 18. In some embodiments of an IL-2 conjugate described herein, m in the compounds of Formula (XVI) and Formula (XVII) is 19. In some embodiments of an IL-2 conjugate described herein, m in the compounds of Formula (XVI) and Formula (XVII) is 20.

In some embodiments, the methods use an IL-2 conjugate comprising the amino acid sequence of SEQ ID NO: 3 in which n in the compounds of Formula (XVI) and Formula (XVII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

In some embodiments, the methods use an IL-2 conjugate comprising the amino acid sequence of SEQ ID NO: 3 in which m in the compounds of Formula (XVI) and Formula (XVII) is an integer from 1 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is an integer from 2 to 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is an integer from 2 to 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 1, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 2, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 3, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 4, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 5, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 6, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 7, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 8, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 9, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVI), m is 10, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 11, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 12, and n is an integer selected from 113, 114, 227, 228, 340, 341, 454, 455, 568, 569, 680, 681, 682, 794, 795, 796, 908, 909, 910, 1021, 1022, 1023, 1135, 1136, and 1137. In some embodiments of an IL-2 conjugate described herein in the compounds of Formula (XVI) and Formula (XVII), m is 2, and n is an integer selected from 680, 681, 682, 794, 795, 796, 908, 909, 910, 1021, 1022, 1023, 1135, 1136, and 1137.

In some embodiments, the methods use an IL-2 conjugate comprising the amino acid sequence of SEQ ID NO: 3 in which n in the compounds of Formula (XVI) and Formula (XVII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546. In some embodiments of an IL-2 conjugate described herein, the position of the structure of Formula (XVI) or Formula (XVII), or a mixture of Formula (XVI) and Formula (XVII), in the amino acid sequence of the IL-2 conjugate is selected from K34, F41, F43, K42, E61, P64, R37, T40, E67, Y44, V68, and L71, wherein the position of the structure of Formula (I) in the amino acid sequence of the IL-2 conjugate is in reference to the positions in SEQ ID NO: 3. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is selected from K34, F41, F43, K42, E61, P64, R37, T40, E67, Y44, V68, and L71. In some embodiments of an IL-2 conjugate described herein, the position of the structure of Formula (XVI) or Formula (XVII), or a mixture of Formula (XVI) and Formula (XVII) in the amino acid sequence of the IL-2 conjugate of SEQ ID NO: 3 is at position K34. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position F41. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position F43. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position K42. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position E61. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position P64. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position R37. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position T40. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position E67. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position Y44. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position V68. In some embodiments of an IL-2 conjugate described herein, the position of the structure of Formula (XVI) or Formula (XVII), or a mixture of Formula (XVI) and Formula (XVII), in the amino acid sequence of the IL-2 conjugate of SEQ ID NO: 3 is at position L71.

In some embodiments of an IL-2 conjugate described herein, the ratio of the amount of the structure of Formula (XVI) to the amount of the structure of Formula (XVII) comprising the total amount of the IL-2 conjugate is about 1:1. In some embodiments of an IL-2 conjugate described herein, the ratio of the amount of the structure of Formula (XVI) to the amount of the structure of Formula (XVII) comprising the total amount of the IL-2 conjugate is greater than 1:1. In some embodiments of an IL-2 conjugate described herein, the ratio of the amount of the structure of Formula (XVI) to the amount of the structure of Formula (XVII) comprising the total amount of the IL-2 conjugate is less than 1:1.

In some embodiments, the methods use an IL-2 conjugate comprising the amino acid sequence of SEQ ID NO: 3 in which the at least one amino acid residue in the IL-2 conjugate is replaced by the structure of Formula (XVI) or Formula (XVII), or a mixture of Formula (XVI) and Formula (XVII), is selected from K34, F41, F43, K42, E61, P64, R37, T40, E67, Y44, V68, and L71, and wherein n is an integer from 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575. In some embodiments of an IL-2 conjugate described herein, n in the compounds of Formula (XVI) and Formula (XVII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), is selected from F41, F43, K42, E61, and P64, and wherein n is an integer from about 450 to about 800, or from about 454 to about 796, or from about 454 to about 682, or from about 568 to about 909. In some embodiments of an IL-2 conjugate described herein, n in the compounds of Formula (XVI) and Formula (XVII) is an integer selected from 454, 455, 568, 569, 680, 681, 682, 794, 795, 796, 908, 909, 910, 1021, 1022, 1023, 1135, 1136, 1137, and 1249.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), is selected from E61 and P64, and wherein n is an integer from about 450 to about 800, or from about 454 to about 796, or from about 454 to about 682, or from about 568 to about 909. In some embodiments of an IL-2 conjugate described herein, n in the compounds of Formula (XVI) and Formula (XVII) is an integer selected from 454, 455, 568, 569, 680, 681, 682, 794, 795, 796, 908, 909, and 910.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), that is replaced is E61, and wherein n is an integer from about 450 to about 800, or from about 454 to about 796, or from about 454 to about 682, or from about 568 to about 909. In some embodiments of an IL-2 conjugate described herein, n in the compounds of Formula (XVI) and Formula (XVII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments described herein are IL-2 conjugates comprising the amino acid sequence of SEQ ID NO: 3 in which the at least one amino acid residue in the IL-2 conjugate is replaced by the structure of Formula (XVI) or Formula (XVII), or a mixture of Formula (XVI) and Formula (XVII), that is replaced is P64, and wherein n is an integer from about 450 to about 800, or from about 454 to about 796, or from about 454 to about 682, or from about 568 to about 909. In some embodiments of an IL-2 conjugate described herein, n in the compounds of Formula (XVI) and Formula (XVII) is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Described herein are IL-2 conjugates comprising the amino acid sequence of SEQ ID NO: 3 in which at least one amino acid residue in the IL-2 conjugate is replaced by the structure of Formula (XVI) or Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons. Described herein are IL-2 conjugates comprising the amino acid sequence of SEQ ID NO: 3 in which at least one amino acid residue in the IL-2 conjugate is replaced by the structure of Formula (XVI) or Formula (XVII), or a mixture of Formula (XVI) and Formula (XVII), wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

In some embodiments described herein are IL-2 conjugates comprising the amino acid sequence of SEQ ID NO: 3 in which the at least one amino acid residue in the IL-2 conjugate is replaced by the structure of Formula (XVI) or Formula (XVII), or a mixture of Formula (XVI) and Formula (XVII), is selected from F41, F43, K42, E61, and P64, m is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XVI) and Formula (XVII), m is 2, and n is an integer selected from 454, 455, 568, 569, 680, 681, 682, 794, 795, 796, 908, 909, 910, 1021, 1022, 1023, 1135, 1136, 1137, and 1249.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), is selected from E61 and P64, and wherein m is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XVI) and Formula (XVII), m is 2, and n is an integer selected from 454, 455, 568, 569, 680, 681, 682, 794, 795, 796, 908, 909, and 910.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), that is replaced is E61, and wherein m is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XVI) and Formula (XVII), m is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments described herein are IL-2 conjugates comprising the amino acid sequence of SEQ ID NO: 3 in which the at least one amino acid residue in the IL-2 conjugate replaced by the structure of Formula (XVI) or Formula (XVII), or a mixture of Formula (XVI) and Formula (XVII), is P64, and wherein m is an integer from 1 to 6, and n is an integer from about 450 to about 800, or from about 454 to about 796, or from about 454 to about 682, or from about 568 to about 909. In some embodiments of an IL-2 conjugate described herein in the compounds of Formula (XVI) and Formula (XVII), m is 2, and n is an integer selected from 454, 455, 568, 569, 680, 681, 682, 794, 795, 796, 908, 909, and 910. In some embodiments, n is from about 500 to about 1000. In some embodiments, n is from about 550 to about 800. In some embodiments, n is about 681.

In some embodiments, described herein is a method of treating a proliferative disease or condition in a subject in need thereof, which comprises administering to the subject a therapeutically effective amount of (a) a cytokine conjugate (e.g., an IL-2 conjugate) described Table 1, and (b) one or more additional agents. In some embodiments, the IL-2 conjugate comprises SEQ ID NOs.: 1-98. In some embodiments, the IL-2 conjugate comprises SEQ ID NOs.: 1-84. In some embodiments, the IL-2 conjugate comprises SEQ ID NOs.: 15-29. In some embodiments, the IL-2 conjugate comprises SEQ ID NOs.: 40-54. In some embodiments, the IL-2 conjugate comprises SEQ ID NOs.: 55-69. In some embodiments, the IL-2 conjugate comprises SEQ ID NOs.: 70-84. In some embodiments, the IL-2 conjugate comprises SEQ ID NOs.: 85-98. In some embodiments, the IL-2 conjugate comprises SEQ ID NO: 1. In some embodiments, the IL-2 conjugate comprises SEQ ID NO: 2. In some embodiments, the IL-2 conjugate comprises SEQ ID NO: 3. In some embodiments, the IL-2 conjugate comprises SEQ ID NO: 4. In some embodiments, the IL-2 conjugate comprises SEQ ID NO: 5. In some embodiments, the IL-2 conjugate comprises SEQ ID NO: 6. In some embodiments, the IL-2 conjugate comprises SEQ ID NO: 7. In some embodiments, the IL-2 conjugate comprises SEQ ID NO: 8. In some embodiments, the IL-2 conjugate comprises SEQ ID NO: 9. In some embodiments, the IL-2 conjugate comprises SEQ ID NO: 10. In some embodiments, the IL-2 conjugate comprises SEQ ID NO: 11. In some embodiments, the IL-2 conjugate comprises SEQ ID NO: 12. In some embodiments, the IL-2 conjugate comprises SEQ ID NO: 13. In some embodiments, the IL-2 conjugate comprises SEQ ID NO: 14. In some embodiments, the IL-2 conjugate comprises SEQ ID NO: 15. In some embodiments, the IL-2 conjugate comprises SEQ ID NO: 16. In some embodiments, the IL-2 conjugate comprises SEQ ID NO: 17. In some embodiments, the IL-2 conjugate comprises SEQ ID NO: 18. In some embodiments, the IL-2 conjugate comprises SEQ ID NO: 19. In some embodiments, the IL-2 conjugate comprises SEQ ID NO: 20. In some embodiments, the IL-2 conjugate comprises SEQ ID NO: 21. In some embodiments, the IL-2 conjugate comprises SEQ ID NO: 22. In some embodiments, the IL-2 conjugate comprises SEQ ID NO: 23. In some embodiments, the IL-2 conjugate comprises SEQ ID NO: 24. In some embodiments, the IL-2 conjugate comprises SEQ ID NO: 25. In some embodiments, the IL-2 conjugate comprises SEQ ID NO: 26. In some embodiments, the IL-2 conjugate comprises SEQ ID NO: 27. In some embodiments, the IL-2 conjugate comprises SEQ ID NO: 28. In some embodiments, the IL-2 conjugate comprises SEQ ID NO: 24. In some embodiments, the IL-2 conjugate comprises SEQ ID NO: 25. In some embodiments, the IL-2 conjugate comprises SEQ ID NO: 26. In some embodiments, the IL-2 conjugate comprises SEQ ID NO: 27. In some embodiments, the IL-2 conjugate comprises SEQ ID NO: 28. In some embodiments, the IL-2 conjugate comprises SEQ ID NO: 29. In some embodiments, the IL-2 conjugate comprises SEQ ID NO: 30. In some embodiments, the IL-2 conjugate comprises SEQ ID NO: 31. In some embodiments, the IL-2 conjugate comprises SEQ ID NO: 32. In some embodiments, the IL-2 conjugate comprises SEQ ID NO: 33. In some embodiments, the IL-2 conjugate comprises SEQ ID NO: 34. In some embodiments, the IL-2 conjugate comprises SEQ ID NO: 35. In some embodiments, the IL-2 conjugate comprises SEQ ID NO: 36. In some embodiments, the IL-2 conjugate comprises SEQ ID NO: 37. In some embodiments, the IL-2 conjugate comprises SEQ ID NO: 38. In some embodiments, the IL-2 conjugate comprises SEQ ID NO: 39. In some embodiments, the IL-2 conjugate comprises SEQ ID NO: 40. In some embodiments, the IL-2 conjugate comprises SEQ ID NO: 41. In some embodiments, the IL-2 conjugate comprises SEQ ID NO: 42. In some embodiments, the IL-2 conjugate comprises SEQ ID NO: 43. In some embodiments, the IL-2 conjugate comprises SEQ ID NO: 44. In some embodiments, the IL-2 conjugate comprises SEQ ID NO: 45. In some embodiments, the IL-2 conjugate comprises SEQ ID NO: 46. In some embodiments, the IL-2 conjugate comprises SEQ ID NO: 47. In some embodiments, the IL-2 conjugate comprises SEQ ID NO: 48. In some embodiments, the IL-2 conjugate comprises SEQ ID NO: 49. In some embodiments, the IL-2 conjugate comprises SEQ ID NO: 50. In some embodiments, the IL-2 conjugate comprises SEQ ID NO: 51. In some embodiments, the IL-2 conjugate comprises SEQ ID NO: 52. In some embodiments, the IL-2 conjugate comprises SEQ ID NO: 53. In some embodiments, the IL-2 conjugate comprises SEQ ID NO: 54. In some embodiments, the IL-2 conjugate comprises SEQ ID NO: 55. In some embodiments, the IL-2 conjugate comprises SEQ ID NO: 56. In some embodiments, the IL-2 conjugate comprises SEQ ID NO: 57. In some embodiments, the IL-2 conjugate comprises SEQ ID NO: 58. In some embodiments, the IL-2 conjugate comprises SEQ ID NO: 59. In some embodiments, the IL-2 conjugate comprises SEQ ID NO: 60. In some embodiments, the IL-2 conjugate comprises SEQ ID NO: 61. In some embodiments, the IL-2 conjugate comprises SEQ ID NO: 62. In some embodiments, the IL-2 conjugate comprises SEQ ID NO: 63. In some embodiments, the IL-2 conjugate comprises SEQ ID NO: 64. In some embodiments, the IL-2 conjugate comprises SEQ ID NO: 65. In some embodiments, the IL-2 conjugate comprises SEQ ID NO: 66. In some embodiments, the IL-2 conjugate comprises SEQ ID NO: 67. In some embodiments, the IL-2 conjugate comprises SEQ ID NO: 68. In some embodiments, the IL-2 conjugate comprises SEQ ID NO: 69. In some embodiments, the IL-2 conjugate comprises SEQ ID NO: 70. In some embodiments, the IL-2 conjugate comprises SEQ ID NO: 71 In some embodiments, the IL-2 conjugate comprises SEQ ID NO: 72. In some embodiments, the IL-2 conjugate comprises SEQ ID NO: 73. In some embodiments, the IL-2 conjugate comprises SEQ ID NO: 74. In some embodiments, the IL-2 conjugate comprises SEQ ID NO: 75. In some embodiments, the IL-2 conjugate comprises SEQ ID NO: 76. In some embodiments, the IL-2 conjugate comprises SEQ ID NO: 77. In some embodiments, the IL-2 conjugate comprises SEQ ID NO: 78. In some embodiments, the IL-2 conjugate comprises SEQ ID NO: 79. In some embodiments, the IL-2 conjugate comprises SEQ ID NO: 80. In some embodiments, the IL-2 conjugate comprises SEQ ID NO: 81. In some embodiments, the IL-2 conjugate comprises SEQ ID NO: 82. In some embodiments, the IL-2 conjugate comprises SEQ ID NO: 83. In some embodiments, the IL-2 conjugate comprises SEQ ID NO: 84. In some embodiments, the IL-2 conjugate comprises SEQ ID NO: 85. In some embodiments, the IL-2 conjugate comprises SEQ ID NO: 86. In some embodiments, the IL-2 conjugate comprises SEQ ID NO: 87. In some embodiments, the IL-2 conjugate comprises SEQ ID NO: 88. In some embodiments, the IL-2 conjugate comprises SEQ ID NO: 89. In some embodiments, the IL-2 conjugate comprises SEQ ID NO: 90. In some embodiments, the IL-2 conjugate comprises SEQ ID NO: 91. In some embodiments, the IL-2 conjugate comprises SEQ ID NO: 92. In some embodiments, the IL-2 conjugate comprises SEQ ID NO: 93. In some embodiments, the IL-2 conjugate comprises SEQ ID NO: 94. In some embodiments, the IL-2 conjugate comprises SEQ ID NO: 95. In some embodiments, the IL-2 conjugate comprises SEQ ID NO: 96. In some embodiments, the IL-2 conjugate comprises SEQ ID NO: 97. In some embodiments, the IL-2 conjugate comprises SEQ ID NO: 98.

In some embodiments, the IL-2 conjugate comprises a structure of Formula (I). In some embodiments, the IL-2 conjugate comprises a structure of Formula (II). In some embodiments, the IL-2 conjugate comprises a structure of Formula (III). In some embodiments, the IL-2 conjugate comprises a structure of Formula (IV). In some embodiments, the IL-2 conjugate comprises a structure of Formula (V). In some embodiments, the IL-2 conjugate comprises a structure of Formula (VI). In some embodiments, the IL-2 conjugate comprises a structure of Formula (VII). In some embodiments, the IL-2 conjugate comprises a structure of Formula (VIII). In some embodiments, the IL-2 conjugate comprises a structure of Formula (IX). In some embodiments, the IL-2 conjugate comprises a structure of Formula (X). In some embodiments, the IL-2 conjugate comprises a structure of Formula (XI). In some embodiments, the IL-2 conjugate comprises a structure of Formula (XII). In some embodiments, the IL-2 conjugate comprises a structure of Formula (XIII). In some embodiments, the IL-2 conjugate comprises a structure of Formula (XIV). In some embodiments, the IL-2 conjugate comprises a structure of Formula (XV). In some embodiments, the IL-2 conjugate comprises a structure of Formula (XVI). In some embodiments, the IL-2 conjugate comprises a structure of Formula (XV). In some embodiments, the IL-2 conjugate comprises a structure of Formula (XVI). In some embodiments, the IL-2 conjugate comprises a structure of Formula (XVII).

In some embodiments, the IL-2 conjugate comprises the amino acid sequence of any one of SEQ ID NOS: 86, 88, 90, 92, 94, 96, and 98. In any of these embodiments, the structure of Formula (I), or any variation thereof, such as Formula (II)-Formula (XVII) or any variation thereof, is incorporated into the site comprising the unnatural amino acid.

In some embodiments, described herein are IL-2 conjugates modified at an amino acid position. In some instances, the modification is to a natural amino acid. In some instances, the modification is to an unnatural amino acid. In some instances, described herein is an isolated and modified IL-2 polypeptide that comprises at least one unnatural amino acid. In some cases, the IL-2 polypeptide comprises about 80%, 85%, 90%, 95%, 96%, 97%, 98%, or 99% sequence identity to any one of SEQ ID NOS: 3 to 84. In some cases, the IL-2 polypeptide comprises about 80%, 85%, 90%, 95%, 96%, 97%, 98%, or 99% sequence identity to any one of SEQ ID NOS: 3 to 98.

In some instances, the IL-2 conjugate further comprises an additional mutation. In some cases, the additional mutation is at an amino acid position selected from K35, T37, R38, T41, F42, K43, F44, Y45, E61, E62, E68, K64, P65, V69, L72, and Y107. In such cases, the amino acid is conjugated to an additional conjugating moiety for increase in serum half-life, stability, or a combination thereof. Alternatively, the amino acid is first mutated to a natural amino acid such as lysine, cysteine, histidine, arginine, aspartic acid, glutamic acid, serine, threonine, or tyrosine; or to an unnatural amino acid prior to binding to the additional conjugating moiety.

In some cases, the PEG group is not limited to a particular structure. In some cases, the PEG is linear (e.g., an end capped, e.g., alkoxy PEG or a bifunctional PEG), branched or multi-armed (e.g., forked PEG or PEG attached to a polyol core), a dendritic (or star) architecture, each with or without one or more degradable linkages. Moreover, the internal structure of the water-soluble polymer can be organized in any number of different repeat patterns and can be selected from the group consisting of homopolymer, alternating copolymer, random copolymer, block copolymer, alternating tripolymer, random tripolymer, and block tripolymer.

PEGs will typically comprise a number of (OCH₂CH₂) monomers [or (CH₂CH₂O) monomers, depending on how the PEG is defined]. As used herein, the number of repeating units is identified by the subscript “n” in “(OCH₂CH₂)ₙ.” Thus, the value of (n) typically falls within one or more of the following ranges: from 2 to about 3400, from about 100 to about 2300, from about 100 to about 2270, from about 136 to about 2050, from about 225 to about 1930, from about 450 to about 1930, from about 1200 to about 1930, from about 568 to about 2727, from about 660 to about 2730, from about 795 to about 2730, from about 795 to about 2730, from about 909 to about 2730, and from about 1,200 to about 1,900. For any given polymer in which the molecular weight is known, it is possible to determine the number of repeating units (i.e., “n”) by dividing the total weight-average molecular weight of the polymer by the molecular weight of the repeating monomer.

In some instances, the PEG is an end-capped polymer, that is, a polymer having at least one terminus capped with a relatively inert group, such as a lower C₁₋₆ alkoxy group, or a hydroxyl group. When the polymer is PEG, for example, a methoxy-PEG (commonly referred to as mPEG) may be used, which is a linear form of PEG wherein one terminus of the polymer is a methoxy (—OCH₃) group, while the other terminus is a hydroxyl or other functional group that can be optionally chemically modified.

In some embodiments, the PEG group comprising the IL-2 conjugates disclosed herein is a linear or branched PEG group. In some embodiments, the PEG group is a linear PEG group. In some embodiments, the PEG group is a branched PEG group. In some embodiments, the PEG group is a methoxy PEG group. In some embodiments, the PEG group is a linear or branched methoxy PEG group. In some embodiments, the PEG group is a linear methoxy PEG group. In some embodiments, the PEG group is a branched methoxy PEG group. In some embodiments, the PEG group is a linear or branched PEG group having an average molecular weight of from about 100 Daltons to about 150,000 Daltons. Exemplary ranges include, for example, weight-average molecular weights in the range of greater than 5,000 Daltons to about 100,000 Daltons, in the range of from about 6,000 Daltons to about 90,000 Daltons, in the range of from about 10,000 Daltons to about 85,000 Daltons, in the range of greater than 10,000 Daltons to about 85,000 Daltons, in the range of from about 20,000 Daltons to about 85,000 Daltons, in the range of from about 53,000 Daltons to about 85,000 Daltons, in the range of from about 25,000 Daltons to about 120,000 Daltons, in the range of from about 29,000 Daltons to about 120,000 Daltons, in the range of from about 35,000 Daltons to about 120,000 Daltons, and in the range of from about 40,000 Daltons to about 120,000 Daltons. Exemplary weight-average molecular weights for the PEG group include about 100 Daltons, about 200 Daltons, about 300 Daltons, about 400 Daltons, about 500 Daltons, about 600 Daltons, about 700 Daltons, about 750 Daltons, about 800 Daltons, about 900 Daltons, about 1,000 Daltons, about 1,500 Daltons, about 2,000 Daltons, about 2,200 Daltons, about 2,500 Daltons, about 3,000 Daltons, about 4,000 Daltons, about 4,400 Daltons, about 4,500 Daltons, about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear PEG group having an average molecular weight as disclosed above. In some embodiments, the PEG group is a branched PEG group having an average molecular weight as disclosed above. In some embodiments, the PEG group comprising the IL-2 conjugates disclosed herein is a linear or branched PEG group having a defined molecular weight±10%, or 15% or 20% or 25%. For example, included within the scope of the present disclosure are IL-2 conjugates comprising a PEG group having a molecular weight of 30,000 Da 3000 Da, or 30,000 Da 4,500 Da, or 30,000 Da 6,000 Da.

In some embodiments, the PEG group comprising the IL-2 conjugates disclosed herein is a linear or branched PEG group having an average molecular weight of from about 5,000 Daltons to about 60,000 Daltons. In some embodiments, the PEG group is a linear or branched PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear or branched PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a linear or branched PEG group having an average molecular weight of about 5,000 Daltons, about 30,000 Daltons, about 50,000 Daltons, or about 60,000 Daltons. In some embodiments, the PEG group is a linear PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched PEG group having an average molecular weight of about 5,000 Daltons, about 10,000 Daltons, about 20,000 Daltons, about 30,000 Daltons, about 50,000 Daltons, or about 60,000 Daltons.

In some embodiments, the PEG group comprising the IL-2 conjugates disclosed herein is a linear methoxy PEG group having an average molecular weight of from about 5,000 Daltons to about 60,000 Daltons. In some embodiments, the PEG group is a linear methoxy PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a linear methoxy PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a linear methoxy PEG group having an average molecular weight of about 5,000 Daltons, about 30,000 Daltons, about 50,000 Daltons, or about 60,000 Daltons. In some embodiments, the PEG group is a linear methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a linear methoxy PEG group having an average molecular of about 5,000 Daltons. In some embodiments, the PEG group is a linear methoxy PEG group having an average molecular of about 10,000 Daltons. In some embodiments, the PEG group is a linear methoxy PEG group having an average molecular of about 20,000 Daltons. In some embodiments, the PEG group is a linear methoxy PEG group having an average molecular of about 30,000 Daltons. In some embodiments, the PEG group is a linear methoxy PEG group having an average molecular of about 50,000 Daltons. In some embodiments, the PEG group is a linear methoxy PEG group having an average molecular of about 60,000 Daltons. In some embodiments, the PEG group comprising the IL-2 conjugates disclosed herein is a linear methoxy PEG group having a defined molecular weight ±10%, or 15% or 20% or 25%. For example, included within the scope of the present disclosure are IL-2 conjugates comprising a linear methoxy PEG group having a molecular weight of 30,000 Da±3000 Da, or 30,000 Da±4,500 Da, or 30,000 Da±6,000 Da.

In some embodiments, the PEG group comprising the IL-2 conjugates disclosed herein is a branched methoxy PEG group having an average molecular weight of from about 5,000 Daltons to about 60,000 Daltons. In some embodiments, the PEG group is a branched methoxy PEG group having an average molecular weight of about 5,000 Daltons, about 5,500 Daltons, about 6,000 Daltons, about 7,000 Daltons, about 7,500 Daltons, about 8,000 Daltons, about 9,000 Daltons, about 10,000 Daltons, about 11,000 Daltons, about 12,000 Daltons, about 13,000 Daltons, about 14,000 Daltons, about 15,000 Daltons, about 20,000 Daltons, about 22,500 Daltons, about 25,000 Daltons, about 30,000 Daltons, about 35,000 Daltons, about 40,000 Daltons, about 45,000 Daltons, about 50,000 Daltons, about 55,000 Daltons, about 60,000 Daltons, about 65,000 Daltons, about 70,000 Daltons, about 75,000 Daltons, about 80,000 Daltons, about 90,000 Daltons, about 95,000 Daltons, and about 100,000 Daltons. In some embodiments, the PEG group is a branched methoxy PEG group having an average molecular weight of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular weight of about 5,000 Daltons, about 30,000 Daltons, about 50,000 Daltons, or about 60,000 Daltons. In some embodiments, the PEG group is a branched methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular of about 5,000 Daltons, about 10,000 Daltons, about 20,000 Daltons, about 30,000 Daltons, about 50,000 Daltons, or about 60,000 Daltons. In some embodiments, the PEG group is a branched methoxy PEG group having an average molecular of about 5,000 Daltons. In some embodiments, the PEG group is a branched methoxy PEG group having an average molecular of about 10,000 Daltons. In some embodiments, the PEG group is a branched methoxy PEG group having an average molecular of about 20,000 Daltons. In some embodiments, the PEG group is a branched methoxy PEG group having an average molecular of about 30,000 Daltons. In some embodiments, the PEG group is a branched methoxy PEG group having an average molecular of about 50,000 Daltons. In some embodiments, the PEG group is a branched methoxy PEG group having an average molecular of about 60,000 Daltons. In some embodiments, the PEG group comprising the IL-2 conjugates disclosed herein is a branched methoxy PEG group having a defined molecular weight±10%, or 15% or 20% or 25%. For example, included within the scope of the present disclosure are IL-2 conjugates comprising a branched methoxy PEG group having a molecular weight of 30,000 Da±3000 Da, or 30,000 Da±4,500 Da, or 30,000 Da±6,000 Da.

In some embodiments, exemplary PEG groups include, but are not limited to, linear or branched discrete PEG (dPEG) from Quanta Biodesign, Ltd; linear, branched, or forked PEGs from Nektar Therapeutics; and Y-shaped PEG derivatives from JenKem Technology.

### Conjugation Chemistry

### Conjugation Chemistry

Various conjugation reactions are used to conjugate linkers, conjugation moieties, and unnatural amino acids incorporated into cytokine peptides described herein. Such conjugation reactions are often compatible with aqueous conditions, such as “bioorthogonal” reactions. In some embodiments, conjugation reactions are mediated by chemical reagents such as catalysts, light, or reactive chemical groups found on linkers, conjugation moieties, or unnatural amino acids. In some embodiments, conjugation reactions are mediated by enzymes. In some embodiments, a conjugation reaction used herein is described in Gong, Y., Pan, L. Tett. Lett. 2015, 56, 2123. In some embodiments, a conjugation reaction used herein is described in Chen, X.; Wu. Y-W. Org. Biomol. Chem. 2016, 14, 5417. The disclosure of each of these references is incorporated herein by reference.

In some embodiments described herein, a conjugation reaction described herein comprises a 1,3-dipolar cycloaddition reaction. In some embodiments, the 1,3-dipolar cycloaddition reaction comprises reaction of an azide and a phosphine (“Click” reaction). In some embodiments, the conjugation reaction is catalyzed by copper. In some embodiments, a conjugation reaction described herein results in cytokine peptide comprising a linker or conjugation moiety attached via a triazole. In some embodiments, a conjugation reaction described herein comprises reaction of an azide with a strained olefin. In some embodiments, a conjugation reaction described herein comprises reaction of an azide with a strained alkyne. In some embodiments, a conjugation reaction described herein comprises reaction of an azide with a cycloalkyne, for example DBCO.

In some embodiments described herein, a conjugation reaction described herein comprises the reaction outlined in Scheme S1, wherein X is the position in the IL-2 conjugate comprising an unnatural amino acid, such as in any one of SEQ ID NOS: 5, 6, 7, 8, 9, 30, 31, 32, 33, and 34.

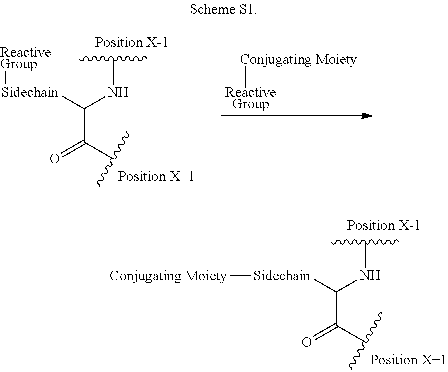

In some embodiments, the conjugating moiety comprises a water soluble polymer. In some embodiments, a reactive group comprises an alkyne or azide.

In some embodiments described herein, a conjugation reaction described herein comprises the reaction outlined in Scheme S2, wherein X is the position in the IL-2 conjugate comprising an unnatural amino acid, such as in any one of SEQ ID NOS: 5, 6, 7, 8, 9, 30, 31, 32, 33, and 34.

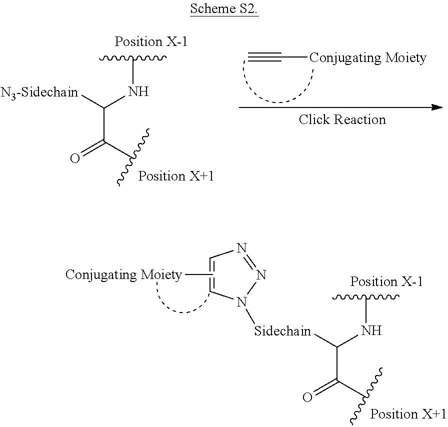

In some embodiments described herein, a conjugation reaction described herein comprises the reaction outlined in Scheme S3, wherein X is the position in the IL-2 conjugate comprising an unnatural amino acid, such as in any one of SEQ ID NOS: 5, 6, 7, 8, 9, 30, 31, 32, 33, and 34.

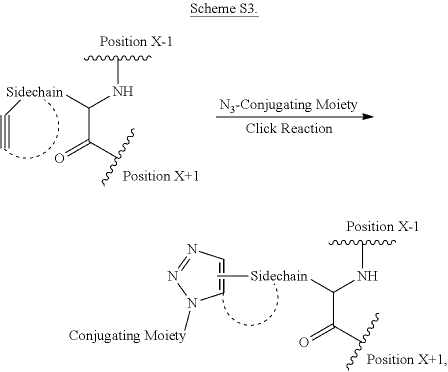

In some embodiments described herein, a conjugation reaction described herein comprises the reaction outlined in Scheme S4, wherein X is the position in the IL-2 conjugate comprising an unnatural amino acid, such as in any one of SEQ ID NOS: 5, 6, 7, 8, 9, 30, 31, 32, 33, and 34.

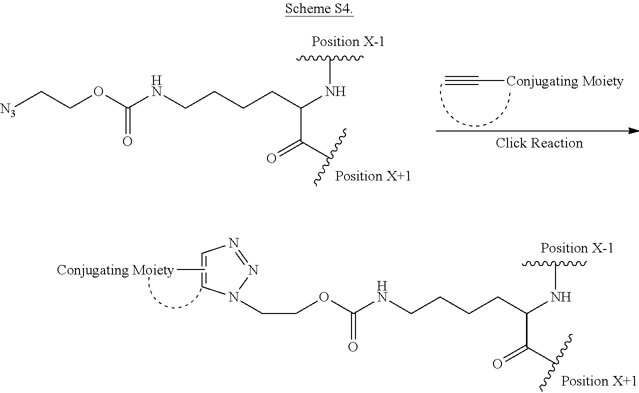

In some embodiments described herein, a conjugation reaction described herein comprises a cycloaddition reaction between an azide moiety, such as that contained in a protein containing an amino acid residue derived from N6-((2-azidoethoxy)-carbonyl)-L-lysine (AzK), and a strained cycloalkyne, such as that derived from DBCO, which is a chemical moiety comprising a dibenzocyclooctyne group. PEG groups comprising a DBCO moiety are commercially available or may be prepared by methods know to those of ordinary skill in the art. An exemplary reaction is shown in Schemes S5 and S6.

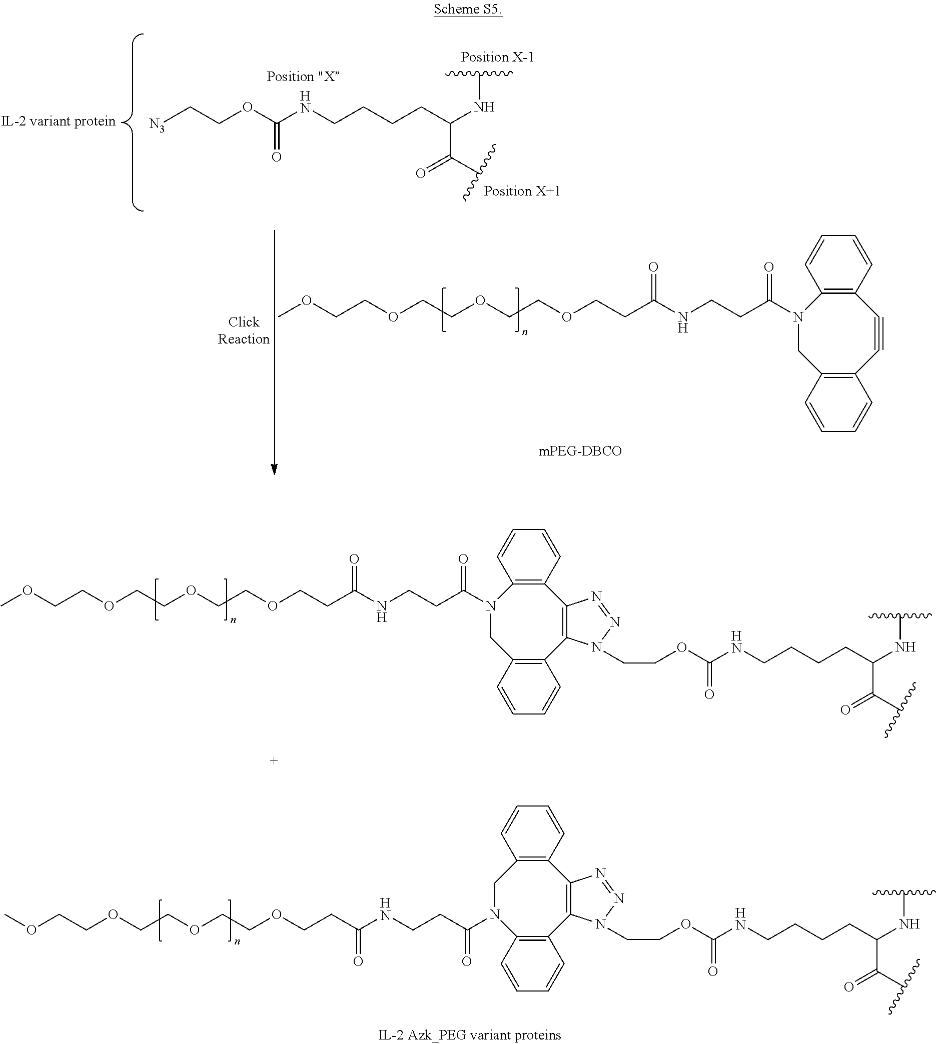

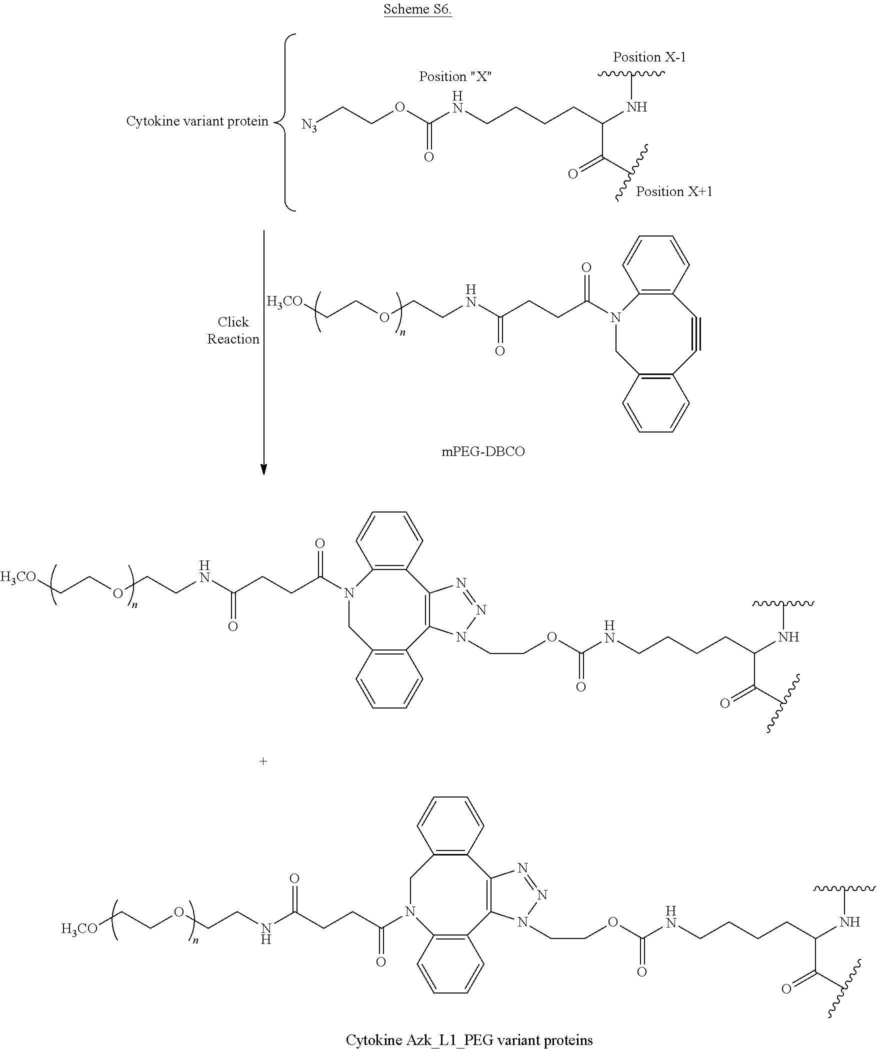

Conjugation reactions such as a click reaction described herein may generate a single regioisomer, or a mixture of regioisomers. In some instances the ratio of regioisomers is about 1:1. In some instances the ratio of regioisomers is about 2:1. In some instances the ratio of regioisomers is about 1.5:1. In some instances the ratio of regioisomers is about 1.2:1. In some instances the ratio of regioisomers is about 1.1:1. In some instances the ratio of regioisomers is greater than 1:1.

### Cytokine Polypeptide Production

In some instances, the IL-2 conjugates described herein, either containing a natural amino acid mutation or an unnatural amino acid mutation, are generated recombinantly or are synthesized chemically. In some instances, IL-2 conjugates described herein are generated recombinantly, for example, either by a host cell system, or in a cell-free system. In any of the embodiments or variations described herein, the amino acid may be an L-amino acid or a D-amino acid. In some embodiments, the amino acid is an L-amino acid. In other embodiments, the amino acid is a D-amino acid.

In some instances, IL-2 conjugates are generated recombinantly through a host cell system. In some cases, the host cell is a eukaryotic cell (e.g., mammalian cell, insect cells, yeast cells or plant cell) or a prokaryotic cell (e.g., gram-positive bacterium or a gram-negative bacterium). In some cases, a eukaryotic host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cell line, 293A cell line, 293FT cell line, 293F cells, 293 H cells, A549 cells, MDCK cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some embodiments, a eukaryotic host cell is an insect host cell. Exemplary insect host cell include Drosophila S2 cells, Sf9 cells, Sf21 cells, High Five™ cells, and expresSF+® cells.

In some embodiments, a eukaryotic host cell is a yeast host cell. Exemplary yeast host cells include Pichia pastoris (K. phaffii) yeast strains such as GS115, KM71H, SMD1168, SMD1168H, and X-33, and Saccharomyces cerevisiae yeast strain such as INVSc1.

In some embodiments, a eukaryotic host cell is a plant host cell. In some instances, the plant cells comprise a cell from algae. Exemplary plant cell lines include strains from Chlamydomonas reinhardtii 137c, or Synechococcus elongatus PPC 7942.

In some embodiments, a host cell is a prokaryotic host cell. Exemplary prokaryotic host cells include BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™.

In some instances, suitable polynucleic acid molecules or vectors for the production of an IL-2 polypeptide described herein include any suitable vectors derived from either a eukaryotic or prokaryotic source. Exemplary polynucleic acid molecules or vectors include vectors from bacteria (e.g., E. coli), insects, yeast (e.g., Pichia pastoris, K. phaffii), algae, or mammalian source. Bacterial vectors include, for example, pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Insect vectors include, for example, pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway®pDEST™ 14 vector, Gateway®pDEST™ 15 vector, Gateway®pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris (K. phaffii) vector, pGAPZA, B, & C Pichia pastoris (K. phaffii) vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is used for the production of a cytokine (e.g., IL-2) polypeptide described herein. In some cases, a cell-free system comprises a mixture of cytoplasmic and/or nuclear components from a cell and is suitable for in vitro nucleic acid synthesis. In some instances, a cell-free system utilizes prokaryotic cell components. In other instances, a cell-free system utilizes eukaryotic cell components. Nucleic acid synthesis is obtained in a cell-free system based on, for example, Drosophila cell, Xenopus egg, Archaea, or HeLa cells. Exemplary cell-free systems include E. coli S30 Extract system, E. coli T7 S30 system, or PURExpress®, XpressCF, and XpressCF+.

Cell-free translation systems variously comprise components such as plasmids, mRNA, DNA, tRNAs, synthetases, release factors, ribosomes, chaperone proteins, translation initiation and elongation factors, natural and/or unnatural amino acids, and/or other components used for protein expression. Such components are optionally modified to improve yields, increase synthesis rate, increase protein product fidelity, or incorporate unnatural amino acids. In some embodiments, cytokines described herein are synthesized using cell-free translation systems described in U.S. Pat. No. 8,778,631; US 2017/0283469; US 2018/0051065; US 2014/0315245; or U.S. Pat. No. 8,778,631. In some embodiments, cell-free translation systems comprise modified release factors, or even removal of one or more release factors from the system. In some embodiments, cell-free translation systems comprise a reduced protease concentration. In some embodiments, cell-free translation systems comprise modified tRNAs with re-assigned codons used to code for unnatural amino acids. In some embodiments, the synthetases described herein for the incorporation of unnatural amino acids are used in cell-free translation systems. In some embodiments, tRNAs are pre-loaded with unnatural amino acids using enzymatic or chemical methods before being added to a cell-free translation system. In some embodiments, components for a cell-free translation system are obtained from modified organisms, such as modified bacteria, yeast, or other organism.

In some embodiments, a cytokine (e.g., IL-2) polypeptide is generated as a circularly permuted form, either via an expression host system or through a cell-free system.

### Production of Cytokine Polypeptide Comprising an Unnatural Amino Acid

An orthogonal or expanded genetic code can be used in the present disclosure, in which one or more specific codons present in the nucleic acid sequence of a cytokine (e.g., IL-2) polypeptide are allocated to encode the unnatural amino acid so that it can be genetically incorporated into the cytokine (e.g., IL-2) by using an orthogonal tRNA synthetase/tRNA pair. The orthogonal tRNA synthetase/tRNA pair is capable of charging a tRNA with an unnatural amino acid and is capable of incorporating that unnatural amino acid into the polypeptide chain in response to the codon.

In some instances, the codon is the codon amber, ochre, opal or a quadruplet codon. In some cases, the codon corresponds to the orthogonal tRNA which will be used to carry the unnatural amino acid. In some cases, the codon is amber. In other cases, the codon is an orthogonal codon.

In some instances, the codon is a quadruplet codon, which can be decoded by an orthogonal ribosome ribo-Q1. In some cases, the quadruplet codon is as illustrated in Neumann, et al., “Encoding multiple unnatural amino acids via evolution of a quadruplet-decoding ribosome,” Nature, 464(7287): 441-444 (2010), the disclosure of which is incorporated herein by reference.

In some instances, a codon used in the present disclosure is a recoded codon, e.g., a synonymous codon or a rare codon that is replaced with alternative codon. In some cases, the recoded codon is as described in Napolitano, et al., “Emergent rules for codon choice elucidated by editing rare arginine codons in Escherichia coli,” PNAS, 113(38): E5588-5597 (2016). In some cases, the recoded codon is as described in Ostrov et al., “Design, synthesis, and testing toward a 57-codon genome,” Science 353(6301): 819-822 (2016). The disclosure of each of these references is incorporated herein by reference.

In some instances, unnatural nucleic acids are utilized leading to incorporation of one or more unnatural amino acids into the cytokine (e.g., IL-2). Exemplary unnatural nucleic acids include, but are not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifiuoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C≡C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][,4]benzothiazin-2(3H)-one), G-clamps, phenoxazine cytidine (e.g. 9-(2-aminoethoxy)-H-pyrimido[5,4-b][,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base is replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, 2-amino-2′-deoxyadenosine, and those described in U.S. Pat. Nos. 3,687,808; 4,845,205; 4,910,300; 4,948,882; 5,093,232; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653 and 6,005,096; WO 99/62923; Kandimalla et al., (2001) Bioorg. Med. Chem. 9:807-813; The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; and Sanghvi, Chapter 15, Antisense Research and Applications, Crooke and Lebleu Eds., CRC Press, 1993, 273-288. Additional base modifications can be found, for example, in U.S. Pat. No. 3,687,808; Englisch et al., Angewandte Chemie, International Edition, 1991, 30, 613; and Sanghvi, Chapter 15, Antisense Research and Applications, pages 289-302, Crooke and Lebleu ed., CRC Press, 1993. The disclosure of each of these references is incorporated herein by reference.

Unnatural nucleic acids comprising various heterocyclic bases and various sugar moieties (and sugar analogs) are available in the art, and the nucleic acids in some cases include one or several heterocyclic bases other than the principal five base components of naturally-occurring nucleic acids. For example, the heterocyclic base includes, in some cases, uracil-5-yl, cytosin-5-yl, adenin-7-yl, adenin-8-yl, guanin-7-yl, guanin-8-yl, 4-aminopyrrolo [2.3-d] pyrimidin-5-yl, 2-amino-4-oxopyrolo [2, 3-d] pyrimidin-5-yl, 2-amino-4-oxopyrrolo [2.3-d] pyrimidin-3-yl groups, where the purines are attached to the sugar moiety of the nucleic acid via the 9-position, the pyrimidines via the 1-position, the pyrrolopyrimidines via the 7-position and the pyrazolopyrimidines via the 1-position.

In some embodiments, nucleotide analogs are also modified at the phosphate moiety. Modified phosphate moieties include, but are not limited to, those with modification at the linkage between two nucleotides and contains, for example, a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkage between two nucleotides are through a 3′-5′ linkage or a 2′-5′ linkage, and the linkage contains inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, the disclosures of each of which are incorporated herein by reference.

In some embodiments, unnatural nucleic acids include 2′,3′-dideoxy-2′,3′-didehydro-nucleosides (PCT/US2002/006460), 5′-substituted DNA and RNA derivatives (PCT/US2011/033961; Saha et al., J. Org Chem., 1995, 60, 788-789; Wang et al., Bioorganic & Medicinal Chemistry Letters, 1999, 9, 885-890; and Mikhailov et al., Nucleosides & Nucleotides, 1991, 10(1-3), 339-343; Leonid et al., 1995, 14(3-5), 901-905; and Eppacher et al., Helvetica Chimica Acta, 2004, 87, 3004-3020; PCT/JP2000/004720; PCT/JP2003/002342; PCT/JP2004/013216; PCT/JP2005/020435; PCT/JP2006/315479; PCT/JP2006/324484; PCT/JP2009/056718; PCT/JP2010/067560), or 5′-substituted monomers made as the monophosphate with modified bases (Wang et al., Nucleosides Nucleotides & Nucleic Acids, 2004, 23 (1 & 2), 317-337). The disclosure of each of these references is incorporated herein by reference.

In some embodiments, unnatural nucleic acids include modifications at the 5′-position and the 2′-position of the sugar ring (PCT/US94/02993), such as 5′-CH₂-substituted 2′-O-protected nucleosides (Wu et al., Helvetica Chimica Acta, 2000, 83, 1127-1143 and Wu et al., Bioconjugate Chem. 1999, 10, 921-924). In some cases, unnatural nucleic acids include amide linked nucleoside dimers have been prepared for incorporation into oligonucleotides wherein the 3′ linked nucleoside in the dimer (5′ to 3′) comprises a 2′-OCH₃ and a 5′-(S)-CH₃ (Mesmaeker et al., Synlett, 1997, 1287-1290). Unnatural nucleic acids can include 2′-substituted 5′-CH₂ (or O) modified nucleosides (PCT/US92/01020). Unnatural nucleic acids can include 5′-methylenephosphonate DNA and RNA monomers, and dimers (Bohringer et al., Tet. Lett., 1993, 34, 2723-2726; Collingwood et al., Synlett, 1995, 7, 703-705; and Hutter et al., Helvetica Chimica Acta, 2002, 85, 2777-2806). Unnatural nucleic acids can include 5′-phosphonate monomers having a 2′-substitution (US2006/0074035) and other modified 5′-phosphonate monomers (WO1997/35869). Unnatural nucleic acids can include 5′-modified methylenephosphonate monomers (EP614907 and EP629633). Unnatural nucleic acids can include analogs of 5′ or 6′-phosphonate ribonucleosides comprising a hydroxyl group at the 5′ and/or 6′-position (Chen et al., Phosphorus, Sulfur and Silicon, 2002, 777, 1783-1786; Jung et al., Bioorg. Med. Chem., 2000, 8, 2501-2509; Gallier et al., Eur. J. Org. Chem., 2007, 925-933; and Hampton et al., J. Med. Chem., 1976, 19(8), 1029-1033). Unnatural nucleic acids can include 5′-phosphonate deoxyribonucleoside monomers and dimers having a 5′-phosphate group (Nawrot et al., Oligonucleotides, 2006, 16(1), 68-82). Unnatural nucleic acids can include nucleosides having a 6′-phosphonate group wherein the 5′ or/and 6′-position is unsubstituted or substituted with a thio-tert-butyl group (SC(CH₃)₃) (and analogs thereof); a methyleneamino group (CH₂NH₂) (and analogs thereof) or a cyano group (CN) (and analogs thereof) (Fairhurst et al., Synlett, 2001, 4, 467-472; Kappler et al., J. Med. Chem., 1986, 29, 1030-1038; Kappler et al., J. Med. Chem., 1982, 25, 1179-1184; Vrudhula et al., J. Med. Chem., 1987, 30, 888-894; Hampton et al., J. Med. Chem., 1976, 19, 1371-1377; Geze et al., J. Am. Chem. Soc, 1983, 105(26), 7638-7640; and Hampton et al., J. Am. Chem. Soc, 1973, 95(13), 4404-4414). The disclosure of each of these references is incorporated herein by reference.

In some embodiments, unnatural nucleic acids also include modifications of the sugar moiety. In some cases, nucleic acids contain one or more nucleosides wherein the sugar group has been modified. Such sugar modified nucleosides may impart enhanced nuclease stability, increased binding affinity, or some other beneficial biological property. In certain embodiments, nucleic acids comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids (BNA); replacement of the ribosyl ring oxygen atom with S, N(R), or C(R₁)(R₂) (R═H, C₁-C₁₂ alkyl or a protecting group); and combinations thereof. Examples of chemically modified sugars can be found in WO2008/101157, US2005/0130923, and WO2007/134181, the disclosures of each of which are incorporated herein by reference.

In some instances, a modified nucleic acid comprises modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety may be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-undine or 2′-O-methyl-cytidine. Sugar modifications include 2′-O-alkyl-substituted deoxyribonucleosides and 2′-O-ethyleneglycol like ribonucleosides. The preparation of these sugars or sugar analogs and the respective “nucleosides” wherein such sugars or analogs are attached to a heterocyclic base (nucleic acid base) is known. Sugar modifications may also be made and combined with other modifications.

Modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C₁₀, alkyl or C₂ to C₁₀ alkenyl and alkynyl. 2′ sugar modifications also include but are not limited to —O[(CH₂)ₙO]m CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m are from 1 to about 10.

Other modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Modified sugars also include those that contain modifications at the bridging ring oxygen, such as CH₂ and S. Nucleotide sugar analogs may also have sugar mimetics such as cyclobutyl moieties in place of the pentofuranosyl sugar. There are numerous United States patents that teach the preparation of such modified sugar structures and which detail and describe a range of base modifications, such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,700,920, each of which is herein incorporated by reference in its entirety.

Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein include one or more bicyclic nucleic acids. In certain such embodiments, the bicyclic nucleic acid comprises a bridge between the 4′ and the 2′ ribosyl ring atoms. In certain embodiments, nucleic acids provided herein include one or more bicyclic nucleic acids wherein the bridge comprises a 4′ to 2′ bicyclic nucleic acid. Examples of such 4′ to 2′ bicyclic nucleic acids include, but are not limited to, one of the Formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof (see, U.S. Pat. No. 7,399,845); 4′-C(CH₃)(CH₃)—O-2′ and analogs thereof, (see WO2009/006478, WO2008/150729, US2004/0171570, U.S. Pat. No. 7,427,672, Chattopadhyaya et al., J. Org. Chem., 209, 74, 118-134, and WO2008/154401). Also see, for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invens. Drugs, 2001, 2, 558-561; Braasch et al., Chem. Biol, 2001, 8, 1-7; Oram et al., Curr. Opinion Mol. Ther., 2001, 3, 239-243; U.S. Pat. Nos. 4,849,513; 5,015,733; 5,118,800; 5,118,802; 7,053,207; 6,268,490; 6,770,748; 6,794,499; 7,034,133; 6,525,191; 6,670,461; and 7,399,845; International Publication Nos. WO2004/106356, WO1994/14226, WO2005/021570, WO2007/090071, and WO2007/134181; U.S. Patent Publication Nos. US2004/0171570, US2007/0287831, and US2008/0039618; U.S. Provisional Application Nos. 60/989,574, 61/026,995, 61/026,998, 61/056,564, 61/086,231, 61/097,787, and 61/099,844; and International Applications Nos. PCT/US2008/064591, PCT US2008/066154, PCT US2008/068922, and PCT/DK98/00393, the disclosures of each of which are incorporated herein by reference.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P═S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

Backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and may be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in vivo.

In some instances, a phosphorous derivative (or modified phosphate group) is attached to the sugar or sugar analog moiety in and can be a monophosphate, diphosphate, triphosphate, alkylphosphonate, phosphorothioate, phosphorodithioate, phosphoramidate or the like. Exemplary polynucleotides containing modified phosphate linkages or non-phosphate linkages can be found in Peyrottes et al., 1996, Nucleic Acids Res. 24: 1841-1848; Chaturvedi et al., 1996, Nucleic Acids Res. 24:2318-2323; and Schultz et al., (1996) Nucleic Acids Res. 24:2966-2973; Matteucci, 1997, “Oligonucleotide Analogs: an Overview” in Oligonucleotides as Therapeutic Agents, (Chadwick and Cardew, ed.) John Wiley and Sons, New York, N.Y.; Zon, 1993, “Oligonucleoside Phosphorothioates” in Protocols for Oligonucleotides and Analogs, Synthesis and Properties, Humana Press, pp. 165-190; Miller et al., 1971, JACS 93:6657-6665; Jager et al., 1988, Biochem. 27:7247-7246; Nelson et al., 1997, JOC 62:7278-7287; U.S. Pat. No. 5,453,496; and Micklefield, 2001, Curr. Med. Chem. 8: 1157-1179, the disclosures of each of which are incorporated herein by reference.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos (Micklefield, 2001, Current Medicinal Chemistry 8: 1157-1179, the disclosure of which is incorporated herein by reference). A modified nucleic acid may comprise a chimeric or mixed backbone comprising one or more modifications, e.g. a combination of phosphate linkages such as a combination of phosphodiester and phosphorothioate linkages.

Substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, the disclosures of each of which are incorporated herein by reference. It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. See also Nielsen et al., Science, 1991, 254, 1497-1500. It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. KY. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EM5OJ, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1-di-O-hexadecyl-rac-glycero-S-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochem. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937). Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941. The disclosure of each of these references is incorporated herein by reference.

In some cases, the unnatural nucleic acids further form unnatural base pairs. Exemplary unnatural nucleotides capable of forming an unnatural DNA or RNA base pair (UBP) under conditions in vivo includes, but is not limited to, TAT1, dTAT1, 5FM, d5FM, TPT3, dTPT3, 5SICS, d5SICS, NaM, dNaM, CNMO, dCNMO, and combinations thereof. In some embodiments, unnatural nucleotides include:

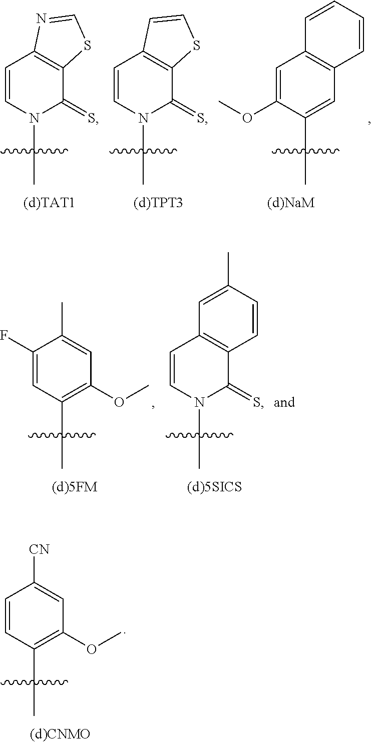

Exemplary unnatural base pairs include: (d)TPT3-(d)NaM; (d)5SICS-(d)NaM; (d)CNMO-(d)TAT1; (d)NaM-(d)TAT1; (d)CNMO-(d)TPT3; and (d)5FM-(d)TAT1.

Other examples of unnatural nucleotides capable of forming unnatural UBPs that may be used to prepare the IL-2 conjugates disclosed herein may be found in Dien et al., J Am Chem Soc., 2018, 140:16115-16123; Feldman et al., J Am Chem Soc, 2017, 139:11427-11433; Ledbetter et al., J Am Chem Soc., 2018, 140:758-765; Dhami et al., Nucleic Acids Res. 2014, 42:10235-10244; Malyshev et al., Nature, 2014, 509:385-388; Betz et al., J Am Chem Soc., 2013, 135:18637-18643; Lavergne et al., J Am Chem Soc. 2013, 135:5408-5419; and Malyshev et al. Proc Natl Acad Sci USA, 2012, 109:12005-12010, the disclosures of each of which are incorporated herein by reference. In some embodiments, unnatural nucleotides include:

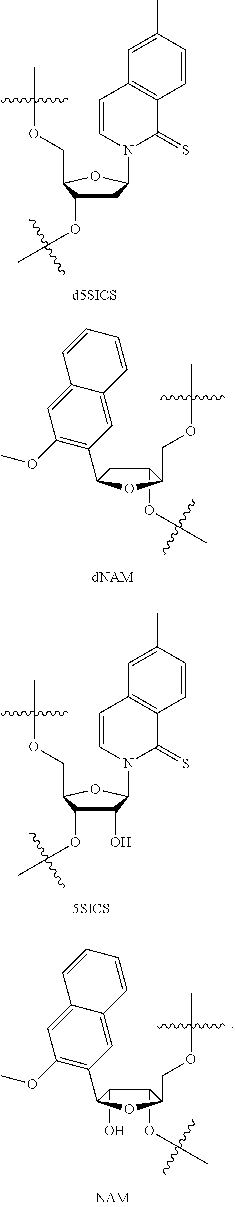

In some embodiments, the unnatural nucleotides that may be used to prepare the IL-2 conjugates disclosed herein may be derived from a compound of the Formula

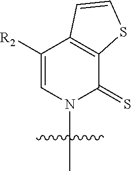

wherein R2 is selected from the group consisting of hydrogen, alkyl, alkenyl, alkynyl, methoxy, methanethiol, methaneseleno, halogen, cyano, and azido; and

the wavy line indicates a bond to a ribosyl or 2′-deoxyribosyl, wherein the 5′-hydroxy group of the ribosyl or 2′-deoxyribosyl moiety is in free form, is optionally bonded to a monophosphate, a diphosphate, or a triphosphate group, or is included in an RNA or a DNA or in an RNA analog or a DNA analog.

In some embodiments, the unnatural nucleotides that may be used to prepare the IL-2 conjugates disclosed herein may be derived from a compound of the Formula

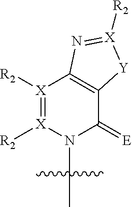

wherein:

each X is independently carbon or nitrogen;

R2 is absent when X is nitrogen, and is present when X is carbon and is independently hydrogen, alkyl, alkenyl, alkynyl, methoxy, methanethiol, methaneseleno, halogen, cyano, or azide;

Y is sulfur, oxygen, selenium, or secondary amine:

E is oxygen, sulfur, or selenium; and

the wavy line indicates a point of bonding to a ribosyl, deoxyribosyl, or dideoxyribosyl moiety or an analog thereof, wherein the ribosyl, deoxyribosyl, or dideoxyribosyl moiety or analog thereof is in free form, is connected to a mono-phosphate, diphosphate, triphosphate, α-thiotriphosphate, β-thiotriphosphate, or γ-thiotriphosphate group, or is included in an RNA or a DNA or in an RNA analog or a DNA analog.

In some embodiments, each X is carbon. In some embodiments, at least one X is carbon. In some embodiments, one X is carbon. In some embodiments, at least two X are carbon. In some embodiments, two X are carbon. In some embodiments, at least one X is nitrogen. In some embodiments, one X is nitrogen. In some embodiments, at least two X are nitrogen. In some embodiments, two X are nitrogen.

In some embodiments, Y is sulfur. In some embodiments, Y is oxygen. In some embodiments, Y is selenium. In some embodiments, Y is a secondary amine.

In some embodiments, E is sulfur. In some embodiments, E is oxygen. In some embodiments, E is selenium.

In some embodiments, R2 is present when X is carbon. In some embodiments, R² is absent when X is nitrogen. In some embodiments, each R₂, where present, is hydrogen. In some embodiments, R₂ is alkyl, such as methyl, ethyl, or propyl. In some embodiments, R₂ is alkenyl, such as —CH₂═CH₂. In some embodiments, R₂ is alkynyl, such as ethynyl. In some embodiments, R₂ is methoxy. In some embodiments, R₂ is methanethiol. In some embodiments, R₂ is methaneseleno. In some embodiments, R₂ is halogen, such as chloro, bromo, or fluoro. In some embodiments, R₂ is cyano. In some embodiments, R₂ is azide.

In some embodiments, E is sulfur, Y is sulfur, and each X is independently carbon or nitrogen. In some embodiments, E is sulfur, Y is sulfur, and each X is carbon.

In some embodiments, the unnatural nucleotides that may be used to prepare the IL-2 conjugates disclosed herein may be derived from

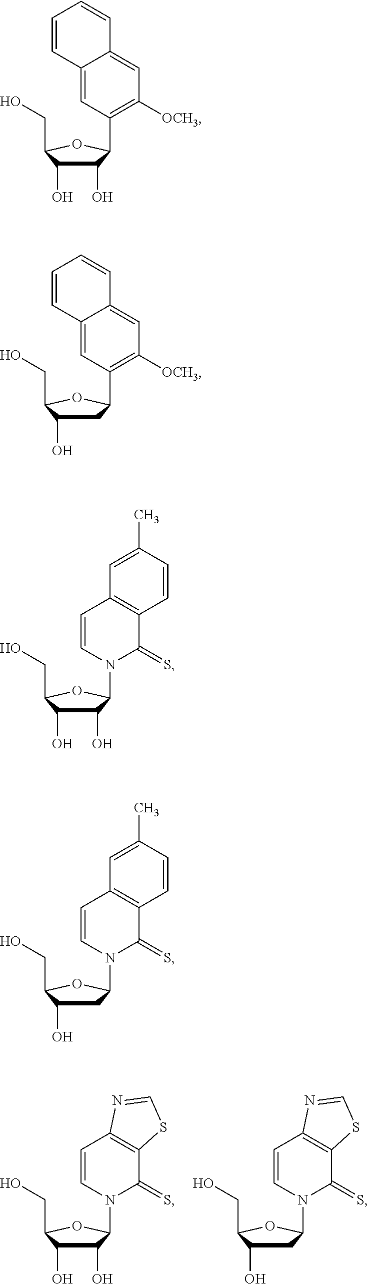

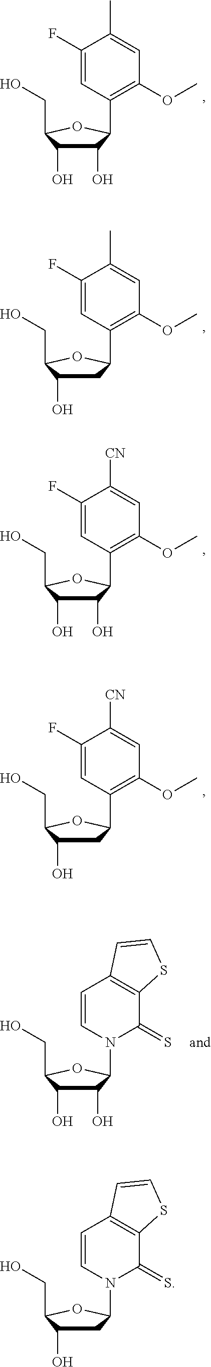

In some embodiments, the unnatural nucleotides that may be used to prepare the IL-2 conjugates disclosed herein include

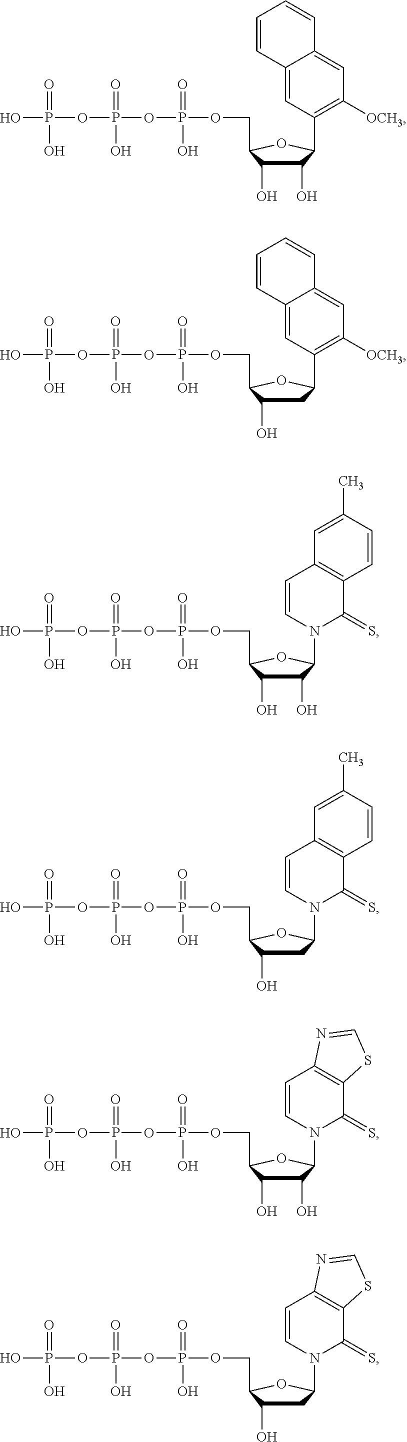

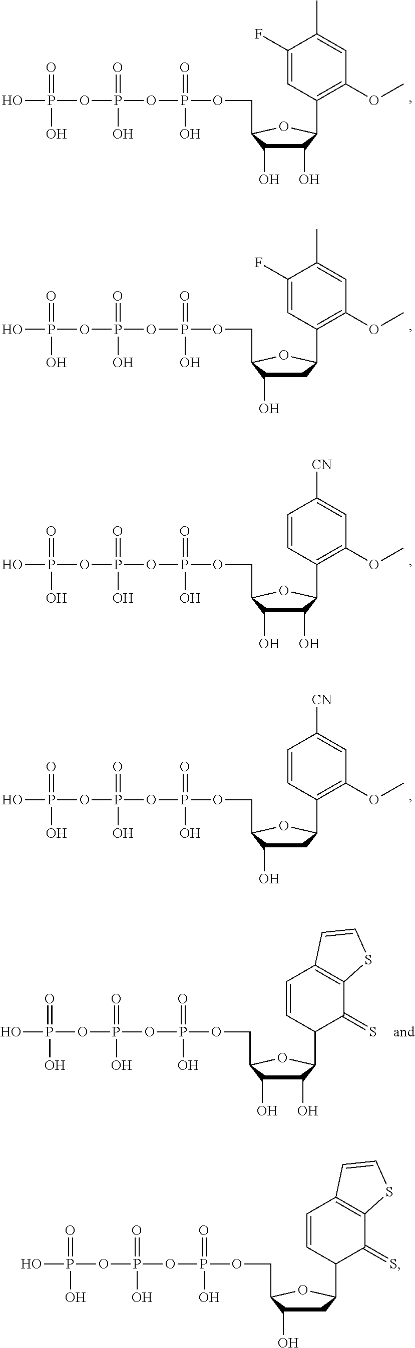

or salts thereof.

In some embodiments, an unnatural base pair generate an unnatural amino acid described in Dumas et al., “Designing logical codon reassignment—Expanding the chemistry in biology,” Chemical Science, 6: 50-69 (2015), the disclosure of which is incorporated herein by reference.

In some embodiments, the unnatural amino acid is incorporated into the cytokine (e.g., the IL polypeptide) by a synthetic codon comprising an unnatural nucleic acid. In some instances, the unnatural amino acid is incorporated into the cytokine by an orthogonal, modified synthetase/tRNA pair. Such orthogonal pairs comprise an unnatural synthetase that is capable of charging the unnatural tRNA with the unnatural amino acid, while minimizing charging of a) other endogenous amino acids onto the unnatural tRNA and b) unnatural amino acids onto other endogenous tRNAs. Such orthogonal pairs comprise tRNAs that are capable of being charged by the unnatural synthetase, while avoiding being charged with a) other endogenous amino acids by endogenous synthetases. In some embodiments, such pairs are identified from various organisms, such as bacteria, yeast, Archaea, or human sources. In some embodiments, an orthogonal synthetase/tRNA pair comprises components from a single organism. In some embodiments, an orthogonal synthetase/tRNA pair comprises components from two different organisms. In some embodiments, an orthogonal synthetase/tRNA pair comprising components that prior to modification, promote translation of two different amino acids. In some embodiments, an orthogonal synthetase is a modified alanine synthetase. In some embodiments, an orthogonal synthetase is a modified arginine synthetase. In some embodiments, an orthogonal synthetase is a modified asparagine synthetase. In some embodiments, an orthogonal synthetase is a modified aspartic acid synthetase. In some embodiments, an orthogonal synthetase is a modified cysteine synthetase. In some embodiments, an orthogonal synthetase is a modified glutamine synthetase. In some embodiments, an orthogonal synthetase is a modified glutamic acid synthetase. In some embodiments, an orthogonal synthetase is a modified alanine glycine. In some embodiments, an orthogonal synthetase is a modified histidine synthetase. In some embodiments, an orthogonal synthetase is a modified leucine synthetase. In some embodiments, an orthogonal synthetase is a modified isoleucine synthetase. In some embodiments, an orthogonal synthetase is a modified lysine synthetase. In some embodiments, an orthogonal synthetase is a modified methionine synthetase. In some embodiments, an orthogonal synthetase is a modified phenylalanine synthetase. In some embodiments, an orthogonal synthetase is a modified proline synthetase. In some embodiments, an orthogonal synthetase is a modified serine synthetase. In some embodiments, an orthogonal synthetase is a modified threonine synthetase. In some embodiments, an orthogonal synthetase is a modified tryptophan synthetase. In some embodiments, an orthogonal synthetase is a modified tyrosine synthetase. In some embodiments, an orthogonal synthetase is a modified valine synthetase. In some embodiments, an orthogonal synthetase is a modified phosphoserine synthetase. In some embodiments, an orthogonal tRNA is a modified alanine tRNA. In some embodiments, an orthogonal tRNA is a modified arginine tRNA. In some embodiments, an orthogonal tRNA is a modified asparagine tRNA. In some embodiments, an orthogonal tRNA is a modified aspartic acid tRNA. In some embodiments, an orthogonal tRNA is a modified cysteine tRNA. In some embodiments, an orthogonal tRNA is a modified glutamine tRNA. In some embodiments, an orthogonal tRNA is a modified glutamic acid tRNA. In some embodiments, an orthogonal tRNA is a modified alanine glycine. In some embodiments, an orthogonal tRNA is a modified histidine tRNA. In some embodiments, an orthogonal tRNA is a modified leucine tRNA. In some embodiments, an orthogonal tRNA is a modified isoleucine tRNA. In some embodiments, an orthogonal tRNA is a modified lysine tRNA. In some embodiments, an orthogonal tRNA is a modified methionine tRNA. In some embodiments, an orthogonal tRNA is a modified phenylalanine tRNA. In some embodiments, an orthogonal tRNA is a modified proline tRNA. In some embodiments, an orthogonal tRNA is a modified serine tRNA. In some embodiments, an orthogonal tRNA is a modified threonine tRNA. In some embodiments, an orthogonal tRNA is a modified tryptophan tRNA. In some embodiments, an orthogonal tRNA is a modified tyrosine tRNA. In some embodiments, an orthogonal tRNA is a modified valine tRNA. In some embodiments, an orthogonal tRNA is a modified phosphoserine tRNA.

In some embodiments, the unnatural amino acid is incorporated into the cytokine (e.g., the IL polypeptide) by an aminoacyl (aaRS or RS)-tRNA synthetase-tRNA pair. Exemplary aaRS-tRNA pairs include, but are not limited to, Methanococcus jannaschii (Mj-Tyr) aaRS/tRNA pairs, E. coli TyrRS (Ec-Tyr)/B. stearothermophilus tRNACUA pairs, E. coli LeuRS (Ec-Leu)/B. stearothermophilus tRNACUA pairs, and pyrrolysyl-tRNA pairs. In some instances, the unnatural amino acid is incorporated into the cytokine (e.g., the IL polypeptide) by a Mj-TyrRS/tRNA pair. Exemplary UAAs that can be incorporated by a Mj-TyrRS/tRNA pair include, but are not limited to, para-substituted phenylalanine derivatives such asp-aminophenylalanine and p-methoyphenylalanine; meta-substituted tyrosine derivatives such as 3-aminotyrosine, 3-nitrotyrosine, 3,4-dihydroxyphenylalanine, and 3-iodotyrosine; phenylselenocysteine; p-boronophenylalanine; and o-nitrobenzyltyrosine.

In some instances, the unnatural amino acid is incorporated into the cytokine (e.g., the IL polypeptide) by a Ec-Tyr/tRNACUA or a Ec-Leu/tRNACUA pair. Exemplary UAAs that can be incorporated by a Ec-Tyr/tRNACUA or a Ec-Leu/tRNACUA pair include, but are not limited to, phenylalanine derivatives containing benzophenone, ketone, iodide, or azide substituents; O-propargyltyrosine; α-aminocaprylic acid, O-methyl tyrosine, O-nitrobenzyl cysteine; and 3-(naphthalene-2-ylamino)-2-amino-propanoic acid.

In some instances, the unnatural amino acid is incorporated into the cytokine (e.g., the IL polypeptide) by a pyrrolysyl-tRNA pair. In some cases, the PylRS is obtained from an archaebacterial, e.g., from a methanogenic archaebacterial. In some cases, the PylRS is obtained from Methanosarcina barkeri, Methanosarcina mazei, or Methanosarcina acetivorans. Exemplary UAAs that can be incorporated by a pyrrolysyl-tRNA pair include, but are not limited to, amide and carbamate substituted lysines such as 2-amino-6-((R)-tetrahydrofuran-2-carboxamido)hexanoic acid, N-ε-D-prolyl-L-lysine, and N-ε-cyclopentyloxycarbonyl-L-lysine; N-ε-Acryloyl-L-lysine; N-ε-[(1-(6-nitrobenzo[d][1,3]dioxol-5-yl)ethoxy)carbonyl]-L-lysine; and N-ε-(1-methylcyclopro-2-enecarboxamido)lysine. In some embodiments, the IL-2 conjugates disclosed herein may be prepared by use of M. mazei tRNA which is selectively charged with a non-natural amino acid such as N6-((2-azidoethoxy)-carbonyl)-L-lysine (AzK) by the M. barkeri pyrrolysyl-tRNA synthetase (Mb PylRS). Other methods are known to those of ordinary skill in the art, such as those disclosed in Zhang et al., Nature 2017, 551(7682): 644-647, the disclosure of which is incorporated herein by reference.

In some instances, an unnatural amino acid is incorporated into a cytokine described herein (e.g., the IL polypeptide) by a synthetase disclosed in U.S. Pat. Nos. 9,988,619 and 9,938,516, the disclosures of each of which are incorporated herein by reference.

The host cell into which the constructs or vectors disclosed herein are introduced is cultured or maintained in a suitable medium such that the tRNA, the tRNA synthetase and the protein of interest are produced. The medium also comprises the unnatural amino acid(s) such that the protein of interest incorporates the unnatural amino acid(s). In some embodiments, a nucleoside triphosphate transporter (NTT) from bacteria, plant, or algae is also present in the host cell. In some embodiments, the IL-2 conjugates disclosed herein are prepared by use of a host cell that expresses a NTT. In some embodiments, the nucleotide nucleoside triphosphate transporter used in the host cell may be selected from TpNTT1, TpNTT2, TpNTT3, TpNTT4, TpNTT5, TpNTT6, TpNTT7, TpNTT8 (T. pseudonana), PtNTT1, PtNTT2, PtNTT3, PtNTT4, PtNTT5, PtNTT6 (P. tricornutum), GsNTT (Galdieria sulphuraria), AtNTT1, AtNTT2 (Arabidopsis thaliana), CtNTT1, CtNTT2 (Chlamydia trachomatis), PamNTT1, PamNTT2 (Protochlamydia amoebophila), CcNTT (Caedibacter caryophilus), RpNTT (Rickettsia prowazekii). In some embodiments, the NTT is selected from PtNTT1, PtNTT2, PtNTT3, PtNTT4, PtNTT5, and PtNTT6. In some embodiments, the NTT is PtNTT1. In some embodiments, the NTT is PtNTT2. In some embodiments, the NTT is PtNTT3. In some embodiments, the NTT is PtNTT4. In some embodiments, the NTT is PtNTT5. In some embodiments, the NTT is PtNTT6. Other NTTs that may be used are disclosed in Zhang et al., Nature 2017, 551(7682): 644-647; Malyshev et al. Nature 2014 (509(7500), 385-388; and Zhang et al. Proc Natl Acad Sci USA, 2017, 114:1317-1322, the disclosures of each of which are incorporated herein by reference.

The orthogonal tRNA synthetase/tRNA pair charges a tRNA with an unnatural amino acid and incorporates the unnatural amino acid into the polypeptide chain in response to the codon. Exemplary aaRS-tRNA pairs include, but are not limited to, Mehanococcus jannaschii (Mj-Tyr) aaRS/tRNA pairs, E. coli TyrRS (Ec-Tyr)/B. stearothermophilus tRNACUA pairs, E. coli LeuRS (Ec-Leu)/B. stearothermophilus tRNACUA pairs, and pyrrolysyl-tRNA pairs. Other aaRS-tRNA pairs that may be used according to the present disclosure include those derived from M. mazei those described in Feldman et al., J Am Chem Soc., 2018 140:1447-1454; and Zhang et al. Proc Natl Acad Sci USA, 2017, 114:1317-1322, the disclosures of each of which are incorporated herein by reference.

In some embodiments are provided methods of preparing the IL-2 conjugates disclosed herein in a cellular system that expresses a NTT and a tRNA synthetase. In some embodiments described herein, the NTT is selected from PtNTT1, PtNTT2, PtNTT3, PtNTT4, PtNTT5, and PtNTT6, and the tRNA synthetase is selected from Methanococcus jannaschii, E coli TyrRS (c-Tyr)/B. stearothermophilus, and M. mazei. In some embodiments, the NTT is PtNTT1 and the tRNA synthetase is derived from Methanococcus jannaschii, E. coli TyrRS (Ec-Tyr)/B. stearothermophilus, or M. mazei. In some embodiments, the NTT is PtNTT2 and the tRNA synthetase is derived from Methanococcus jannaschii, E coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT3 and the tRNA synthetase is derived from Methanococcus jannaschii, E. coli TyrRS (Ec-Tyr)/B. stearothermophilus, or M. mazei. In some embodiments, the NTT is PtNTT4 and the tRNA synthetase is derived from Methanococcus jannaschii, E coli TyrRS (Ec-Tyr)/B. stearothermophilus, or M. mazei. In some embodiments, the NTT is PtNTT5 and the tRNA synthetase is derived from Methanococcus jannaschii, E. coli TyrRS (Ec-Tyr)/B. stearothermophilus, or M. mazei. In some embodiments, the NTT is PtNTT6 and the tRNA synthetase is derived from Methanococcus jannaschii, E coli TyrRS (Ec-Tyr)/B. stearothermophilus, or A mazei.

In some embodiments, the IL-2 conjugates disclosed herein may be prepared in a cell, such as E. coli, comprising (a) nucleotide triphosphate transporter PtNTT2 (including a truncated variant in which the first 65 amino acid residues of the full-length protein are deleted), (b) a plasmid comprising a double-stranded oligonucleotide that encodes an IL-2 variant having a desired amino acid sequence and that contains a unnatural base pair comprising a first unnatural nucleotide and a second unnatural nucleotide to provide a codon at the desired position at which an unnatural amino acid, such as N6-((2-azidoethoxy)-carbonyl)-L-lysine (AzK), will be incorporated, (c) a plasmid encoding a tRNA derived from M. mazei and which comprises an unnatural nucleotide to provide a recognized anticodon (to the codon of the IL-2 variant) in place of its native sequence, and (d) a plasmid encoding a M. barkeri derived pyrrolysyl-tRNA synthetase (Mb PylRS), which may be the same plasmid that encodes the tRNA or a different plasmid. In some embodiments, the cell is further supplemented with deoxyribo triphosphates comprising one or more unnatural bases. In some embodiments, the cell is further supplemented with ribo triphosphates comprising one or more unnatural bases. In some embodiments, the cells is further supplemented with one or more unnatural amino acids, such as N6-((2-azidoethoxy)-carbonyl)-L-lysine (AzK). In some embodiments, the double-stranded oligonucleotide that encodes the amino acid sequence of the desired IL-2 variant contains a codon AXC at, for example, position 34, 37, 40, 41, 42, 43, 44, 61, 64, 68, or 71 of the sequence that encodes the protein having SEQ ID NO: 3, or at position 35, 38, 41, 42, 43, 45, 62, 65, 69, or 72 of the sequence that encodes the protein having SEQ ID NO: 4, wherein X is an unnatural nucleotide. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide that is complementary and may be the same or different as the unnatural nucleotide in the codon. In some embodiments, the unnatural nucleotide in the codon is different than and complimentary to the unnatural nucleotide in the anti-codon. In some embodiments, the unnatural nucleotide in the codon is the same as the unnatural nucleotide in the anti-codon. In some embodiments, the first and second unnatural nucleotides comprising the unnatural base pair in the double-stranded oligonucleotide may be derived from

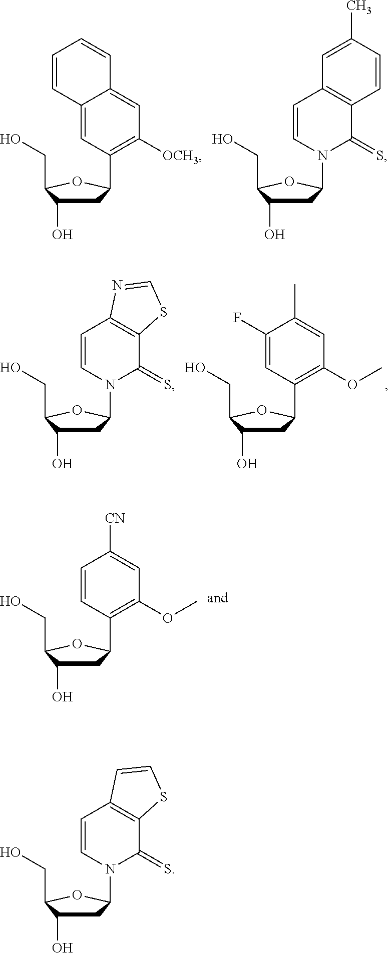

In some embodiments, the first and second unnatural nucleotides comprising the unnatural base pair in the double-stranded oligonucleotide may be derived from

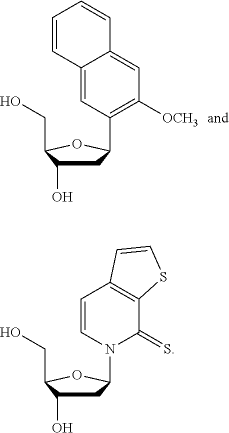

In some embodiments, the first and second unnatural nucleotides comprising the unnatural base pair in the double-stranded oligonucleotide may be derived from

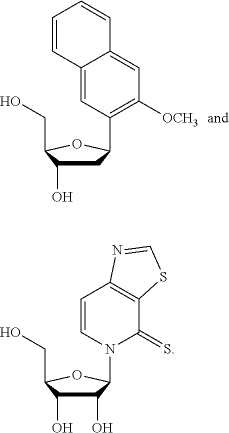

In some embodiments, the triphosphates of the first and second unnatural nucleotides include,

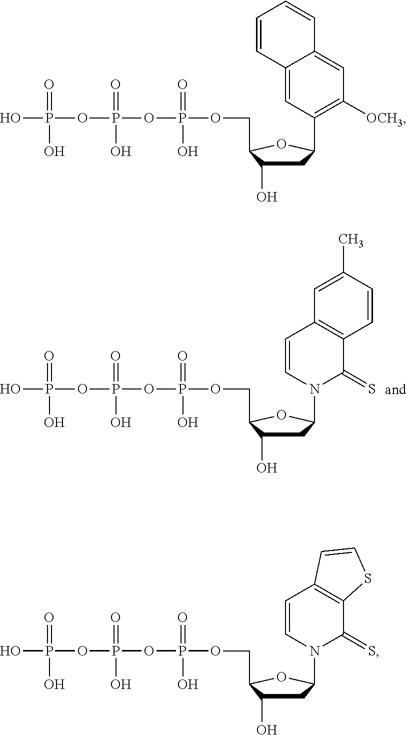

or salts thereof. In some embodiments, the triphosphates of the first and second unnatural nucleotides include

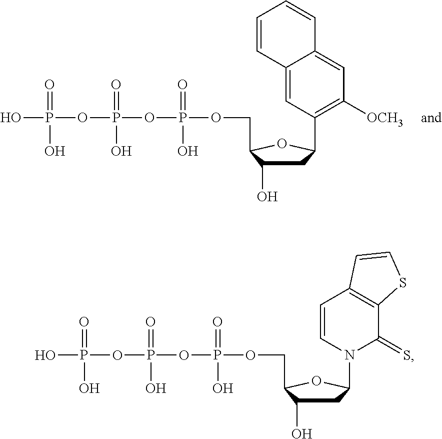

or salts thereof. In some embodiments, the triphosphates of the first and second unnatural nucleotides include

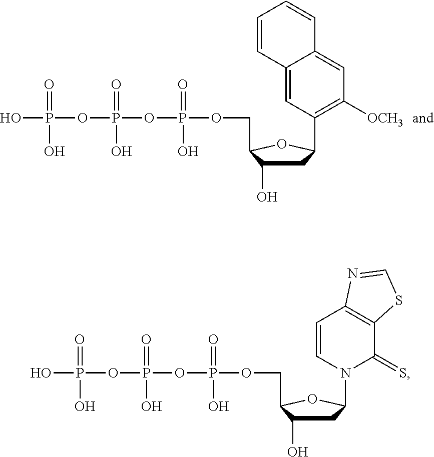

or salts thereof. In some embodiments, the mRNA derived the double-stranded oligonucleotide comprising a first unnatural nucleotide and a second unnatural nucleotide may comprise a codon comprising an unnatural nucleotide derived from

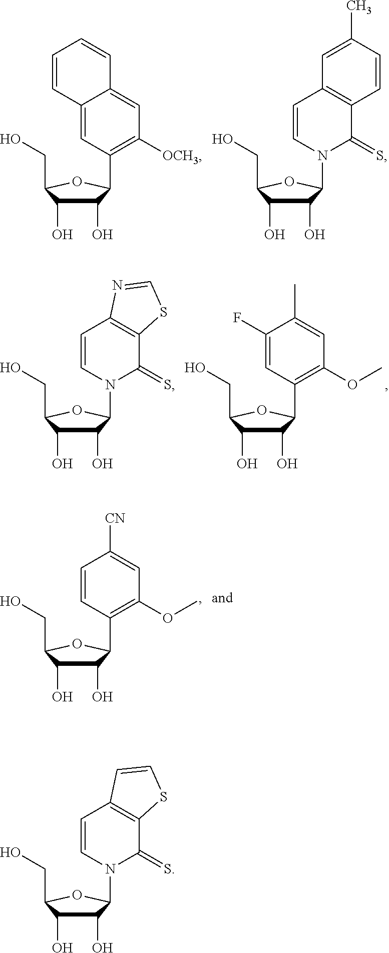

In some embodiments, the M. mazei tRNA may comprise an anti-codon comprising an unnatural nucleotide that recognizes the codon comprising the unnatural nucleotide of the mRNA. The anti-codon in the M. mazei tRNA may comprise an unnatural nucleotide derived from

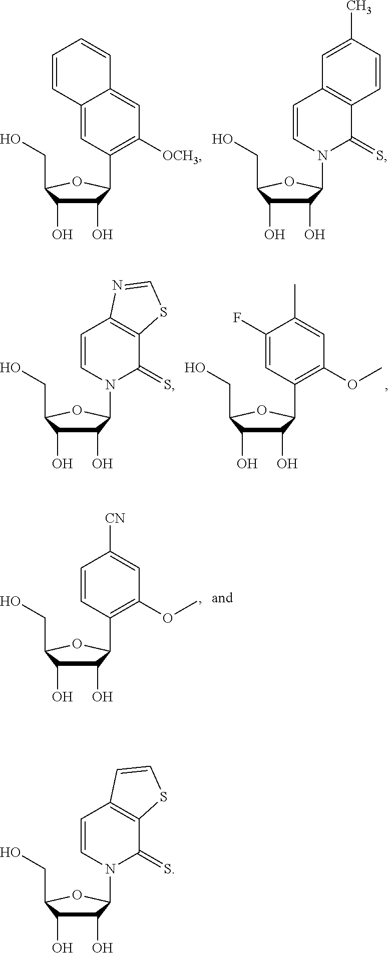

In some embodiments, the mRNA comprises an unnatural nucleotide derived from

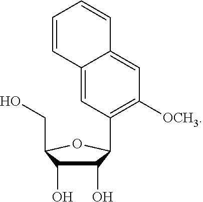

In some embodiments, the mRNA comprises an unnatural nucleotide derived from

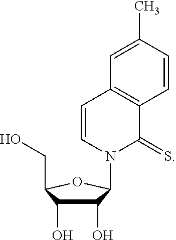

In some embodiments, the mRNA comprises an unnatural nucleotide derived from

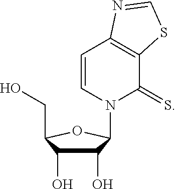

In some embodiments, the mRNA comprises an unnatural nucleotide derived from

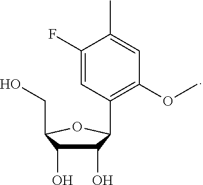

In some embodiments, the mRNA comprises an unnatural nucleotide derived from

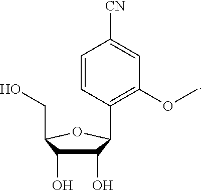

In some embodiments, the mRNA comprises an unnatural nucleotide derived from

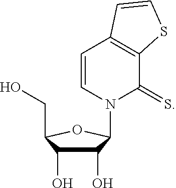

In some embodiments, the tRNA comprises an unnatural nucleotide derived from

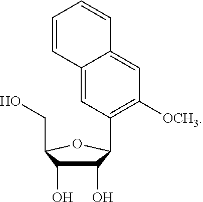

In some embodiments, the tRNA comprises an unnatural nucleotide derived from

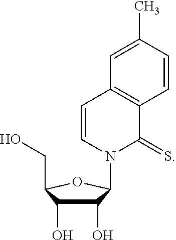

In some embodiments, the tRNA comprises an unnatural nucleotide derived from

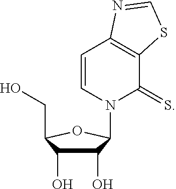

In some embodiments, the tRNA comprises an unnatural nucleotide derived from

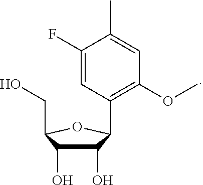

In some embodiments, the tRNA comprises an unnatural nucleotide derived from

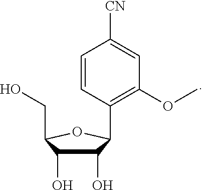

In some embodiments, the tRNA comprises an unnatural nucleotide derived from

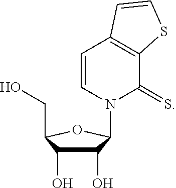

In some embodiments, the mRNA comprises an unnatural nucleotide derived from

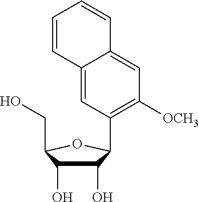

and the tRNA comprises an unnatural nucleotide derived from

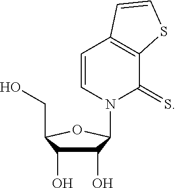

In some embodiments, the mRNA comprises an unnatural nucleotide derived from

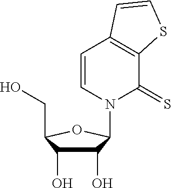

and the tRNA comprises an unnatural nucleotide derived from

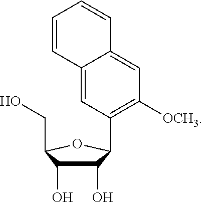

In some embodiments, the mRNA comprises an unnatural nucleotide derived from

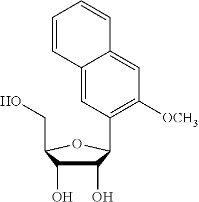

and the tRNA comprises an unnatural nucleotide derived from

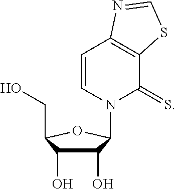

In some embodiments, the mRNA comprises an unnatural nucleotide derived from

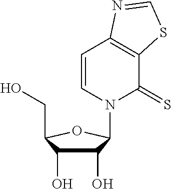

and the tRNA comprises an unnatural nucleotide derived from

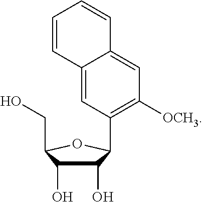

The host cell is cultured in a medium containing appropriate nutrients, and is supplemented with (a) the triphosphates of the deoxyribo nucleosides comprising one or more unnatural bases that are necessary for replication of the plasmid(s) encoding the cytokine gene harboring the codon, (b) the triphosphates of the ribo nucleosides comprising one or more unnatural bases necessary for transcription of (i) the mRNA corresponding to the coding sequence of the cytokine and containing the codon comprising one or more unnatural bases, and (ii) the tRNA containing the anticodon comprising one or more unnatural bases, and (c) the unnatural amino acid(s) to be incorporated in to the polypeptide sequence of the cytokine of interest. The host cells are then maintained under conditions which permit expression of the protein of interest.

The resulting AzK-containing protein that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262: WO 2019014267; WO 2019028419; and WO2019/028425, the disclosures of each of which are incorporated herein by reference.

The resulting protein comprising the one or more unnatural amino acids, Azk for example, that is expressed may be purified by methods known to those of ordinary skill in the art and may then be allowed to react with an alkyne, such as DBCO comprising a PEG chain having a desired average molecular weight as disclosed herein, under conditions known to those of ordinary skill in the art, to afford the IL-2 conjugates disclosed herein. Other methods are known to those of ordinary skill in the art, such as those disclosed in Zhang et al., Nature 2017, 551(7682): 644-647; WO 2015157555; WO 2015021432; WO 2016115168; WO 2017106767; WO 2017223528; WO 2019014262; WO 2019014267; WO 2019028419; and WO2019/028425, the disclosures of each of which are incorporated herein by reference.

Alternatively, a cytokine (e.g., IL-2) polypeptide comprising an unnatural amino acid(s) are prepared by introducing the nucleic acid constructs described herein comprising the tRNA and aminoacyl tRNA synthetase and comprising a nucleic acid sequence of interest with one or more in-frame orthogonal (stop) codons into a host cell. The host cell is cultured in a medium containing appropriate nutrients, is supplemented with (a) the triphosphates of the deoxyribo nucleosides comprising one or more unnatural bases required for replication of the plasmid(s) encoding the cytokine gene harboring the new codon and anticodon, (b) the triphosphates of the ribo nucleosides required for transcription of the mRNA corresponding to (i) the cytokine sequence containing the codon, and (ii) the orthogonal tRNA containing the anticodon, and (c) the unnatural amino acid(s). The host cells are then maintained under conditions which permit expression of the protein of interest. The unnatural amino acid(s) is incorporated into the polypeptide chain in response to the unnatural codon. For example, one or more unnatural amino acids are incorporated into the cytokine (e.g., IL-2) polypeptide. Alternatively, two or more unnatural amino acids may be incorporated into the cytokine (e.g., IL-2) polypeptide at two or more sites in the protein.

Once the cytokine (e.g., IL-2) polypeptide incorporating the unnatural amino acid(s) has been produced in the host cell it can be extracted therefrom by a variety of techniques known in the art, including enzymatic, chemical and/or osmotic lysis and physical disruption. The cytokine (e.g., IL-2) polypeptide can be purified by standard techniques known in the art such as preparative ion exchange chromatography, hydrophobic chromatography, affinity chromatography, or any other suitable technique known to those of ordinary skill in the art.

Suitable host cells may include bacterial cells (e.g., E. coli, BL21(DE3)), but most suitably host cells are eukaryotic cells, for example insect cells (e.g. Drosophila such as Drosophila melanogasler), yeast cells, nematodes (e.g. C. elegans), mice (e.g. Mus musculus), or mammalian cells (such as Chinese hamster ovary cells (CHO) or COS cells, human 293T cells, HeLa cells, NIH 3T3 cells, and mouse erythroleukemia (MEL) cells) or human cells or other eukaryotic cells. Other suitable host cells are known to those skilled in the art. Suitably, the host cell is a mammalian cell—such as a human cell or an insect cell. In some embodiments, the suitable host cells comprise E. coli.

Other suitable host cells which may be used generally in the embodiments of the invention are those mentioned in the examples section. Vector DNA can be introduced into host cells via conventional transformation or transfection techniques. As used herein, the terms “transformation” and “transfection” are intended to refer to a variety of well-recognized techniques for introducing a foreign nucleic acid molecule (e.g., DNA) into a host cell, including calcium phosphate or calcium chloride co-precipitation, DEAE-dextran-mediated transfection, lipofection, or electroporation. Suitable methods for transforming or transfecting host cells are well known in the art.

When creating cell lines, it is generally preferred that stable cell lines are prepared. For stable transfection of mammalian cells for example, it is known that, depending upon the expression vector and transfection technique used, only a small fraction of cells may integrate the foreign DNA into their genome. In order to identify and select these integrants, a gene that encodes a selectable marker (for example, for resistance to antibiotics) is generally introduced into the host cells along with the gene of interest. Preferred selectable markers include those that confer resistance to drugs, such as G418, hygromycin, or methotrexate. Nucleic acid molecules encoding a selectable marker can be introduced into a host cell on the same vector or can be introduced on a separate vector. Cells stably transfected with the introduced nucleic acid molecule can be identified by drug selection (for example, cells that have incorporated the selectable marker gene will survive, while the other cells die).

In one embodiment, the constructs described herein are integrated into the genome of the host cell. An advantage of stable integration is that the uniformity between individual cells or clones is achieved. Another advantage is that selection of the best producers may be carried out. Accordingly, it is desirable to create stable cell lines. In another embodiment, the constructs described herein are transfected into a host cell. An advantage of transfecting the constructs into the host cell is that protein yields may be maximized. In one aspect, there is described a cell comprising the nucleic acid construct or the vector described herein.

### Additional Agents

In some embodiments, described herein is a method of treating a proliferative disease or condition in a subject in need thereof, which comprises administering to the subject a therapeutically effective amount of a cytokine conjugate (e.g., an IL-2 conjugate) described herein. In some embodiments, described herein is a method of treating cancer in a subject in need thereof, which comprises administering to the subject a therapeutically effective amount of a cytokine conjugate (e.g., an IL-2 conjugate) described herein in combination with one or more additional agents. In some embodiments, described herein is a method of treating cancer in a subject in need thereof, which comprises administering to the subject a therapeutically effective amount of a cytokine conjugate (e.g., an IL-2 conjugate) described herein in combination with one or more immune checkpoint inhibitors.

In some embodiment, the one or more additional agents comprises one or more immune checkpoint inhibitors selected from PD-1 inhibitors. In some embodiment, the one or more additional agents comprises one or more PD-1 inhibitors. In some embodiments, the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen), BI-754091 (Boehringer Ingelheim), CC-90006 (Celgene), JTX-4014 (Jounce), PF-06801591 (Pfizer), and genolimzumab (Apollomics/Genor BioPharma). In some embodiments, the one or more PD-1 inhibitors is pembrolizumab. In some embodiments, the one or more PD-1 inhibitors is nivolumab. In some embodiments, the one or more PD-1 inhibitors is cemiplimab. In some embodiments, the one or more PD-1 inhibitors is lambrolizumab. In some embodiments, the one or more PD-1 inhibitors is AMP-224. In some embodiments, the one or more PD-1 inhibitors is sintilimab. In some embodiments, the one or more PD-1 inhibitors is toripalimab. In some embodiments, the one or more PD-1 inhibitors is camrelizumab. In some embodiments, the one or more PD-1 inhibitors is tislelizumab.

In some embodiments, the one or more additional agents comprises immune checkpoint inhibitors selected from PD-L1 inhibitors. In some embodiments, the one or more PD-L1 inhibitors is selected from atezolizumab, avelumab, and durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics). In some embodiments, the one or more PD-L1 inhibitors is atezolizumab. In some embodiments, the one or more PD-L1 inhibitors is avelumab. In some embodiments, the one or more PD-L1 inhibitors is durvalumab. In some embodiments, the one or more immune checkpoint inhibitors is selected from CTLA-4 inhibitors. In some embodiments, the one or more CTLA-4 inhibitors is selected from tremelimumab, ipilimumab, and AGEN-1884 (Agenus). In some embodiments, the one or more CTLA-4 inhibitors is tremelimumab. In some embodiments, the one or more CTLA-4 inhibitors is ipilimumab.

In some embodiments, the one or more additional agents comprises immune checkpoint inhibitors selected from CTLA-4 inhibitors. In some embodiments, the CTLA-4 inhibitor is selected from tremelimumab and ipilimumab. In some embodiments, the CTLA-4 inhibitor is tremelimumab. In some embodiments, the CTLA-4 inhibitor is ipilimumab.

### Methods of Treatment

Described herein are methods of treating cancer in a subject in need thereof, comprising administering to the subject an effective amount of: (a) an IL-2 conjugate as described herein, and (b) one or more additional agents. In some embodiments, described herein are methods of treating cancer in a subject in need thereof, comprising administering to the subject an effective amount of: (a) an IL-2 conjugate as described herein, and (b) one or more immune checkpoint inhibitors.

### Cancer Types

Described herein are methods of treating cancer in a subject, comprising administering to a subject in need thereof an effective amount of an IL-2 conjugate described herein. In some embodiments of a method of treating cancer described herein, the cancer in the subject is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

In some embodiments of a method of treating cancer described herein, the cancer in the subject is selected from renal cell carcinoma (RCC), non-small cell lung cancer (NSCLC), urothelial carcinoma, melanoma, Merkel cell carcinoma (MCC), and head and neck squamous cell cancer (HNSCC). In one embodiment, the cancer is renal cell carcinoma (RCC). In one embodiment, the cancer is non-small cell lung cancer (NSCLC). In one embodiment, the cancer is urothelial carcinoma. In one embodiment, the cancer is melanoma. In one embodiment, the cancer is Merkel cell carcinoma (MCC). In one embodiment, the cancer is head and neck squamous cell cancer (HNSCC).

In some embodiments are provided the methods described herein wherein the one or more additional agents comprises one or more immune checkpoint inhibitors.

In some embodiments, the one or more immune checkpoint inhibitors is selected from the group consisting of PD-1 inhibitors, PD-L1 inhibitors, PD-L2 inhibitors, CTLA-4 inhibitors, OX40 agonists and 4-1BB agonists.

In some embodiments, the one or more immune checkpoint inhibitors is selected from PD-1 inhibitors. In some embodiments, the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma). In some embodiments, the one or more PD-1 inhibitors is pembrolizumab. In some embodiments, the one or more PD-1 inhibitors is nivolumab. In some embodiments, the one or more PD-1 inhibitors is cemiplimab. In some embodiments, the one or more PD-1 inhibitors is lambrolizumab. In some embodiments, the one or more PD-1 inhibitors is AMP-224. In some embodiments, the one or more PD-1 inhibitors is sintilimab. In some embodiments, the one or more PD-1 inhibitors is toripalimab. In some embodiments, the one or more PD-1 inhibitors is camrelizumab. In some embodiments, the one or more PD-1 inhibitors is tislelizumab.

In some embodiments, the one or more PD-L inhibitors is selected from atezolizumab, avelumab, and durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics). In some embodiments, the one or more PD-L1 inhibitors is atezolizumab. In some embodiments, the one or more PD-L1 inhibitors is avelumab. In some embodiments, the one or more PD-L1 inhibitors is durvalumab. In some embodiments, the one or more immune checkpoint inhibitors is selected from CTLA-4 inhibitors. In some embodiments, the one or more CTLA-4 inhibitors is selected from tremelimumab, ipilimumab, and AGEN-1884 (Agenus). In some embodiments, the one or more CTLA-4 inhibitors is tremelimumab. In some embodiments, the one or more CTLA-4 inhibitors is ipilimumab.

In some embodiments, the one or more immune checkpoint inhibitors is selected from CTLA-4 inhibitors. In some embodiments, the CTLA-4 inhibitor is selected from tremelimumab and ipilimumab. In some embodiments, the CTLA-4 inhibitor is tremelimumab. In some embodiments, the CTLA-4 inhibitor is ipilimumab.

In some embodiments, the cancer is in the form of a solid tumor. In some embodiments, the cancer is in the form of a liquid tumor.

In some embodiments, the IL-2 conjugate is administered to the subject prior to the administration to the subject of the one or more additional agents. In some embodiments, the one or more additional agents is administered to the subject prior to the administration to the subject of the IL-2 conjugate. In some embodiments, the IL-2 conjugate and the one or more additional agents are simultaneously administered to the subject.

In some embodiments, the method further comprises administering to the subject a therapeutically effective amount of one or more vascular endothelial cell growth factor (VEGF) pathway or mammalian target of rapamycin (mTOR) inhibitors in addition to one or more checkpoint inhibitors. In some embodiments, the subject is administered one or more VEGF pathway inhibitors. In some embodiments, the one or more VEGF pathway inhibitors is selected from a group consisting of vascular endothelial cell growth factor receptor (VEGFR) tyrosine kinase inhibitors (TKIs) and anti-VEGF monoclonal antibodies. In some embodiments, the one or more VEGF pathway inhibitors is selected from one or more VEGFR TKIs. In some embodiments, the one or more VEGFR TKI is selected from a group consisting of cabozantinib, axitinib, pazopanib, sunitinib, or sorafenib. In some embodiments, the one or more VEGFR TKIs is cabozantinib. In some embodiments, the one or more VEGFR TKIs is axitinib. In some embodiments, the one or more VEGFR TKIs is pazopanib. In some embodiments, the one or more VEGFR TKIs is sunitinib. In some embodiments, wherein the one or more VEGFR TKIs is sorafenib. In some embodiments, the one or more VEGF pathway inhibitors is selected from one or more anti-VEGF monoclonal antibodies. In some embodiments, the one or more anti-VEGF monoclonal antibodies is bevacizumab.

In some embodiments, the one or more mTOR inhibitors is selected from a group consisting of rapamycin, everolimus, temsirolimus, ridaforolimus, and deforolimus. In some embodiments, the one or more mTOR inhibitors is rapamycin. In some embodiments, the one or more mTOR inhibitors is everolimus. In some embodiments, the one or more mTOR inhibitors is temsirolimus. In some embodiments, the one or more mTOR inhibitors is ridaforolimus. In some embodiments, the one or more mTOR inhibitors is deforolimus. In some embodiments, the cancer in the subject is renal cell carcinoma (RCC).

In some embodiments, the methods further comprise administering to the subject a therapeutically effective amount of one or more poly-ADP ribose polymerase (PARP) inhibitors in addition to one or more checkpoint inhibitors. In some embodiments, the PARP inhibitors are selected from the group consisting of olaparib, niraparib, rucaparib, talazoparib, veliparib, CEP-9722, and E7016. In some embodiments, the PARP inhibitor is olaparib. In some embodiments, the PARP inhibitor is niraparib. In some embodiments, the PARP inhibitor is rucaparib. In some embodiments, the PARP inhibitor is talazoparib. In some embodiments, the PARP inhibitor is veliparib. In some embodiments, the PARP inhibitor is CEP-9722. In some embodiments, the PARP inhibitor is E7016.

In some embodiments, the methods further comprise administering to the subject a therapeutically effective amount of a nonsteroidal antiandrogen compound (NSAA) in addition to one or more checkpoint inhibitors. In some embodiments, the NSAA is flutamide, nilutamide, bicalutamide, topilutamide, apalutamide, or enzalutamide. In some embodiments, the NSAA is flutamide. In some embodiments, the NSAA is nilutamide. In some embodiments, the NSAA is bicalutamide. In some embodiments, the NSAA is topilutamide. In some embodiments, the NSAA is apalutamide. In some embodiments, the NSAA is enzalutamide.

In some embodiments, the methods further comprise administering to the subject a therapeutically effective amount of one or more poly-ADP ribose polymerase (PARP) inhibitors and a nonsteroidal antiandrogen compound (NSAA) in addition to one or more checkpoint inhibitors, wherein the PARP inhibitors and NSAA may independently selected from those set forth above.

In some embodiments, the one or more additional agents further comprises one or more chemotherapeutic agents, in addition to one or more checkpoint inhibitors. In some embodiments, the one or more chemotherapeutic agents comprises one or more platinum-based chemotherapeutic agents. In some embodiments, the one or more chemotherapeutic agents comprises carboplatin and pemetrexed. In some embodiments, the one or more chemotherapeutic agents comprises carboplatin and nab-paclitaxel. In some embodiments, the one or more chemotherapeutic agents comprises carboplatin and docetaxel. In some embodiments, the cancer in the subject is non-small cell lung cancer (NSCLC).

In some embodiments, the one or more additional agents is one or more chemotherapeutic agents. In some embodiments, the one or more chemotherapeutic agents comprises one or more platinum based chemotherapeutic agents. In some embodiments, the subject has tested positive for human papillomavirus (HPV) prior to administration of the IL-2 conjugate and one or more additional agents. In some embodiments, the cancer in the subject is head and neck squamous cell cancer (HNSCC). In some embodiments, the method further comprises the subject testing positive for human papillomavirus (HPV+), followed by administration of the IL-2 conjugate and one or more additional agents.

### Administration

In some embodiments, following administration of the IL-2 conjugate and the one or more additional agents, the subject experiences a response as measured by the Immune-related Response Evaluation Criteria in Solid Tumors (iRECIST).

In some embodiments, the response is a complete response, a partial response or stable disease. In some embodiments, the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration. In some embodiments, the IL-2 conjugate is administered to a subject by intravenous, subcutaneous, or intramuscular administration. In some embodiments, the IL-2 conjugate is administered to a subject by intravenous administration. In some embodiments, the IL-2 conjugate is administered to a subject by subcutaneous administration. In some embodiments, the IL-2 conjugate is administered to a subject by intramuscular administration.

In some embodiments, an effective amount of the IL-2 conjugate is administered to a subject in need thereof once per week, once every two weeks, once every three weeks, once every 4 weeks, once every 5 weeks, once every 6 weeks, once every 7 weeks, once every 8 weeks, once every 9 weeks, once every 10 weeks, once every 11 weeks, once every 12 weeks, once every 13 weeks, once every 14 weeks, once every 15 weeks, once every 16 weeks, once every 17 weeks, once every 18 weeks, once every 19 weeks, once every 20 weeks, once every 21 weeks, once every 22 weeks, once every 23 weeks, once every 24 weeks, once every 25 weeks, once every 26 weeks, once every 27 weeks, or once every 28 weeks. In some embodiments, an effective amount of the IL-2 conjugate is administered to a subject in need thereof once per week. In some embodiments, an effective amount of the IL-2 conjugate is administered to a subject in need thereof once every two weeks. In some embodiments, an effective amount of the IL-2 conjugate is administered to a subject in need thereof once every three weeks. In some embodiments, an effective amount of the IL-2 conjugate is administered to a subject in need thereof once every 4 weeks. In some embodiments, an effective amount of the IL-2 conjugate is administered to a subject in need thereof once every 5 weeks. In some embodiments, an effective amount of the IL-2 conjugate is administered to a subject in need thereof once every 6 weeks. In some embodiments, an effective amount of the IL-2 conjugate is administered to a subject in need thereof once every 7 weeks. In some embodiments, an effective amount of the IL-2 conjugate is administered to a subject in need thereof once every 8 weeks. In some embodiments, an effective amount of the IL-2 conjugate is administered to a subject in need thereof once every 9 weeks. In some embodiments, an effective amount of the IL-2 conjugate is administered to a subject in need thereof once every 10 weeks. In some embodiments, an effective amount of the IL-2 conjugate is administered to a subject in need thereof once every 11 weeks. In some embodiments, an effective amount of the IL-2 conjugate is administered to a subject in need thereof once every 12 weeks. In some embodiments, an effective amount of the IL-2 conjugate is administered to a subject in need thereof once every 13 weeks. In some embodiments, an effective amount of the IL-2 conjugate is administered to a subject in need thereof once every 14 weeks. In some embodiments, an effective amount of the IL-2 conjugate is administered to a subject in need thereof once every 15 weeks. In some embodiments, an effective amount of the IL-2 conjugate is administered to a subject in need thereof once every 16 weeks. In some embodiments, an effective amount of the IL-2 conjugate is administered to a subject in need thereof once every 17 weeks. In some embodiments, an effective amount of the IL-2 conjugate is administered to a subject in need thereof once every 18 weeks. In some embodiments, an effective amount of the IL-2 conjugate is administered to a subject in need thereof once every 19 weeks. In some embodiments, an effective amount of the IL-2 conjugate is administered to a subject in need thereof once every 20 weeks. In some embodiments, an effective amount of the IL-2 conjugate is administered to a subject in need thereof once every 21 weeks. In some embodiments, an effective amount of the IL-2 conjugate is administered to a subject in need thereof once every 22 weeks. In some embodiments, an effective amount of the IL-2 conjugate is administered to a subject in need thereof once every 23 weeks. In some embodiments, an effective amount of the IL-2 conjugate is administered to a subject in need thereof once every 24 weeks.

In some embodiments, the amount of a given agent that correspond to such an amount varies depending upon factors such as the particular compound, the severity of the disease, the identity (e.g., weight) of the subject or host in need of treatment, but nevertheless is routinely determined in a manner known in the art according to the particular circumstances surrounding the case, including, e.g., the specific agent being administered, the route of administration, and the subject or host being treated. In some instances, the desired dose is conveniently presented in a single dose or as divided doses administered simultaneously (or over a short period of time) or at appropriate intervals, for example as two, three, four or more sub-doses per day.

In some embodiments, the methods include the dosing of an IL-2 conjugate to a subject in need thereof at a dose in the range from 1 μg of the IL-2 conjugate per kg of the subject's body weight to about 200 μg of the IL-2 conjugate per kg of the subject's body weight, or from about 2 μg of the IL-2 conjugate per kg of the subject's body weight to about 200 μg of the IL-2 conjugate per kg of the subject's body weight, or from about 4 μg of the IL-2 conjugate per kg of the subject's body weight to about 200 μg of the IL-2 conjugate per kg of the subject's body weight, or from about 6 μg of the IL-2 conjugate per kg of the subject's body weight to about 200 μg of the IL-2 conjugate per kg of the subject's body weight, or from about 8 μg of the IL-2 conjugate per kg of the subject's body weight to about 200 μg of the IL-2 conjugate per kg of the subject's body weight, or from about 10 μg of the IL-2 conjugate per kg of the subject's body weight to about 200 μg of the IL-2 conjugate per kg of the subject's body weight, or from about 12 μg of the IL-2 conjugate per kg of the subject's body weight to about 200 μg of the IL-2 conjugate per kg of the subject's body weight, or from about 14 μg of the IL-2 conjugate per kg of the subject's body weight to about 200 μg of the IL-2 conjugate per kg of the subject's body weight, or from about 16 μg of the IL-2 conjugate per kg of the subject's body weight to about 200 μg of the IL-2 conjugate per kg of the subject's body weight, or from about 18 μg of the IL-2 conjugate per kg of the subject's body weight to about 200 μg of the IL-2 conjugate per kg of the subject's body weight, or from about 20 μg of the IL-2 conjugate per kg of the subject's body weight to about 200 μg of the IL-2 conjugate per kg of the subject's body weight, or from about 22 μg of the IL-2 conjugate per kg of the subject's body weight to about 200 μg of the IL-2 conjugate per kg of the subject's body weight, or from about 24 μg of the IL-2 conjugate per kg of the subject's body weight to about 200 μg of the IL-2 conjugate per kg of the subject's body weight, or from about 26 μg of the IL-2 conjugate per kg of the subject's body weight to about 200 μg of the IL-2 conjugate per kg of the subject's body weight, or from about 28 μg of the IL-2 conjugate per kg of the subject's body weight to about 200 μg of the IL-2 conjugate per kg of the subject's body weight, or from about 32 μg of the IL-2 conjugate per kg of the subject's body weight to about 200 μg of the IL-2 conjugate per kg of the subject's body weight, or from about 34 μg of the IL-2 conjugate per kg of the subject's body weight to about 200 μg of the IL-2 conjugate per kg of the subject's body weight, or from about 36 μg of the IL-2 conjugate per kg of the subject's body weight to about 200 μg of the IL-2 conjugate per kg of the subject's body weight, or from about 40 μg of the IL-2 conjugate per kg of the subject's body weight to about 200 μg of the IL-2 conjugate per kg of the subject's body weight, or from about 45 μg of the IL-2 conjugate per kg of the subject's body weight to about 200 μg of the IL-2 conjugate per kg of the subject's body weight, or from about 50 μg of the IL-2 conjugate per kg of the subject's body weight to about 200 μg of the IL-2 conjugate per kg of the subject's body weight, or from about 55 μg of the IL-2 conjugate per kg of the subject's body weight to about 200 μg of the IL-2 conjugate per kg of the subject's body weight, or from about 60 μg of the IL-2 conjugate per kg of the subject's body weight to about 200 μg of the IL-2 conjugate per kg of the subject's body weight, or from about 65 μg of the IL-2 conjugate per kg of the subject's body weight to about 200 μg of the IL-2 conjugate per kg of the subject's body weight, or from about 70 μg of the IL-2 conjugate per kg of the subject's body weight to about 200 μg of the IL-2 conjugate per kg of the subject's body weight, or from about 75 μg of the IL-2 conjugate per kg of the subject's body weight to about 200 μg of the IL-2 conjugate per kg of the subject's body weight, or from about 80 μg of the IL-2 conjugate per kg of the subject's body weight to about 200 μg of the IL-2 conjugate per kg of the subject's body weight, or from about 85 μg of the IL-2 conjugate per kg of the subject's body weight to about 200 μg of the IL-2 conjugate per kg of the subject's body weight, or from about 90 μg of the IL-2 conjugate per kg of the subject's body weight to about 200 μg of the IL-2 conjugate per kg of the subject's body weight, or from about 95 μg of the IL-2 conjugate per kg of the subject's body weight to about 200 μg of the IL-2 conjugate per kg of the subject's body weight, or from about 100 μg of the IL-2 conjugate per kg of the subject's body weight to about 200 μg of the IL-2 conjugate per kg of the subject's body weight, or from about 110 μg of the IL-2 conjugate per kg of the subject's body weight to about 200 μg of the IL-2 conjugate per kg of the subject's body weight, or from about 120 μg of the IL-2 conjugate per kg of the subject's body weight to about 200 μg of the IL-2 conjugate per kg of the subject's body weight, or from about 130 μg of the IL-2 conjugate per kg of the subject's body weight to about 200 μg of the IL-2 conjugate per kg of the subject's body weight, or from about 140 μg of the IL-2 conjugate per kg of the subject's body weight to about 200 μg of the IL-2 conjugate per kg of the subject's body weight, or from about 150 μg of the IL-2 conjugate per kg of the subject's body weight to about 200 μg of the IL-2 conjugate per kg of the subject's body weight, or from about 160 μg of the IL-2 conjugate per kg of the subject's body weight to about 200 μg of the IL-2 conjugate per kg of the subject's body weight, or from about 170 μg of the IL-2 conjugate per kg of the subject's body weight to about 200 μg of the IL-2 conjugate per kg of the subject's body weight, or from about 180 μg of the IL-2 conjugate per kg of the subject's body weight to about 200 μg of the IL-2 conjugate per kg of the subject's body weight, or from about 190 μg of the IL-2 conjugate per kg of the subject's body weight to about 200 μg of the IL-2 conjugate per kg of the subject's body weight. The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner. In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some embodiments, the methods include the dosing of an IL-2 conjugate to a subject in need thereof at a dose of about 1 μg of the IL-2 conjugate per kg of the subject's body weight, or about 2 μg of the IL-2 conjugate per kg of the subject's body weight, about 4 μg of the IL-2 conjugate per kg of the subject's body weight, about 6 μg of the IL-2 conjugate per kg of the subject's body weight, about 8 μg of the IL-2 conjugate per kg of the subject's body weight, about 10 μg of the IL-2 conjugate per kg of the subject's body weight, about 12 μg of the IL-2 conjugate per kg of the subject's body weight, about 14 μg of the IL-2 conjugate per kg of the subject's body weight, about 16 μg of the IL-2 conjugate per kg of the subject's body weight, about 18 μg of the IL-2 conjugate per kg of the subject's body weight, about 20 μg of the IL-2 conjugate per kg of the subject's body weight, about 22 μg of the IL-2 conjugate per kg of the subject's body weight, about 24 μg of the IL-2 conjugate per kg of the subject's body weight, about 26 μg of the IL-2 conjugate per kg of the subject's body weight, about 28 μg of the IL-2 conjugate per kg of the subject's body weight, about 30 μg of the IL-2 conjugate per kg of the subject's body weight, about 32 μg of the IL-2 conjugate per kg of the subject's body weight, about 34 μg of the IL-2 conjugate per kg of the subject's body weight, about 36 μg of the IL-2 conjugate per kg of the subject's body weight, about 38 μg of the IL-2 conjugate per kg of the subject's body weight, about 40 μg of the IL-2 conjugate per kg of the subject's body weight, about 42 μg of the IL-2 conjugate per kg of the subject's body weight, about 44 μg of the IL-2 conjugate per kg of the subject's body weight, about 46 μg of the IL-2 conjugate per kg of the subject's body weight, about 48 μg of the IL-2 conjugate per kg of the subject's body weight, about 50 μg of the IL-2 conjugate per kg of the subject's body weight, about 55 μg of the IL-2 conjugate per kg of the subject's body weight, about 60 μg of the IL-2 conjugate per kg of the subject's body weight, about 65 μg of the IL-2 conjugate per kg of the subject's body weight, about 70 μg of the IL-2 conjugate per kg of the subject's body weight, about 75 μg of the IL-2 conjugate per kg of the subject's body weight, about 80 μg of the IL-2 conjugate per kg of the subject's body weight, about 85 μg of the IL-2 conjugate per kg of the subject's body weight, about 90 μg of the IL-2 conjugate per kg of the subject's body weight, about 95 μg of the IL-2 conjugate per kg of the subject's body weight, about 100 μg of the IL-2 conjugate per kg of the subject's body weight, about 110 μg of the IL-2 conjugate per kg of the subject's body weight, about 120 μg of the IL-2 conjugate per kg of the subject's body weight, about 130 μg of the IL-2 conjugate per kg of the subject's body weight, about 140 μg of the IL-2 conjugate per kg of the subject's body weight, about 150 μg of the IL-2 conjugate per kg of the subject's body weight, about 160 μg of the IL-2 conjugate per kg of the subject's body weight, about 170 μg of the IL-2 conjugate per kg of the subject's body weight, about 180 μg of the IL-2 conjugate per kg of the subject's body weight, about 190 μg of the IL-2 conjugate per kg of the subject's body weight, or about 200 μg of the IL-2 conjugate per kg of the subject's body weight. The foregoing ranges are merely suggestive, as the number of variables in regard to an individual treatment regime is large, and considerable excursions from these recommended values are not uncommon. Such dosages are altered depending on a number of variables, not limited to the activity of the compound used, the disease or condition to be treated, the mode of administration, the requirements of the individual subject, the severity of the disease or condition being treated, and the judgment of the practitioner. In some embodiments, toxicity and therapeutic efficacy of such therapeutic regimens are determined by standard pharmaceutical procedures in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index and it is expressed as the ratio between LD50 and ED50. Compounds exhibiting high therapeutic indices are preferred. The data obtained from cell culture assays and animal studies are used in formulating a range of dosage for use in human. The dosage of such compounds lies preferably within a range of circulating concentrations that include the ED50 with minimal toxicity. The dosage varies within this range depending upon the dosage form employed and the route of administration utilized.

In some embodiments, the additional agent may be administered at a dose and using a dosing regimen that has been determined to be safe and efficacious for that additional agent. For example, pembrolizumab may be administered to a subject in need thereof according to the methods described herein at a dose of about 200 mg every 3 weeks. In another example, nivolumab may be administered to a subject in need thereof according to the methods described herein at a dose of about 240 mg every 2 weeks or at a dose of about 480 mg every 4 weeks. In another example, cemiplimab may be administered to a subject in need thereof according to the methods described herein at a dose of about 350 mg as an intravenous infusion over 30 minutes every 3 weeks. In another example, atezolizumab may be administered to a subject according to the methods described herein at a dose of 840 mg every 2 weeks, 1200 mg every 3 weeks, or 1680 mg every 4 weeks. In another example, avelumab may be administered to a subject according to the methods described herein at a dose of 800 mg every 2 weeks. In another example, durvalumab may be administered to a subject according to the methods described herein at a dose of 10 mg per kg of the subject's body weight very 2 weeks. In another example, ipilimumab may be administered to a subject for the treatment of melanoma according to the methods described herein at a dose of about 3 mg per kg of the subject's body weight over 90 minutes every three weeks for a total of 4 doses, or about 10 mg per kg of the subject's body weight over 90 minutes for a total of 4 doses, followed by 10 mg per kg of the subject's body weight for up to 3 years. For advanced renal cell carcinoma, ipilimumab may be administered according to the methods described herein at a dose of 1 mg per kg of the subject's body weight over 30 minutes.

In some embodiments of a method of treating cancer described herein, administration of the effective amount of the IL-2 conjugate to the subject does not require the availability of an intensive care facility or skilled specialists in cardiopulmonary or intensive care medicine. In some embodiments of a method of treating cancer described herein, administration of the effective amount of the IL-2 conjugate to the subject does not require the availability of an intensive care facility or skilled specialists in cardiopulmonary or intensive care medicine. In some embodiments of a method of treating cancer described herein, administration of the effective amount of the IL-2 conjugate to the subject does not require the availability of an intensive care facility. In some embodiments of a method of treating cancer described herein, administration of the effective amount of the IL-2 conjugate to the subject does not require the availability of skilled specialists in cardiopulmonary or intensive care medicine.

### Effects of Administration

In some embodiments of a method of treating cancer described herein, administration of the effective amount of the IL-2 conjugate to the subject does not cause vascular leak syndrome in the subject. In some embodiments of a method of treating cancer described herein, administration of the effective amount of the IL-2 conjugate to the subject does not cause Grade 2, Grade 3, or Grade 4 vascular leak syndrome in the subject. In some embodiments of a method of treating cancer described herein, administration of the effective amount of the IL-2 conjugate to the subject does not cause Grade 2 vascular leak syndrome in the subject. In some embodiments of a method of treating cancer described herein, administration of the effective amount of the IL-2 conjugate to the subject does not cause Grade 3 vascular leak syndrome in the subject. In some embodiments of a method of treating cancer described herein, administration of the effective amount of the IL-2 conjugate to the subject does not cause Grade 4 vascular leak syndrome in the subject.

In some embodiments of a method of treating cancer described herein, administration of the effective amount of the IL-2 conjugate to the subject does not cause loss of vascular tone in the subject.

In some embodiments of a method of treating cancer described herein, administration of the effective amount of the IL-2 conjugate to the subject does not cause extravasation of plasma proteins and fluid into the extravascular space in the subject.

In some embodiments of a method of treating cancer described herein, administration of the effective amount of the IL-2 conjugate to the subject does not cause hypotension and reduced organ perfusion in the subject.

In some embodiments of a method of treating cancer described herein, administration of the effective amount of the IL-2 conjugate to the subject does not cause impaired neutrophil function in the subject. In some embodiments of a method of treating cancer described herein, administration of the effective amount of the IL-2 conjugate to the subject does not cause reduced chemotaxis in the subject.

In some embodiments of a method of treating cancer described herein, administration of the effective amount of the IL-2 conjugate to the subject is not associated with an increased risk of disseminated infection in the subject. In some embodiments of a method of treating cancer described herein, the disseminated infection is sepsis or bacterial endocarditis. In some embodiments of a method of treating cancer described herein, the disseminated infection is sepsis. In some embodiments of a method of treating cancer described herein, the disseminated infection is bacterial endocarditis. In some embodiments of a method of treating cancer described herein, the subject is treated for any preexisting bacterial infections prior to administration of the IL-2 conjugate. In some embodiments of a method of treating cancer described herein, the subject is treated with an antibacterial agent selected from oxacillin, nafcillin, ciprofloxacin, and vancomycin prior to administration of the IL-2 conjugate.

In some embodiments of a method of treating cancer described herein, administration of the effective amount of the IL-2 conjugate to the subject does not exacerbate a pre-existing or initial presentation of an autoimmune disease or an inflammatory disorder in the subject. In some embodiments of a method of treating cancer described herein, the administration of the effective amount of the IL-2 conjugate to the subject does not exacerbate a pre-existing or initial presentation of an autoimmune disease in the subject. In some embodiments of a method of treating cancer described herein, the administration of the effective amount of the IL-2 conjugate to the subject does not exacerbate a pre-existing or initial presentation of an inflammatory disorder in the subject. In some embodiments of a method of treating cancer described herein, the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid. In some embodiments of a method of treating cancer described herein, the autoimmune disease or inflammatory disorder in the subject is Crohn's disease. In some embodiments of a method of treating cancer described herein, the autoimmune disease or inflammatory disorder in the subject is scleroderma. In some embodiments of a method of treating cancer described herein, the autoimmune disease or inflammatory disorder in the subject is thyroiditis. In some embodiments of a method of treating cancer described herein, the autoimmune disease or inflammatory disorder in the subject is inflammatory arthritis. In some embodiments of a method of treating cancer described herein, the autoimmune disease or inflammatory disorder in the subject is diabetes mellitus. In some embodiments of a method of treating cancer described herein, the autoimmune disease or inflammatory disorder in the subject is oculo-bulbar myasthenia gravis. In some embodiments of a method of treating cancer described herein, the autoimmune disease or inflammatory disorder in the subject is crescentic IgA glomerulonephritis. In some embodiments of a method of treating cancer described herein, the autoimmune disease or inflammatory disorder in the subject is cholecystitis. In some embodiments of a method of treating cancer described herein, the autoimmune disease or inflammatory disorder in the subject is cerebral vasculitis. In some embodiments of a method of treating cancer described herein, the autoimmune disease or inflammatory disorder in the subject is Stevens-Johnson syndrome. In some embodiments of a method of treating cancer described herein, the autoimmune disease or inflammatory disorder in the subject is bullous pemphigoid.

In some embodiments of a method of treating cancer described herein, administration of the effective amount of the IL-2 conjugate to the subject does not cause changes in mental status, speech difficulties, cortical blindness, limb or gait ataxia, hallucinations, agitation, obtundation, or coma in the subject. In some embodiments of a method of treating cancer described herein, administration of the effective amount of the IL-2 conjugate to the subject does not cause seizures in the subject. In some embodiments of a method of treating cancer described herein, administration of the effective amount of the IL-2 conjugate to the subject is not contraindicated in subjects having a known seizure disorder.

In some embodiments of a method of treating cancer described herein, administration of the effective amount of the IL-2 conjugate to the subject does not cause capillary leak syndrome in the subject. In some embodiments of a method of treating cancer described herein, administration of the effective amount of the IL-2 conjugate to the subject does not cause Grade 2, Grade 3, or Grade 4 capillary leak syndrome in the subject. In some embodiments of a method of treating cancer described herein, administration of the effective amount of the IL-2 conjugate to the subject does not cause Grade 2 capillary leak syndrome in the subject. In some embodiments of a method of treating cancer described herein, administration of the effective amount of the IL-2 conjugate to the subject does not cause Grade 3 capillary leak syndrome in the subject. In some embodiments of a method of treating cancer described herein, administration of the effective amount of the IL-2 conjugate to the subject does not cause Grade 4 capillary leak syndrome in the subject.

In some embodiments of a method of treating cancer described herein, administration of the effective amount of the IL-2 conjugate to the subject does not cause a drop in mean arterial blood pressure in the subject following administration of the IL-2 conjugate to the subject. In some embodiments of a method of treating cancer described herein, administration of the effective amount of the IL-2 conjugate to the subject does not cause hypotension in the subject following administration of the IL-2 conjugate to the subject. In some embodiments of a method of treating cancer described herein, administration of the effective amount of the IL-2 conjugate to the subject does not cause the subject to experience a systolic blood pressure below 90 mm Hg or a 20 mm Hg drop from baseline systolic pressure following administration of the IL-2 conjugate to the subject.

In some embodiments of a method of treating cancer described herein, administration of the effective amount of the IL-2 conjugate to the subject does not cause edema in the subject following administration of the IL-2 conjugate to the subject. In some embodiments of a method of treating cancer described herein, administration of the effective amount of the IL-2 conjugate to the subject does not cause impairment of kidney or liver function in the subject following administration of the IL-2 conjugate to the subject.

In some embodiments of a method of treating cancer described herein, administration of the effective amount of the IL-2 conjugate to the subject does not cause eosinophilia in the subject following administration of the IL-2 conjugate to the subject. In some embodiments of a method of treating cancer described herein, administration of the effective amount of the IL-2 conjugate to the subject does not cause the eosinophil count in the peripheral blood of the subject to exceed 500 per L following administration of the IL-2 conjugate to the subject. In some embodiments of a method of treating cancer described herein, administration of the effective amount of the IL-2 conjugate to the subject does not cause the eosinophil count in the peripheral blood of the subject to exceed 500 μL to 1500 per μL following administration of the IL-2 conjugate to the subject. In some embodiments of a method of treating cancer described herein, administration of the effective amount of the IL-2 conjugate to the subject does not cause the eosinophil count in the peripheral blood of the subject to exceed 1500 per μL to 5000 per μL following administration of the IL-2 conjugate to the subject. In some embodiments of a method of treating cancer described herein, administration of the effective amount of the IL-2 conjugate to the subject does not cause the eosinophil count in the peripheral blood of the subject to exceed 5000 per μL following administration of the IL-2 conjugate to the subject. In some embodiments of a method of treating cancer described herein, administration of the effective amount of the IL-2 conjugate to the subject is not contraindicated in subjects on an existing regimen of psychotropic drugs.

In some embodiments of a method of treating cancer described herein, administration of the effective amount of the IL-2 conjugate to the subject is not contraindicated in subjects on an existing regimen of nephrotoxic, myelotoxic, cardiotoxic, or hepatotoxic drugs. In some embodiments of a method of treating cancer described herein, administration of the effective amount of the IL-2 conjugate to the subject is not contraindicated in subjects on an existing regimen of aminoglycosides, cytotoxic chemotherapy, doxorubicin, methotrexate, or asparaginase. In some embodiments of a method of treating cancer described herein, administration of the effective amount of the IL-2 conjugate to the subject is not contraindicated in subjects receiving combination regimens containing antineoplastic agents. In some embodiments of a method of treating cancer described herein, the antineoplastic agent is selected from dacarbazine, cis-platinum, tamoxifen and interferon-alfa.

In some embodiments of a method of treating cancer described herein, administration of the effective amount of the IL-2 conjugate to the subject does not cause one or more Grade 4 adverse events in the subject following administration of the IL-2 conjugate to the subject. In some embodiments of a method of treating cancer described herein, the one or more Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis. In some embodiments of a method of treating cancer described herein, administration of the effective amount of the IL-2 conjugate to a group of subjects does not cause one or more Grade 4 adverse events in greater than 1% of the subjects following administration of the IL-2 conjugate to the subjects. In some embodiments of a method of treating cancer described herein, the one or more Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

In some embodiments of a method of treating cancer described herein, administration of the effective amount of the IL-2 conjugate to a group of subjects does not cause one or more adverse events in greater than 1% of the subjects following administration of the IL-2 conjugate to the subjects, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

In some embodiments of a method of treating cancer described herein, administration of the effective amount of the IL-2 conjugate to a group of subjects does not cause one or more adverse events in greater than 1% of the subjects following administration of the IL-2 conjugate to the subjects, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

In some embodiments of a method of treating cancer described herein, administration of the IL-2 conjugate to the subject increases the number of peripheral CD8+T and NK cells in the subject without increasing the number of peripheral CD4+ regulatory T cells in the subject. In some embodiments of a method of treating cancer described herein, administration of the IL-2 conjugate to the subject increases the number of peripheral CD8+T and NK cells in the subject without increasing the number of peripheral eosinophils in the subject. In some embodiments of a method of treating cancer described herein, administration of the IL-2 conjugate to the subject increases the number of peripheral CD8+ T and NK cells in the subject without increasing the number of intratumoral CD8+ T and NK cells in the subject without increasing the number of intratumoral CD4+ regulatory T cells in the subject.

### Pharmaceutical Compositions and Formulations

Described herein are pharmaceutical compositions comprising an effective amount of an IL-2 conjugate described herein and one or more pharmaceutically acceptable excipients.

In some embodiments, the pharmaceutical composition and formulations comprising a cytokine conjugate (e.g., IL-2 conjugate) described herein are administered to a subject by multiple administration routes, including but not limited to, parenteral, oral, buccal, rectal, sublingual, or transdermal administration routes. In some cases, parenteral administration comprises intravenous, subcutaneous, intramuscular, intracerebral, intranasal, intra-arterial, intra-articular, intradermal, intravitreal, intraosseous infusion, intraperitoneal, or intratechal administration. In some instances, the pharmaceutical composition is formulated for local administration. In other instances, the pharmaceutical composition is formulated for systemic administration. In some embodiments, the pharmaceutical composition and formulations described herein are administered to a subject by intravenous, subcutaneous, and intramuscular administration. In some embodiments, the pharmaceutical composition and formulations described herein are administered to a subject by intravenous administration. In some embodiments, the pharmaceutical composition and Formulations described herein are administered to a subject by administration. In some embodiments, the pharmaceutical composition and formulations described herein are administered to a subject by intramuscular administration.

In some embodiments, the pharmaceutical formulations include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999), the disclosures of each of which are incorporated herein by reference.

In some cases, the pharmaceutical composition is formulated as an immunoliposome, which comprises a plurality of IL-2 conjugates bound either directly or indirectly to lipid bilayer of liposomes. Exemplary lipids include, but are not limited to, fatty acids; phospholipids; sterols such as cholesterols; sphingolipids such as sphingomyelin; glycosphingolipids such as gangliosides, globocides, and cerebrosides; surfactant amines such as stearyl, oleyl, and linoleyl amines. In some instances, the lipid comprises a cationic lipid. In some instances, the lipid comprises a phospholipid. Exemplary phospholipids include, but are not limited to, phosphatidic acid (“PA”), phosphatidylcholine (“PC”), phosphatidylglycerol (“PG”), phophatidylethanolamine (“PE”), phophatidylinositol (“PI”), and phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), distearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DMPG”), dioleoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DMPI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), didecanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, and 1-palmitoyl-2-oleoyl-sphingomyelin.

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as Avicel®, dibasic calcium phosphate, dicalcium phosphate dihydrate, tricalcium phosphate, calcium phosphate, anhydrous lactose, spray-dried lactose, pregelatinized starch, compressible sugar, such as Di-Pac® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like. In some embodiments, the IL-2 conjugates disclosed herein may be used in pharmaceutical formulations comprising histidine, sorbitol, and polysorbate 80, or any combination that affords a stable Formulation and can be administered to subjects in need thereof. In one embodiment, the IL-2 conjugates disclosed herein may be presented as a finished drug product in a suitable container, such as a vial, as follows: IL-2 conjugate (about 2 mg to about 10 mg); L-histidine (about 0.5 mg to about 2 mg); L-histidine hydrochloride (about 1 mg to about 2 mg); sorbitol (about 20 mg to about 80 mg); and polysorbate 80 (about 0.1 mg to about 0.2 mg); with a sufficient quantity of water for injection to provide a liquid Formulation suitable for use in the disclosed methods.

In some cases, the pharmaceutical formulations include disintegration agents or disintegrants to facilitate the breakup or disintegration of a substance. The term “disintegrate” includes both the dissolution and dispersion of the dosage form when contacted with gastrointestinal fluid. Examples of disintegration agents include a starch, e.g., a natural starch such as corn starch or potato starch, a pregelatinized starch such as National 1551 or Amijel®, or sodium starch glycolate such as Promogel® or Explotab®, a cellulose such as a wood product, methylcrystalline cellulose, e.g., Avicel®, Avicel® PH101, Avicel®PH102, Avicel® PH105, Elcema® P100, Emcocel®, Vivacel®, Ming Tia, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or Tween® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Additional surfactants include polyoxyethylene fatty acid glycerides and vegetable oils, e.g., polyoxyethylene (60) hydrogenated castor oil, and polyoxyethylene alkylethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some instances, a pharmaceutical composition comprising an IL-2 conjugate (such as those of Formulas (I-XVII)) and an immune checkpoint inhibitor is administered as a formulation comprising both drugs. In some instances, the percent by weight of the IL-2 conjugate to the immune checkpoint inhibitor or vice versa is between 10:1 to 1:10. In some instances, the percent by weight of the IL-2 conjugate to the immune checkpoint inhibitor or vice versa is between 7:1 to 1:2. In some instances, the percent by weight of the IL-2 conjugate to the immune checkpoint inhibitor or vice versa is between 5:1 to 1:5. In some instances, the percent by weight of the IL-2 conjugate to the immune checkpoint inhibitor or vice versa is between 3:1 to 1:3. In some instances, the percent by weight of IL-2 conjugate to the immune checkpoint inhibitor or vice versa is about 10:1, or about 9:1, about 8:1, about 7:1, about 6:1, about 5:1, about 4:1, about 3:1, about 2:1, or about 1:1. In some instances, the percent by weight of IL-2 conjugate to the immune checkpoint inhibitor or vice versa is 10:1 to 1:1, 7:1 to 2:1, 5:1 to 1:1, or 3:1 to 1:1.

In certain embodiments, the combination of an immune checkpoint inhibitor (such as a PD-1 inhibitor) an IL-2 conjugate (such as those of Formulas (I-XVII)) as described herein is administered as a pure chemical. In other embodiments, the combination of an immune checkpoint inhibitor and an IL-2 conjugate described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005), the disclosure of which is incorporated herein by reference). In some embodiments, an immune checkpoint inhibitor and an IL-2 conjugate described herein are each administered as individual compositions. In some embodiments, individual compositions of an immune checkpoint inhibitor and/or an IL-2 conjugate described herein are combined with a suitable or acceptable excipient. In some embodiments, an immune checkpoint inhibitor and an IL-2 conjugate described herein are administered as a single, combined composition.

Provided herein are pharmaceutical compositions comprising an IL-2 conjugate described herein, and an immune checkpoint inhibitor together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition. In certain embodiments, an immune checkpoint inhibitor and an IL-2 conjugate described herein is substantially pure, in that it contains less than about 5%, or less than about 1%, or less than about 0.1%, of other organic small molecules, such as unreacted intermediates or synthesis by-products that are created, for example, in one or more of the steps of a synthesis method. In some instances, the pharmaceutical compositions comprise an immune checkpoint inhibitor and an IL-2 conjugate described herein, and one or more pharmaceutically acceptable excipients. In some instances, pharmaceutical compositions comprising an immune checkpoint inhibitor and an IL-2 conjugate described herein, or combination thereof comprise (by way of non-limiting example) excipients such as 0.9% sodium chloride injection USP, dehydrated alcohol, dl-alpha tocopherol, anhydrous citric acid, polysorbate 80, polyethylene glycol 400, propylene glycol, benzyl alcohol, sodium citrate, sodium sulfite, cremophor EL, albumin, or any combination thereof. In some instances, the pharmaceutical compositions comprise nanoparticles. In some instances, the pharmaceutical compositions comprise other excipients commonly used in injectable compositions. In some instances, the pharmaceutical compositions comprise a contrast agent to aid in visualization of the delivery of the pharmaceutical composition. In some instances, the pharmaceutical compositions comprise a liquid, a suspension, a solution, or a gel. In some instances, the pharmaceutical compositions comprising an immune checkpoint inhibitor and an IL-2 conjugate described herein, or combination thereof are injectable. In some instances, pharmaceutical compositions comprise excipients that solubilize an immune checkpoint inhibitor and an IL-2 conjugate described herein, or a combination thereof. In another embodiment, the pharmaceutical compositions comprising an immune checkpoint inhibitor and an IL-2 conjugate described herein are provided in a dosage form for parenteral administration, which comprises one or more pharmaceutically acceptable excipients or carriers. In some instances, the pharmaceutical compositions comprising an immune checkpoint inhibitor and an IL-2 conjugate described herein, or combination thereof are injectable. Where pharmaceutical compositions are Formulated for intravenous, cutaneous or subcutaneous injection, the active ingredient is in the form of a parenterally acceptable aqueous solution, which is pyrogen-free and has a suitable pH, isotonicity, and stability. Those of relevant skill in the art are well able to prepare suitable solutions using, for example, isotonic vehicles, such as Sodium Chloride injection, Ringer's injection, or Lactated Ringer's injection. In some embodiments, preservatives, stabilizers, excipients, buffers, antioxidants, and/or other additives are included.

### Kits/Article of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### EXEMPLARY EMBODIMENTS

The present disclosure is further described by the following embodiments. The features of each of the embodiments are combinable with any of the other embodiments where appropriate and practical.

Embodiment 1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (I):

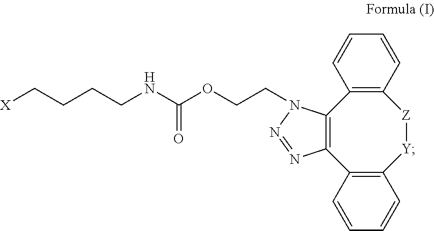

wherein:

### Z is CH₂ and Y is

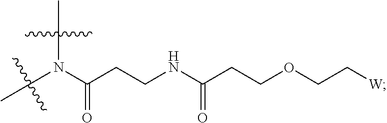

### Y is CH₂ and Z is

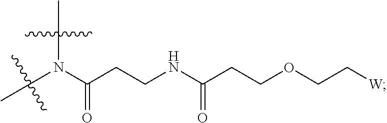

### Z is CH₂ and Y is

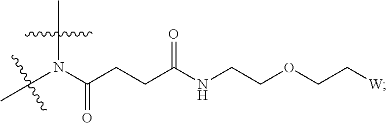

or

### Y is CH₂ and Z is

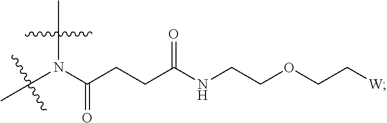

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

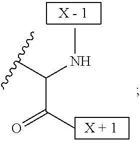

or a pharmaceutically acceptable salt, solvate, or hydrate thereof.

Embodiment 1.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (I):

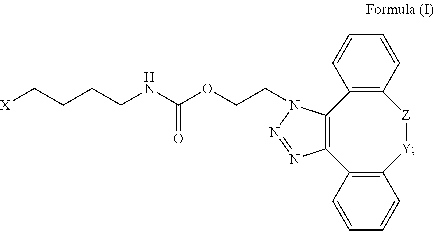

wherein:

### Z is CH₂ and Y is

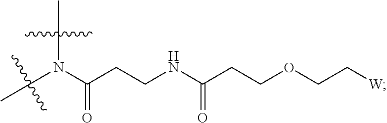

### Y is CH₂ and Z is

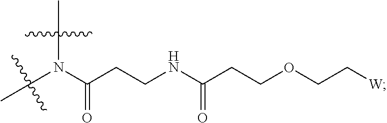

### Z is CH₂ and Y is

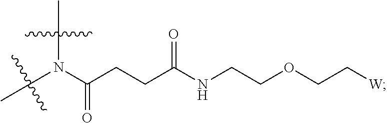

or

### Y is CH₂ and Z is

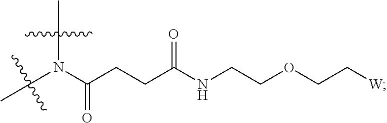

W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

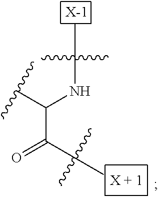

X−1 indicates the point of attachment to the preceding amino acid residue; and
X+1 indicates the point of attachment to the following amino acid residue;
or a pharmaceutically acceptable salt, solvate, or hydrate thereof.

Embodiment 2. The method according to embodiment 1 or 1.1, wherein in the IL-2 conjugate Z is CH₂ and Y is

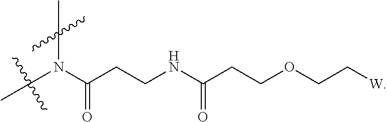

Embodiment 3. The method according to embodiment 1 or 1.1, wherein in the IL-2 conjugate Y is CH₂ and Z is

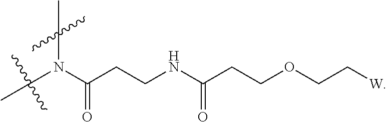

Embodiment 4. The method according to embodiment 1 or 1.1, wherein in the IL-2 conjugate Z is CH₂ and Y is

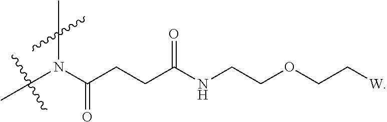

Embodiment 5. The method according to embodiment 1 or 1.1, wherein in the IL-2 conjugate Z is CH₂ and Y is

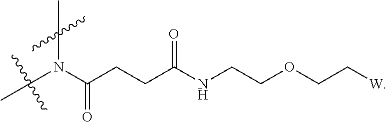

Embodiment 6. The method according to embodiment 1 or 1.1, wherein in the IL-2 conjugate Y is CH₂ and Z is

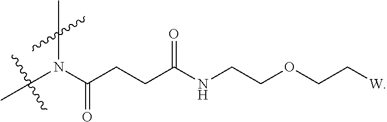

Embodiment 7. The method according to embodiment 1 or 1.1, wherein in the IL-2 conjugate the PEG group has an average molecular weight selected from 5 kDa, 10 kDa, 20 kDa and 30 kDa.

Embodiment 8. The method according to embodiment 1 or 1.1, wherein in the IL-2 conjugate the PEG group has an average molecular weight of 10 kDa, 20 kDa, or 30 kDa or a pharmaceutically acceptable salt, solvate, or hydrate thereof.

Embodiment 9. The method according to embodiment 1 or 1.1, wherein in the IL-2 conjugate the PEG group has an average molecular weight of 30 kDa.

Embodiment 10. The method according to embodiment 1 or 1.1, wherein in the IL-2 conjugate the position of the structure of Formula (I) in the amino acid sequence of the IL-2 conjugate is selected from K34, F41, F43, K42, E61, P64, R37, T40, E67, Y44, V68, and L71.

Embodiment 11. The method according to embodiment 1 or 1.1, wherein in the IL-2 conjugate the position of the structure of Formula (I) in the amino acid sequence of the IL-2 conjugate is selected from F41, E61, and P64.

Embodiment 12. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] has the structure of Formula (II) or Formula (III), or a mixture of Formula (II) and Formula (III):

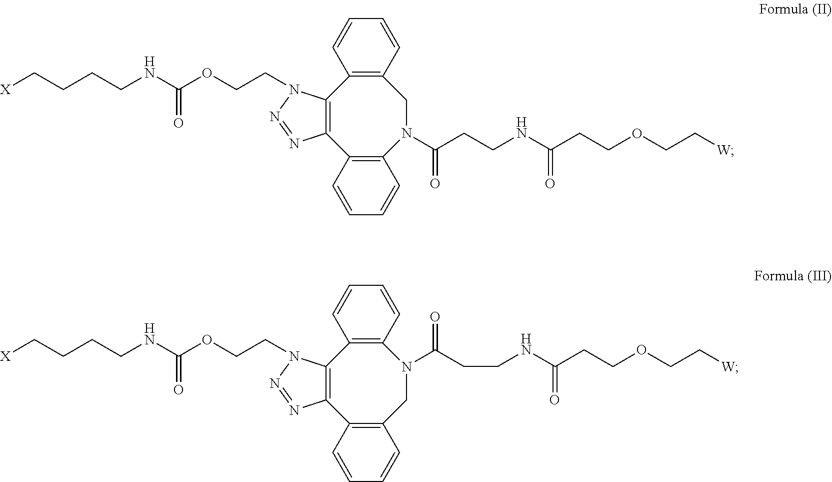

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure

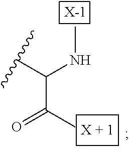

or a pharmaceutically acceptable salt, solvate, or hydrate thereof.

Embodiment 12.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] has the structure of Formula (II) or Formula (III), or a mixture of Formula II and Formula III):

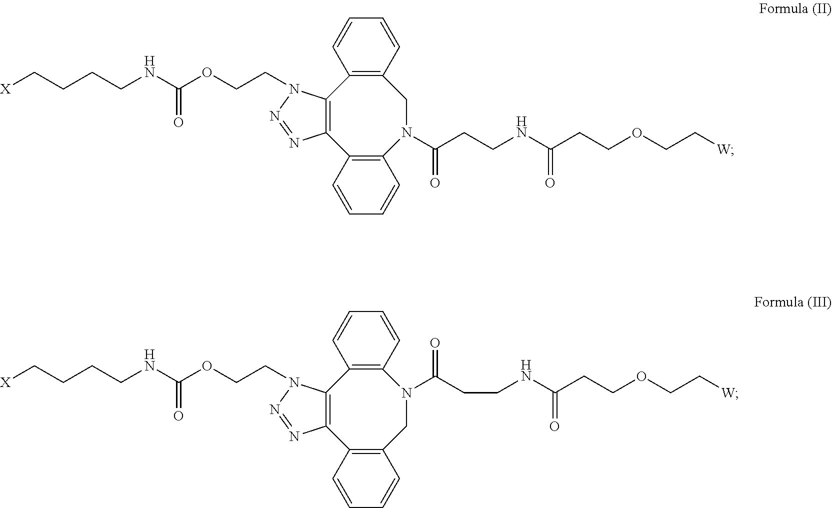

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

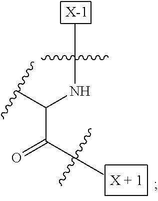

X−1 indicates the point of attachment to the preceding amino acid residue; and
X+1 indicates the point of attachment to the following amino acid residue;
or a pharmaceutically acceptable salt, solvate, or hydrate thereof.

Embodiment 13. The method according to embodiment 12 or 12.1, wherein the [AzK_PEG] is a mixture of Formula (II) and Formula (III).

Embodiment 14. The method according to embodiment 12 or 12.1, wherein the [AzK_PEG] has the structure of formula (II):

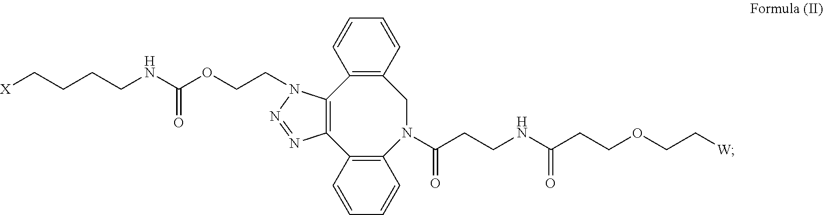

or a pharmaceutically acceptable salt, solvate, or hydrate thereof.

Embodiment 15. The method according to embodiment 14, wherein the IL-2 conjugate has the amino acid sequence of SEQ ID NO: 15.

Embodiment 16. The method according to embodiment 15, wherein W is a PEG group having an average molecular weight selected from 5 kDa, 10 kDa, 15 kDa, 20 kDa, 25 kDa, and 30 kDa.

Embodiment 17. The method according to embodiment 16, wherein W is a PEG group having an average molecular weight selected from 5 kDa and 30 kDa.

Embodiment 18. The method according to embodiment 17, wherein W is a PEG group having an average molecular weight of 5 kDa.

Embodiment 19. The method according to embodiment 17, wherein W is a PEG group having an average molecular weight of 30 kDa.

Embodiment 20. The method according to embodiment 12 or 12.1, wherein the IL-2 conjugate has the amino acid sequence of SEQ ID NO: 16.

Embodiment 21. The method according to embodiment 20, wherein W is a PEG group having an average molecular weight selected from 5 kDa, 10 kDa, 15 kDa, 20 kDa, 25 kDa, and 30 kDa.

Embodiment 22. The method according to embodiment 21, wherein W is a PEG group having an average molecular weight selected from 5 kDa and 30 kDa.

Embodiment 23. The method according to embodiment 22, wherein W is a PEG group having an average molecular weight of 5 kDa.

Embodiment 24. The method according to embodiment 22, wherein W is a PEG group having an average molecular weight of 30 kDa.

Embodiment 25. The method according to embodiment 12 or 12.1, wherein the IL-2 conjugate has the amino acid sequence of SEQ ID NO: 17.

Embodiment 26. The method according to embodiment 25, wherein W is a PEG group having an average molecular weight selected from 5 kDa, 10 kDa, 15 kDa, 20 kDa, 25 kDa, and 30 kDa.

Embodiment 27. The method according to embodiment 26, wherein W is a PEG group having an average molecular weight selected from 5 kDa and 30 kDa.

Embodiment 28. The method according to embodiment 27, wherein W is a PEG group having an average molecular weight of 5 kDa.

Embodiment 29. The method according to embodiment 27, wherein W is a PEG group having an average molecular weight of 30 kDa.

Embodiment 30. The method according to embodiment 12 or 12.1, wherein the IL-2 conjugate has the amino acid sequence of SEQ ID NO: 18.

Embodiment 31. The method according to embodiment 30, wherein W is a PEG group having an average molecular weight selected from 5 kDa, 10 kDa, 15 kDa, 20 kDa, 25 kDa, and 30 kDa.

Embodiment 32. The method according to embodiment 31, wherein W is a PEG group having an average molecular weight selected from 5 kDa and 30 kDa.

Embodiment 33. The method according to embodiment 32, wherein W is a PEG group having an average molecular weight of 5 kDa.

Embodiment 34. The method according to embodiment 32, wherein W is a PEG group having an average molecular weight of 30 kDa.

Embodiment 35. The method according to embodiment 12 or 12.1, wherein the IL-2 conjugate has the amino acid sequence of SEQ ID NO: 19.

Embodiment 36. The method according to embodiment 35, wherein W is a PEG group having an average molecular weight selected from 5 kDa, 10 kDa, 15 kDa, 20 kDa, 25 kDa, and 30 kDa.

Embodiment 37. The method according to embodiment 36, wherein W is a PEG group having an average molecular weight selected from 5 kDa and 30 kDa.

Embodiment 38. The method according to embodiment 37, wherein W is a PEG group having an average molecular weight of 5 kDa.

Embodiment 39. The method according to embodiment 37, wherein W is a PEG group having an average molecular weight of 30 kDa.

Embodiment 40. The method according to embodiment 12 or 12.1, wherein the [AzK_PEG] has the structure of formula (III)

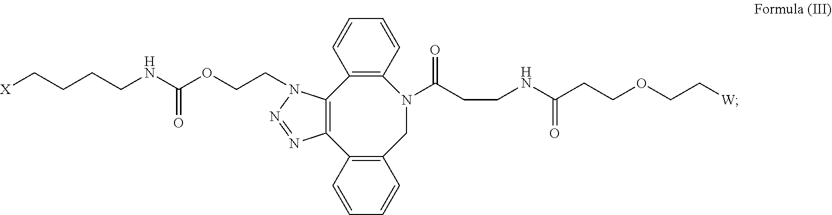

or a pharmaceutically acceptable salt, solvate, or hydrate thereof.

Embodiment 41. The method according to embodiment 40, wherein the IL-2 conjugate has the amino acid sequence of SEQ ID NO: 15.

Embodiment 42. The method according to embodiment 41, wherein W is a PEG group having an average molecular weight selected from 5 kDa, 10 kDa, 15 kDa, 20 kDa, 25 kDa, and 30 kDa.

Embodiment 43. The method according to embodiment 42, wherein W is a PEG group having an average molecular weight selected from 5 kDa and 30 kDa.

Embodiment 44. The method according to embodiment 43, wherein W is a PEG group having an average molecular weight of 5 kDa.

Embodiment 45. The method according to embodiment 43, wherein W is a PEG group having an average molecular weight of 30 kDa.

Embodiment 46. The method according to embodiment 12 or 12.1, wherein the IL-2 conjugate has the amino acid sequence of SEQ ID NO: 16.

Embodiment 47. The method according to embodiment 46, wherein W is a PEG group having an average molecular weight selected from 5 kDa, 10 kDa, 15 kDa, 20 kDa, 25 kDa, and 30 kDa.

Embodiment 48. The method according to embodiment 47, wherein W is a PEG group having an average molecular weight selected from 5 kDa and 30 kDa.

Embodiment 49. The method according to embodiment 48, wherein W is a PEG group having an average molecular weight of 5 kDa.

Embodiment 50. The method according to embodiment 48, wherein W is a PEG group having an average molecular weight of 30 kDa.

Embodiment 51. The method according to embodiment 12 or 12.1, wherein the IL-2 conjugate has the amino acid sequence of SEQ ID NO: 17.

Embodiment 52. The method according to embodiment 51, wherein W is a PEG group having an average molecular weight selected from 5 kDa, 10 kDa, 15 kDa, 20 kDa, 25 kDa, and 30 kDa.

Embodiment 53. The method according to embodiment 52, wherein W is a PEG group having an average molecular weight selected from 5 kDa and 30 kDa.

Embodiment 54. The method according to embodiment 53, wherein W is a PEG group having an average molecular weight of 5 kDa.

Embodiment 55. The method according to embodiment 53, wherein W is a PEG group having an average molecular weight of 30 kDa.

Embodiment 56. The method according to embodiment 12 or 12.1, wherein the IL-2 conjugate has the amino acid sequence of SEQ ID NO: 18.

Embodiment 57. The method according to embodiment 56, wherein W is a PEG group having an average molecular weight selected from 5 kDa, 10 kDa, 15 kDa, 20 kDa, 25 kDa, and 30 kDa.

Embodiment 58. The method according to embodiment 57, wherein W is a PEG group having an average molecular weight selected from 5 kDa and 30 kDa.

Embodiment 59. The method according to embodiment 58, wherein W is a PEG group having an average molecular weight of 5 kDa.

Embodiment 60. The method according to claim embodiment 58, wherein W is a PEG group having an average molecular weight of 30 kDa.

Embodiment 61. The method according to embodiment 12 or 12.1, wherein the IL-2 conjugate has the amino acid sequence of SEQ ID NO: 19.

Embodiment 62. The method according to embodiment 61, wherein W is a PEG group having an average molecular weight selected from 5 kDa, 10 kDa, 15 kDa, 20 kDa, 25 kDa, and 30 kDa.

Embodiment 63. The method according to embodiment 62, wherein W is a PEG group having an average molecular weight selected from 5 kDa and 30 kDa.

Embodiment 64. The method according to embodiment 63, wherein W is a PEG group having an average molecular weight of 5 kDa.

Embodiment 65. The method according to embodiment 63, wherein W is a PEG group having an average molecular weight of 30 kDa.

Embodiment 66. The method according to any one of embodiments 1 to 65, wherein W is a linear or branched PEG group.

Embodiment 67. The method according to any one of embodiments 1 to 65, wherein W is a linear PEG group.

Embodiment 68. The method according to any one of embodiments 1 to 65, wherein W is a branched PEG group.

Embodiment 69. The method according to any one of embodiments 1 to 65, wherein W is a methoxy PEG group.

Embodiment 70. The method according to embodiment 69, wherein the methoxy PEG group is linear or branched.

Embodiment 71. The method according to embodiment 70, wherein the methoxy PEG group is linear.

Embodiment 72. The method according to embodiment 70, wherein the methoxy PEG group is branched.

Embodiment 73. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20-24, wherein [AzK_PEG5kD] has the structure of Formula (II) or Formula (III), or a mixture of Formula (II) and Formula (III):

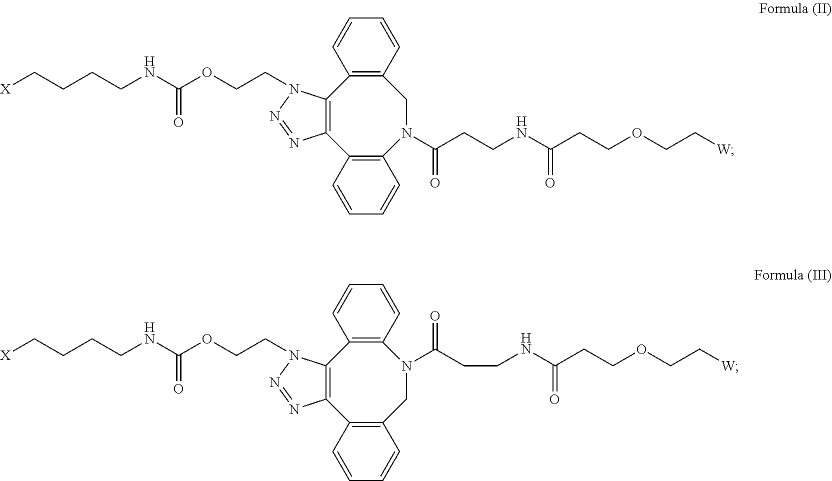

wherein:
W is a PEG group having an average molecular weight of 5 kDa; and
X has the structure:

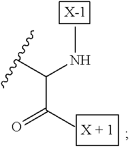

or a pharmaceutically acceptable salt, solvate, or hydrate thereof.

Embodiment 73.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20-24, wherein [AzK_PEG5kD] has the structure of Formula (II) or Formula (III), or a mixture of Formula (II) and Formula (II):

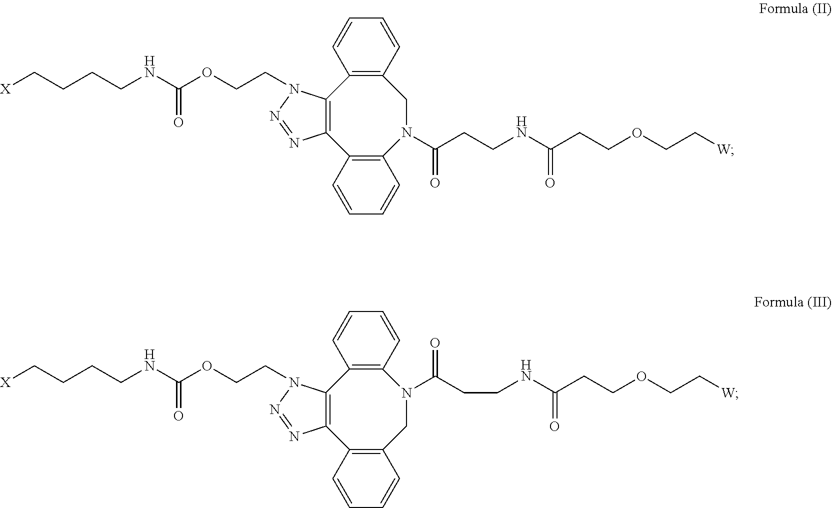

wherein:
W is a PEG group having an average molecular weight of 5 kDa; and
X has the structure:

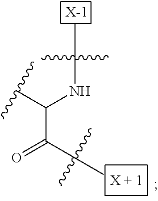

X−1 indicates the point of attachment to the preceding amino acid residue; and
X+1 indicates the point of attachment to the following amino acid residue;
or a pharmaceutically acceptable salt, solvate, or hydrate thereof.

Embodiment 74. The method according to embodiment 73 or 73.1, wherein the IL-2 conjugate has the amino acid sequence of SEQ ID NO: 20.

Embodiment 75. The method according to embodiment 73 or 73.1, wherein the IL-2 conjugate has the amino acid sequence of SEQ ID NO: 21.

Embodiment 76. The method according to embodiment 73 or 73.1, wherein the IL-2 conjugate has the amino acid sequence of SEQ ID NO: 22.

Embodiment 77. The method according to embodiment 73 or 73.1, wherein the IL-2 conjugate has the amino acid sequence of SEQ ID NO: 23.

Embodiment 78. The method according to embodiment 73 or 73.1, wherein the IL-2 conjugate has the amino acid sequence of SEQ ID NO: 24.

Embodiment 79. The method according to embodiment 73 or 73.1, wherein the [AzK_PEG5kD] has the structure of formula (II)

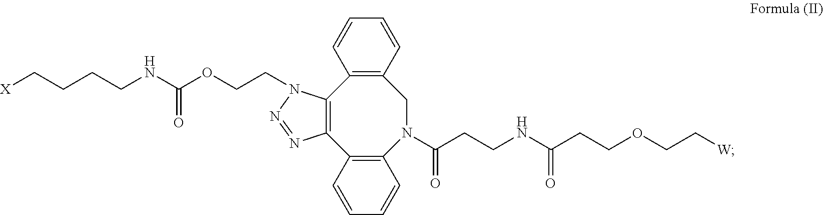

or a pharmaceutically acceptable salt, solvate, or hydrate thereof.

Embodiment 80. The method according to embodiment 79, wherein the IL-2 conjugate has the amino acid sequence of SEQ ID NO: 20.

Embodiment 81. The method according to embodiment 79, wherein the IL-2 conjugate has the amino acid sequence of SEQ ID NO: 21.

Embodiment 82. The method according to embodiment 79, wherein the IL-2 conjugate has the amino acid sequence of SEQ ID NO: 22.

Embodiment 83. The method according to embodiment 79, wherein the IL-2 conjugate has the amino acid sequence of SEQ ID NO: 23.

Embodiment 84. The method according to embodiment 79, wherein the IL-2 conjugate has the amino acid sequence of SEQ ID NO: 24.

Embodiment 85. The method according to embodiment 73 or 73.1, wherein the [AzK_PEG5kD] has the structure of formula (III)

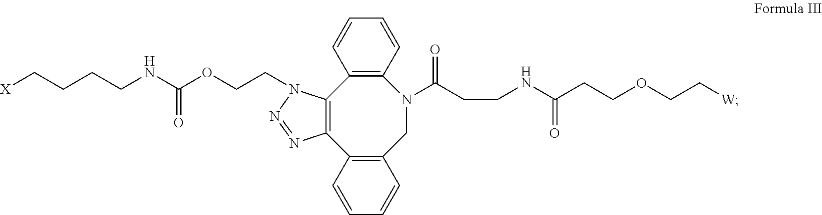

or a pharmaceutically acceptable salt, solvate, or hydrate thereof.

Embodiment 86. The method according to embodiment 85, wherein the IL-2 conjugate has the amino acid sequence of SEQ ID NO: 20.

Embodiment 87. The method according to embodiment 85, wherein the IL-2 conjugate has the amino acid sequence of SEQ ID NO: 21.

Embodiment 88. The method according to embodiment 85, wherein the IL-2 conjugate has the amino acid sequence of SEQ ID NO: 22.

Embodiment 89. The method according to embodiment 85, wherein the IL-2 conjugate has the amino acid sequence of SEQ ID NO: 23.

Embodiment 90. The method according to embodiment 85, wherein the IL-2 conjugate has the amino acid sequence of SEQ ID NO: 24.

Embodiment 91. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] has the structure of Formula (II) or Formula (III), or is a mixture of the structures of Formula (II) and Formula (III):

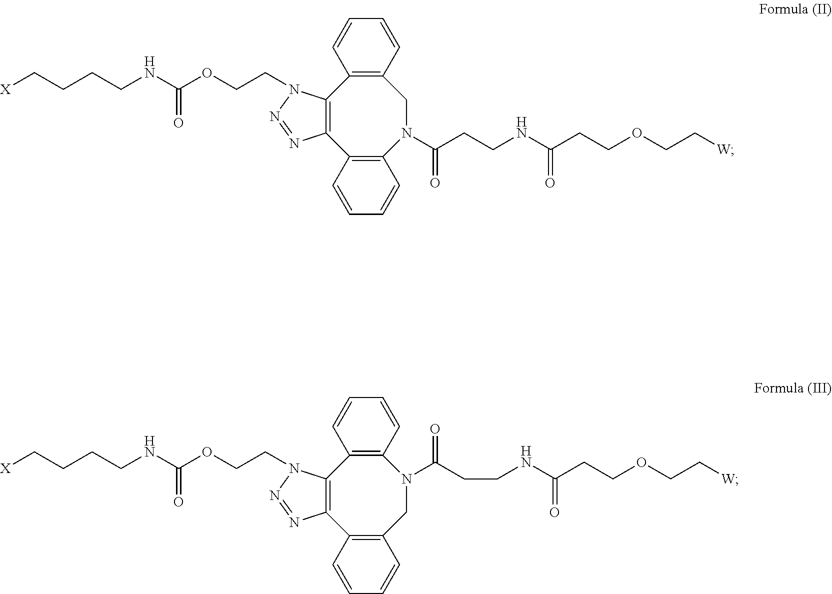

wherein:
W is a PEG group having an average molecular weight of 30 kDa; and
X has the structure:

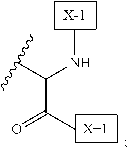

or a pharmaceutically acceptable salt, solvate, or hydrate thereof.

Embodiment 91.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] has the structure of Formula (II) or Formula (III), or is a mixture of the structures of Formula (II) and Formula (III):

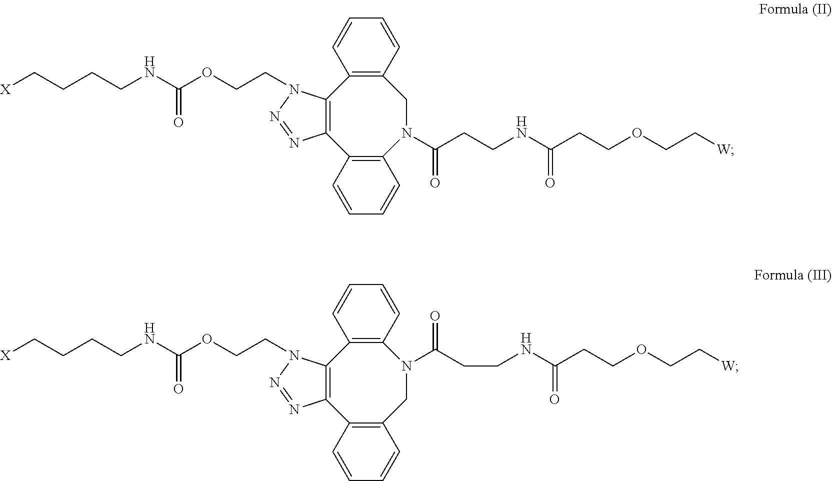

wherein:
W is a PEG group having an average molecular weight of 30 kDa; and
X has the structure:

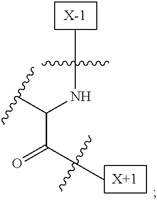

X−1 indicates the point of attachment to the preceding amino acid residue; and
X+1 indicates the point of attachment to the following amino acid residue;
or a pharmaceutically acceptable salt, solvate, or hydrate thereof.

Embodiment 92. The method according to embodiment 91 or 91.1, wherein the IL-2 conjugate has the amino acid sequence of SEQ ID NO: 25.

Embodiment 93. The method according to embodiment 91 or 91.1, wherein the IL-2 conjugate has the amino acid sequence of SEQ ID NO: 26.

Embodiment 94. The method according to embodiment 91 or 91.1, wherein the IL-2 conjugate has the amino acid sequence of SEQ ID NO: 27.

Embodiment 95. The method according to embodiment 91 or 91.1, wherein the IL-2 conjugate has the amino acid sequence of SEQ ID NO: 28.

Embodiment 96. The method according to embodiment 91 or 91.1, wherein the IL-2 conjugate has the amino acid sequence of SEQ ID NO: 29.

Embodiment 97. The method according to embodiment 91 or 91.1, wherein the [AzK_PEG30kD] has the structure of formula (II):

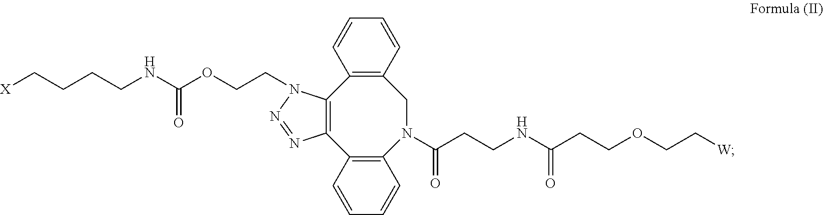

or a pharmaceutically acceptable salt, solvate, or hydrate thereof.

Embodiment 98. The method according to embodiment 97, wherein the IL-2 conjugate has the amino acid sequence of SEQ ID NO: 25.

Embodiment 99. The method according to embodiment 97, wherein the IL-2 conjugate has the amino acid sequence of SEQ ID NO: 26.

Embodiment 100. The method according to embodiment 97, wherein the IL-2 conjugate has the amino acid sequence of SEQ ID NO: 27.

Embodiment 101. The method according to embodiment 97, wherein the IL-2 conjugate has the amino acid sequence of SEQ ID NO: 28.

Embodiment 102. The method according to embodiment 97, wherein the IL-2 conjugate has the amino acid sequence of SEQ ID NO: 29.

Embodiment 103. The method according to embodiment 91 or 91.1, wherein the [AzK_PEG30kD] has the structure of formula (III)

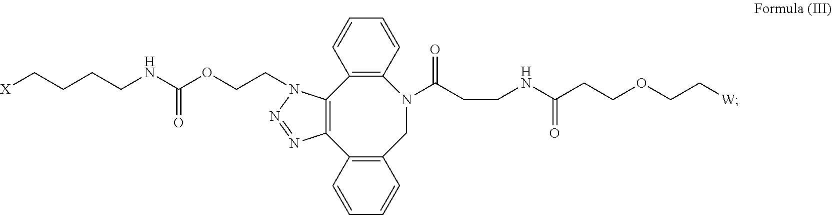

or a pharmaceutically acceptable salt, solvate, or hydrate thereof.

Embodiment 104. The method according to embodiment 103, wherein the IL-2 conjugate has the amino acid sequence of SEQ ID NO: 25.

Embodiment 105. The method according to embodiment 103, wherein the IL-2 conjugate has the amino acid sequence of SEQ ID NO: 26.

Embodiment 106. The method according to embodiment 103, wherein the IL-2 conjugate has the amino acid sequence of SEQ ID NO: 27.

Embodiment 107. The method according to embodiment 103, wherein the IL-2 conjugate has the amino acid sequence of SEQ ID NO: 28.

Embodiment 108. The method according to embodiment 103, wherein the IL-2 conjugate has the amino acid sequence of SEQ ID NO: 29.

Embodiment 109. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] is a mixture of the structures of Formula (II) and Formula (III):

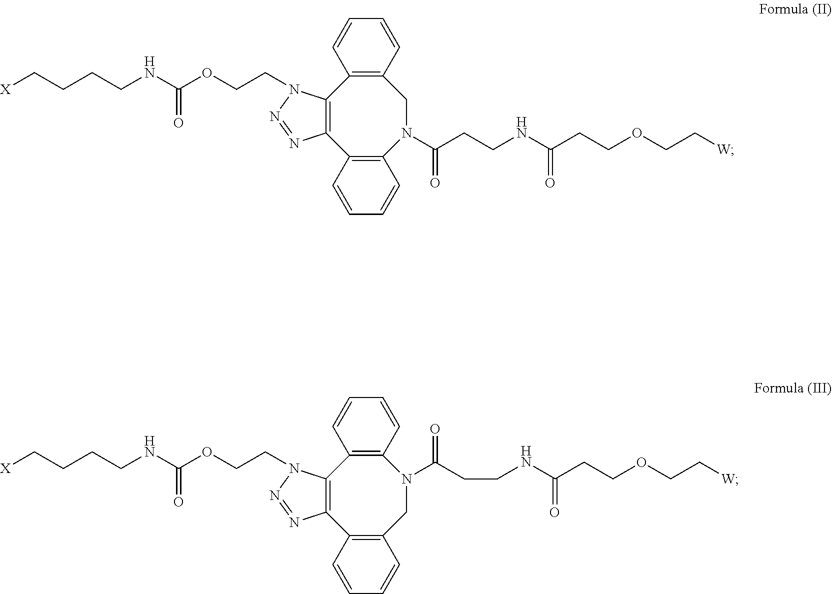

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

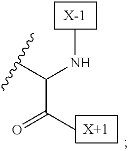

or a pharmaceutically acceptable salt, solvate, or hydrate thereof.

Embodiment 109.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 15-19, wherein [AzK_PEG] is a mixture of the structures of Formula (II) and Formula (III):

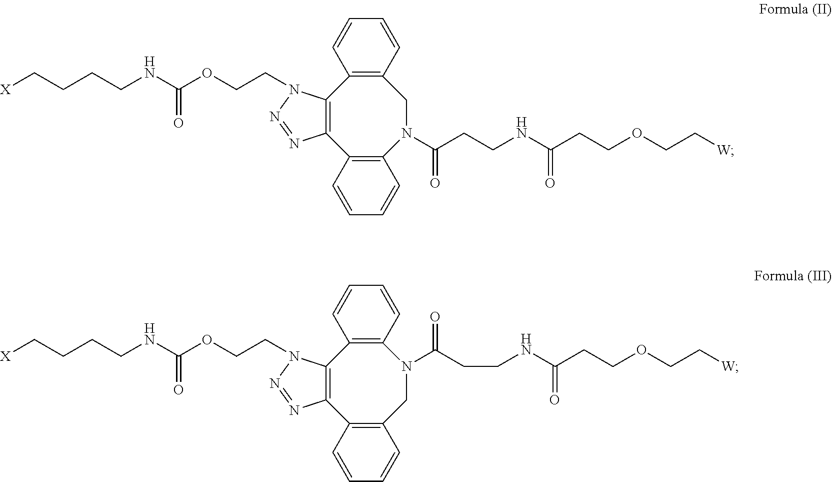

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

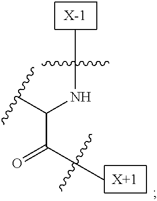

X−1 indicates the point of attachment to the preceding amino acid residue; and
X+1 indicates the point of attachment to the following amino acid residue;
or a pharmaceutically acceptable salt, solvate, or hydrate thereof.

Embodiment 110. The method according to embodiment 109 or 109.1, wherein the ratio of the amount of the structure of Formula (II) to the amount of the structure of Formula (III) comprising the total amount of [AzK_PEG] in the IL-2 conjugate is about 1:1.

Embodiment 111. The method according to embodiment 109 or 109.1, wherein the ratio of the amount of the structure of Formula (II) to the amount of the structure of Formula (III) comprising the total amount of [AzK_PEG] in the IL-2 conjugate is greater than 1:1.

Embodiment 112. The method according to embodiment 109 or 109.1, wherein the ratio of the amount of the structure of Formula (II) to the amount of the structure of Formula (III) comprising the total amount of [AzK_PEG] in the IL-2 conjugate is less than 1:1.

Embodiment 113. The method according to anyone of embodiments 109 to 112, wherein W is a linear or branched PEG group.

Embodiment 114. The method according to anyone of embodiments 109 to 112, wherein W is a linear PEG group.

Embodiment 115. The method according to anyone of embodiments 109 to 112, wherein W is a branched PEG group.

Embodiment 116. The method according to anyone of embodiments 109 to 112, wherein W is a methoxy PEG group.

Embodiment 117. The method according to embodiment 116, wherein the methoxy PEG group is linear or branched.

Embodiment 118. The method according to embodiment 117, wherein the methoxy PEG group is linear.

Embodiment 119. The method according to embodiment 117, wherein the methoxy PEG group is branched.

Embodiment 120. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20 to 24, wherein [AzK_PEG5kD] is a mixture of the structures of Formula (II) and Formula (III):

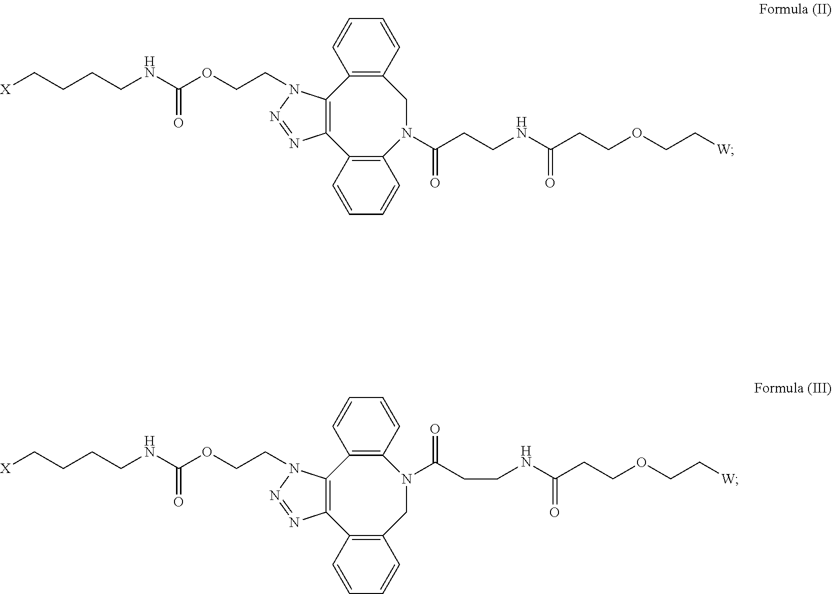

wherein:
W is a PEG group having an average molecular weight of 5 kDa; and
X has the structure:

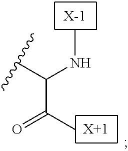

or a pharmaceutically acceptable salt, solvate, or hydrate thereof.

Embodiment 120.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 20 to 24, wherein [AzK_PEG5kD] is a mixture of the structures of Formula (II) and Formula (III):

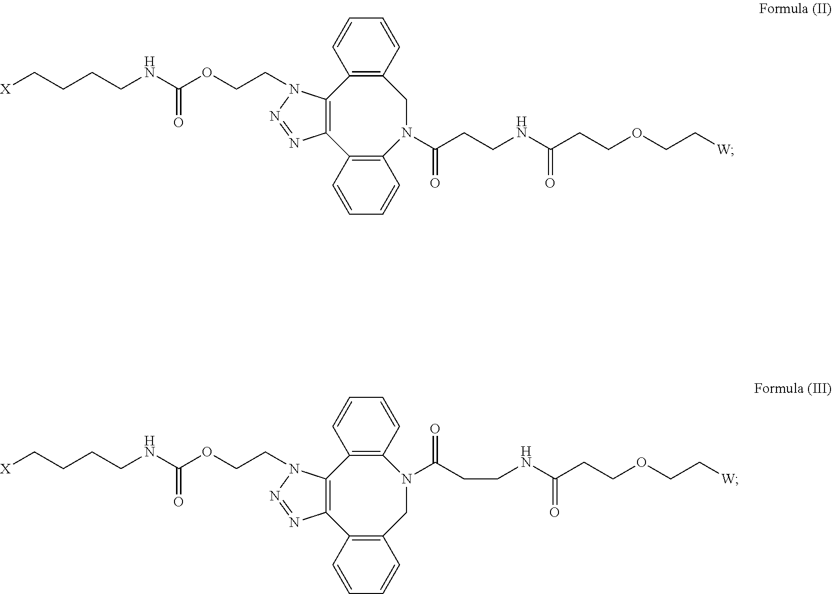

wherein:
W is a PEG group having an average molecular weight of 5 kDa; and
X has the structure:

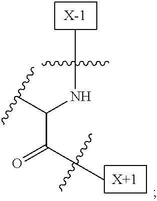

X−1 indicates the point of attachment to the preceding amino acid residue; and
X+1 indicates the point of attachment to the following amino acid residue;
or a pharmaceutically acceptable salt, solvate, or hydrate thereof.

Embodiment 121. The method according to embodiment 120 or 120.1, wherein the ratio of the amount of the structure of Formula (II) to the amount of the structure of Formula (III) comprising the total amount of [AzK_PEG5kD] in the IL-2 conjugate is about 1:1.

Embodiment 122. The method according to embodiment 120 or 120.1, wherein the ratio of the amount of the structure of Formula (II) to the amount of the structure of Formula (III) comprising the total amount of [AzK_PEG5kD] in the IL-2 conjugate is greater than 1:1.

Embodiment 123. The method according to embodiment 120 or 120.1, wherein the ratio of the amount of the structure of Formula (II) to the amount of the structure of Formula (III) comprising the total amount of [AzK_PEG5kD] in the IL-2 conjugate is less than 1:1.

Embodiment 124. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] is a mixture of the structures of Formula (II) and Formula (III):

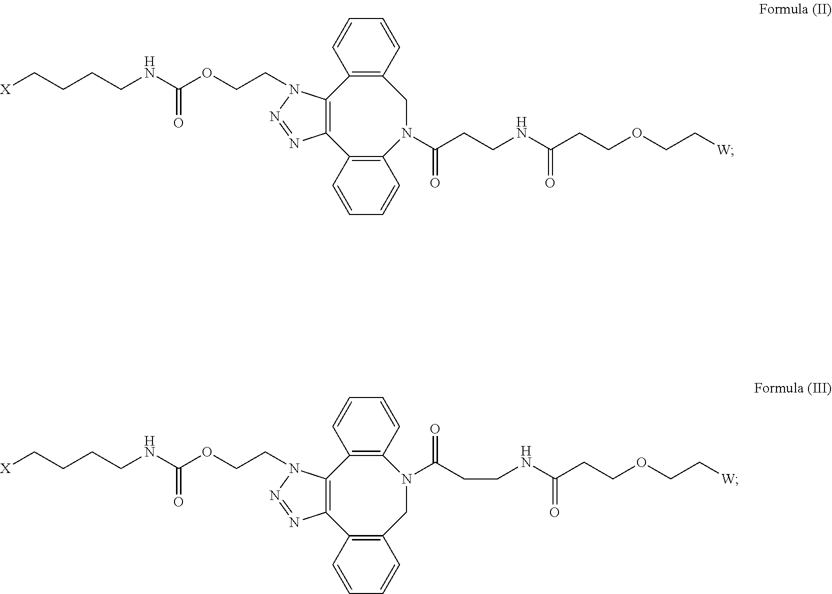

wherein:
W is a PEG group having an average molecular weight of 30 kDa; and
X has the structure:

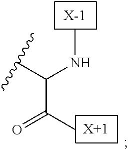

or a pharmaceutically acceptable salt, solvate, or hydrate thereof.

Embodiment 124.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 25-29, wherein [AzK_PEG30kD] is a mixture of the structures of Formula (II) and Formula (III):

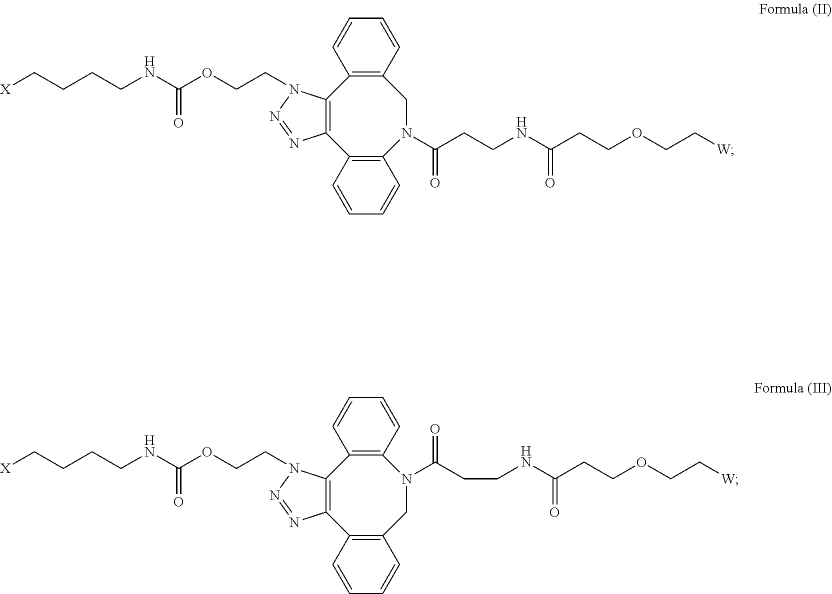

wherein:
W is a PEG group having an average molecular weight of 30 kDa; and
X has the structure:

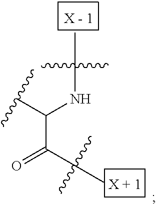

X−1 indicates the point of attachment to the preceding amino acid residue; and
X+1 indicates the point of attachment to the following amino acid residue;
or a pharmaceutically acceptable salt, solvate, or hydrate thereof.

Embodiment 125. The method according to embodiment 124 or 124.1, wherein the ratio of the amount of the structure of Formula (II) to the amount of the structure of Formula (III) comprising the total amount of [AzK_PEG30kD] in the IL-2 conjugate is about 1:1.

Embodiment 126. The method according to embodiment 124 or 124.1, wherein the ratio of the amount of the structure of Formula (II) to the amount of the structure of Formula (III) comprising the total amount of [AzK_PEG30kD] in the IL-2 conjugate is greater than 1:1.

Embodiment 127. The method according to embodiment 124 or 124.1, wherein the ratio of the amount of the structure of Formula (II) to the amount of the structure of Formula (III) comprising the total amount of [AzK_PEG30kD] in the IL-2 conjugate is less than 1:1.

Embodiment 128. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [AzK_L1_PEG] has the structure of Formula (IV) or Formula (V), or a mixture of Formula (IV) and Formula (V):

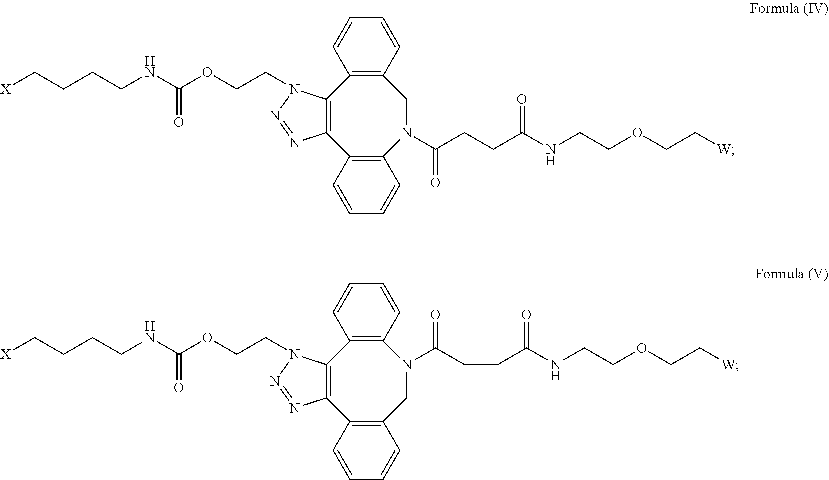

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

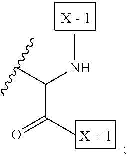

or a pharmaceutically acceptable salt, solvate, or hydrate thereof.

Embodiment 128.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [AzK_L1_PEG] has the structure of Formula (IV) or Formula (V), or a mixture of Formula (IV) and Formula (V):

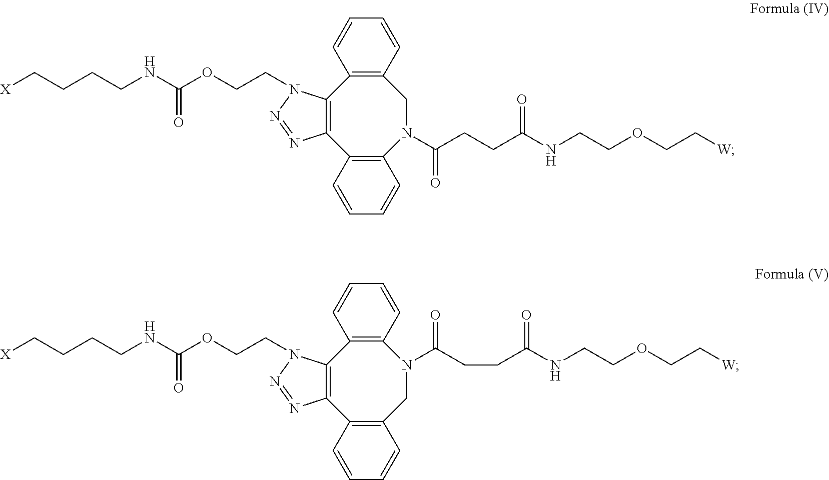

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

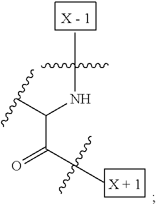

X−1 indicates the point of attachment to the preceding amino acid residue; and
X+1 indicates the point of attachment to the following amino acid residue;
or a pharmaceutically acceptable salt, solvate, or hydrate thereof.

Embodiment 129. The method according to embodiment 128 or 128.1, wherein the [AzK_L1_PEG] is a mixture of Formula (IV) and Formula (V).

Embodiment 130. The method according to embodiment 128 or 128.1, wherein the [AzK_L1_PEG] has the structure of Formula (IV):

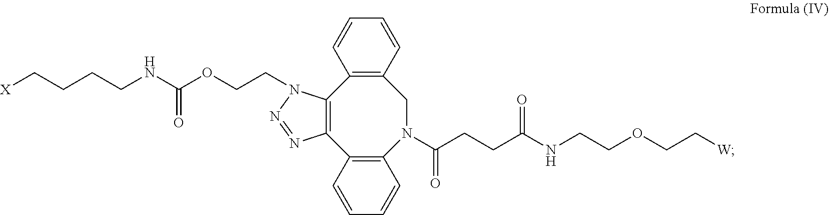

or a pharmaceutically acceptable salt, solvate, or hydrate thereof.

Embodiment 131. The method according to embodiment 128 or 128.1, wherein the IL-2 conjugate has the amino acid sequence of SEQ ID NO: 40.

Embodiment 132. The method according to embodiment 131, wherein W is a PEG group having an average molecular weight selected from 5 kDa, 10 kDa, 15 kDa, 20 kDa, 25 kDa, and 30 kDa.

Embodiment 133. The method according to embodiment 132, wherein W is a PEG group having an average molecular weight selected from 5 kDa and 30 kDa.

Embodiment 134. The method according to embodiment 133, wherein W is a PEG group having an average molecular weight of 5 kDa.

Embodiment 135. The method according to embodiment 133, wherein W is a PEG group having an average molecular weight of 30 kDa.

Embodiment 136. The method according to embodiment 128 or 128.1, wherein the IL-2 conjugate has the amino acid sequence of SEQ ID NO: 41.

Embodiment 137. The method according to embodiment 136, wherein W is a PEG group having an average molecular weight selected from 5 kDa, 10 kDa, 15 kDa, 20 kDa, 25 kDa, and 30 kDa.

Embodiment 138. The method according to embodiment 137, wherein W is a PEG group having an average molecular weight selected from 5 kDa and 30 kDa.

Embodiment 139. The method according to embodiment 138, wherein W is a PEG group having an average molecular weight of 5 kDa.

Embodiment 140. The method according to embodiment 138, wherein W is a PEG group having an average molecular weight of 30 kDa.

Embodiment 141. The method according to embodiment 128 or 128.1, wherein the IL-2 conjugate has the amino acid sequence of SEQ ID NO: 42.

Embodiment 142. The method according to embodiment 141, wherein W is a PEG group having an average molecular weight selected from 5 kDa, 10 kDa, 15 kDa, 20 kDa, 25 kDa, and 30 kDa.

Embodiment 143. The method according to embodiment 142, wherein W is a PEG group having an average molecular weight selected from 5 kDa and 30 kDa.

Embodiment 144. The method according to embodiment 143, wherein W is a PEG group having an average molecular weight of 5 kDa.

Embodiment 145. The method according to embodiment 143, wherein W is a PEG group having an average molecular weight of 30 kDa.

Embodiment 146. The method according to embodiment 128 or 128.1, wherein the IL-2 conjugate has the amino acid sequence of SEQ ID NO: 43.

Embodiment 147. The method according to embodiment 146, wherein W is a PEG group having an average molecular weight selected from 5 kDa, 10 kDa, 15 kDa, 20 kDa, 25 kDa, and 30 kDa.

Embodiment 148. The method according to embodiment 147, wherein W is a PEG group having an average molecular weight selected from 5 kDa and 30 kDa.

Embodiment 149. The method according to embodiment 148, wherein W is a PEG group having an average molecular weight of 5 kDa.

Embodiment 150. The method according to embodiment 148, wherein W is a PEG group having an average molecular weight of 30 kDa.

Embodiment 151. The method according to embodiment 128 or 128.1, wherein the IL-2 conjugate has the amino acid sequence of SEQ ID NO: 44.

Embodiment 152. The method according to embodiment 151, wherein W is a PEG group having an average molecular weight selected from 5 kDa, 10 kDa, 15 kDa, 20 kDa, 25 kDa, and 30 kDa.

Embodiment 153. The method according to embodiment 152, wherein W is a PEG group having an average molecular weight selected from 5 kDa and 30 kDa.

Embodiment 154. The method according to embodiment 153, wherein W is a PEG group having an average molecular weight of 5 kDa.

Embodiment 155. The method according to embodiment 153, wherein W is a PEG group having an average molecular weight of 30 kDa.

Embodiment 156. The method according to embodiment 128 or 128.1, wherein the [AzK_L1_PEG] has the structure of Formula (V)

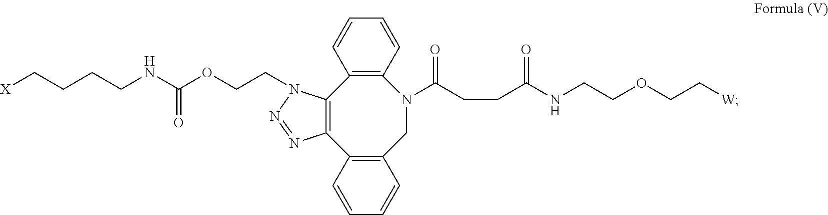

or a pharmaceutically acceptable salt, solvate, or hydrate thereof.

Embodiment 157. The method according to embodiment 156, wherein the IL-2 conjugate has the amino acid sequence of SEQ ID NO: 40.

Embodiment 158. The method according to embodiment 156, wherein W is a PEG group having an average molecular weight selected from 5 kDa, 10 kDa, 15 kDa, 20 kDa, 25 kDa, and 30 kDa.

Embodiment 159. The method according to embodiment 158, wherein W is a PEG group having an average molecular weight selected from 5 kDa and 30 kDa.

Embodiment 160. The method according to embodiment 159, wherein W is a PEG group having an average molecular weight of 5 kDa.

Embodiment 161. The method according to embodiment 159, wherein W is a PEG group having an average molecular weight of 30 kDa.

Embodiment 162. The method according to embodiment 156, wherein the IL-2 conjugate has the amino acid sequence of SEQ ID NO: 41.

Embodiment 163. The method according to embodiment 162, wherein W is a PEG group having an average molecular weight selected from 5 kDa, 10 kDa, 15 kDa, 20 kDa, 25 kDa, and 30 kDa.

Embodiment 164. The method according to embodiment 163, wherein W is a PEG group having an average molecular weight selected from 5 kDa and 30 kDa.

Embodiment 165. The method according to Embodiment 164, wherein W is a PEG group having an average molecular weight of 5 kDa.

Embodiment 166. The method according to embodiment 164, wherein W is a PEG group having an average molecular weight of 30 kDa.

Embodiment 167. The method according to embodiment 156, wherein the IL-2 conjugate has the amino acid sequence of SEQ ID NO: 42.

Embodiment 168. The method according to embodiment 167, wherein W is a PEG group having an average molecular weight selected from 5 kDa, 10 kDa, 15 kDa, 20 kDa, 25 kDa, and 30 kDa.

Embodiment 169. The method according to embodiment 168, wherein W is a PEG group having an average molecular weight selected from 5 kDa and 30 kDa.

Embodiment 170. The method according to embodiment 169, wherein W is a PEG group having an average molecular weight of 5 kDa.

Embodiment 171. The method according to embodiment 169, wherein W is a PEG group having an average molecular weight of 30 kDa.

Embodiment 172. The method according to embodiment 156, wherein the IL-2 conjugate has the amino acid sequence of SEQ ID NO: 43.

Embodiment 173. The method according to embodiment 172, wherein W is a PEG group having an average molecular weight selected from 5 kDa, 10 kDa, 15 kDa, 20 kDa, 25 kDa, and 30 kDa.

Embodiment 174. The method according to embodiment 173, wherein W is a PEG group having an average molecular weight selected from 5 kDa and 30 kDa.

Embodiment 175. The method according to embodiment 174, wherein W is a PEG group having an average molecular weight of 5 kDa.

Embodiment 176. The method according to embodiment 174, wherein W is a PEG group having an average molecular weight of 30 kDa.

Embodiment 177. The method according to embodiment 156, wherein the IL-2 conjugate has the amino acid sequence of SEQ ID NO: 44.

Embodiment 178. The method according to embodiment 177, wherein W is a PEG group having an average molecular weight selected from 5 kDa, 10 kDa, 15 kDa, 20 kDa, 25 kDa, and 30 kDa.

Embodiment 179. The method according to embodiment 178, wherein W is a PEG group having an average molecular weight selected from 5 kDa and 30 kDa.

Embodiment 180. The method according to embodiment 179, wherein W is a PEG group having an average molecular weight of 5 kDa.

Embodiment 181. The method according to embodiment 179, wherein W is a PEG group having an average molecular weight of 30 kDa.

Embodiment 182. The method according to any one of embodiments 128 to 181, wherein W is a linear or branched PEG group.

Embodiment 183. The method according to anyone of embodiments 128 to 181, wherein W is a linear PEG group.

Embodiment 184. The method according to anyone of embodiments 128 to 181, wherein W is a branched PEG group.

Embodiment 185. The method according to anyone of embodiments 128 to 181, wherein W is a methoxy PEG group.

Embodiment 186. The method according to embodiment 185, wherein the methoxy PEG group is linear or branched.

Embodiment 187. The method according to embodiment 186, wherein the methoxy PEG group is linear.

Embodiment 188. The method according to embodiment 186, wherein the methoxy PEG group is branched.

Embodiment 189. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45-49, wherein [AzK_L1_PEG5kD] has the structure of Formula (IV) or Formula (V), or a mixture of Formula (IV) and Formula (V):

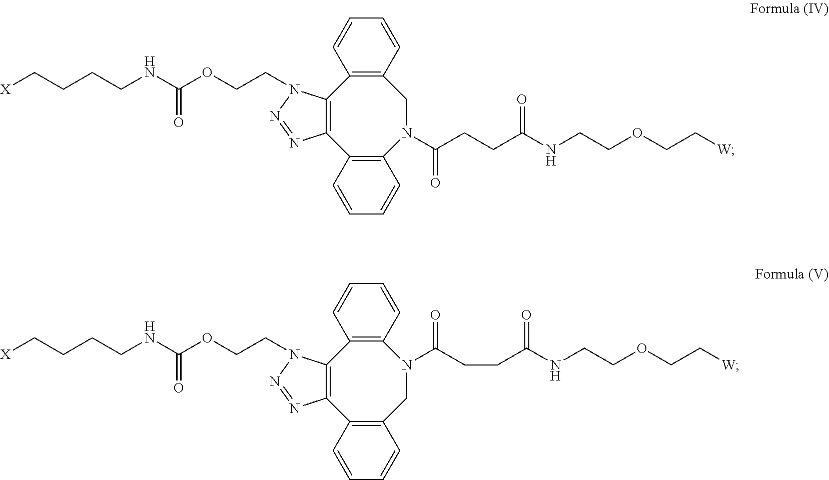

wherein:
W is a PEG group having an average molecular weight of 5 kDa; and
X has the structure:

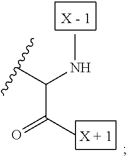

or a pharmaceutically acceptable salt, solvate, or hydrate thereof.

Embodiment 189.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45-49, wherein [AzK_L1_PEG5kD] has the structure of Formula (IV) or Formula (V), or a mixture of Formula (IV) and Formula (V):

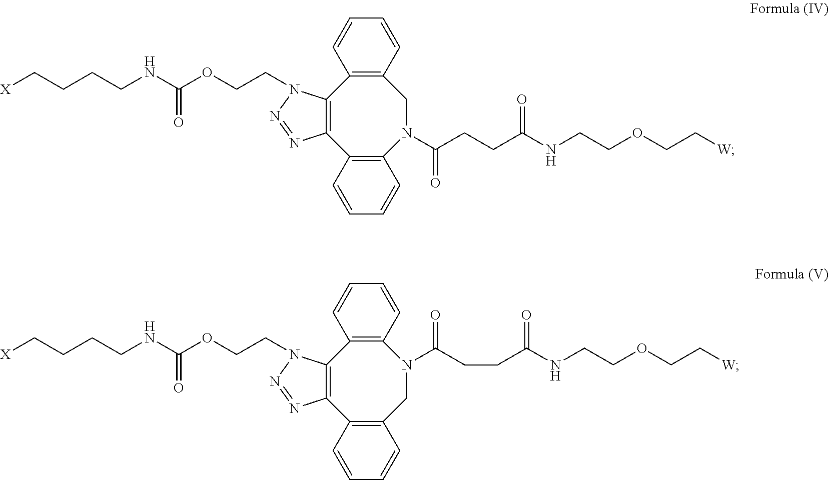

wherein:
W is a PEG group having an average molecular weight of 5 kDa; and
X has the structure:

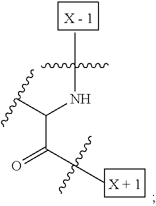

X−1 indicates the point of attachment to the preceding amino acid residue; and
X+1 indicates the point of attachment to the following amino acid residue;
or a pharmaceutically acceptable salt, solvate, or hydrate thereof.

Embodiment 190. The method according to embodiment 189 or 189.1, wherein the IL-2 conjugate has the amino acid sequence of SEQ ID NO: 45.

Embodiment 191. The method according to embodiment 189 or 189.1, wherein the IL-2 conjugate has the amino acid sequence of SEQ ID NO: 46.

Embodiment 192. The method according to embodiment 189 or 189.1, wherein the IL-2 conjugate has the amino acid sequence of SEQ ID NO: 47.

Embodiment 193. The method according to embodiment 189 or 189.1, wherein the IL-2 conjugate has the amino acid sequence of SEQ ID NO: 48.

Embodiment 194. The method according to embodiment 189 or 189.1, wherein the IL-2 conjugate has the amino acid sequence of SEQ ID NO: 49.

Embodiment 195. The method according to embodiment 189 or 189.1, wherein the [AzK_L1_PEG5kD] has the structure of Formula (IV)

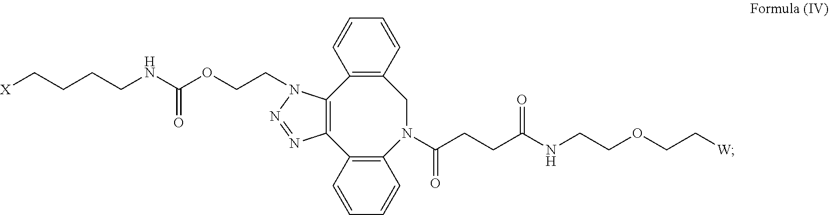

or a pharmaceutically acceptable salt, solvate, or hydrate thereof.

Embodiment 196. The method according to embodiment 195, wherein the IL-2 conjugate has the amino acid sequence of SEQ ID NO: 45.

Embodiment 197. The method according to embodiment 195, wherein the IL-2 conjugate has the amino acid sequence of SEQ ID NO: 46.

Embodiment 198. The method according to embodiment 195, wherein the IL-2 conjugate has the amino acid sequence of SEQ ID NO: 47.

Embodiment 199. The method according to embodiment 195, wherein the IL-2 conjugate has the amino acid sequence of SEQ ID NO: 48.

Embodiment 200. The method according to embodiment 195, wherein the IL-2 conjugate has the amino acid sequence of SEQ ID NO: 49.

Embodiment 201. The method according to embodiment 189 or 189.1, wherein the [AzK_L1_PEG5kD] has the structure of Formula (V)

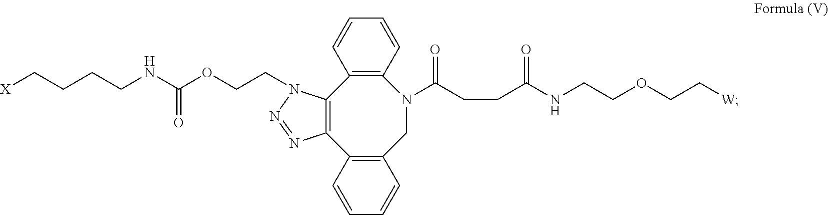

or a pharmaceutically acceptable salt, solvate, or hydrate thereof.

Embodiment 202. The method according to embodiment 201, wherein the IL-2 conjugate has the amino acid sequence of SEQ ID NO: 45.

Embodiment 203. The method according to embodiment 201, wherein the IL-2 conjugate has the amino acid sequence of SEQ ID NO: 46.

Embodiment 204. The method according to embodiment 201, wherein the IL-2 conjugate has the amino acid sequence of SEQ ID NO: 47.

Embodiment 205. The method according to embodiment 201, wherein the IL-2 conjugate has the amino acid sequence of SEQ ID NO: 48.

Embodiment 206. The method according to embodiment 201, wherein the IL-2 conjugate has the amino acid sequence of SEQ ID NO: 49.

Embodiment 207. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 50-54, wherein [AzK_L1_PEG30kD] has the structure of Formula (IV) or Formula (V), or is a mixture of the structures of Formula (IV) and Formula (V):

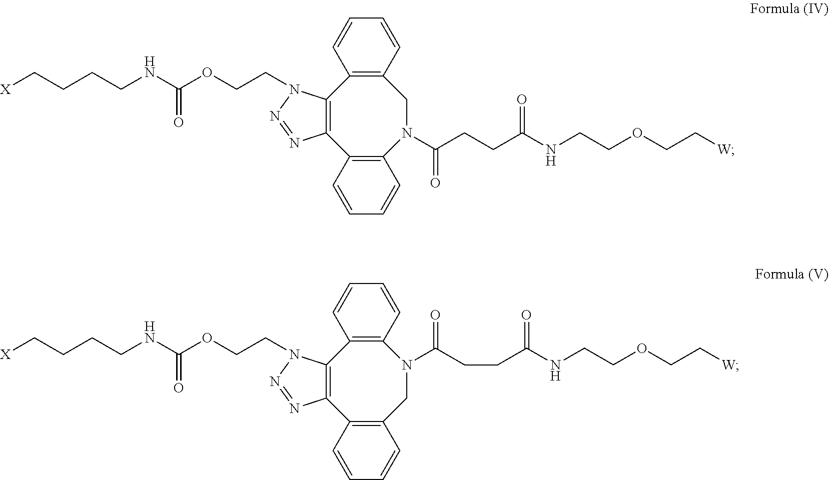

wherein:
W is a PEG group having an average molecular weight of 30 kDa; and
X has the structure:

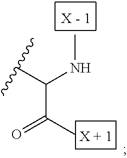

or a pharmaceutically acceptable salt, solvate, or hydrate thereof.

Embodiment 207.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 50-54, wherein [AzK_L1_PEG30kD] has the structure of Formula (IV) or Formula (V), or is a mixture of the structures of Formula (IV) and Formula (V):

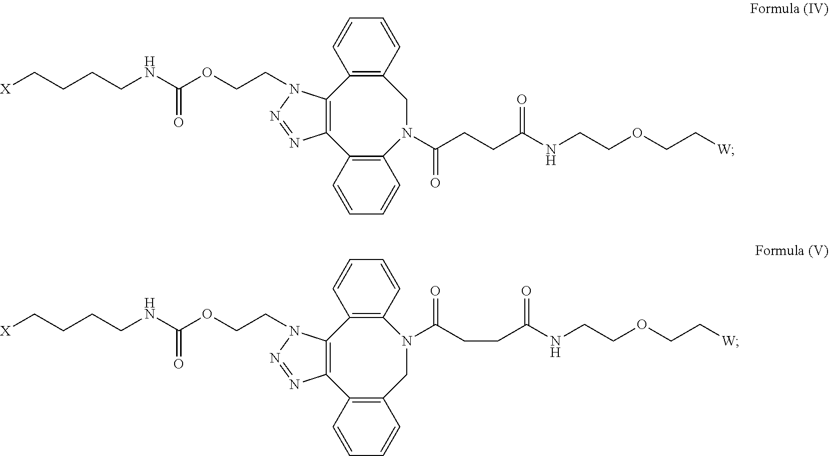

wherein:
W is a PEG group having an average molecular weight of 30 kDa; and
X has the structure:

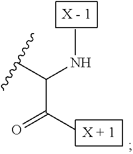 [SMILES: *C(NC)C(C)=O]

or a pharmaceutically acceptable salt, solvate, or hydrate thereof.

Embodiment 208. The method according to embodiment 207 or 207.1, wherein the IL-2 conjugate has the amino acid sequence of SEQ ID NO: 50.

Embodiment 209. The method according to embodiment 207 or 207.1, wherein the IL-2 conjugate has the amino acid sequence of SEQ ID NO: 51.

Embodiment 210. The method according to embodiment 207 or 207.1, wherein the IL-2 conjugate has the amino acid sequence of SEQ ID NO: 52.

Embodiment 211. The method according to embodiment 207 or 207.1, wherein the IL-2 conjugate has the amino acid sequence of SEQ ID NO: 53.

Embodiment 212. The method according to embodiment 207 or 207.1, wherein the IL-2 conjugate has the amino acid sequence of SEQ ID NO: 54.

Embodiment 213. The method according to embodiment 207 or 207.1, wherein the [AzK_L1_PEG30kD] has the structure of Formula (IV):

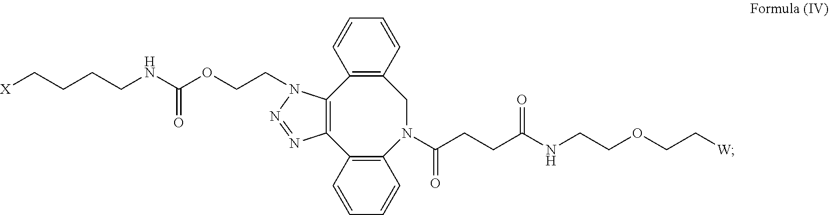

or a pharmaceutically acceptable salt, solvate, or hydrate thereof.

Embodiment 214. The method according to embodiment 213, wherein the IL-2 conjugate has the amino acid sequence of SEQ ID NO: 50.

Embodiment 215. The method according to embodiment 213, wherein the IL-2 conjugate has the amino acid sequence of SEQ ID NO: 51.

Embodiment 216. The method according to embodiment 213, wherein the IL-2 conjugate has the amino acid sequence of SEQ ID NO: 52.

Embodiment 217. The method according to embodiment 213, wherein the IL-2 conjugate has the amino acid sequence of SEQ ID NO: 53.

Embodiment 218. The method according to embodiment 213, wherein the IL-2 conjugate has the amino acid sequence of SEQ ID NO: 54.

Embodiment 219. The method according to embodiment 207 or 207.1, wherein the [AzK_L1_PEG30kD] has the structure of Formula (V)

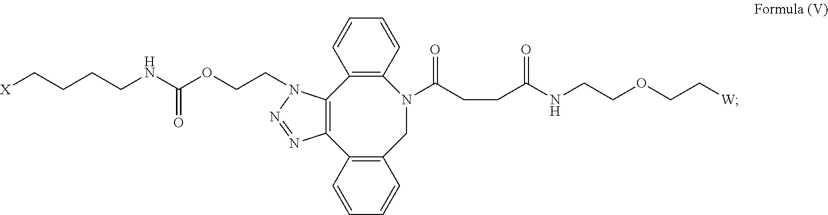

or a pharmaceutically acceptable salt, solvate, or hydrate thereof.

Embodiment 220. The method according to embodiment 219, wherein the IL-2 conjugate has the amino acid sequence of SEQ ID NO: 50.

Embodiment 221. The method according to embodiment 219, wherein the IL-2 conjugate has the amino acid sequence of SEQ ID NO: 51.

Embodiment 222. The method according to embodiment 219, wherein the IL-2 conjugate has the amino acid sequence of SEQ ID NO: 52.

Embodiment 223. The method according to embodiment 219, wherein the IL-2 conjugate has the amino acid sequence of SEQ ID NO: 53.

Embodiment 224. The method according to embodiment 219, wherein the IL-2 conjugate has the amino acid sequence of SEQ ID NO: 54.

Embodiment 225. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [Azk_L1_PEG] is a mixture of the structures of Formula (IV) and Formula (V):

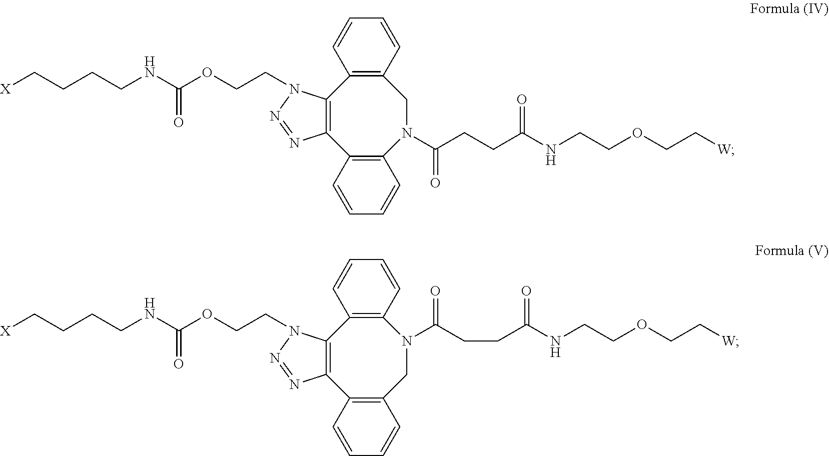

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

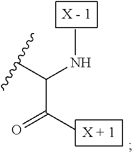 [SMILES: *C(NC)C(C)=O]

or a pharmaceutically acceptable salt, solvate, or hydrate thereof.

Embodiment 225.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 40-44, wherein [Azk_L1_PEG] is a mixture of the structures of Formula (IV) and Formula (V):

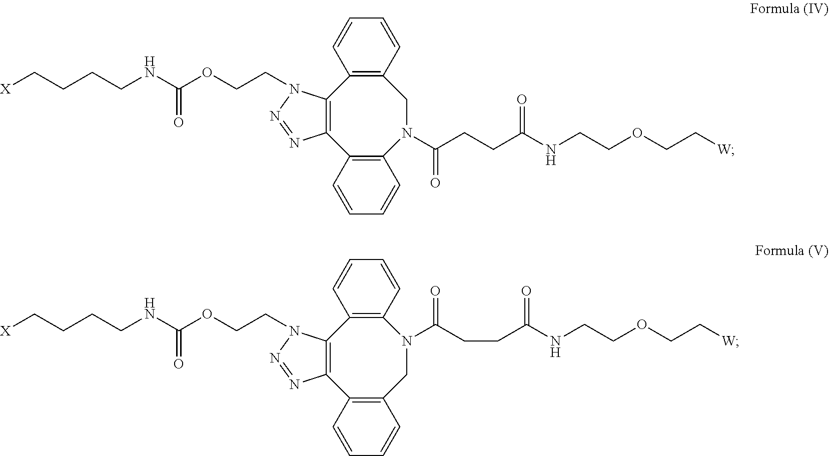

wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa; and
X has the structure:

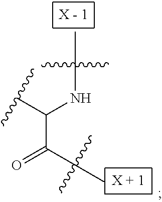 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 226. The method according to embodiment 225 or 225.1, wherein the ratio of the amount of the structure of Formula (IV) to the amount of the structure of Formula (V) comprising the total amount of [AzK_L1_PEG] in the IL-2 conjugate is about 1:1.

Embodiment 227. The method according to embodiment 225 or 225.1, wherein the ratio of the amount of the structure of Formula (IV) to the amount of the structure of Formula (V) comprising the total amount of [AzK_L1_PEG] in the IL-2 conjugate is greater than 1:1.

Embodiment 228. The method according to embodiment 225 or 225.1, wherein the ratio of the amount of the structure of Formula (IV) to the amount of the structure of Formula (V) comprising the total amount of [AzK_L1_PEG] in the IL-2 conjugate is less than 1:1.

Embodiment 229. The method according to any one of embodiments 225 to 228, wherein W is a linear or branched PEG group.

Embodiment 230. The method according to any one of embodiments 225 to 228, wherein W is a linear PEG group.

Embodiment 231. The method according to any one of embodiments 225 to 228, wherein W is a branched PEG group.

Embodiment 232. The method according to any one of embodiments 225 to 228, wherein W is a methoxy PEG group.

Embodiment 233. The method according to embodiment 232, wherein the methoxy PEG group is linear or branched.

Embodiment 234. The method according to embodiment 233, wherein the methoxy PEG group is linear.

Embodiment 235. The method according to embodiment 233, wherein the methoxy PEG group is branched.

Embodiment 236. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45 to 49, wherein [AzK_L1_PEG5kD] is a mixture of the structures of Formula (IV) and Formula (V):

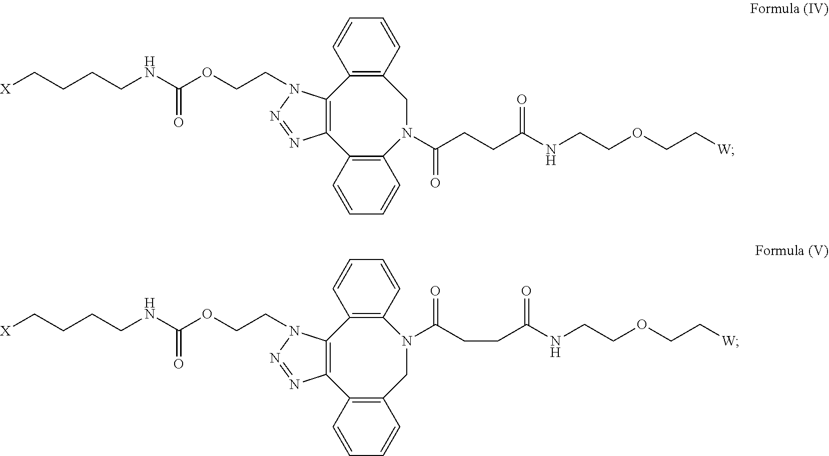

wherein:
W is a PEG group having an average molecular weight of 5 kDa; and
X has the structure:

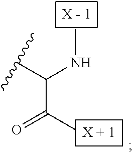 [SMILES: *C(NC)C(C)=O]

or a pharmaceutically acceptable salt, solvate, or hydrate thereof.

Embodiment 236.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one of SEQ ID NOS: 45 to 49, wherein [AzK_L1_PEG5kD] is a mixture of the structures of Formula (IV) and Formula (V):

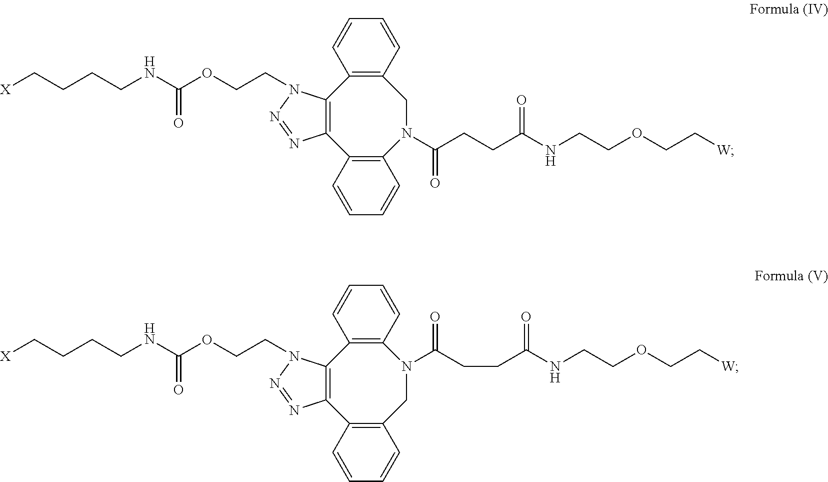

wherein:
W is a PEG group having an average molecular weight of 5 kDa; and
X has the structure:

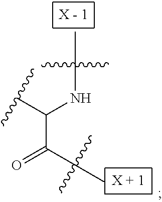 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 237. The method according to embodiment 236 or 236.1, wherein the ratio of the amount of the structure of Formula (IV) to the amount of the structure of Formula (V) comprising the total amount of [AzK_L1_PEG5kD] in the IL-2 conjugate is about 1:1.

Embodiment 238. The method according to embodiment 236 or 236.1, wherein the ratio of the amount of the structure of Formula (IV) to the amount of the structure of Formula (V) comprising the total amount of [AzK_L1_PEG5kD] in the IL-2 conjugate is greater than 1:1.

Embodiment 239. The method according to embodiment 236 or 236.1, wherein the ratio of the amount of the structure of Formula (IV) to the amount of the structure of Formula (V) comprising the total amount of [AzK_L1_PEG5kD] in the IL-2 conjugate is less than 1:1.

Embodiment 240. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one SEQ ID NOS: 50-54, wherein [AzK_L1 PEG30kD] is a mixture of the structures of Formula (IV) and Formula (V):

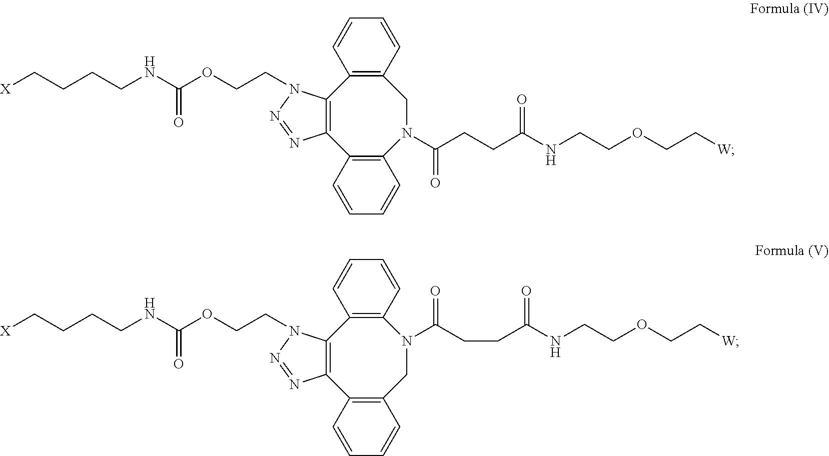

wherein:
W is a PEG group having an average molecular weight of 30 kDa; and
X has the structure:

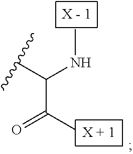 [SMILES: *C(NC)C(C)=O]

or a pharmaceutically acceptable salt, solvate, or hydrate thereof.

Embodiment 240.1. A method of treating cancer in a subject, comprising administering to a subject in need thereof a therapeutically effective amount of (a) an IL-2 conjugate, and (b) one or more additional agents, wherein the IL-2 conjugate comprises the amino acid sequence of any one SEQ ID NOS: 50-54, wherein [AzK_L1 PEG30kD] is a mixture of the structures of Formula (IV) and Formula (V):

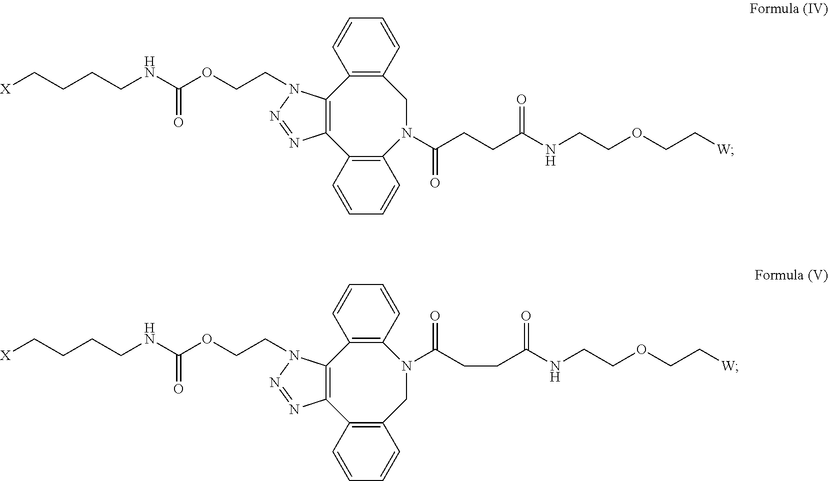

wherein:
W is a PEG group having an average molecular weight of 30 kDa; and
X has the structure:

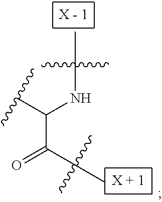 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 241. The method according to embodiment 240 or 240.1, wherein the ratio of the amount of the structure of Formula (IV) to the amount of the structure of Formula (V) comprising the total amount of [AzK_L1_PEG30kD] in the IL-2 conjugate is about 1:1.

Embodiment 242. The method according to embodiment 240 or 240.1, wherein the ratio of the amount of the structure of Formula (IV) to the amount of the structure of Formula (V) comprising the total amount of [AzK_L1_PEG30kD] in the IL-2 conjugate is greater than 1:1.

Embodiment 243. The method according to embodiment 240 or 240.1, wherein the ratio of the amount of the structure of Formula (IV) to the amount of the structure of Formula (V) comprising the total amount of [AzK_L1_PEG30kD] in the IL-2 conjugate is less than 1:1.

Embodiment 244. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (VI) or (VII), or a mixture of (VI) and (VII):

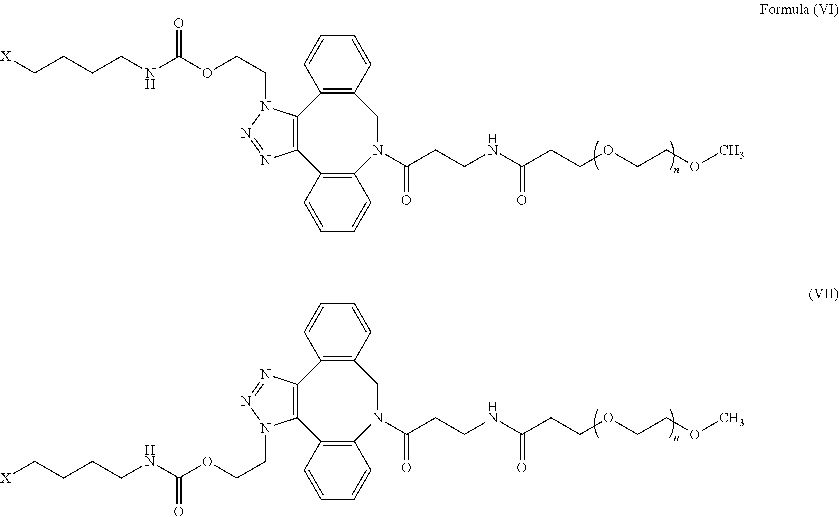

wherein:
n is an integer in the range from about 2 to about 5000; and
X has the structure:

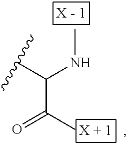 [SMILES: *C(NC)C(C)=O]

or a pharmaceutically acceptable salt, solvate, or hydrate thereof.

Embodiment 244.1. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of

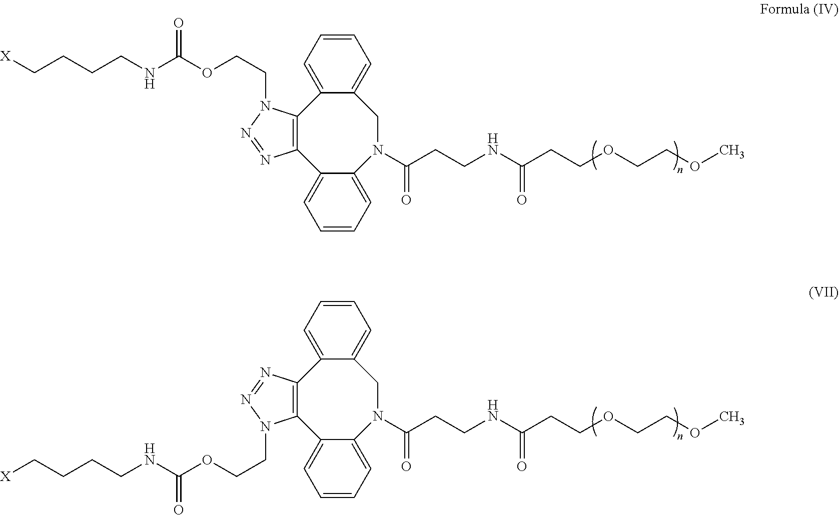

wherein:
n is an integer in the range from about 2 to about 5000; and
X has the structure:

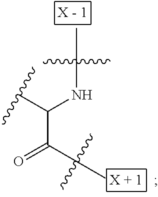 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 245. The method according to embodiment 244 or 244.1, wherein n in the compounds of Formula (VI) and (VII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

Embodiment 246. The method according to embodiment 244 or 244.1, wherein n in the compounds of Formula (VI) and (VII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

Embodiment 247. The method according to any one of embodiments 244 to 246, wherein the position of the structure of Formula (VI), Formula (VII), or a mixture of Formula (VI) and (VII) in the amino acid sequence of the IL-2 conjugate is selected from K34, F41, F43, K42, E61, P64, R37, T40, E67, Y44, V68, and L71.

Embodiment 248. The method according to embodiment 247, wherein the position of the structure of Formula (VI), Formula (VII), or a mixture of Formula (VI) and (VII) in the amino acid sequence of the IL-2 conjugate is K34.

Embodiment 249. The method according to embodiment 247, wherein the position of the structure of Formula (VI), Formula (VII), or a mixture of Formula (VI) and (VII) in the amino acid sequence of the IL-2 conjugate is F41.

Embodiment 250. The method according to embodiment 247, wherein the position of the structure of Formula (VI), Formula (VII), or a mixture of Formula (VI) and (VII) in the amino acid sequence of the IL-2 conjugate is F43.

Embodiment 251. The method according to embodiment 247, wherein the position of the structure of Formula (VI), Formula (VII), or a mixture of Formula (VI) and (VII) in the amino acid sequence of the IL-2 conjugate is K42.

Embodiment 252. The method according to embodiment 247, wherein the position of the structure of Formula (VI), Formula (VII), or a mixture of Formula (VI) and (VII) in the amino acid sequence of the IL-2 conjugate is E61.

Embodiment 253. The method according to embodiment 247, wherein the position of the structure of Formula (VI), Formula (VII), or a mixture of Formula (VI) and (VII) in the amino acid sequence of the IL-2 conjugate is P64.

Embodiment 254. The method according to embodiment 247, wherein the position of the structure of Formula (VI), Formula (VII), or a mixture of Formula (VI) and (VII) in the amino acid sequence of the IL-2 conjugate is R37.

Embodiment 255. The method according to embodiment 247, wherein the position of the structure of Formula (VI), Formula (VII), or a mixture of Formula (VI) and (VII) in the amino acid sequence of the IL-2 conjugate is T40.

Embodiment 256. The method according to embodiment 247, wherein the position of the structure of Formula (VI), Formula (VII), or a mixture of Formula (VI) and (VII) in the amino acid sequence of the IL-2 conjugate is E67.

Embodiment 257. The method according to embodiment 247, wherein the position of the structure of Formula (VI), Formula (VII), or a mixture of Formula (VI) and (VII) in the amino acid sequence of the IL-2 conjugate is Y44.

Embodiment 258. The method according to embodiment 247, wherein the position of the structure of Formula (VI), Formula (VII), or a mixture of Formula (VI) and (VII) in the amino acid sequence of the IL-2 conjugate is V68.

Embodiment 259. The method according to embodiment 247, wherein the position of the structure of Formula (VI), Formula (VII), or a mixture of Formula (VI) and (VII) in the amino acid sequence of the IL-2 conjugate is and L71.

Embodiment 260. The method according to any one of embodiments 244 to 259, wherein the ratio of the amount of the structure of Formula (VI) to the amount of the structure of Formula (VII) comprising the total amount of the IL-2 conjugate is greater than 1:1.

Embodiment 261. The method according to any one of embodiments 244 to 259, wherein the ratio of the amount of the structure of Formula (VI) to the amount of the structure of Formula (VII) comprising the total amount of the IL-2 conjugate is less than 1:1.

Embodiment 262. The method according to embodiment 244 or 244.1, wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909.

Embodiment 263. The method according to Embodiment 244 or 244.1, wherein the amino acid residue in SEQ ID NO: 3 that is replaced is selected from F41, F43, K42, E61, and P64, and n is an integer selected from 454, 455, 568, 569, 680, 681, 682, 794, 795, 796, 908, 909, 910, 1021, 1022, 1023, 1135, 1136, 1137, and 1249.

Embodiment 264. The method according to embodiment 244 or 244.1, wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909.

Embodiment 265. The method according embodiment 264, wherein n in the compounds of formula (VI) and (VII) is an integer selected from 454, 455, 568, 569, 680, 681, 682, 794, 795, 796, 908, 909, and 910.

Embodiment 266. The method according to embodiment 244 or 244.1, wherein the amino acid residue in SEQ ID NO: 3 that is replaced is E61, and wherein n is an integer from about 450 to about 800, or from about 454 to about 796, or from about 454 to about 682, or from about 568 to about 909.

Embodiment 267. The method according to embodiment 266, wherein n in the compounds of formula (VI) and (VII) is an integer selected from 454, 455, 568, 569, 680, 681, 682, 794, 795, 796, 908, 909, and 910.

Embodiment 268. The method according to embodiment 266, wherein the amino acid residue in SEQ ID NO: 3 that is replaced is E61, and wherein n is an integer from about 500 to about 1000.

Embodiment 269. The method according to embodiment 266, wherein the amino acid residue in SEQ ID NO: 3 that is replaced is E61, and wherein n is an integer from about 550 to about 800.

Embodiment 270. The method according to embodiment 267, wherein the amino acid residue in SEQ ID NO: 3 that is replaced is E61, and wherein n is 681.

Embodiment 271. The method according to embodiment 244 or 244.1, wherein the amino acid residue in in SEQ ID NO: 3 that is replaced is P64, and wherein n is an integer from about 450 to about 800, or from about 454 to about 796, or from about 454 to about 682, or from about 568 to about 909.

Embodiment 272. The method according to embodiment 271, wherein the amino acid residue in in SEQ ID NO: 3 that is replaced is P64, and wherein n in the compounds of formula (VI) and (VII) is an integer selected from 454, 455, 568, 569, 680, 681, 682, 794, 795, 796, 908, 909, and 910.

Embodiment 273. The method according to embodiment 271, wherein the amino acid residue in in SEQ ID NO: 3 that is replaced is P64, and wherein n is an integer from about 500 to about 1000.

Embodiment 274. The method according to embodiment 273, wherein the amino acid residue in in SEQ ID NO: 3 that is replaced is P64, and wherein n is an integer from about 550 to about 800.

Embodiment 275. The method according to embodiment 271, wherein the amino acid residue in in SEQ ID NO: 3 that is replaced is P64, and wherein n is 681.

Embodiment 276. The method according to embodiment 244 or 244.1, wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Embodiment 277. The method according to embodiment 244 or 244.1, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

Embodiment 278. The method according to embodiment 244 or 244.1, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Embodiment 279. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (VIII) or (IX), or a mixture of (VIII) and (IX):

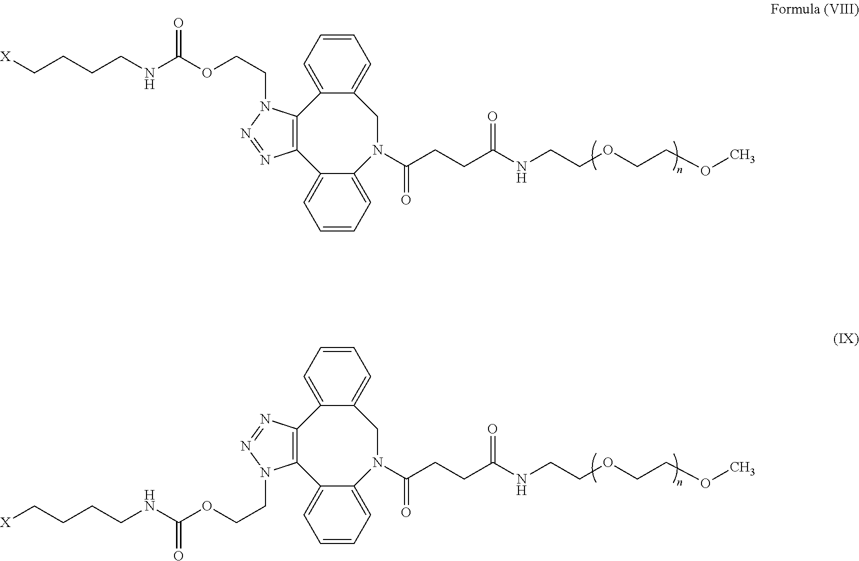

wherein:
n is an integer in the range from about 2 to about 5000; and
X has the structure:

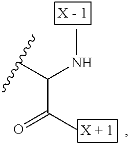 [SMILES: *C(NC)C(C)=O]

or a pharmaceutically acceptable salt, solvate, or hydrate thereof.

Embodiment 279.1. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (VIII) or (IX), or a mixture of (VIII) and (IX):

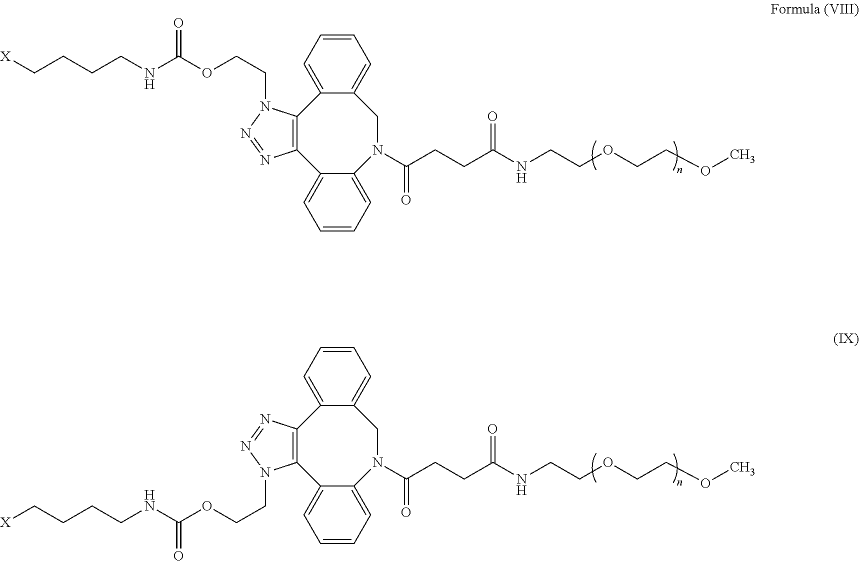

wherein:
n is an integer in the range from about 2 to about 5000; and
X has the structure:

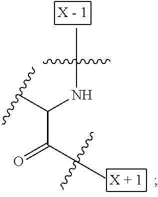 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 280. The method according to embodiment 279 or 279.1, wherein n in the compounds of Formula (VIII) and (IX) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

Embodiment 281. The method according to embodiment 279 or 279.1, wherein n in the compounds of Formula (VIII) and (IX) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

Embodiment 282. The method according to anyone of embodiments 279 to 281, wherein the position of the structure of Formula (VIII), Formula (IX), or a mixture of Formula (VIII) and (IX) in the amino acid sequence of the IL-2 conjugate is selected from K34, F41, F43, K42, E61, P64, R37, T40, E67, Y44, V68, and L71.

Embodiment 283. The method according to embodiment 282, wherein the position of the structure of Formula (VIII), Formula (IX), or a mixture of Formula (VIII) and (IX) in the amino acid sequence of the IL-2 conjugate is K34.

Embodiment 284. The method according to embodiment 282, wherein the position of the structure of Formula (VIII), Formula (IX), or a mixture of Formula (VIII) and (IX) in the amino acid sequence of the IL-2 conjugate is F41.

Embodiment 285. The method according to embodiment 282, wherein the position of the structure of Formula (VIII), Formula (IX), or a mixture of Formula (VIII) and (IX) in the amino acid sequence of the IL-2 conjugate is F43.

Embodiment 286. The method according to embodiment 282, wherein the position of the structure of Formula (VIII), Formula (IX), or a mixture of Formula (VIII) and (IX) in the amino acid sequence of the IL-2 conjugate is K42.

Embodiment 287. The method according to embodiment 282, wherein the position of the structure of Formula (VIII), Formula (IX), or a mixture of Formula (VIII) and (IX) in the amino acid sequence of the IL-2 conjugate is E61.

Embodiment 288. The method according to embodiment 282, wherein the position of the structure of Formula (VIII), Formula (IX), or a mixture of Formula (VIII) and (IX) in the amino acid sequence of the IL-2 conjugate is P64.

Embodiment 289. The method according to embodiment 282, wherein the position of the structure of Formula (VIII), Formula (IX), or a mixture of Formula (VIII) and (IX) in the amino acid sequence of the IL-2 conjugate is R37.

Embodiment 290. The method according to embodiment 282, wherein the position of the structure of Formula (VIII), Formula (IX), or a mixture of Formula (VIII) and (IX) in the amino acid sequence of the IL-2 conjugate is T40.

Embodiment 291. The method according to embodiment 282, wherein the position of the structure of Formula (VIII), Formula (IX), or a mixture of Formula (VIII) and (IX) in the amino acid sequence of the IL-2 conjugate is E67.

Embodiment 292. The method according to embodiment 282, wherein the position of the structure of Formula (VIII), Formula (IX), or a mixture of Formula (VIII) and (IX) in the amino acid sequence of the IL-2 conjugate is Y44.

Embodiment 293. The method according to embodiment 282, wherein the position of the structure of Formula (VIII), Formula (IX), or a mixture of Formula (VIII) and (IX) in the amino acid sequence of the IL-2 conjugate is V68.

Embodiment 294. The method according to embodiment 282, wherein the position of the structure of Formula (VIII), Formula (IX), or a mixture of Formula (VIII) and (IX) in the amino acid sequence of the IL-2 conjugate is L71.

Embodiment 295. The method according to any one of embodiments 279 to 294, wherein the ratio of the amount of the structure of Formula (VIII) to the amount of the structure of Formula (IX) comprising the total amount of the IL-2 conjugate is greater than 1:1.

Embodiment 296. The method according to any one of embodiments 279 to 294, wherein the ratio of the amount of the structure of Formula (VIII) to the amount of the structure of Formula (IX) comprising the total amount of the IL-2 conjugate is less than 1:1.

Embodiment 297. The method according to embodiment 279 or 279.1, wherein the amino acid residue in SEQ ID NO: 3 that is replaced is selected from F41, F43, K42, E61, and P64, and wherein n is an integer from about 450 to about 800, or from about 454 to about 796, or from about 454 to about 682, or from about 568 to about 909.

Embodiment 298. The method according to embodiment 279 or 279.1, wherein the amino acid residue in SEQ ID NO: 3 that is replaced is selected from F41, F43, K42, E61, and P64, and n is an integer selected from 454, 455, 568, 569, 680, 681, 682, 794, 795, 796, 908, 909, 910, 1021, 1022, 1023, 1135, 1136, 1137, and 1249.

Embodiment 299. The method according to embodiment 279 or 279.1, wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909.

Embodiment 300. The method according embodiment 299, wherein n in the compounds of formula (VIII) and (IX) is an integer selected from 454, 455, 568, 569, 680, 681, 682, 794, 795, 796, 908, 909, and 910.

Embodiment 301. The method according to embodiment 279 or 279.1, wherein the amino acid residue in SEQ ID NO: 3 that is replaced is E61, and wherein n is an integer from about 450 to about 800, or from about 454 to about 796, or from about 454 to about 682, or from about 568 to about 909.

Embodiment 302. The method according to embodiment 300, wherein the amino acid residue in SEQ ID NO: 3 that is replaced is E61, and wherein n in the compounds of formula (VIII) and (IX) is an integer selected from 454, 455, 568, 569, 680, 681, 682, 794, 795, 796, 908, 909, and 910.

Embodiment 303. The method according to embodiment 279 or 279.1, wherein the amino acid residue in SEQ ID NO: 3 that is replaced is E61, and wherein n is an integer from about 500 to about 1000.

Embodiment 304. The method according to embodiment 303, wherein the amino acid residue in SEQ ID NO: 3 that is replaced is E61, and wherein n is an integer from about 550 to about 800.

Embodiment 305. The method according to embodiment 302, wherein the amino acid residue in SEQ ID NO: 3 that is replaced is E61, and wherein n is 681.

Embodiment 306. The method according to embodiment 279 or 279.1, wherein the amino acid residue in in SEQ ID NO: 3 that is replaced is P64, and wherein n is an integer from about 450 to about 800, or from about 454 to about 796, or from about 454 to about 682, or from about 568 to about 909.

Embodiment 307. The method according to embodiment 279 or 279.1, wherein the amino acid residue in in SEQ ID NO: 3 that is replaced is P64, and wherein n in the compounds of formula (VIII) and (IX) is an integer selected from 454, 455, 568, 569, 680, 681, 682, 794, 795, 796, 908, 909, and 910.

Embodiment 308. The method according to embodiment 279 or 279.1, wherein the amino acid residue in in SEQ ID NO: 3 that is replaced is P64, and wherein n is an integer from about 500 to about 1000.

Embodiment 309. The method according to embodiment 279 or 279.1, wherein the amino acid residue in in SEQ ID NO: 3 that is replaced is P64, and wherein n is an integer from about 550 to about 800.

Embodiment 310. The method according to embodiment 307, wherein the amino acid residue in in SEQ ID NO: 3 that is replaced is P64, and wherein n is 681.

Embodiment 311. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (X) or (XI), or a mixture of (X) and (XI):

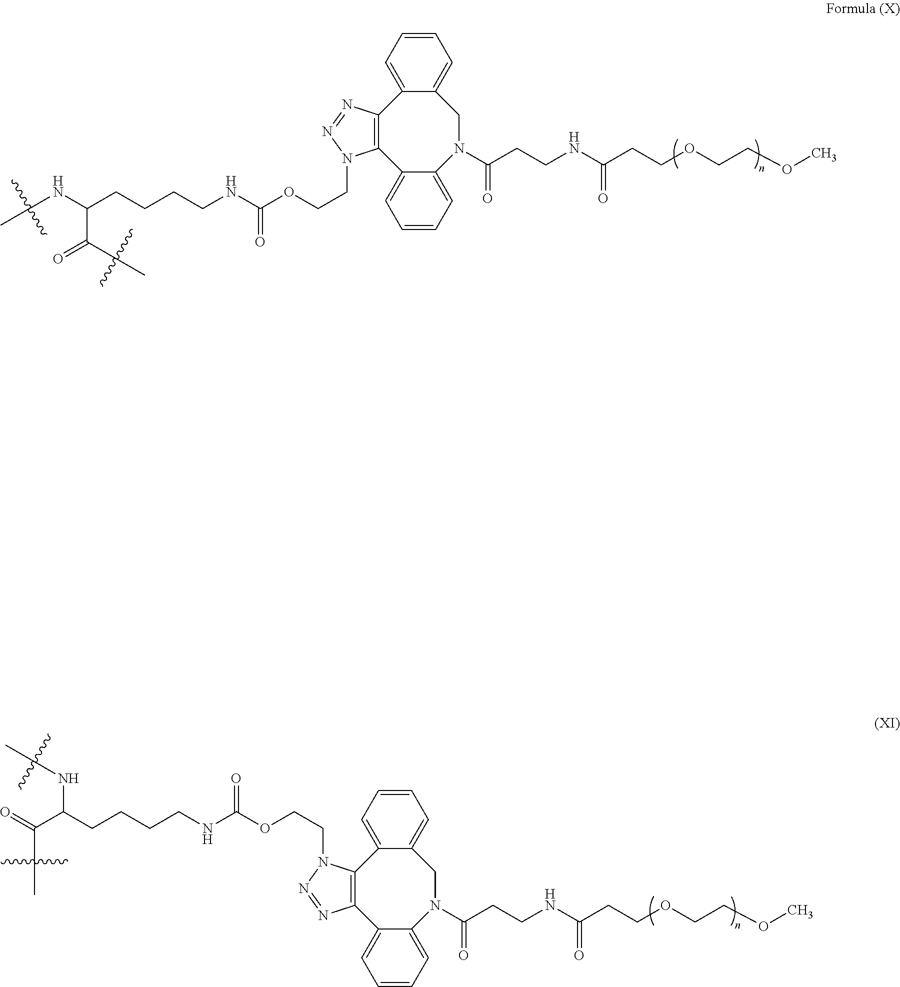

wherein:
n is an integer in the range from about 2 to about 5000; and the wavy lines indicate covalent bonds to amino acid residues within SEQ ID NO: 3 that are not replaced.

Embodiment 312. The method according to embodiment 311, wherein n in the compounds of Formula (X) and (XI) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

Embodiment 313. The method according to embodiment 311, wherein n in the compounds of Formula (X) and (XI) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

Embodiment 314. The method according to any one of embodiments 311 to 313, wherein the position of the structure of Formula (X), Formula (XI), or a mixture of Formula (X) and (XI) in the amino acid sequence of the IL-2 conjugate is selected from K34, F41, F43, K42, E61, P64, R37, T40, E67, Y44, V68, and L71.

Embodiment 315. The method according to embodiment 314, wherein the position of the structure of Formula (X), Formula (XI), or a mixture of Formula (X) and (XI) in the amino acid sequence of the IL-2 conjugate is K34.

Embodiment 316. The method according to embodiment 314, wherein the position of the structure of Formula (X), Formula (XI), or a mixture of Formula (X) and (XI) in the amino acid sequence of the IL-2 conjugate is F41.

Embodiment 317. The method according to embodiment 314, wherein the position of the structure of Formula (X), Formula (XI), or a mixture of Formula (X) and (XI) in the amino acid sequence of the IL-2 conjugate is F43.

Embodiment 318. The method according to embodiment 314, wherein the position of the structure of Formula (X), Formula (XI), or a mixture of Formula (X) and (XI) in the amino acid sequence of the IL-2 conjugate is K42.

Embodiment 319. The method according to embodiment 314, wherein the position of the structure of Formula (X), Formula (XI), or a mixture of Formula (X) and (XI) in the amino acid sequence of the IL-2 conjugate is E61.

Embodiment 320. The method according to embodiment 314, wherein the position of the structure of Formula (X), Formula (XI), or a mixture of Formula (X) and (XI) in the amino acid sequence of the IL-2 conjugate is P64.

Embodiment 321. The method according to embodiment 314, wherein the position of the structure of Formula (X), Formula (XI), or a mixture of Formula (X) and (XI) in the amino acid sequence of the IL-2 conjugate is R37.

Embodiment 322. The method according to embodiment 314, wherein the position of the structure of Formula (X), Formula (XI), or a mixture of Formula (X) and (XI) in the amino acid sequence of the IL-2 conjugate is T40.

Embodiment 323. The method according to embodiment 314, wherein the position of the structure of Formula (X), Formula (XI), or a mixture of Formula (X) and (XI) in the amino acid sequence of the IL-2 conjugate is E67.

Embodiment 324. The method according to embodiment 314, wherein the position of the structure of Formula (X), Formula (XI), or a mixture of Formula (X) and (XI) in the amino acid sequence of the IL-2 conjugate is Y44.

Embodiment 325. The method according to embodiment 314, wherein the position of the structure of Formula (X), Formula (XI), or a mixture of Formula (X) and (XI) in the amino acid sequence of the IL-2 conjugate is V68.

Embodiment 326. The method according to embodiment 314, wherein the position of the structure of Formula (X), Formula (XI), or a mixture of Formula (X) and (XI) in the amino acid sequence of the IL-2 conjugate is and L71.

Embodiment 327. The method according to anyone of embodiments 311 to 326, wherein the ratio of the amount of the structure of Formula (X) to the amount of the structure of Formula (XI) comprising the total amount of the IL-2 conjugate is greater than 1:1.

Embodiment 328. The method according to any one of embodiments 311 to 326, wherein the ratio of the amount of the structure of Formula (X) to the amount of the structure of Formula (XI) comprising the total amount of the IL-2 conjugate is less than 1:1.

Embodiment 329. The method according to embodiment 311, wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909.

Embodiment 330. The method according to embodiment 311, wherein the amino acid residue in SEQ ID NO: 3 that is replaced is selected from F41, F43, K42, E61, and P64, and n is an integer selected from 454, 455, 568, 569, 680, 681, 682, 794, 795, 796, 908, 909, 910, 1021, 1022, 1023, 1135, 1136, 1137, and 1249.

Embodiment 331. The method according to embodiment 311, wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909.

Embodiment 332. The method according embodiment 330, wherein the amino acid residue in SEQ ID NO: 3 that is replaced is selected from F41, F43, K42, E61, and P64, and wherein n in the compounds of formula (X) and (XI) is an integer selected from 454, 455, 568, 569, 680, 681, 682, 794, 795, 796, 908, 909, and 910.

Embodiment 333. The method according to embodiment 311, wherein the amino acid residue in SEQ ID NO: 3 that is replaced is E61, and wherein n is an integer from about 450 to about 800, or from about 454 to about 796, or from about 454 to about 682, or from about 568 to about 909.

Embodiment 334. The method according to embodiment 311, wherein n in the compounds of formula (X) and (XI) is an integer selected from 454, 455, 568, 569, 680, 681, 682, 794, 795, 796, 908, 909, and 910.

Embodiment 335. The method according to embodiment 311, wherein n is from about 500 to about 1000.

Embodiment 336. The method according to embodiment 335, wherein n is from about 550 to about 800.

Embodiment 337. The method according to embodiment 332, wherein n is 681.

Embodiment 338. The method according to embodiment 311, wherein the amino acid residue in SEQ ID NO: 3 that is replaced is P64, and wherein n is an integer from about 450 to about 800, or from about 454 to about 796, or from about 454 to about 682, or from about 568 to about 909.

Embodiment 339. The method according to embodiment 311, wherein the amino acid residue in SEQ ID NO: 3 that is replaced is P64, and wherein n in the compounds of formula (X) and (XI) is an integer selected from 454, 455, 568, 569, 680, 681, 682, 794, 795, 796, 908, 909, and 910.

Embodiment 340. The method according to embodiment 311, wherein the amino acid residue in SEQ ID NO: 3 that is replaced is P64, and wherein n is an integer from about 500 to about 1000.

Embodiment 341. The method according to embodiment 340, wherein the amino acid residue in in SEQ ID NO: 3 that is replaced is P64, and wherein n is an integer from about 550 to about 800.

Embodiment 342. The method according to embodiment 339, wherein the amino acid residue in SEQ ID NO: 3 that is replaced is P64, and wherein n is 681.

Embodiment 343. The method according to embodiment 311, wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Embodiment 344. The method according to embodiment 311, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

Embodiment 345. The method according to embodiment 311, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Embodiment 346. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one amino acid residue in the IL-2 conjugate is replaced by the structure of Formula (XII) or (XIII), or a mixture of (XII) and (XIII):

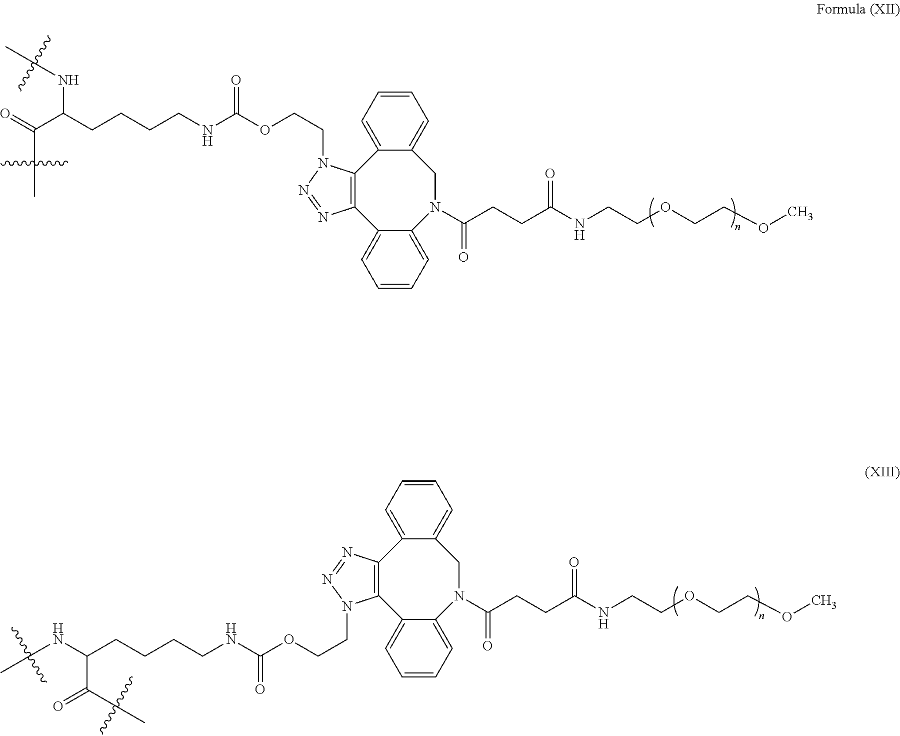

wherein:
n is an integer in the range from about 2 to about 5000; and
the wavy lines indicate covalent bonds to amino acid residues within SEQ ID NO: 3 that are not replaced.

Embodiment 347. The method according to embodiment 346, wherein n in the compounds of Formula (XII) and (XIII) is in the range from about 5 to about 4600, or from about 10 to about 4000, or from about 20 to about 3000, or from about 100 to about 3000, or from about 100 to about 2900, or from about 150 to about 2900, or from about 125 to about 2900, or from about 100 to about 2500, or from about 100 to about 2000, or from about 100 to about 1900, or from about 100 to about 1850, or from about 100 to about 1750, or from about 100 to about 1650, or from about 100 to about 1500, or from about 100 to about 1400, or from about 100 to about 1300, or from about 100 to about 1250, or from about 100 to about 1150, or from about 100 to about 1100, or from about 100 to about 1000, or from about 100 to about 900, or from about 100 to about 750, or from about 100 to about 700, or from about 100 to about 600, or from about 100 to about 575, or from about 100 to about 500, or from about 100 to about 450, or from about 100 to about to about 350, or from about 100 to about 275, or from about 100 to about 230, or from about 150 to about 475, or from about 150 to about 340, or from about 113 to about 340, or from about 450 to about 800, or from about 454 to about 796, or from about 454 to about 682, or from about 340 to about 795, or from about 341 to about 682, or from about 568 to about 909, or from about 227 to about 1500, or from about 225 to about 2280, or from about 460 to about 2160, or from about 460 to about 2050, or from about 341 to about 1820, or from about 341 to about 1710, or from about 341 to about 1250, or from about 225 to about 1250, or from about 341 to about 1250, or from about 341 to about 1136, or from about 341 to about 1023, or from about 341 to about 910, or from about 341 to about 796, or from about 341 to about 682, or from about 341 to about 568, or from about 114 to about 1000, or from about 114 to about 950, or from about 114 to about 910, or from about 114 to about 800, or from about 114 to about 690, or from about 114 to about 575.

Embodiment 348. The method according to embodiment 346, wherein n in the compounds of Formula (XII) and (XIII) is an integer selected from 2, 5, 10, 11, 22, 23, 113, 114, 227, 228, 340, 341, 454, 455, 568, 569, 680, 681, 682, 794, 795, 796, 908, 909, 910, 1021, 1022, 1023, 1135, 1136, 1137, 1249, 1250, 1251, 1362, 1363, 1364, 1476, 1477, 1478, 1589, 1590, 1591, 1703, 1704, 1705, 1817, 1818, 1819, 1930, 1931, 1932, 2044, 2045, 2046, 2158, 2159, 2160, 2271, 2272, 2273, 2839, 2840, 2841, 2953, 2954, 2955, 3408, 3409, 3410, 3976, 3977, 3978, 4544, 4545, and 4546.

Embodiment 349. The method according to any one of embodiments 346 to 348, wherein the position of the structure of Formula (XII), Formula (XIII), or a mixture of Formula (XII) and (XIII) in the amino acid sequence of the IL-2 conjugate is selected from K34, F41, F43, K42, E61, P64, R37, T40, E67, Y44, V68, and L71.

Embodiment 350. The method according to embodiment 349, wherein the position of the structure of Formula (XII), Formula (XIII), or a mixture of Formula (XII) and (XIII) in the amino acid sequence of the IL-2 conjugate is K34.

Embodiment 351. The method according to embodiment 349, wherein the position of the structure of Formula (XII), Formula (XIII), or a mixture of Formula (XII) and (XIII) in the amino acid sequence of the IL-2 conjugate is F41.

Embodiment 352. The method according to embodiment 349, wherein the position of the structure of Formula (XII), Formula (XIII), or a mixture of Formula (XII) and (XIII) in the amino acid sequence of the IL-2 conjugate is F43.

Embodiment 353. The method according to embodiment 349, wherein the position of the structure of Formula (XII), Formula (XIII), or a mixture of Formula (XII) and (XIII) in the amino acid sequence of the IL-2 conjugate is K42.

Embodiment 354. The method according to embodiment 349, wherein the position of the structure of Formula (XII), Formula (XIII), or a mixture of Formula (XII) and (XIII) in the amino acid sequence of the IL-2 conjugate is E61.

Embodiment 355. The method according to embodiment 349, wherein the position of the structure of Formula (XII), Formula (XIII), or a mixture of Formula (XII) and (XIII) in the amino acid sequence of the IL-2 conjugate is P64.

Embodiment 356. The method according to embodiment 349, wherein the position of the structure of Formula (XII), Formula (XIII), or a mixture of Formula (XII) and (XIII) in the amino acid sequence of the IL-2 conjugate is R37.

Embodiment 357. The method according to embodiment 349, wherein the position of the structure of Formula (XII), Formula (XIII), or a mixture of Formula (XII) and (XIII) in the amino acid sequence of the IL-2 conjugate is T40.

Embodiment 358. The method according to embodiment 349, wherein the position of the structure of Formula (XII), Formula (XIII), or a mixture of Formula (XII) and (XIII) in the amino acid sequence of the IL-2 conjugate is E67.

Embodiment 359. The method according to embodiment 349, wherein the position of the structure of Formula (XII), Formula (XIII), or a mixture of Formula (XII) and (XIII) in the amino acid sequence of the IL-2 conjugate is Y44.

Embodiment 360. The method according to embodiment 349, wherein the position of the structure of Formula (XII), Formula (XIII), or a mixture of Formula (XII) and (XIII) in the amino acid sequence of the IL-2 conjugate is V68.

Embodiment 361. The method according to embodiment 349, wherein the position of the structure of Formula (XII), Formula (XIII), or a mixture of Formula (XII) and (XIII) in the amino acid sequence of the IL-2 conjugate is and L71.

Embodiment 362. The method according to anyone of embodiments 346 to 361, wherein the ratio of the amount of the structure of Formula (XII) to the amount of the structure of Formula (XIII) comprising the total amount of the IL-2 conjugate is greater than 1:1.

Embodiment 363. The method according to any one of embodiments 346 to 361, wherein the ratio of the amount of the structure of Formula (XII) to the amount of the structure of Formula (XIII) comprising the total amount of the IL-2 conjugate is less than 1:1.

Embodiment 364. The method according to embodiment 346, wherein the amino acid residue in in SEQ ID NO: 3 that is replaced is selected from F41, F43, K42, E61, and P64, and wherein n is an integer from about 450 to about 800, or from about 454 to about 796, or from about 454 to about 682, or from about 568 to about 909.

Embodiment 365. The method according to embodiment 346, wherein the amino acid residue in SEQ ID NO: 3 that is replaced is selected from F41, F43, K42, E61, and P64, and n is an integer selected from 454, 455, 568, 569, 680, 681, 682, 794, 795, 796, 908, 909, 910, 1021, 1022, 1023, 1135, 1136, 1137, and 1249.

Embodiment 366. The method according to embodiment 346, wherein the amino acid residue in SEQ ID NO: 3 that is replaced is selected from E61 and P64, and wherein n is an integer from about 450 to about 800, or from about 454 to about 796, or from about 454 to about 682, or from about 568 to about 909.

Embodiment 367. The method according embodiment 365, wherein the amino acid residue in SEQ ID NO: 3 that is replaced is selected from F41, F43, K42, E61, and P64, and wherein n in the compounds of formula (XII) and (XIII) is an integer selected from 454, 455, 568, 569, 680, 681, 682, 794, 795, 796, 908, 909, and 910.

Embodiment 368. The method according to embodiment 346, wherein the amino acid residue in SEQ ID NO: 3 that is replaced is E61, and wherein n is an integer from about 450 to about 800, or from about 454 to about 796, or from about 454 to about 682, or from about 568 to about 909.

Embodiment 369. The method according to embodiment 346, wherein n in the compounds of formula (XII) and (XIII) is an integer selected from 454, 455, 568, 569, 680, 681, 682, 794, 795, 796, 908, 909, and 910.

Embodiment 370. The method according to embodiment 346, wherein n is from about 500 to about 1000.

Embodiment 371. The method according to embodiment 370, wherein n is from about 550 to about 800.

Embodiment 372. The method according to embodiment 369, wherein n is 681.

Embodiment 373. The method according to embodiment 346, wherein the amino acid residue in SEQ ID NO: 3 that is replaced is P64, and wherein n is an integer from about 450 to about 800, or from about 454 to about 796, or from about 454 to about 682, or from about 568 to about 909.

Embodiment 374. The method according to embodiment 346, wherein the amino acid residue in SEQ ID NO: 3 that is replaced is P64, and wherein n in the compounds of formula (XII) and (XIII) is an integer selected from 454, 455, 568, 569, 680, 681, 682, 794, 795, 796, 908, 909, and 910.

Embodiment 375. The method according to embodiment 346, wherein the amino acid residue in SEQ ID NO: 3 that is replaced is P64, and wherein n is an integer from about 500 to about 1000.

Embodiment 376. The method according to embodiment 375, wherein the amino acid residue in SEQ ID NO: 3 that is replaced is P64, and wherein n is from about an integer 550 to about 800.

Embodiment 377. The method according to embodiment 374, wherein the amino acid residue in SEQ ID NO: 3 that is replaced is P64, and wherein n is 681.

Embodiment 378. The method according to embodiment 346, wherein n is an integer such that the molecular weight of the PEG moiety is in the range from about 1,000 Daltons about 200,000 Daltons, or from about 2,000 Daltons to about 150,000 Daltons, or from about 3,000 Daltons to about 125,000 Daltons, or from about 4,000 Daltons to about 100,000 Daltons, or from about 5,000 Daltons to about 100,000 Daltons, or from about 6,000 Daltons to about 90,000 Daltons, or from about 7,000 Daltons to about 80,000 Daltons, or from about 8,000 Daltons to about 70,000 Daltons, or from about 5,000 Daltons to about 70,000 Daltons, or from about 5,000 Daltons to about 65,000 Daltons, or from about 5,000 Daltons to about 60,000 Daltons, or from about 5,000 Daltons to about 50,000 Daltons, or from about 6,000 Daltons to about 50,000 Daltons, or from about 7,000 Daltons to about 50,000 Daltons, or from about 7,000 Daltons to about 45,000 Daltons, or from about 7,000 Daltons to about 40,000 Daltons, or from about 8,000 Daltons to about 40,000 Daltons, or from about 8,500 Daltons to about 40,000 Daltons, or from about 8,500 Daltons to about 35,000 Daltons, or from about 9,000 Daltons to about 50,000 Daltons, or from about 9,000 Daltons to about 45,000 Daltons, or from about 9,000 Daltons to about 40,000 Daltons, or from about 9,000 Daltons to about 35,000 Daltons, or from about 9,000 Daltons to about 30,000 Daltons, or from about 9,500 Daltons to about 35,000 Daltons, or from about 9,500 Daltons to about 30,000 Daltons, or from about 10,000 Daltons to about 50,000 Daltons, or from about 10,000 Daltons to about 45,000 Daltons, or from about 10,000 Daltons to about 40,000 Daltons, or from about 10,000 Daltons to about 35,000 Daltons, or from about 10,000 Daltons to about 30,000 Daltons, or from about 15,000 Daltons to about 50,000 Daltons, or from about 15,000 Daltons to about 45,000 Daltons, or from about 15,000 Daltons to about 40,000 Daltons, or from about 15,000 Daltons to about 35,000 Daltons, or from about 15,000 Daltons to about 30,000 Daltons, or from about 20,000 Daltons to about 50,000 Daltons, or from about 20,000 Daltons to about 45,000 Daltons, or from about 20,000 Daltons to about 40,000 Daltons, or from about 20,000 Daltons to about 35,000 Daltons, or from about 20,000 Daltons to about 30,000 Daltons.

Embodiment 379. The method according to embodiment 346, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, about 50,000 Daltons, about 60,000 Daltons, about 70,000 Daltons, about 80,000 Daltons, about 90,000 Daltons, about 100,000 Daltons, about 125,000 Daltons, about 150,000 Daltons, about 175,000 Daltons or about 200,000 Daltons.

Embodiment 380. The method according to embodiment 346, wherein n is an integer such that the molecular weight of the PEG moiety is about 5,000 Daltons, about 7,500 Daltons, about 10,000 Daltons, about 15,000 Daltons, about 20,000 Daltons, about 25,000 Daltons, about 30,000 Daltons, about 35,000 Daltons, about 40,000 Daltons, about 45,000 Daltons, or about 50,000 Daltons.

Embodiment 381. The method according to any one of embodiments 1 to 380, wherein the one or more additional agents is one or more immune checkpoint inhibitors selected from the group consisting of PD-1 inhibitors, PD-L1 inhibitors, PD-L2 inhibitors, CTLA-4 inhibitors, OX40 agonists and 4-1BB agonists.

Embodiment 382. The method according to embodiment 381, wherein the one or more immune checkpoint inhibitors is selected from PD-1 inhibitors.

Embodiment 383. The method according to embodiment 382, wherein the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma).

Embodiment 384. The method according to embodiment 383, wherein the one or more PD-1 inhibitors is pembrolizumab.

Embodiment 385. The method according to embodiment 383, wherein the one or more PD-1 inhibitors is nivolumab.

Embodiment 386. The method according to embodiment 383, wherein the one or more PD-1 inhibitors is cemiplimab.

Embodiment 387. The method according to embodiment 383, wherein the one or more PD-1 inhibitors is lambrolizumab.

Embodiment 388. The method according to embodiment 383, wherein the one or more PD-1 inhibitors is AMP-224.

Embodiment 389. The method according to embodiment 383, wherein the one or more PD-1 inhibitors is sintilimab.

Embodiment 390. The method according to embodiment 383, wherein the one or more PD-1 inhibitors is toripalimab.

Embodiment 391. The method according to embodiment 383, wherein the one or more PD-1 inhibitors is camrelizumab.

Embodiment 392. The method according to embodiment 383, wherein the one or more PD-1 inhibitors is tislelizumab

Embodiment 393. The method according to embodiment 381, wherein the one or more immune checkpoint inhibitors is selected from PD-L1 inhibitors.

Embodiment 394. The method according to embodiment 393, wherein the one or more PD-L1 inhibitors is selected from atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics).

Embodiment 395. The method according to embodiment 394, wherein the one or more PD-L1 inhibitors is atezolizumab.

Embodiment 396. The method according to embodiment 394, wherein the one or more PD-L1 inhibitors is avelumab.

Embodiment 397. The method according to embodiment 394, wherein the one or more PD-L1 inhibitors is durvalumab.

Embodiment 398. The method according to embodiment 381, wherein the one or more immune checkpoint inhibitors is selected from CTLA-4 inhibitors.

Embodiment 399. The method according to embodiment 398, wherein the one or more CTLA-4 inhibitors is selected from tremelimumab, ipilimumab, and AGEN-1884 (Agenus).

Embodiment 400. The method according to embodiment 399, wherein the one or more CTLA-4 inhibitors is tremelimumab.

Embodiment 401. The method according to embodiment 399, wherein the one or more CTLA-4 inhibitors is ipilimumab.

Embodiment 402. The method according to any one of embodiments 1 to 401, wherein the cancer in the subject is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, cervical cancer, hepatocellular carcinoma (HCC), Merkel cell carcinoma (MCC), melanoma, small cell lung cancer (SCLC), esophageal, glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

Embodiment 403. The method according to embodiment 402, wherein the cancer in the subject is selected from renal cell carcinoma (RCC), non-small cell lung cancer (NSCLC), urothelial carcinoma, and melanoma.

Embodiment 404. The method according to any one of embodiments 1 to 403, wherein the IL-2 conjugate is administered to the subject in need thereof once per week, once every two weeks, once every three weeks, once every 4 weeks, once every 5 weeks, once every 6 weeks, once every 7 weeks, or once every 8 weeks.

Embodiment 405. The method according to embodiment 404, wherein the IL-2 conjugate is administered to the subject in need thereof once per week, once every two weeks, or once every three weeks.

Embodiment 406. The method according to embodiment 405, wherein the IL-2 conjugate is administered to the subject in need thereof once every two weeks.

Embodiment 407. The method according to embodiment 405, wherein the IL-2 conjugate is administered to the subject in need thereof once every three weeks.

Embodiment 408. The method according to any one of embodiments 1 to 407, wherein administration of the effective amount of the IL-2 conjugate to the subject does not cause vascular leak syndrome in the subject.

Embodiment 409. The method according to embodiment 408, wherein administration of the effective amount of the IL-2 conjugate to the subject does not cause Grade 2, Grade 3, or Grade 4 vascular leak syndrome in the subject.

Embodiment 410. The method according to embodiment 409, wherein administration of the effective amount of the IL-2 conjugate to the subject does not cause Grade 2 vascular leak syndrome in the subject.

Embodiment 411. The method according to embodiment 409, wherein administration of the effective amount of the IL-2 conjugate to the subject does not cause Grade 3 vascular leak syndrome in the subject.

Embodiment 412. The method according to embodiment 409, wherein administration of the effective amount of the IL-2 conjugate to the subject does not cause Grade 4 vascular leak syndrome in the subject.

Embodiment 413. The method according to any one of embodiments 1 to 412, wherein administration of the effective amount of the IL-2 conjugate to the subject does not cause loss of vascular tone in the subject.

Embodiment 414. The method according to any one of embodiments 1 to 413, wherein administration of the effective amount of the IL-2 conjugate to the subject does not cause extravasation of plasma proteins and fluid into the extravascular space in the subject.

Embodiment 415. The method according to any one of embodiments 1 to 414, wherein administration of the effective amount of the IL-2 conjugate to the subject does not cause hypotension and reduced organ perfusion in the subject.

Embodiment 416. The method according to any one of embodiments 1 to 415, wherein administration of the effective amount of the IL-2 conjugate to the subject does not cause impaired neutrophil function in the subject.

Embodiment 417. The method according to any one of embodiments 1 to 415, wherein administration of the effective amount of the IL-2 conjugate to the subject does not cause reduced chemotaxis in the subject.

Embodiment 418. The method according to any one of embodiments 1 to 417, wherein administration of the effective amount of the IL-2 conjugate to the subject is not associated with an increased risk of disseminated infection in the subject.

Embodiment 419. The method according to embodiment 418, wherein the disseminated infection is sepsis or bacterial endocarditis.

Embodiment 420. The method according to embodiment 419, wherein the disseminated infection is sepsis.

Embodiment 421. The method according to embodiment 419, wherein the disseminated infection is bacterial endocarditis.

Embodiment 422. A method of treating cancer in a subject according to any one of embodiments 1 to 421, wherein the subject is treated for any preexisting bacterial infections prior to administration of the IL-2 conjugate.

Embodiment 423. The method according to embodiment 422, wherein the subject is treated with an antibacterial agent selected from oxacillin, nafcillin, ciprofloxacin, and vancomycin prior to administration of the IL-2 conjugate.

Embodiment 424. The method according to any one of embodiments 1 to 423, wherein administration of the effective amount of the IL-2 conjugate to the subject does not exacerbate a pre-existing or initial presentation of an autoimmune disease or an inflammatory disorder in the subject.

Embodiment 425. The method according to embodiment 424, wherein the administration of the effective amount of the IL-2 conjugate to the subject does not exacerbate a pre-existing or initial presentation of an autoimmune disease in the subject.

Embodiment 426. The method according to embodiment 424, wherein the administration of the effective amount of the IL-2 conjugate to the subject does not exacerbate a pre-existing or initial presentation of an inflammatory disorder in the subject.

Embodiment 427. The method according to embodiment 424, wherein the autoimmune disease or inflammatory disorder in the subject is selected from Crohn's disease, scleroderma, thyroiditis, inflammatory arthritis, diabetes mellitus, oculo-bulbar myasthenia gravis, crescentic IgA glomerulonephritis, cholecystitis, cerebral vasculitis, Stevens-Johnson syndrome and bullous pemphigoid.

Embodiment 428. The method according to embodiment 427, wherein the autoimmune disease or inflammatory disorder in the subject is Crohn's disease.

Embodiment 429. The method according to embodiment 427, wherein the autoimmune disease or inflammatory disorder in the subject is scleroderma.

Embodiment 430. The method according to embodiment 427, wherein the autoimmune disease or inflammatory disorder in the subject is thyroiditis.

Embodiment 431. The method according to embodiment 427, wherein the autoimmune disease or inflammatory disorder in the subject is inflammatory arthritis.

Embodiment 432. The method according to embodiment 427, wherein the autoimmune disease or inflammatory disorder in the subject is diabetes mellitus.

Embodiment 433. The method according to embodiment 427, wherein the autoimmune disease or inflammatory disorder in the subject is oculo-bulbar myasthenia gravis.

Embodiment 434. The method according to embodiment 427, wherein the autoimmune disease or inflammatory disorder in the subject is crescentic IgA glomerulonephritis.

Embodiment 435. The method according to embodiment 427, wherein the autoimmune disease or inflammatory disorder in the subject is cholecystitis.

Embodiment 436. The method according to embodiment 427, wherein the autoimmune disease or inflammatory disorder in the subject is cerebral vasculitis.

Embodiment 437. The method according to embodiment 427, wherein the autoimmune disease or inflammatory disorder in the subject is Stevens-Johnson syndrome.

Embodiment 438. The method according to embodiment 427, wherein the autoimmune disease or inflammatory disorder in the subject is bullous pemphigoid.

Embodiment 439. The method according to any one of embodiments 1 to 438, wherein administration of the effective amount of the IL-2 conjugate to the subject does not cause changes in mental status, speech difficulties, cortical blindness, limb or gait ataxia, hallucinations, agitation, obtundation, or coma in the subject.

Embodiment 440. The method according to any one of embodiments 1 to 439, wherein administration of the effective amount of the IL-2 conjugate to the subject does not cause seizures in the subject.

Embodiment 441. The method according to anyone of embodiments 1 to 440, wherein administration of the effective amount of the IL-2 conjugate to the subject is not contraindicated in subjects having a known seizure disorder.

Embodiment 442. The method according to any one of embodiments 1 to 441, wherein administration of the effective amount of the IL-2 conjugate to the subject does not cause capillary leak syndrome in the subject.

Embodiment 443. The method according to embodiment 442, wherein administration of the effective amount of the IL-2 conjugate to the subject does not cause Grade 2, Grade 3, or Grade 4 capillary leak syndrome in the subject.

Embodiment 444. The method according to embodiment 443, wherein administration of the effective amount of the IL-2 conjugate to the subject does not cause Grade 2 capillary leak syndrome in the subject.

Embodiment 445. The method according to embodiment 443, wherein administration of the effective amount of the IL-2 conjugate to the subject does not cause Grade 3 capillary leak syndrome in the subject.

Embodiment 446. The method according to embodiment 443, wherein administration of the effective amount of the IL-2 conjugate to the subject does not cause Grade 4 capillary leak syndrome in the subject.

Embodiment 447. The method according to any one of embodiments 1 to 446, wherein administration of the effective amount of the IL-2 conjugate to the subject does not cause a drop in mean arterial blood pressure in the subject following administration of the IL-2 conjugate to the subject.

Embodiment 448. The method according to any one of embodiments 1 to 447, wherein administration of the effective amount of the IL-2 conjugate to the subject does not cause hypotension in the subject following administration of the IL-2 conjugate to the subject.

Embodiment 449. The method according to embodiment 448, wherein administration of the effective amount of the IL-2 conjugate to the subject does not cause the subject to experience a systolic blood pressure below 90 mm Hg or a 20 mm Hg drop from baseline systolic pressure following administration of the IL-2 conjugate to the subject.

Embodiment 450. The method according to any one of embodiments 1 to 449, wherein administration of the effective amount of the IL-2 conjugate to the subject does not cause edema in the subject following administration of the IL-2 conjugate to the subject.

Embodiment 451. The method according to any one of embodiments 1 to 450, wherein administration of the effective amount of the IL-2 conjugate to the subject does not cause impairment of kidney or liver function in the subject following administration of the IL-2 conjugate to the subject.

Embodiment 452. The method according to anyone of embodiments 1 to 451, wherein administration of the effective amount of the IL-2 conjugate to the subject does not cause eosinophilia in the subject following administration of the IL-2 conjugate to the subject.

Embodiment 453. The method according to embodiment 452, wherein administration of the effective amount of the IL-2 conjugate to the subject does not cause the eosinophil count in the peripheral blood of the subject to exceed 500 per μL following administration of the IL-2 conjugate to the subject.

Embodiment 454. The method according to embodiment 452, wherein administration of the effective amount of the IL-2 conjugate to the subject does not cause the eosinophil count in the peripheral blood of the subject to exceed 500 μL to 1500 per μL following administration of the IL-2 conjugate to the subject.

Embodiment 455. The method according to embodiment 452, wherein administration of the effective amount of the IL-2 conjugate to the subject does not cause the eosinophil count in the peripheral blood of the subject to exceed 1500 per μL to 5000 per L following administration of the IL-2 conjugate to the subject.

Embodiment 456. The method according to embodiment 452, wherein administration of the effective amount of the IL-2 conjugate to the subject does not cause the eosinophil count in the peripheral blood of the subject to exceed 5000 per μL following administration of the IL-2 conjugate to the subject.

Embodiment 457. The method according to any one of embodiments 1 to 456, wherein administration of the effective amount of the IL-2 conjugate to the subject is not contraindicated in subjects on an existing regimen of psychotropic drugs.

Embodiment 458. The method according to any one of embodiments 1 to 457, wherein administration of the effective amount of the IL-2 conjugate to the subject is not contraindicated in subjects on an existing regimen of nephrotoxic, myelotoxic, cardiotoxic, or hepatotoxic drugs.

Embodiment 459. The method according to embodiment 458, wherein administration of the effective amount of the IL-2 conjugate to the subject is not contraindicated in subjects on an existing regimen of aminoglycosides, cytotoxic chemotherapy, doxorubicin, methotrexate, or asparaginase.

Embodiment 460. The method according to any one of embodiments 1 to 459, wherein administration of the effective amount of the IL-2 conjugate to the subject is not contraindicated in subjects receiving combination regimens containing antineoplastic agents.

Embodiment 461. The method according to embodiment 460, wherein the antineoplastic agent is selected from dacarbazine, cis-platinum, tamoxifen and interferon-alfa.

Embodiment 462. The method according to anyone of embodiments 1 to 461, wherein administration of the effective amount of the IL-2 conjugate to the subject does not cause one or more Grade 4 adverse events in the subject following administration of the IL-2 conjugate to the subject.

Embodiment 463. The method of embodiment 462, wherein the one or more Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hyperuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

Embodiment 464. The method according to any one of embodiments 1 to 463, wherein administration of the effective amount of the IL-2 conjugate to a group of subjects does not cause one or more Grade 4 adverse events in greater than 1% of the subjects following administration of the IL-2 conjugate to the subjects.

Embodiment 465. The method according to embodiment 464, wherein the one or more Grade 4 adverse events are selected from hypothermia; shock; bradycardia; ventricular extrasystoles; myocardial ischemia; syncope; hemorrhage; atrial arrhythmia; phlebitis; AV block second degree; endocarditis; pericardial effusion; peripheral gangrene; thrombosis; coronary artery disorder; stomatitis; nausea and vomiting; liver function tests abnormal; gastrointestinal hemorrhage; hematemesis; bloody diarrhea; gastrointestinal disorder; intestinal perforation; pancreatitis; anemia; leukopenia; leukocytosis; hypocalcemia; alkaline phosphatase increase; blood urea nitrogen (BUN) increase; hypeuricemia; non-protein nitrogen (NPN) increase; respiratory acidosis; somnolence; agitation; neuropathy; paranoid reaction; convulsion; grand mal convulsion; delirium; asthma, lung edema; hyperventilation; hypoxia; hemoptysis; hypoventilation; pneumothorax; mydriasis; pupillary disorder; kidney function abnormal; kidney failure; and acute tubular necrosis.

Embodiment 466. The method according to any one of embodiments 1 to 465, wherein administration of the effective amount of the IL-2 conjugate to a group of subjects does not cause one or more adverse events in greater than 1% of the subjects following administration of the IL-2 conjugate to the subjects, wherein the one or more adverse events is selected from duodenal ulceration; bowel necrosis; myocarditis; supraventricular tachycardia; permanent or transient blindness secondary to optic neuritis; transient ischemic attacks; meningitis; cerebral edema; pericarditis; allergic interstitial nephritis; and tracheo-esophageal fistula.

Embodiment 467. The method according to any one of embodiments 1 to 466, wherein administration of the effective amount of the IL-2 conjugate to a group of subjects does not cause one or more adverse events in greater than 1% of the subjects following administration of the IL-2 conjugate to the subjects, wherein the one or more adverse events is selected from malignant hyperthermia; cardiac arrest; myocardial infarction; pulmonary emboli; stroke; intestinal perforation; liver or renal failure; severe depression leading to suicide; pulmonary edema; respiratory arrest; respiratory failure.

Embodiment 468. The method according to any one of embodiments 1 to 467, wherein administration of the IL-2 conjugate to the subject increases the number of peripheral CD8+ T and NK cells in the subject without increasing the number of peripheral CD4+ regulatory T cells in the subject.

Embodiment 469. The method according to any one of embodiments 1 to 468, wherein administration of the IL-2 conjugate to the subject increases the number of peripheral CD8+ T and NK cells in the subject without increasing the number of peripheral eosinophils in the subject.

Embodiment 470. The method according to any one of embodiments 1 to 469, wherein administration of the IL-2 conjugate to the subject increases the number of intratumoral CD8+ T and NK cells in the subject without increasing the number of intratumoral CD4+ regulatory T cells in the subject

Embodiment 471. The method according to any one of embodiments 1 to 470, wherein administration of the effective amount of the IL-2 conjugate to the subject does not require the availability of an intensive care facility or skilled specialists in cardiopulmonary or intensive care medicine.

Embodiment 472. The method according to embodiment 471, wherein administration of the effective amount of the IL-2 conjugate to the subject does not require the availability of an intensive care facility.

Embodiment 473. The method according to embodiment 471, wherein administration of the effective amount of the IL-2 conjugate to the subject does not require the availability of skilled specialists in cardiopulmonary or intensive care medicine.

Embodiment 474. The method according to any one of embodiments 1 to 473, wherein the cancer is in the form of a solid tumor.

Embodiment 475. The method according to any one of embodiments 1 to 473, wherein the cancer is in the form of a liquid tumor.

Embodiment 476. The method according to any one of embodiments 381 to 475, wherein the IL-2 conjugate is administered to the subject prior to the administration to the subject of the one or more immune checkpoint inhibitors.

Embodiment 477. The method according to any one of embodiments 381 to 475, wherein the one or more immune checkpoint inhibitors is administered to the subject prior to the administration to the subject of the IL-2 conjugate.

Embodiment 478. The method according to any one of embodiments 381 to 475, wherein the IL-2 conjugate and the one or more immune checkpoint inhibitors are simultaneously administered to the subject.

Embodiment 479. A method of treating cancer in a subject, comprising administering to a subject in need thereof a therapeutically effective amount of (a) a IL-2 conjugate, and (b) one or more additional agents, wherein the IL-2 conjugate comprising the amino acid sequence of SEQ ID NO: 1, SEQ ID NO: 3, or SEQ ID NO: 4 in which at least one amino acid residue in the IL-2 conjugate is replaced by a cysteine covalently bonded to a PEG group.

Embodiment 480. The method according to embodiment 479, wherein the PEG group has a molecular weight selected from 5 kDa, 10 kDa, 15 kDa, 20 kDa, 25 kDa, 30 kDa, 35 kDa, 40 kDa, 45 kDa, 50 kDa, and 60 kDa.

Embodiment 481. The method according to embodiment 479, wherein the PEG group has a molecular weight of 5 kDa.

Embodiment 482. The method according to embodiment 479, wherein the PEG group has a molecular weight of 10 kDa.

Embodiment 483. The method according to embodiment 479, wherein the PEG group has a molecular weight of 15 kDa.

Embodiment 484. The method according to embodiment 479, wherein the PEG group has a molecular weight of 20 kDa.

Embodiment 485. The method according to embodiment 479, wherein the PEG group has a molecular weight of 25 kDa.

Embodiment 486. The method according to embodiment 479, wherein the PEG group has a molecular weight of 30 kDa.

Embodiment 487. The method according to embodiment 479, wherein the PEG group has a molecular weight of 35 kDa.

Embodiment 488. The method according to embodiment 479, wherein the PEG group has a molecular weight of 40 kDa.

Embodiment 489. The method according to embodiment 479, wherein the PEG group has a molecular weight of 45 kDa.

Embodiment 490. The method according to embodiment 479, wherein the PEG group has a molecular weight of 50 kDa.

Embodiment 491. The method according to embodiment 479, wherein the PEG group has a molecular weight of 60 kDa.

Embodiment 492. The method according to anyone of embodiments 479 to 491, wherein the IL-2 conjugate comprises the amino acid sequence of SEQ ID NO: 3 and the at least one amino acid residue in the IL-2 conjugate that is replaced by a cysteine is selected from K34, T36, R37, T40, F41, K42, F43, Y44, E60, E61, E67, K63, P64, V68, L71, and Y106.

Embodiment 493. The method according to any one of embodiments 479 to 491, wherein the IL-2 conjugate comprises the amino acid sequence of SEQ ID NO: 3 and the at least one amino acid residue in the IL-2 conjugate that is replaced by a cysteine is selected from K34, T40, F41, K42, Y44, E60, E61, E67, K63, P64, V68, and L71.

Embodiment 494. The method according to anyone of embodiments 479 to 491, wherein the IL-2 conjugate comprises the amino acid sequence of SEQ ID NO: 4 and the at least one amino acid residue in the IL-2 conjugate that is replaced by a cysteine is selected from K35, T37, R38, T41, F42, K43, F44, Y45, E61, E62, E68, K64, P65, V69, L72, and Y107.

Embodiment 495. The method according to any one of embodiments 479 to 494, wherein the one or more additional agents is one or more immune checkpoint inhibitors is selected from the group consisting of PD-1 inhibitors, PD-L1 inhibitors, PD-L2 inhibitors, CTLA-4 inhibitors, OX40 agonists and 4-1BB agonists.

Embodiment 496. The method according to embodiment 495, wherein the one or more immune checkpoint inhibitors is selected from PD-1 inhibitors.

Embodiment 497. The method according to embodiment 496, wherein the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma).

Embodiment 498. The method according to embodiment 495, wherein the one or more immune checkpoint inhibitors is selected from PD-L1 inhibitors.

Embodiment 499. The method according to embodiment 498, wherein the one or more PD-L1 inhibitors is selected from atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics).

Embodiment 500. The method according to embodiment 495, wherein the one or more immune checkpoint inhibitors is selected from CTLA-4 inhibitors.

Embodiment 501. The method according to embodiment 500, wherein the one or more CTLA-4 inhibitors is selected from tremelimumab, ipilimumab, and AGEN-1884 (Agenus).

Embodiment 502. A method of treating cancer in a subject, comprising administering to a subject in need thereof a therapeutically effective amount of (a) a IL-2 conjugate, and (b) one or more additional agents, wherein the IL-2 conjugate comprises the amino acid sequence of SEQ ID NO: 3 in which at least one non-lysine residue is replaced by a lysine comprising a linker and a water-soluble polymer.

Embodiment 503. The method of embodiment 502, wherein the water-soluble polymer is a PEG group.

Embodiment 504. The method according to embodiment 502 or 503, wherein the one or more additional agents is one or more immune checkpoint inhibitors is selected from the group consisting of PD-1 inhibitors, PD-L1 inhibitors, PD-L2 inhibitors, CTLA-4 inhibitors, OX40 agonists and 4-1BB agonists.

Embodiment 505. The method according to embodiment 504, wherein the one or more immune checkpoint inhibitors is selected from PD-1 inhibitors.

Embodiment 506. The method according to embodiment 505, wherein the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma).

Embodiment 507. The method according to embodiment 506, wherein the one or more immune checkpoint inhibitors is selected from PD-L1 inhibitors.

Embodiment 508. The method according to embodiment 507, wherein the PD-L1 inhibitors is selected from atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics).

Embodiment 509. The method according to embodiment 508, wherein the one or more immune checkpoint inhibitors is selected from CTLA-4 inhibitors.

Embodiment 510. The method according to embodiment 509, wherein the one or more CTLA-4 inhibitors is selected from tremelimumab, ipilimumab, and AGEN-1884 (Agenus).

Embodiment 511. The method of anyone of embodiments 1 to 510, wherein the IL-2 conjugate comprises a PEG group covalently bonded via a non-releasable linkage.

Embodiment 512. The method of any one of embodiments 11 to 511, wherein the IL-2 conjugate comprises a non-releasable, covalently bonded PEG group.

Embodiment 513. The method according to any one of embodiments 1 to 512, wherein following administration of the IL-2 conjugate and the one or more additional agents, the subject experiences a response as measured by the Immune-related Response Evaluation Criteria in Solid Tumors (iRECIST).

Embodiment 514. The method according to embodiment 513, wherein the response is a complete response, a partial response or stable disease.

Embodiment 515. The method according to any one of embodiments 1 to 514, wherein the IL-2 conjugate is administered to the subject by intravenous, subcutaneous, intramuscular, intracerebral, intranasal, intra-arterial, intra-articular, intradermal, intravitreal, intraosseous infusion, intraperitoneal, or intrathecal administration.

Embodiment 516. The method according to embodiment 515, wherein the IL-2 conjugate is administered to a subject by intravenous, subcutaneous, or intramuscular administration.

Embodiment 517. The method according to embodiment 515, wherein the IL-2 conjugate is administered to a subject by intravenous administration.

Embodiment 518. The method according to embodiment 515, wherein the IL-2 conjugate is administered to a subject by subcutaneous administration.

Embodiment 519. The method according to embodiment 515, wherein the IL-2 conjugate is administered to a subject by intramuscular administration.

Embodiment 520. A method of treating cancer in a subject, comprising administering to a subject in need thereof a therapeutically effective amount of (a) a IL-2 conjugate, and (b) one or more additional agents, wherein the IL-2 conjugate is an IL-2 conjugate having SEQ ID NO: 3 wherein a non-lysine amino acid in the IL-2 conjugate is replaced by a lysine residue, and wherein the lysine residue comprises one or more water soluble polymers and a covalent linker.

Embodiment 521. The method of embodiment 520, wherein the lysine residue is located in the region K34-Y106 of SEQ ID NO: 3.

Embodiment 522. The method of embodiment 521, wherein the lysine residue is located at K34.

Embodiment 523. The method of embodiment 521, wherein the lysine residue is located at F41.

Embodiment 524. The method of embodiment 521, wherein the lysine residue is located at F43.

Embodiment 525. The method of embodiment 521, wherein the lysine residue is located at K42.

Embodiment 526. The method of embodiment 521, wherein the lysine residue is located at E61.

Embodiment 527. The method of embodiment 521, wherein the lysine residue is located at P64.

Embodiment 528. The method of embodiment 521, wherein the lysine residue is located at R37.

Embodiment 529. The method of embodiment 521, wherein the lysine residue is located at T40.

Embodiment 530. The method of embodiment 521, wherein the lysine residue is located at E67.

Embodiment 531. The method of embodiment 521, wherein the lysine residue is located at Y44.

Embodiment 532. The method of embodiment 521, wherein the lysine residue is located at V68.

Embodiment 533. The method of embodiment 521, wherein the lysine residue is located at L71.

Embodiment 534. A method of treating cancer in a subject, comprising administering to a subject in need thereof a therapeutically effective amount of (a) a IL-2 conjugate, and (b) one or more additional agents, wherein the IL-2 conjugate is an interleukin-2 (IL-2) variant wherein a non-lysine amino acid in the amino acid sequence of the IL-2 variant is replaced by an amino acid comprising: (a) a lysine; (b) a covalent linker; and (3) and one or more water-soluble polymers.

Embodiment 535. The method of any one of embodiments 520 to 534, wherein one or more water-soluble polymers comprises a PEG group.

Embodiment 536. The method of embodiment 535, wherein the PEG group is a branched or linear PEG group.

Embodiment 537. The method according to any one of embodiments 520 to 536, wherein the one or more additional agents is one or more immune checkpoint inhibitors is selected from the group consisting of PD-1 inhibitors, PD-L1 inhibitors, PD-L2 inhibitors, CTLA-4 inhibitors, OX40 agonists and 4-1BB agonists.

Embodiment 538. The method according to embodiment 537, wherein the one or more immune checkpoint inhibitors is selected from PD-1 inhibitors.

Embodiment 539. The method according to embodiment 538, wherein the one or more PD-1 inhibitors is selected from pembrolizumab, nivolumab, cemiplimab, lambrolizumab, AMP-224, sintilimab, toripalimab, camrelizumab, tislelizumab, dostarlimab (GSK), PDR001 (Novartis), MGA012 (Macrogenics/Incyte), GLS-010 (Arcus/Wuxi), AGEN2024 (Agenus), cetrelimab (Janssen), ABBV-181 (Abbvie), AMG-404 (Amgen). BI-754091 (Boehringer Ingelheim), CC-90006 (Celgene), JTX-4014 (Jounce), PF-06801591 (Pfizer), and genolimzumab (Apollomics/Genor BioPharma).

Embodiment 540. The method according to embodiment 539, wherein the one or more immune checkpoint inhibitors is selected from PD-L1 inhibitors.

Embodiment 541. The method according to embodiment 540, wherein the PD-L1 inhibitors is selected from atezolizumab, avelumab, durvalumab, ASC22 (Alphamab/Ascletis), CX-072 (Cytomx), CS1001 (Cstone), cosibelimab (Checkpoint Therapeutics), INCB86550 (Incyte), and TG-1501 (TG Therapeutics).

Embodiment 542. The method according to embodiment 541, wherein the one or more immune checkpoint inhibitors is selected from CTLA-4 inhibitors.

Embodiment 543. The method according to embodiment 542, wherein the CTLA-4 inhibitors is selected from tremelimumab, ipilimumab, and AGEN-1884 (Agenus).

Embodiment 544. The method according to any one of embodiments 381 to 543, wherein the method further comprises administering to the subject a therapeutically effective amount of one or more vascular endothelial cell growth factor (VEGF) pathway or mammalian target of rapamycin (mTOR) inhibitors.

Embodiment 545. The method according to embodiment 544, wherein the subject is administered one or more VEGF pathway inhibitors.

Embodiment 546. The method according to embodiment 545, wherein the one or more VEGF pathway inhibitors is selected from a group consisting of vascular endothelial cell growth factor receptor (VEGFR) tyrosine kinase inhibitors (TKIs) and anti-VEGF monoclonal antibodies.

Embodiment 547. The method according to embodiment 546, wherein the one or more VEGF pathway inhibitors is selected from one or more VEGFR TKIs.

Embodiment 548. The method according to embodiment 547, wherein the one or more VEGFR TKI is selected from a group consisting of cabozantinib, axitinib, pazopanib, sunitinib, or sorafenib.

Embodiment 549. The method according to embodiment 548, wherein the one or more VEGFR TKIs is cabozantinib

Embodiment 550. The method according to embodiment 548, wherein the one or more VEGFR TKIs is axitinib.

Embodiment 551. The method according to embodiment 548, wherein the one or more VEGFR TKIs is pazopanib.

Embodiment 552. The method according to embodiment 548, wherein the one or more VEGFR TKIs is sunitinib.

Embodiment 553. The method according to embodiment 548, wherein the one or more VEGFR TKIs is sorafenib.

Embodiment 554. The method according to embodiment 546, wherein the one or more VEGF pathway inhibitors is selected from one or more anti-VEGF monoclonal antibodies.

Embodiment 555. The method according to embodiment 554, wherein the one or more anti-VEGF monoclonal antibodies is bevacizumab.

Embodiment 556. The method according to embodiment 544, wherein the one or more mTOR inhibitors is selected from a group consisting of rapamycin, everolimus, temsirolimus, ridaforolimus, and deforolimus.

Embodiment 557. The method according to embodiment 556, wherein the one or more mTOR inhibitors is rapamycin.

Embodiment 558. The method according to embodiment 556, wherein the one or more mTOR inhibitors is everolimus.

Embodiment 559. The method according to embodiment 556, wherein the one or more mTOR inhibitors is temsirolimus.

Embodiment 560. The method according to embodiment 556, wherein the one or more mTOR inhibitors is ridaforolimus.

Embodiment 561. The method according to embodiment 556, wherein the one or more mTOR inhibitors is deforolimus.

Embodiment 562. The method according to anyone of embodiments 544 to 561, wherein the cancer in the subject is renal cell carcinoma (RCC).

Embodiment 563. The method according to embodiment 562, wherein the one or more VEGFR TKIs is axitinib or cabozantinib.

Embodiment 564. The method according to embodiment 562, wherein the one or more VEGFR TKIs cabozantinib.

Embodiment 565. The method according to any one of embodiments 381 to 543, wherein the one or more additional agents further comprises one or more chemotherapeutic agents.

Embodiment 566. The method according to embodiment 565, wherein the one or more chemotherapeutic agents comprises one or more platinum-based chemotherapeutic agents.

Embodiment 567. The method according to embodiment 565, wherein the one or more chemotherapeutic agents comprises carboplatin and pemetrexed

Embodiment 568. The method according to embodiment 565, wherein the one or more chemotherapeutic agents comprises carboplatin and nab-paclitaxel.

Embodiment 569. The method according to embodiment 565 wherein the one or more chemotherapeutic agents comprises carboplatin and docetaxel.

Embodiment 570. The method according to anyone of embodiments 565 to 569, wherein the cancer in the subject is non-small cell lung cancer (NSCLC).

Embodiment 571. The method according to anyone of embodiments 1 to 570, wherein the one or more additional agents is one or more chemotherapeutic agents.

Embodiment 572. The method according to embodiment 571, wherein the one or more chemotherapeutic agents comprises one or more platinum based chemotherapeutic agents.

Embodiment 573. The method according to any one of embodiments 1 to 572, wherein the subject has tested positive for human papillomavirus (HPV) prior to administration of the IL-2 conjugate and one or more additional agents.

Embodiment 574. The method according to embodiment 572, wherein the cancer in the subject is head and neck squamous cell cancer (HNSCC).

Embodiment 575. The method according to any one of embodiments 1 to 572, further comprising the subject testing positive for human papillomavirus (HPV+), followed by administration of the IL-2 conjugate and one or more additional agents.

Embodiment 576. A method of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VIII) or (IX), or a mixture of (VIII) and (IX):

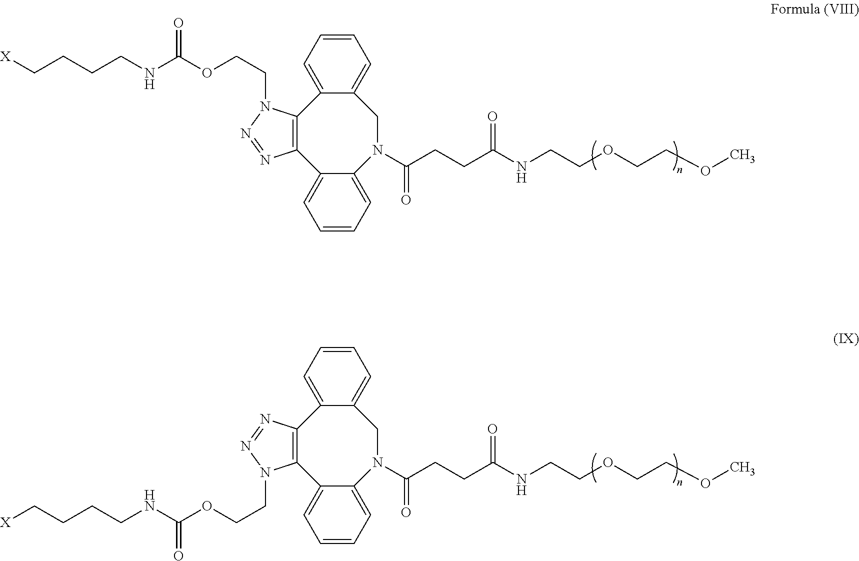

wherein:
n is an integer such that the molecular weight of the PEG group is from about 15,000 Daltons to about 60,000 Daltons; and
X has the structure:

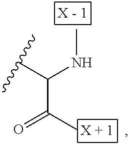 [SMILES: *C(NC)C(C)=O]

or a pharmaceutically acceptable salt, solvate, or hydrate thereof.

Embodiment 576.1. A method of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VIII) or (IX), or a mixture of (VIII) and (IX):

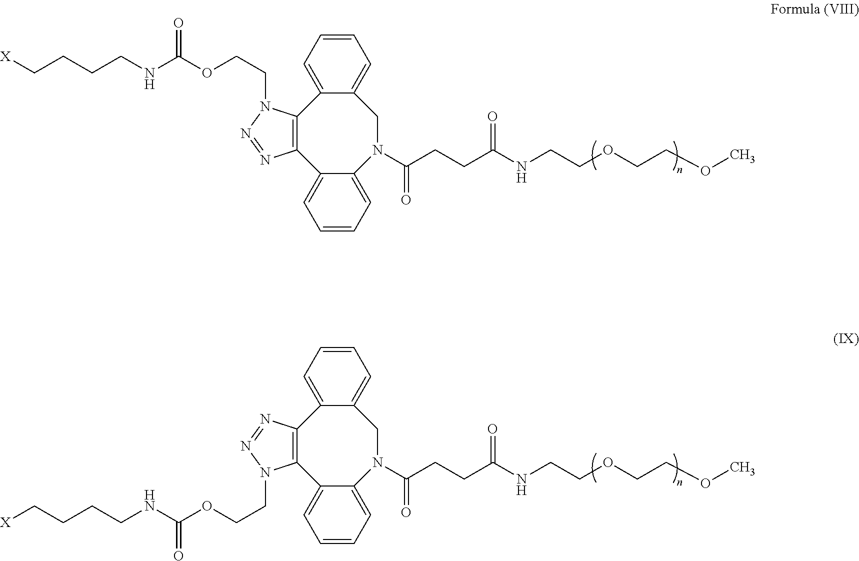

wherein:
n is an integer such that the molecular weight of the PEG group is from about 15,000 Daltons to about 60,000 Daltons; and
X has the structure:

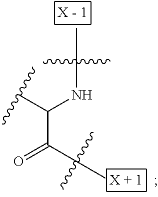 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 577. The method of embodiment 576 or 576.1, wherein the amino acid residue at E61 in the IL-2 conjugate is replaced by the structure of Formula (VIII) or (IX), or a mixture of (VIII) and (IX), and wherein n is an integer such that the molecular weight of the PEG group is from about 20,000 Daltons to about 40,000 Daltons.

Embodiment 578. The method of embodiment 577, wherein n is an integer such that the molecular weight of the PEG group is from about 30,000 Daltons.

Embodiment 579. The method of embodiment 577 or 578, wherein the one or more PD-1 inhibitors is pembrolizumab or nivolumab.

Embodiment 580. The method of embodiment 579, wherein the one or more PD-1 inhibitors is pembrolizumab.

Embodiment 581. The method of embodiment 579, wherein the one or more PD-1 inhibitors is nivolumab.

Embodiment 582. The method of embodiment 576 or 576.1, wherein the amino acid residue at P64 in the IL-2 conjugate is replaced by the structure of Formula (VIII) or (IX), or a mixture of (VIII) and (IX), and wherein n is an integer such that the molecular weight of the PEG group is from about 20,000 Daltons to about 40,000 Daltons.

Embodiment 583. The method of embodiment 582, wherein n is an integer such that the molecular weight of the PEG group is from about 30,000 Daltons.

Embodiment 584. The method of embodiment 582 or 583, wherein the one or more PD-1 inhibitors is pembrolizumab or nivolumab.

Embodiment 585. The method of embodiment 584, wherein the one or more PD-1 inhibitors is pembrolizumab.

Embodiment 586. The method of embodiment 584, wherein the one or more PD-1 inhibitors is nivolumab.

Embodiment 587. A method of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VI) or (VII), or a mixture of (VI) and (VII):

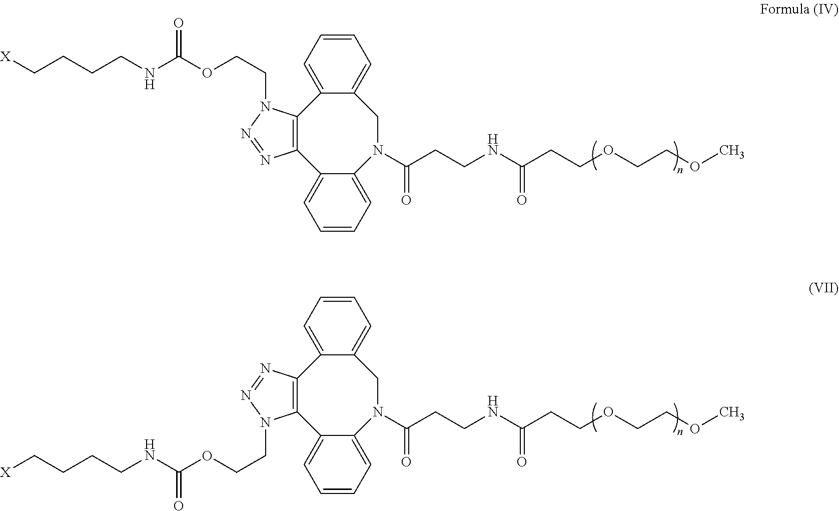

wherein:
n is an integer such that the molecular weight of the PEG group is from about 15,000 Daltons to about 60,000 Daltons; and
X has the structure:

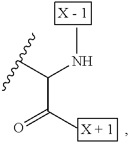 [SMILES: *C(NC)C(C)=O]

or a pharmaceutically acceptable salt, solvate, or hydrate thereof.

Embodiment 587.1. A method of treating cancer in a subject, comprising administering to a subject in need thereof a therapeutically effective amount of (a) a IL-2 conjugate, and (b) one or more PD-1 inhibitors, wherein the IL-2 conjugate comprises the amino acid sequence of SEQ ID NO: 3 in which the amino acid residue at E61 or P64 in the IL-2 conjugate is replaced by the structure of Formula (VI) or (VII), or a mixture of (VI) and (VII):

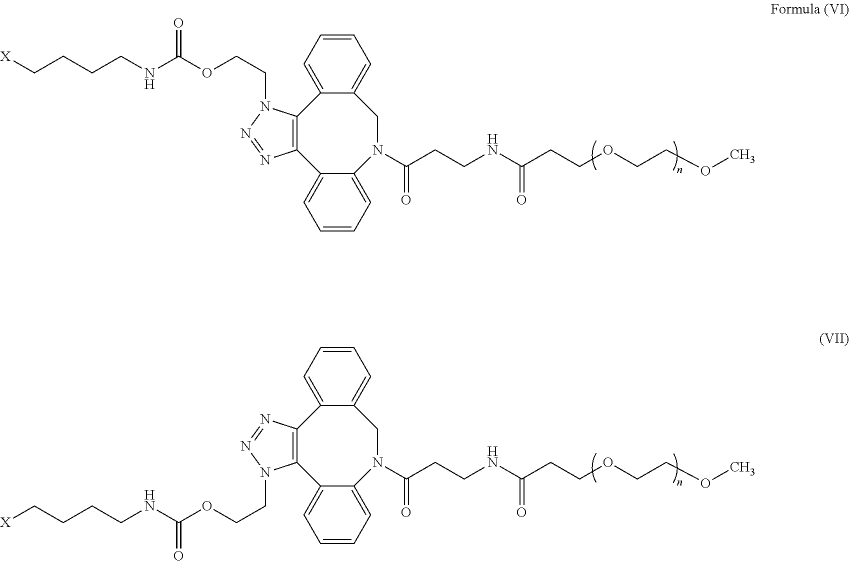

wherein:
n is an integer such that the molecular weight of the PEG group is from about 15,000 Daltons to about 60,000 Daltons; and
X has the structure:

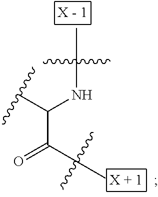 [SMILES: *NC(*)C(*)=O]

X−1 indicates the point of attachment to the preceding amino acid residue; and
X+1 indicates the point of attachment to the following amino acid residue;
or a pharmaceutically acceptable salt, solvate, or hydrate thereof.

Embodiment 588. The method of embodiment 587 or 587.1, wherein the amino acid residue at E61 in the IL-2 conjugate is replaced by the structure of Formula (VI) or (VII), or a mixture of (VI) and (VII), and wherein n is an integer such that the molecular weight of the PEG group is from about 20,000 Daltons to about 40,000 Daltons.

Embodiment 589. The method of embodiment 588, wherein n is an integer such that the molecular weight of the PEG group is from about 30,000 Daltons.

Embodiment 590. The method of embodiment 588 or 589, wherein the one or more PD-1 inhibitors is pembrolizumab or nivolumab.

Embodiment 591. The method of embodiment 590, wherein the one or more PD-1 inhibitors is pembrolizumab.

Embodiment 592. The method of embodiment 590, wherein the one or more PD-1 inhibitors is nivolumab.

Embodiment 593. The method of embodiment 587 or 587.1, wherein the amino acid residue at P64 in the IL-2 conjugate is replaced by the structure of Formula (VI) or (VII), or a mixture of (VI) and (VII), and wherein n is an integer such that the molecular weight of the PEG group is from about 20,000 Daltons to about 40,000 Daltons.

Embodiment 594. The method of embodiment 593, wherein n is an integer such that the molecular weight of the PEG group is from about 30,000 Daltons.

Embodiment 595. The method of embodiment 593 or 594, wherein the one or more PD-1 inhibitors is pembrolizumab or nivolumab.

Embodiment 596. The method of embodiment 595, wherein the one or more PD-1 inhibitors is pembrolizumab.

Embodiment 597. The method of embodiment 595, wherein the one or more PD-1 inhibitors is nivolumab.

Embodiment 598. The method of any one of embodiments 1-597, wherein the IL-2 conjugate is a pharmaceutically acceptable salt, solvate, or hydrate.

Embodiment 599. An IL-2 conjugate for use in the method of any one of embodiments 1-598.

Embodiment 600. Use of an IL-2 conjugate for the manufacture of a medicament for treating cancer according to the method of any one of embodiments 1-598.

### EXAMPLES

These examples are provided for illustrative purposes only and not to limit the scope of the claims provided herein.

Each of the compounds disclosed in Examples 2 to 8 utilized SEQ ID NO: 4 and the [AzK_PEG] moiety, wherein the position of the substituted amino acid in the IL-2 conjugate is in reference to the positions in SEQ ID NO: 4.

For example, the compound labelled “P65_5kD” in Tables 3A and 3B was prepared using methods similar to those disclosed in Example 2, wherein a protein was first prepared having SEQ ID NO: 4 in which the proline at position 65 was replaced by N6-((2-azidoethoxy)-carbonyl)-L-lysine (AzK) (SEQ ID NO: 10). The AzK-containing protein was then allowed to react under click chemistry conditions with DBCO comprising a methoxy, linear PEG group having an average molecular weight of 5 kDa to afford a product having SEQ ID NO: 20 comprising Formula (II), Formula (III), or a mixture of Formula (II) and (III), wherein W is a methoxy, linear PEG group having an average molecular weight of 5 kDa.

In another example, the compound labelled “P65_30kD” in Tables 3A and 3B, which was used in Example 4, Example 5, Example 6, and Example 11 (also called “IL-2_P65[AzK_PEG30kD]” in Example 11, and referred to in Example 11 and in the Figures as “Compound A”), was prepared by first preparing a protein having SEQ ID NO: 4 in which the proline at position 65 was replaced by N6-((2-azidoethoxy)-carbonyl)-L-lysine (AzK) (SEQ ID NO: 10). The AzK-containing protein was then allowed to react under click chemistry conditions with DBCO comprising a methoxy, linear PEG group having an average molecular weight of 30 kDa to afford a product having SEQ ID NO: 25 comprising Formula (II), Formula (III), or a mixture of Formula (II) and (III), wherein W is a methoxy, linear PEG group having an average molecular weight of 30 kDa. The compound can also be defined as comprising the amino acid sequence of SEQ ID NO: 4 in which the proline at position 65 (P65) is replaced by the structure of Formula (VI) or (VII), or a mixture of (VI) and (VII), and wherein n is an integer such that the PEG group has a molecular weight of about 30 kDa. The compound can also be defined as comprising the amino acid sequence of SEQ ID NO: 4 in which the proline at position 65 (P65) is replaced by the structure of Formula (X) or (XI), or a mixture of (X) and (XI), and wherein n is an integer such that the PEG group has a molecular weight of about 30 kDa.

In another example, the compound labelled “E62_5kD” in Tables 3A and 3B was prepared by first preparing a protein having SEQ ID NO: 4 in the glutamic acid at position 62 was replaced by N6-((2-azidoethoxy)-carbonyl)-L-lysine AzK (SEQ ID NO: 11). The AzK-containing protein was then allowed to react under click chemistry conditions with DBCO comprising a methoxy, linear PEG group having an average molecular weight of 5 kDa to afford a product having SEQ ID NO: 21 comprising Formula (II), Formula (III), or a mixture of Formula (II) and (III), wherein W is a methoxy, linear PEG group having an average molecular weight of 5 kDa.

In another example, the compound labelled “E62_30kD” in Tables 3A and 3B, and also used in Example 4, was prepared by first preparing a protein having SEQ ID NO: 4 in which the glutamic acid at position 62 was replaced by N6-((2-azidoethoxy)-carbonyl)-L-lysine (AzK) (SEQ ID NO: 11). The AzK-containing protein was then allowed to react under click chemistry conditions with DBCO comprising a methoxy, linear PEG group having an average molecular weight of 30 kDa to afford a product having SEQ ID NO: 26 comprising Formula (II), Formula (III), or a mixture of Formula (I) and (II), wherein W is a methoxy, linear PEG group having an average molecular weight of 30 kDa. The compound can also be defined as comprising the amino acid sequence of SEQ ID NO: 4 in which the glutamic acid at position 62 (E62) is replaced by the structure of Formula (VI) or (VII), or a mixture of (VI) and (VII), and wherein n is an integer such that the PEG group has a molecular weight of about 30 kDa. The compound can also be defined as comprising the amino acid sequence of SEQ ID NO: 4 in which the glutamic acid at position 62 (E62) is replaced by the structure of Formula (X) or (XI), or a mixture of (X) and (XI), and wherein n is an integer such that the PEG group has a molecular weight of about 30 kDa.

In another example, the compound labelled “K35_30kD,” and used in Example 8, was prepared by first preparing a protein having SEQ ID NO: 4 in which the lysine at position 35 was replaced by N6-((2-azidoethoxy)-carbonyl)-L-lysine (AzK) (SEQ ID NO: 14). The AzK-containing protein was then allowed to react under click chemistry conditions with DBCO comprising a methoxy, linear PEG group having an average molecular weight of 30 kDa to afford a product having SEQ ID NO: 29 comprising Formula (II), Formula (III), or a mixture of Formula (II) and (III), wherein W is a methoxy, linear PEG group having an average molecular weight of 30 kDa. The compound can also be defined as comprising the amino acid sequence of SEQ ID NO: 4 in which the lysine at position 35 (K35) is replaced by the structure of Formula (VI) or (VII), or a mixture of (VI) and (VII), and wherein n is an integer such that the PEG group has a molecular weight of about 30 kDa. The compound can also be defined as comprising the amino acid sequence of SEQ ID NO: 4 in which the lysine at position 35 (K35) is replaced by the structure of Formula (X) or (XI), or a mixture of (X) and (XI), and wherein n is an integer such that the PEG group has a molecular weight of about 30 kDa.

Examples 9 and 10 utilized compound “IL-2_P65_[AzK_L1_PEG30kD]-1” that comprises SEQ ID NO: 50, wherein the proline at position 64 is replaced by AzK_L1_PEG30kD, where AzK_L1_PEG30kD is defined as a structure of Formula (IV) or Formula (V), or a mixture of Formula (IV) and (V), and a 30 kDa, linear mPEG chain. Compound IL-2_P65[AzK_L1_PEG30kD]-1 is also defined as the compound comprising SEQ ID NO: 3 in which the proline residue at position 64 (P64) is replaced by the structure of Formula (VIII) or (IX), or a mixture of (VIII) and (IX), and wherein n is an integer such that the molecular weight of the PEG group is about 30 kDa. Compound IL-2_P65[AzK_L1_PEG30kD]-1 is also defined as the compound comprising SEQ ID NO: 3 in which the proline residue at position 64 (P64) is replaced by the structure of Formula (XII) or (XIII), or a mixture of (XII) and (XIII), and wherein n is an integer such that the molecular weight of the PEG group is about 30 kDa. Compound IL-2_P65[AzK_L1_PEG30kD]-1 is also referred to in Examples 12 et seq. and in the Figures as “Compound B”. The compound was prepared using methods similar to those disclosed in Example 2, wherein a protein was first prepared having SEQ ID NO: 3 in which the proline at position 64 was replaced by N6-((2-azidoethoxy)-carbonyl)-L-lysine AzK (SEQ ID NO: 35). The AzK-containing protein was then allowed to react under click chemistry conditions with DBCO comprising a methoxy, linear PEG group having an average molecular weight of 30 kDa.

Example 11 utilized compound “IL-2_P65[AzK_PEG30kD]” (also called “P65_30kD” herein) which was as described above.

### Example 1

Kinase and Cytokine Receptor Dimerization Assays

### Cell Handling

PathHunter cell lines were expanded from freezer stocks according to standard procedures. Cells were seeded in a total volume of 20 μL into white walled, 384-well microplates and incubated for the appropriate time prior to testing.

### Agonist Format

For agonist determination, cells were incubated with sample to induce response. Intermediate dilution of sample stocks was performed to generate 5× sample in assay buffer. About 5 μL of 5× sample was added to cells and incubated at 37° C. for 6 to 16 hours depending on the assay. Vehicle concentration was 1%.

### Signal Detection

Assay signal was generated through a single addition of 12.5 or 15 μL (50% v/v) of PathHunter Detection reagent cocktail for agonist and antagonist assays respectively, followed by a one hour incubation at room temperature. For some assays, activity was detected using a high sensitivity detection reagent (PathHunter Flash Kit) to improve assay performance. In these assays, an equal volume of detection reagent (25 or 30 μL) was added to the wells, followed by a one hour incubation at room temperature. Microplates were read following signal generation with a PerkinElmer Envision™ instrument for chemiluminescent signal detection.

### Data Analysis

Compound activity was analyzed using CBIS data analysis suite (ChemInnovation, CA). For agonist mode assays, percentage activity was calculated using the following Formula:

% Activity=100%×(mean RLU of test sample−mean RLU of vehicle control)/(mean MAX RLU control ligand−mean RLU of vehicle control).

For antagonist mode assays, percentage inhibition was calculated using the following Formula:

% Inhibition=100%×(1−(mean RLU of test sample−mean RLU of vehicle control)/(mean RLU of EC80 control−mean RLU of vehicle control)).

### Example 2

Cell-Based Screening for Identification of Pegylated IL-2 Compounds with No IL-2Rα Engagement

Exemplary IL-2 conjugates were subjected to functional analysis: K35, F42, K43, E62, and P65. The IL-2 conjugates were expressed as inclusion bodies in E. coli using methods disclosed herein wherein expression plasmids encoding the protein with the desired amino acid sequence were prepared that contained (a) an unnatural base pair comprising a first unnatural nucleotide and a second unnatural nucleotide to provide a codon at the desired position at which an unnatural amino acid N6-((2-azidoethoxy)-carbonyl)-L-lysine (AzK) was incorporated and a matching anticodon in a tRNA, (b) a plasmid encoding a tRNA derived from M. mazei Pyl and which comprises an unnatural nucleotide to provide a matching anticodon in place of its native sequence, (c) a plasmid encoding a M. barkeri derived pyrrolysyl-tRNA synthetase (Mb PylRS), and (d) N6-((2-azidoethoxy)-carbonyl)-L-lysine (AzK). The double-stranded oligonucleotide that encodes the amino acid sequence of the desired IL-2 variant contained a codon AXC at, for example, position 34, 37, 40, 41, 42, 43, 44, 61, 64, 68, or 71 of the sequence that encodes the protein having SEQ ID NO: 3, or at position 35, 38, 41, 42, 43, 45, 62, 65, 69, or 72 of the sequence that encodes the protein having SEQ ID NO: 4, wherein X is an unnatural nucleotide as disclosed herein. In some embodiments, the cell further comprises a plasmid, which may be the protein expression plasmid or another plasmid, that encodes an orthogonal tRNA gene from M. mazei that comprises an AXC-matching anticodon GYT in place of its native sequence, wherein Y is an unnatural nucleotide as disclosed herein and that may be the same or different as the unnatural nucleotide in the codon. X and Y were selected from unnatural nucleotides dTPT3, dNaM and dCNMO as disclosed herein. The expressed protein was purified and re-folded using standard procedures before site-specifically pegylating the AzK-containing IL-2 product using DBCO-mediated copper-free click chemistry to attach stable, covalent mPEG moieties to the AzK (Scheme 1).

Scheme 1. Exemplary synthesis of AzK_PEG interleukin variants (wherein n indicates the number of repeating PEG units). The reaction of the AzK moiety with the DBCO alkynyl moiety may afford one regioisomeric product or a mixture of regioisomeric products.

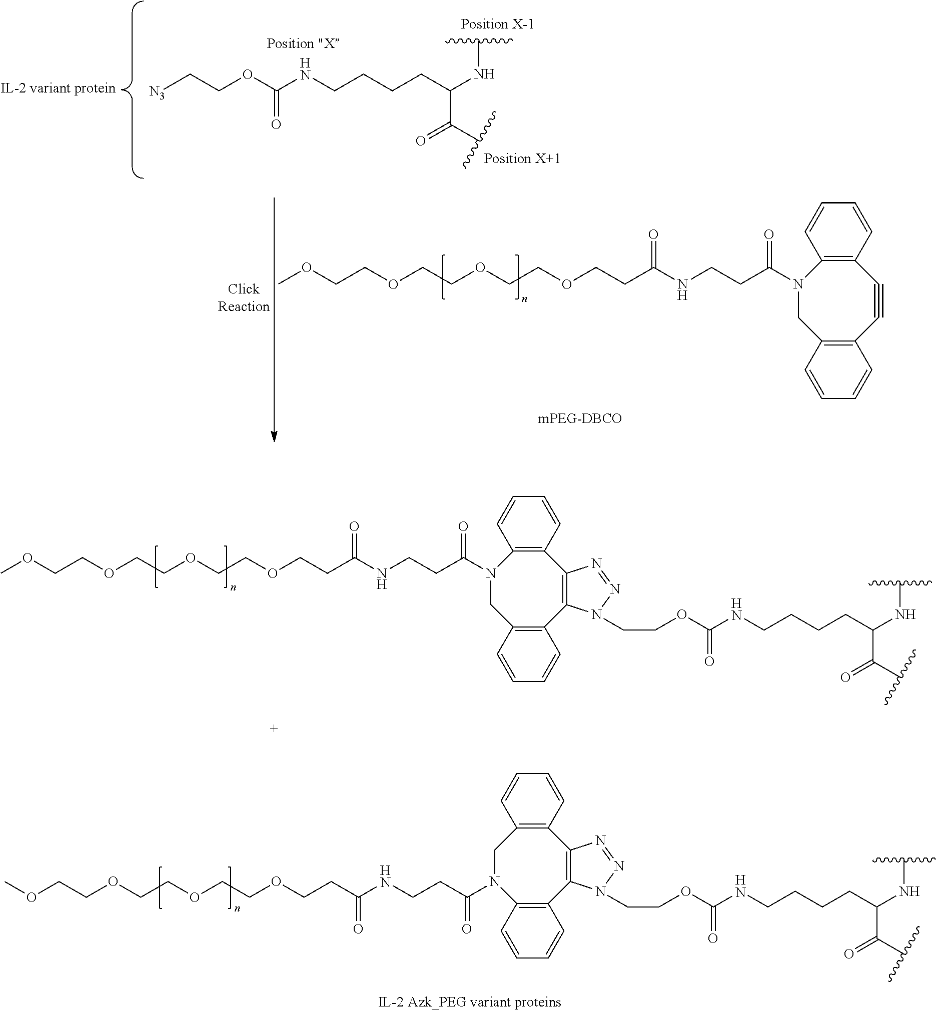

The IL-2 conjugates were screened for functional activity at Discoverx (Fremont, Calif.) using the PathHunter IL-2 Cytokine Receptor assay. This assay uses recombinant human U2OS cell line that expresses the IL-2 receptor β (IL-2R) and γ (IL-2Rγ) subunits, each fused to half of the split reporter enzyme β-galactosidase. A second cell line has been further engineered to express the IL-2Rα subunit. Parallel testing with these two cell lines allows assessment of variant activation of the IL-2 receptor ay as well as the basal βγ complex. IL-2 agonist activity on the IL-2βγ receptor complex stimulates receptor dimerization and reporter β-galactosidase reconstitution that results in a chemiluminescent signal. The assay was run in agonist mode to determine the EC₅₀ of each test article, and comparison of dose-response curve profiles between IL2Rα positive and negative cell types allows determination of the contribution of IL2Rα to the observed activity.

Table 2 shows the EC50 data for IL-2 receptor agonism in the cell-based screen for 10 kD (except where noted) PEGylated IL-2 conjugates.

TABLE 2

| | βγ | αβγ | |
|---|---|---|---|
| Site | EC50 (nM) | EC50 (nM) | βγ/αβγ ratio |
| Native | 1.68 | 0.074 | 23 |
| Ideal | 1.68 | 1.68 | 1 |
| K35 | 6.75 | 0.15 | 45 |
| F42 | 6.09 | 0.515 | 12 |
| K43 | 9.84 | 0.131 | 75 |
| E62 | 3 | 1.5 | 2 |
| P65* | 23.8 | 4.44 | 5 |
| R38 | 4.16 | 0.165 | 25 |
| T41 | 6.37 | 0.0489 | 130 |
| E68 | 7.70 | 0.0893 | 86 |
| Y45* | 9.06 | 0.110 | 83 |
| V69* | 9.99 | 0.083 | 121 |

*Indicates a 30 kD PEGylated II-2 conjugate.

Biochemical Interactions of PEGylated IL-2 with Human IL-2 Receptor Subunits

The kinetics of PEGylated IL-2 compound interactions with human IL-2 receptor subunits were measured using Surface Plasmon Resonance (SPR) at Biosensor Tools LLC (Salt Lake City, Utah). For these studies, human IgG1 Fc-fused IL-2 Rα (Sino Biological #10165-H02H) and β (Sino Biological #10696-H02H) extracellular domains were captured on the surface of a Biacore Protein A-coated CM4 sensor chip. These surfaces were probed in duplicate, with two-fold dilution series starting at 2 μM of either native IL-2 (wild-type IL-2; Thermo #PHC0021), P65_30kD, P65_5kD, E62_30kD, or E62_5kD using a Biacore 2000 SPR instrument. Test samples were injected for 60 seconds to allow measurement of association, followed by buffer only (wash) for 30 s to measure dissociation. Response units (RU, Y-axis) are plotted versus time (s, X-axis).

To evaluate the effect of IL-2 receptor a subunit on IL-2 binding to β, a was captured in about two-fold excess relative to β. To these surfaces, native IL-2 (wild-type IL-2), P65_30kD, P65_5kD, E62_30kD, or E62_5kD were applied in a three-fold dilution series beginning at 2.5 μM. The binding data were fit to a 1:1 interaction model that included a bulk shift, and the extracted kinetic parameters are summarized in Table 3A and Table 3B. As shown in Table 3A and Table 3B, small PEGs abrogate IL2R alpha engagement, but have less non-specific effect on IL2R beta engagement.

TABLE 3A
Kinetic parameters for IL-2 variant interactions with individual
IL-2 receptor subunit surfaces - IL-2 receptor α surface.

| | kₐ (M⁻¹s⁻¹) | | k_{d} (s⁻¹) | K_{D} (μM) |
|---|---|---|---|---|
| IL-2 native | 4.5 ± | 0.3 × 10⁷ | 0.410 ± 0.01 | 0.009 ± 0.002 |
| P65_30 kD | 114 ± | 36 × 10⁷ | 0.018 ± 0.008 | 158 ± 21 |
| P65_5 kD | 797 ± | 226 × 10⁷ | 0.033 ± 0.004 | 42 ± 7 |
| E62_30 kD | 333 ± | 88 × 10⁷ | 0.050 ± 0.01 | 162 ± 7 |
| E62_5 kD | 1010 ± | 41 × 10⁷ | 0.035 ± 0.002 | 34.4 ± 0.3 |

TABLE 3B
Kinetic parameters for IL-2 variant interactions with individual
IL-2 receptor subunit surfaces - IL-2 receptor β surface.

| | kₐ (M⁻¹s⁻¹) | | k_{d} (s⁻¹) | K_{D} (μM) |
|---|---|---|---|---|
| IL-2 native | 1.3 ± | 0.2 × 10⁶ | 0.185 ± 0.009 | 0.145 ± 0.005 |
| P65_30 kD | 1.8 ± | 0.2 × 10⁵ | 0.370 ± 0.01 | 2.09 ± 0.09 |
| P65_5 kD | 9.0 ± | 0.4 × 10⁵ | 0.270 ± 0.01 | 0.305 ± 0.002 |
| E62_30 kD | 1.8 ± | 0.4 × 10⁵ | 0 208 ± 0.006 | 1.14 ± 0.01 |
| E62_5 kD | 6.6 ± | 0.8 × 10⁵ | 0.281 ± 0.004 | 0.428 ± 0.00 |

Ex-Vivo Immune Response Profiling of IL-2 Compounds in Primary Human Leukocyte Reduction System (LRS)-Derived PBMC Samples

To determine how the differential receptor specificity of IL-2 P65_30 kD, K64_30kD, K43_30 kD, K35_30 kD, and F42_30 kD, effects activation of primary immune cell subpopulations, concentration-response profiling of lymphocyte activation in human LRS-derived peripheral blood mononuclear cell (PBMC) samples were performed using multi-color flow cytometry. These studies were performed at PrimityBio LLC (Fremont, Calif.). Fresh LRS-derived samples were treated with native IL-2, IL-2 P65_30 kD, K64_30kD, K43_30 kD, K35_30 kD, and F42_30 kD in 5-fold dilution series starting with a top concentration of 30 μg/mL. After a 45 min incubation, samples were fixed and stained with antibodies to detect the phosphorylated form of the transcription factor STAT5 (pSTAT5), a marker of upstream engagement and activation of IL-2 receptor signaling complexes, and a panel of surface markers to follow pSTAT5 formation in specific Tcell and natural killer (NK) cell subpopulations. Staining panel for flow cytometry study of LRS-derived PBMC samples include markers for Effector T cells (Teff: CD3+, CD4+, CD8+, CD127+), NK cells (CD3-, CD16+), and Regulatory T cells (Treg: CD3+, CD4+, CD8-, IL-2Rα+, CD127-1).

Flow cytometry data were analyzed for activation of different T and NK cell subsets in concentration-response mode, reading pSTAT5 accumulation after treatment with native IL-2. As a result of Treg-specific expression of IL-2 Rα, native IL-2 demonstrated an increased potency for pSTAT5 stimulation in Tregs compared with CD8 Teff and NK cells. Compared to the native compound, the PEGylated variants demonstrated modestly-reduced potencies on CD8 T cells and NK cell populations, but showed differential reduction in potency in IL-2 Rα expressing Treg cells with respect to native IL-2.

Table 4 provides the dose response EC50 for pSTAT5 signaling (EC50) in human LRS samples or CTLL-2 proliferation treated with indicated IL-2 variant.

TABLE 4
Dose response EC50 for pSTAT5 signaling (EC50) in human LRS
samples or CTLL-2 proliferation treated with indicated IL-2 variant.

| | NK | Treg | CD8+ | CD8/Treg | |
|---|---|---|---|---|---|
| treatment | Cells | Cells | T Cells | ratio | CTLL-2 |
| Native IL-2 | 5150.5 | 62.5 | 25703.5 | 411.3 | 846 |
| E62_30 kD | 12834 | 37213 | 66644 | 1.8 | 398,012 |
| E62_5 kD | 5327.5 | 18146 | 41552.5 | 2.3 | 275,590 |
| E62K | 10305 | 11086 | 64037 | 5.8 | 58,213 |
| P65_30 kD | 15741 | 40740.5 | 113638 | 2.8 | 677,198 |
| P65_5 kD | 1920 | 6324.5 | 13769.5 | 2.2 | 194,924 |
| K35_30 kD | 14021 | 358 | 63023 | 176.0 | N.D. |
| F42_30 kD | 16397 | 36856 | 107944 | 2.9 | 123,936 |
| K43_30 kD | 9004 | 4797 | 50504 | 10.5 | N.D. |

N.D. = not determined.

The EC50 values (pg/mL) was calculated from dose response curves generated from MFI plots.

### Example 3

PEG and Residue Substitution Contribute to No-Alpha Pharmacology

To determine whether the PEG and residue substitution impacted the no-alpha pharmacology of IL-2 E62, concentration-response profiling of lymphocyte activation in human LRS-derived peripheral blood mononuclear cell (PBMC) samples were performed using multi-color flow cytometry. These studies were performed at PrimityBio LLC (Fremont, Calif.). Fresh LRS-derived samples were treated with native IL-2, E62K, or E62_30kD in 5-fold dilution series starting with a top concentration of 30 μg/mL. After a 45 min incubation, samples were fixed and stained with antibodies to detect the phosphorylated form of the transcription factor STAT5 (pSTAT5), a marker of upstream engagement and activation of IL-2 receptor signaling complexes, and a panel of surface markers to follow pSTAT5 formation in specific Tcell and natural killer (NK) cell subpopulations. Staining panel for flow cytometry study of LRS-derived PBMC samples include markers CD4, CD4+ memory central, CD4+ memory effect, CD4+ memory T cells, CD4+ Naive T cells, CD4+ T cells, CD8, CD8+ memory central, CD8+ memory effect, CD8+ memory T cells, CD8+ Naive T cells, CD8+ T cells, NK cells, and T regulatory cells.

### Example 4

PK/PD Studies in Naïve (E3826-U1704) and B16-F10 Tumor-Bearing (E3826-U1803) C57BL/6 Mice

The study designs are summarized in Table 5 and Table 6, wherein the dose was calculated by reference to the mass of the protein component not including the mass of the PEG moiety. Terminal blood samples were collected via cardiac puncture at the points indicated. Study E3826-U1704, included 13 time points (0.13, 0.25, 0.5, 1, 2, 4, 8, 12, 24, 48, 72, 96 and 120 h) sacrificing 3 mice per each time point and study E3826-U1803 included 9 time points (2, 8, 12, 24, 48, 72, 120, 168, and 240 h) sacrificing 4-7 mice per each time point. Plasma and blood cells (in both studies) and tumors in study E3826-U1803 were collected for PK and PD analyses.

Bioanalysis of plasma samples was performed using a qualified human IL-2 ELISA assay (Abcam, Cambridge, UK). Concentrations of Aldesleukin, E62_30kD and P65_30kD and the internal standard in samples derived from plasma were determined using an ELISA assay. PK data analysis was performed at NW Solutions (Seattle, Wash.). The PK data were imported into Phoenix WinNonlin v6.4 (Certara/Pharsight, Princeton, N.J.) for analysis. The group mean plasma concentration versus time data were analyzed with noncompartmental methods using an IV bolus administration model.

TABLE 5
PK/PD Study No. E3826-U1704 - Control and Test Treatment groups
in Naïve C57/BL6 Mice.

| Treatment | Dose*(mg/Kg) | Route, Schedule | Time Points | N |
|---|---|---|---|---|
| Control | 0 | IV, single dose | 13 | 3 |
| Aldesleukin | 0.3 | IV, single dose | 13 | 3 |
| P65_30 kD | 0.3 | IV, single dose | 13 | 3 |
| E62_30 kD | 0.3 | IV, single dose | 13 | 3 |

*Dose refers to P65_30 kD IL-2 polypeptide amount, wherein the dose was calculated by reference to the mass of the protein component not including the mass of the PEG moiety

TABLE 6
PK/PD Study No. E3826-U1803 - Control and Test Treatment
groups - B16F-10 Melanoma Tumor-Bearing Mice (wherein the
dose was calculated by reference to the mass of the protein
component not including the mass of the PEG moiety).

| | Dose | | Time | |
|---|---|---|---|---|
| Treatment | (mg/kg) | Route, Schedule | Point | N |
| None (pre-dose) | 0 mg/kg | None | 1 | 6 |
| Vehicle Control | 0 mg/kg | IV, single dose | 9 | 3 |
| P65_30 kD | 1 mg/kg | IV, single dose | 9 | 4 |
| P65_30 kD | 3 mg/kg | IV, single dose | 9 | 4 |

In study E3826-U1704, both P65_30 kD and E62_30 kD exhibit a superior PK profile relative to aldesleukin as summarized in Table 7. Following a single IV bolus dose of aldesleukin, the Tmax was observed at 0.03 h post-dose (the first measured time point after dosing) and mean plasma concentrations were measurable out to 4 h post-dose. After single IV bolus dosing of P65_30 kD and E62_30 kD, the Tmax was observed at 0.03 h post-dose and mean plasma concentrations were measurable out to 120 h post-dose (the last measured time point). In a separate study, after IV dosing of E62_5kD, the Tₘₐₓ was observed at 0.133 hr post-dose and mean plasma concentrations were measurable out to 12 hr post-dose.

Exposure based on Cₘₐₓ and AUC₀₋₄, was as follows: P65_30kD>E62_30kD>>E62_5kD>aldesleukin. E62_5kD with a smaller PEG had a PK profile closer to rIL-2 (Table 7). P65_30 kD exposure was 5.5 and 200 times higher than aldesleukin based on Cmax and AUC0-t, respectively. In addition, P65_30 kD demonstrated 23-fold extended t1/2 (13.3 h vs. 0.57 h) and about 198-fold reduced CL (6.58 vs 1300 mL/h/Kg) compared to the aldesleukin. For both P65_30 kD and E62_30 kD, the distribution volume (82.4 and 92.3 mL/Kg respectively) was about 4.2 to 4.7-fold reduced relative to aldesleukin, and similar to the blood volume in a mouse (85 mL/Kg; [Boersen 2013]). This suggests that P65_30kD and E62_30kD are mostly distributed within systemic circulation.

TABLE 7
P65_30 kD PK Parameters in C57BL/6 Female Mice.

| Parameter | Units | P65_30 kD | E62_30 kD | E62_5 kD | Aldesleukin |
|---|---|---|---|---|---|
| Tₘₐₓ | h | 0.030 | 0.030 | 0.133 | 0.030 |
| Cₘₐₓ | ng/mL | 4,870 | 4,230 | 936 | 884 |
| AUC₀₋ₜ | h*ng/mL | 45,600 | 37,100 | 798 | 229 |
| R² | | 0.992 | 0.986 | 0.851 | 0.900 |
| AUC_{INF} | h*ng/mL | 45,600 | 37,100 | 807 | 230 |
| t½ | h | 13.300 | 14.500 | 2.56 | 0.573 |
| CL | mL/h/Kg | 6.580 | 8.07 | 372 | 1300 |
| Vₛₛ | mL/Kg | 82.4 | 92.3 | 404 | 390 |

Note:
R² is the goodness-of-fit parameter for the terminal phase of each concentration versus time profile.
All parameters shown to 3 significant FIGURES.

### Example 5

Pharmacodynamics Observations in Peripheral Blood Compartment

STAT5 phosphorylation and induction of cell proliferation (the early molecular marker Ki-67 and cell counts) was used as pharmacodynamics readouts to assess the pharmacological profile of P65_30kD relative to its pharmacokinetics. The pSTAT5 PD marker showed good correlation with PK for both P65_30kD and aldesleukin in CD8+ effector T cells (Table 7). Persistent elevation of pSTAT5 was observed in both NK and CD8+ T cells up to 72 h, and up to 24 h in Tregs. pSTAT5 induction returned to baseline after only 2 h in mice dosed with aldesleukin. STAT5 phosphorylation translated into proliferative responses (72-120 hrs) of CD8+ effector T cells and NK cells but not with T regs, Phenotypic analysis of CD8+ effector T cells revealed substantial expansion of CD44+ memory cells within this population.

Pharmacodynamics Observations in Tumor Compartment in B16-F10 Tumor-Bearing (E3826-U1803) C57BL/6 Mice

Table 8 shows the plasma and tumor drug concentration following a single dose of P65_30kD at 3 mg/kg in B16-F10 tumor-bearing mice, wherein the dose was calculated by reference to the mass of the protein component not including the mass of the PEG moiety. The tumor half-life was twice the plasma half-life (24.4 vs 12.6), indicating that the P65_30kD penetrates the tumor and is retained in the tumor. The tail end of the curves cross showing the plasma eliminates faster than the tumor (data not shown). The tumor:plasma AUC ratio was 9.7% and 8.4% for the 1 and 3 mg/kg doses respectively.

TABLE 8
P65_30 kD Plasma and Tumor PK Parameters B16-T10 tumor-bearing
C57BL/6 Female Mice.

| | P65_30 kD (3 mg/kg) | |
|---|---|---|
| Parameter | Plasma | Tumor |
| Tₘₐₓ (h) | *2.00 | 8 |
| Cₘₐₓ (ng/mL) | 40000 | 1550 |
| t½ (h) | 12.60 | 24.4 |
| AUC₀₋ₜ (h*ng/mL) | 656,000 | 55200 |
| R² | 0.974 | 0.988 |
| AUC_{INF} (h*ng/mL) | 656,000 | 55200 |

MTD Study in Balb/c Mice E3826-U1802

A dose ranging study of P65_30kD was conducted in naïve female Balb/c mice at Crown Biosciences, Inc. (San Diego, Calif.). The study design is shown in Table 9, wherein the dose was calculated by reference to the mass of the protein component not including the mass of the PEG moiety. Blood samples were drawn via sub mandibular vein at 8 time points (0.25, 1, 4, 12, 24, 34, 48 & 72 h). Both plasma and blood cells were collected for PK and PD analyses.

All plasma samples were analyzed for human IL-2 as well as mouse IL-2, TNF-α, IFNγ, IL-5, and IL-6 cytokines, employing commercially-available ELISA kits.

TABLE 9
PK/PD and MTD Study No. E3826-U1802 - Control and Test Treatment
groups in Naïve Balb/C Mice.

| Treatment | Dose (mg/kg) | Route, Schedule | Time Point | N |
|---|---|---|---|---|
| Naive | 0 mg/kg | | 0 | 3 |
| Vehicle Control | 0 mg/kg | IV, BID × 3 | 3 | 3 |
| Aldesleukin | 0.01 mg/kg | IV, BID × 3 | 3 | 3 |
| Aldesleukin | 0.03 mg/kg | IV, BID × 3 | 3 | 3 |
| Aldesleukin | 0.1 mg/kg | IV, BID × 3 | 3 | 3 |
| Aldesleukin | 1.0 mg/kg | IV, BID × 3 | 3 | 3 |
| Aldesleukin | 3.0 mg/kg | IV, BID × 3 | 3 | 3 |
| Aldesleukin | 5.0 mg/kg | IV, BID × 3 | 3 | 3 |
| P65_30 kD | 0.01 mg/kg | IV, single dose | 3 | 3 |
| P65_30 kD | 0.03 mg/kg | IV, single dose | 3 | 3 |
| P65_30 kD | 0.1 mg/kg | IV, single dose | 3 | 3 |
| P65_30 kD | 1.0 mg/kg | IV, single dose | 3 | 3 |
| P65_30 kD | 3.0 mg/kg | IV, single dose | 3 | 3 |
| P65_30 kD | 5.0 mg/kg | IV, single dose | 3 | 3 |
| #P65_30 kD | 0.3 mg/kg | IV, single dose | 8 | 3 |

* All time point except the 72 hr time point blood collection was via the sub mandibular vein. The 72 hr time point was terminal blood collection.
#Only the 0.3 mg/kg dose of P65_30 kD was used for the PK/PD evaluation.

Toxicology Observations in the MTD Study Using Balb/c Mice

A major of toxicity associated with high-dose aldesleukin is vascular leak syndrome and associated Cytokine Release Syndrome (CRS). To evaluate the potential for this effect in mice, a single dose IV administration of P65_30kD at doses ranging from 0.01-5.0 mg/kg dose was performed (Table 9), wherein the dose was calculated by reference to the mass of the protein component not including the mass of the PEG moiety. The analysis performed was hematology, histopathology, organ weight, and cytokine analyses. Abnormalities were not observed with hematology, histopathology or body weights relative to the vehicle control mice with both P65_30kD or aldesleukin. With respect to the cytokine analysis, it was observed that aldesleukin elevated plasma IL-5 levels starting at 1 mg/kg to 5 mg/kg. With P65_30kD, a moderate increase in IL-5 (but less compared to aldesleukin) was seen only at 5 mg/kg dose. A transient elevation in the systemic levels of IFNγ was observed with both aldesleukin and P65_30kD.

### Example 6

PK/PD in Cynomolgus Monkeys-Study No.: 20157276

The pharmacokinetic and pharmacodynamics profile of P65_30kD was evaluated in non-naïve cynomolgus monkeys following administration of a single intravenous dose at 0.3 mg/kg, wherein the dose was calculated by reference to the mass of the protein component not including the mass of the PEG moiety. The study was conducted at Charles River Laboratories, Inc. (Reno, Nev.) and PK data analysis was performed at NW Solutions (Seattle, Wash.). Blood samples were collected pre-dose and at 15 time points (0.5, 1, 2, 4, 8, 12, 24, 36, 48, 72, 120, 144, 168, 192 and 240 h post-dose). Both plasma and blood cells were collected for PK and PD analyses. Selected time points were used for PK, PD, cell population and hematology analysis.

All plasma samples were analyzed for human IL-2 (PK readout) employing commercially-available ELISA kits.

Table 10 shows P65_30kD PK Parameters in Cynomolgus monkey.

TABLE 10

| | | | 0.3 mg/kg | | |
|---|---|---|---|---|---|
| | | | Animal 2699 | Animal 2705 | Mean |
| ROA | Parameter | Units | Estimate | | |
| IV | Tₘₐₓ | hr | 0.500 | 0.500 | 0.500 |
| | Cₘₐₓ | ng/mL | 11000 | 11400 | 11200 |
| | AUC₀₋ₜ | hr*ng/mL | 121000 | 120000 | 121000 |
| | t_{1/2} | hr | 13.4 | 13.9 | 13.6 |
| | CL | mL/hr/kg | 2.47 | 2.49 | 2.48 |
| | Vₛₛ | mL/kg | 29.0 | 32.1 | 30.5 |

After single IV bolus dosing Tmax was observed at 0.5 h post-dose (the first measured time point after dosing) and mean plasma concentrations were measurable out to 168 h post-dose (the last measure). The t_{1/2} and AUC for P65_30kD were 13.6 h and 121000 hr*ng/mL respectively.

Hematology Parameters—Cynomolgus Monkeys-Study No.: 20157276

For hematologic parameters the evaluation time points correspond to pre-dose at day-1 and 1, 3, 6, 8, 10, 12, 14, 17, 19, 21 post-dose.

### Example 7

Ex-Vivo Immune Response Profiling of Exemplary IL-2 Compounds in Primary Human Leukocyte Reduction System (LRS)-Derived PBMC Samples

To determine how the differential receptor specificity of exemplary IL-2 compounds affects activation of primary immune cell subpopulations, concentration-response profiling of lymphocyte activation in human LRS-derived peripheral blood mononuclear cell (PBMC) samples were performed using multi-color flow cytometry. Conjugates of Table 12 were synthesized by modification of SEQ NO. 1. These studies were performed at PrimityBio LLC (Fremont, Calif.). Primary lymphocytes derived from human LRS samples were treated with dilutions series of exemplary IL-2 compounds and quantified based on pSTAT5 signaling in each lymphocyte cell type using the panel shown in Table 11.

TABLE 11
Key indicating cell populations.

| Marker | Cell population |
|---|---|
| CD3 | T cells |
| CD4 | Th cells |
| CD8 | T effector cells |
| CD45RA | Naïve T cells |
| CD56 | NK cells |
| CD14/19 | Monocyte/B cells |
| CD25 | Tregs or experienced T cell |
| CD127 | Not Treg |
| CD62L | Memory T vs |
| | effector memory T cell |
| pSTAT5 | Activation marker |
| (Y694) | |

Flow cytometry data were analyzed for activation of different T and NK cell subsets in concentration-response mode, reading pSTAT5 accumulation after treatment with an exemplary IL-2 variant K9_3kD.

Table 12. Dose response EC50 for pSTAT5 signaling (EC50) in human LRS samples or CTLL-2 proliferation treated with indicated IL-2 variant.

TABLE 12
Dose response EC50 for pSTAT5 signaling (EC50) in human LRS samples or CTLL-2
proliferation treated with indicated IL-2 variant.

| | | | | | Fold increase | |
|---|---|---|---|---|---|---|
| | | | | CD8+/Treg | in Treg EC50 | CTLL-2 |
| Compound | NK cells | CD8+ Tcells | Treg cells | ratio | vs native IL-2 | proliferation |
| native IL-2 | 4586 | 31024 | 75 | 414 | 1 | 455.8 |
| K9_30kD | 169578 | 1100679 | 2217 | 496 | 30 | 504 |
| H16_30kD | 2545257 | 12070108 | 34976 | 345 | 466 | 80755 |
| L19_30kD | 6756768 | 22436430 | 93205 | 241 | 1243 | 3510 |
| D20_30kD | 2643930 | 9505217 | 1129455 | 8 | 15059 | 689939 |
| M23_30kD | 143620 | 539824 | 1030 | 524 | 14 | 1102 |
| N26_30kD | 258531 | 1188859 | 2459 | 483 | 33 | 2594 |
| N88_30kD | 3298113 | 11111537 | 323201 | 34 | 4309 | 66606 |
| E100_30kD | 35088 | 195823 | 483 | 405 | 6 | 1676 |
| N119_30kD | 34010 | 143380 | 535 | 268 | 11 | 1215 |
| T123_30kD | 33396 | 152928 | 269 | 569 | 6 | 255 |
| Q126_30kD | 3676807 | 19722480 | 29454 | 670 | 393 | 3584 |
| S127_30kD | 20210 | 92190 | 150 | 615 | 3 | 123 |
| T131_30kD | 24207 | 132922 | 258 | 515 | 3 | 641 |
| N88R/D109_30kD | 2780819 | 12503386 | 175805 | 71 | 3663 | 59577 |
| V91K | 20537 | 102255 | 142 | 720 | 3 | 99.5 |
| N88R | 2312847 | 15025734 | 11082 | 1356 | 148 | 363 |

*Treg potency change compared to native IL-2 (wild-type IL-2) run in each individual experiment.

The EC50 values (pg/ml) were calculated from dose response curves generated from the MFI plots.

### Example 8

PK Study in C57BL/6 Mice

Experimental details are summarized in Table 13, wherein the dose was calculated by reference to the mass of the protein component not including the mass of the PEG moiety.

TABLE 13

| | Number | | | |
|---|---|---|---|---|
| | of | Test/Control | Route, Dosing | |
| Group | Animals | Article (dose) | Regimen | End Point(s) |
| 1 | 9 | Native IL-2 | IV, single dose on | Blood |
| | | (wild-type) | Day 0 at T = 0 | collection at |
| | | (3.0 mg/kg) | at 5 mL/kg | 0.08, 0.25, 0.5, |
| | | Concentration: 0.6 | | 1, 2, 4, 8, 12, |
| | | mg/mL | | and 24 hours |
| 2 | 9 | K35_30kD | IV, single dose on | post dose |
| | | (3.0 mg/kg) | Day 0 at T = 0 | |
| | | Concentration: 0.6 | at 5 mL/kg | |
| | | mg/mL | | |
| 3 | 9 | K35_30kD | IV, single dose on | |
| | | (0.3 mg/kg) | Day 0 at T = 0 | |
| | | Concentration: 0.6 | at 5 mL/kg | |
| | | mg/mL | | |
| Extra | 6 | N/A | N/A | Blank Matrix |
| | | | | Collection |
| | | | | (untimed) |
| Total | 33 | | | |

The pharmacokinetic properties of an exemplary PEGylated IL-2 compound K35_30 kD at two dose levels was evaluated. The lyophilized test article was reconstituted in PBS, and nine male C57BL/6 mice were dosed with 0.3 and 3 mg/kg via intravenous tail vein injection for each dose group (see collection details below). Blood samples were collected at 0.08, 0.25, 0.5, 1, 2, 4, 8, 12, and 24 hours post dose. The hIL-2 ELISA kit from Abcam (ab100566), which does not cross-react with native mouse IL-2, was used for detection and quantitation of test articles. To adjust for ELISA-specific differences in sensitivity of kit detection of native and PEGylated compounds, native IL-2 and K35_30kD test article standard curves were generated using the test article diluent buffer, and data were analyzed with respect to respective standard curves. The data plotted represent the mean and SEM of three individual samples (biological replicates) as described above, and PK parameters for K35_30 kD test articles were extracted and summarized in Table 14.

TABLE 14

| | | | Dose | |
|---|---|---|---|---|
| | | | 0.3 mg/kg | 3 mg/kg |
| Analyte | | Unit | Estimate | |
| IL-2 K35- | Tₘₐₓ | hr | 0.250 | 0.250 |
| mPEG30kD | Cₘₐₓ | ng/mL | 6080 | 57700 |
| | AUCₒ₋ₜ | hr*/ng/mL | 38500 | 425000 |
| | R² | | 0.994 | 0.947 |
| | AUC_{1/2extrap} | % | 35.3 | 37.4 |
| | AUG_{0-∞} | h*ng/mL | 59600 | 679000 |
| | t_{1/2} | hr | 18.2 | 19.5 |
| | C_{max/D} | kg*ng/mL/mg | 20300 | 19200 |
| | AUC_{o-t/D} | hr*ng/mL | 128000 | 142000 |

### Example 9

Characterization of Binding to Human IL-2R Alpha and IL-2R Beta

A study was conducted to characterize the binding of an exemplary IL-2 conjugate IL-2_P65[AzK_L1_PEG30kD]-1 to human IL-2R alpha and IL-2R beta. Compound IL-2_P65[AzK_L1_PEG30kD]-1 was as described earlier and comprises SEQ ID NO: 50, wherein the proline at position 64 is replaced by AzK_L1_PEG30kD, where AzK_L1_PEG30kD is defined as a structure of Formula (IV) or Formula (V), or a mixture of Formula (IV) and Formula (V), and a 30 kDa, linear mPEG chain. Compound IL-2_P65[AzK_L1_PEG30kD]-1 is also defined as the compound comprising SEQ ID NO: 3 in which the proline residue at position 64 (P64) is replaced by the structure of Formula (VIII) or Formula (IX), or a mixture of Formula (VIII) and Formula (IX), and wherein n is an integer such that the molecular weight of the PEG group is about 30 kDa. Compound IL-2_P65[AzK_L1_PEG30kD]-1 is also defined as the compound comprising SEQ ID NO: 3 in which the proline residue at position 64 (P64) is replaced by the structure of Formula (XII) or Formula (XIII), or a mixture of Formula (XII) and Formula (XIII), and wherein n is an integer such that the molecular weight of the PEG group is about 30 kDa.

The compound was prepared using methods similar to those disclosed in Example 2, wherein a protein was first prepared having SEQ ID NO: 3 in which the proline at position 64 was replaced by N6-((2-azidoethoxy)-carbonyl)-L-lysine AzK (SEQ ID NO: 35). The AzK-containing protein was then allowed to react under click chemistry conditions with DBCO comprising a methoxy, linear PEG group having an average molecular weight of 30 kDa. Briefly, IL-2 employed for bioconjugation was expressed as inclusion bodies in E. coli using methods disclosed herein, using: (a) an expression plasmid encoding (i) the protein with the desired amino acid sequence, which gene contains a first unnatural base pair to provide a codon at the desired position at which the unnatural amino acid N6-((2-azidoethoxy)-carbonyl)-L-lysine (AzK) was incorporated and (ii) a tRNA derived from M. mazei Pyl, which gene comprises a second unnatural nucleotide to provide a matching anticodon in place of its native sequence; (b) a plasmid encoding a M. barkeri derived pyrrolysyl-tRNA synthetase (Mb PylRS), (c) AzK; and (d) a truncated variant of nucleotide triphosphate transporter PtNTT2 in which the first 65 amino acid residues of the full-length protein were deleted. The double-stranded oligonucleotide that encodes the amino acid sequence of the IL-2 variant contained a codon AXC as codon 64 of the sequence that encodes the protein having SEQ ID NO: 3 in which P64 is replaced with an unnatural amino acid described herein. The plasmid encoding an orthogonal tRNA gene from M. mazei comprised an AXC-matching anticodon GYT in place of its native sequence, wherein Y is an unnatural nucleotide as disclosed herein. X and Y were selected from unnatural nucleotides dTPT3 and dNaM as disclosed herein. The expressed protein was extracted from inclusion bodies and re-folded using standard procedures before site-specifically pegylating the AzK-containing IL-2 product using DBCO-mediated copper-free click chemistry to attach stable, covalent mPEG moieties (methoxy, linear PEG group having an average molecular weight of 30 kDa) to the AzK (as outlined in Scheme S6 above).

Test article samples binding to IL-2R alpha. The test article samples in solution were tested for binding over the IL-2R alpha receptor surfaces. Response data were processed by subtracting the signals from a reference surface without receptor as well as an average of buffer injections using Scrubber-2 (Biologic Software Pty Ltd). Responses for the rhIL-2 concentration series were globally fit to a 1:1 interaction model including a step for mass transport). A summary of the binding constants is provided in Table 15.

TABLE 15

| | KD (nM) | |
|---|---|---|
| | IL-2 R alpha | IL-2 R beta |
| rhIL-2 | 11 ± 1 | 0.7 ± 1 |
| IL-2_P65[AzK_L1_PEG30kD]-1 | n. d. | 3.1 ± 0.3 |

Capture of Fc-tagged IL-2R beta on Protein coated CM4 sensor chip. A CM4 sensor chip was docked into the Biacore 4000 optical biosensor and the instrument was primed three times with HBS-P running buffer (HBS-P is 1× HBS-N with 0.005% Tween-20 added). Protein A was coupled using standard NHS/EDC coupling conditions. IL-2R beta-Fc was dissolved in water to a concentration of 0.1 mg/ml and then diluted 1/1000 into the HBS-P running buffer. IL-2R beta-Fc was injected for different lengths of time to create 2 different density receptor surfaces (˜750 RU and 1500 RU, data not shown).

Characterization of samples binding to IL-2R beta. The test article samples in solution were tested for binding over the IL-2R beta receptor surfaces. Response data were processed by subtracting the signals from a reference surface without receptor as well as an average of buffer injections using Scrubber-2 (Biologic Software Pty Ltd). Responses for the rhIL-2 (4 uM highest concentration 2-fold dilutions) and IL-2_P65[AzK_L1_PEG30kD]-1 samples (8 uM highest concentration 2-fold dilutions) tested in duplicate were globally fit to a 1:1 interaction model including a step for mass transport. A summary of the binding constants is provided in Table 15.

Results. His-tagged IL-2R alpha was captured at different densities on a nickel charged NTA sensor chip within a Biacore SPR biosensor system. Fc-tagged IL-2R beta was captured at different densities on a Protein A coated CM4 sensor chip. Response data were fit to a 1:1 interaction model to determine binding constants for each interaction. Recombinant human IL-2 (rhIL-2) bound to IL-2R alpha with an affinity of ˜11 nM, while no binding of IL-2_P65[AzK_L1_PEG30kD]-1 samples could be detected to the IL-2R alpha. The rhIL-2 bound to IL-2R beta with an affinity of ˜700 nM, and IL-2_P65[AzK_L1_PEG30kD]-1 bound to IL-2R beta with an affinity of ˜3 uM under these test conditions.

### Example 10

A study was conducted to determine the potency and differential cell-type specificity of IL-2_P65[AzK_L1_PEG30kD]-1 vs. recombinant human interleukin-2 (hIL-2) for the phosphorylated form of the transcription factor STAT5 (pSTAT5) signaling potency human primary immune cell types. Compound IL-2_P65[AzK_L1_PEG30kD]-1 was as described earlier and comprises SEQ ID NO: 50, wherein the proline at position 64 is replaced by AzK_L1_PEG30kD, where AzK_L1_PEG30kD is defined as a structure of Formula (IV) or Formula (V), or a mixture of Formula (IV) and Formula (V), and a 30 kDa, linear mPEG chain. Compound IL-2_P65[AzK_L1_PEG30kD]-1 is also defined as the compound comprising SEQ ID NO: 3 in which the proline residue at position 64 (P64) is replaced by the structure of Formula (VIII) or Formula (IX), or a mixture of Formula (VIII) and Formula (IX), and wherein n is an integer such that the molecular weight of the PEG group is about 30 kDa. Compound IL-2_P65[AzK_L1_PEG30kD]-1 is also defined as the compound comprising SEQ ID NO: 3 in which the proline residue at position 64 (P64) is replaced by the structure of Formula (XII) or Formula (XIII), or a mixture of Formula (XII) and Formula (XIII), and wherein n is an integer such that the molecular weight of the PEG group is about 30 kDa. The compound was prepared using methods similar to those disclosed in Example 2, wherein a protein was first prepared having SEQ ID NO: 3 in which the proline at position 64 was replaced by N6-((2-azidoethoxy)-carbonyl)-L-lysine AzK (SEQ ID NO: 35). The AzK-containing protein was then allowed to react under click chemistry conditions with DBCO comprising a methoxy, linear PEG group having an average molecular weight of 30 kDa.

Human PBMC sample Treatment Methods. Stocks of IL-2 (control, 1 mg/mL), and IL-2_P65[AzK_L1_PEG30 kD]-1 (“lot 1”: 1.27 mg/mL; “lot 2”: 2.29 mg/mL) were stored as stock solutions frozen at −20° C.

The IL-2_P65[AzK_L1_PEG30kD]-1 lot 1 and lot 2 compounds were diluted in PBS and the IL-2 was diluted using PBS+0.1% BSA to create 10× stocks. The 10× IL-2 stock concentration was 5 ug/ml and the GLP-1 and GLP-2 stocks were between 6-300 μg/ml, depending on the experiment. The 10× stocks were diluted in successive 5-fold dilutions to create a 10-point dose titration. The top dose of the IL-2 was 5 μg/ml and the lot 1 and lot 2 stocks were between 6-300 μg/ml depending on the experiment. 10 μl of each stock was added to 90 μl of cell samples to achieve a final top dose for IL-2 of 500 ng/ml and 0.6-30 μg/ml for each of lot 1 and lot 2.

Sample Stimulation. To stimulate, 10 μl of the dose titration outlined above was added to 90 μl of blood sample pre-equilibrated to 37° C. The samples were incubated at 37° C. for 45 minutes. At the end of the incubation period, the red blood cells were lysed and the cells were fixed simultaneously as follows:

100 μl cells were transferred to 900 μl of BD Lyse/Fix Buffer (Beckton Dickinson, Cat #558049) and vortexed immediately. The BD Lyse/Fix was prepared by diluting the stock 1:5 with cell culture water just prior to addition. Samples were incubated 10 minutes at room temperature, then centrifuged at 450×g for 5 minutes to pellet cells. Pelleted cells were washed with PBS+0.5% BSA and stored at −37° C. until analysis.

Staining Protocol.

Step 1. Thawed cells at room temperature. Step 2. Added the Fc Block (TruStain FcX™). Step 3. Incubated at room temperature for 5 minutes. Step 4. Added the following antibodies from Table 16:

TABLE 16
Antibodies for Human panel.

| | Fluorophore |
|---|---|
| CD4 | BUV737 |
| CD56 | BV711 |
| CD16 | BV711 |
| CD8 | BUV805 |
| CD27 | BV786 |
| CD45RA | BUV395 |
| CD127 | FITC |
| CD25 | Biotin |

Step 5. Incubated for 20 minutes at room temperature. Step 6. Washed cells two times with PBS+0.5% BSA. Step 7. Permeabilized cells by adding 10 volumes of methanol to one volume of cells. Step 8. Incubated cells for 10 minutes at 4° C. Step 9. Washed with PBS. Step 10. Washed cells with PBS w/0.5% BSA. Step 11. Added the Fc Block (TruStain FcX™). Step 12. Added the following post-permeabilization staining panel from Table 17.

TABLE 17
Staining reagents.

| | Fluorophore |
|---|---|
| CD3 | PE-Cy7 |
| STAT5 | Ax647 |
| Streptavidin | BV421 |
| FOXp3 | PE |

Flow Cytometry and data analysis. Samples were run on Becton Dickinson Fortessa and LSR II instrument with five lasers (372 nM, 405 nM, 488 nM, 561 nM, and 640 nM). The instruments were equipped with 20 detectors including the scatter parameters. The instruments were regularly calibrated using Becton Dickinson Cytometer Setup & Tracking Beads. The 96 well plates containing the stained samples were run at less than 8,000 cells/second using the 96-well high throughput sampler.

The data was exported as .fcs files to a network drive and compensated to account for spillover of the fluorophores and the fcs files are annotated. The fcs files were then gated. The cells were first gated on singlets using FSC-A by FSC-H to exclude any aggregates or doublets. Within this gate, the cells were gated on mid to high forward scatter (FSC-A) and side scatter (SSC-A) to exclude the red blood cells, debris, and granulocytes. The T cells were then gated as the CD3+, CD56/16 negative population 3 panel. The NK cells were identified as the CD3 negative, CD56/16 high population, 3^{rd} panel. The T cells were then divided into CD4+ T cells and CD8+ T cells. The Treg cells were then gated from the CD4+ T cells as the CD25^{hi}×C127^{lo} population.

Statistics and plotting for derivation of EC50 values. The Median Fluorescence Intensity (MFI) for each of the cell population, donor, and compound treatment was calculated from the signal in the channel detecting phosphorylated. The statistics were analyzed using Spotfire. Within Spotfire, the data was plotted on a log scale for the compound doses and a linear scale for the MFI readings. These data were fit using a 4-parameter logistic regression equation. The EC50 was calculated as the inflection point of the curve.

Results. Human IL-2 and IL-2_P65[AzK_L1_PEG30kD]-1 samples were diluted and tested in triplicate against each of three individual donors as described above. The calculated half-maximal effective concentration (EC50) values are listed in Table 19. Results demonstrate that IL-2_P65[AzK_L1_PEG30kD]-1 is a potent agonist of IL-2 receptor signaling in lymphocytes from human. Consistent with previous in vitro binding studies that showed IL-2_P65[AzK_L1_PEG30kD]-1 specifically engages the IL-2Rα subunit and not IL2Ra, it demonstrated specifically reduced signaling potency in Treg cells that rely on IL-2Rα engagement for potency, compared to Teff and NK cells that do not constitutively express high levels of IL-2Rα.

TABLE 18
Potency Characteristics for hIL-2 and
IL-2_P65 [AzK_L1_PEG30kD]-1
Lots Against Primary CD8 + T Cell, NK Cell, and
Treg Cell Subpopulations from Human donors.
Human Cell EC50 (ng/mL)

| | | | | | CD8/ |
|---|---|---|---|---|---|
| | | Mem | | | Treg |
| Material | CD8 + T | CD8 + T | NK | Treg | ratio |
| Human | 12.4 ± 1.29 | 11.3 ± 1.63 | 2.88 ± 1.63 | 0.027 ± 0.005 | 460 |
| IL-2 | | | | | |
| lot 1* | 224 ± 25.3 | 240 ± 27.3 | 54.7 ± 6.55 | 114 ± 20.3 | 2.0 |
| lot 2* | 265 ± 12.4 | 284 ± 18.3 | 55.7 ± 8.62 | 132 ± 24.0 | 2.0 |

Data are mean ± standard error of the mean (SEM) calculated from triplicate testing of samples from three independent donors.
*IL-2_P65[AzK_L1_PEG30k_D]-1.

### Example 11

Use of Compound A plus a checkpoint inhibitor in the treatment of CT-26 tumor-bearing Balb/c mice. Compound IL-2_P65[AzK_PEG30kD] (also referred to herein as “P65_30kD” and in the Figures as “Compound A”) was prepared according to the methods disclosed herein by first preparing a protein having SEQ ID NO: 4 in which the proline at position 65 was replaced by N6-((2-azidoethoxy)-carbonyl)-L-lysine (AzK) (SEQ ID NO: 10). The AzK-containing protein was then allowed to react under click chemistry conditions with DBCO comprising a methoxy, linear PEG group having an average molecular weight of 30 kDa to afford a product having SEQ ID NO: 25 comprising Formula (II), Formula (III), or a mixture of Formula (II) and Formula (III), wherein W is a methoxy, linear PEG group having an average molecular weight of 30 kDa. The compound can also be defined as comprising the amino acid sequence of SEQ ID NO: 4 in which the proline at position 65 (P65) is replaced by the structure of Formula (VI) or Formula (VII), or a mixture of Formula (VI) and Formula (VII), and wherein n is an integer such that the PEG group has a molecular weight of about 30 kDa. The compound can also be defined as comprising the amino acid sequence of SEQ ID NO: 4 in which the proline at position 65 (P65) is replaced by the structure of Formula (X) or Formula (XI), or a mixture of Formula (X) and Formula (XI), and wherein n is an integer such that the PEG group has a molecular weight of about 30 kDa.

Studies of Compound A as monotherapy and in combination with an anti-PD-1 antibody were undertaken in Balb/c female mice. Balb/c female mice, 6-8 weeks of age, with an average weight of 16 g to 21 g were purchased from Jackson Laboratories (Sacramento, Calif.) for studies 1 and 2. Balb/c female mice, 7-8 weeks of age, with an average weight of 18 to 22 g were purchased from Taconic Biosciences by HD Biosciences for study 3. Cryogenically preserved vials of CT-26 colon cancer cells were purchased from American Tissue Type Collection (ATCC, Manassas, Va.). Cells were thawed and cultured according to the manufacturer's protocol. On the day of tumor cell inoculation, cells were washed in serum-free media, counted, and resuspended in cold serum-free media at a concentration of 250,000 (studies 1 and 2) or 300,000 (study 3) viable cells per 0.1 mL. The CT-26 cells (0.1 mL) were injected subcutaneously into the flanks of individual mice and tumors were allowed to grow.

For studies 1 and 2 in which a combination of Compound A and an anti-PD-1 antibody were used, the antibody used was anti-mouse PD-1 (BioXcell; RMP1-14) and the control antibody was IgG1 isotype antibody (BioXcell; catalog #BP0089, lot #2A3). For study 3 in which an anti-PD-1 antibody was used, the antibody used was anti-mouse PD-1 (BioXcell; catalog #BP0146, RMP1-14, lot #695318A1) and the control antibody was IgG1 isotype antibody (BioXcell; catalog #BP0089, lot #2A3).

Lyophilized Compound A was reconstituted into 10 mg/mL stock with 0.1 M acetic acid. It was then further diluted into working concentration with 1× phosphate buffered saline (PBS). The compound was reconstituted and diluted within an hour of dosing of animals and kept on ice until dosing. The lyophilized compound was stored at −80° C. before use. Vehicle was stored at 4° C.

Three separate efficacy studies were performed using CT-26 tumor-bearing Balb/c mice. The design for study 1, which evaluated Compound A for dose-dependent efficacy as a single agent, is outlined in Table 19. The designs for studies 2 and 3, which evaluated the efficacy of Compound A in combination with an anti-PD-1 antibody, are outlined in Table 20 and Table 21, respectively. The route of administration for Compound A was intravenous (IV). IV dosing in the mice was done via the tail vein. The antibody was administered intraperitoneally (IP). All agents were administered based on the individual body weight of each animal obtained immediately prior to each dosing. Details on the dosing regimen are described below.

TABLE 19
Study #1: Control and Test Treatment
Groups in CT-26 Tumor-Bearing Mice.

| Agent | Dose (mg/kg) | Route, Schedule | No. of mice |
|---|---|---|---|
| Vehicle control | 0 | IV, QWx3 | 10 |
| Compound A | 0.3 | IV, QWx3 | 10 |
| Compound A | 0.3 | IV, Q2Wx2 | 10 |
| Compound A | 1 | IV, QWx3 | 10 |
| Compound A | 1 | IV, Q2Wx2 | 10 |
| Compound A | 1 | IV, QWx3 | 10 |
| Compound A | 3 | IV, Q2Wx2 | 10 |

IV = intravenous;
QWx3 = once a week for a total of 3 doses;
Q2Wx2 = once every 2 weeks for a total of 2 doses.

TABLE 20
Study #2: Control and Test Treatment
Groups in CT-26 Tumor-Bearing Mice.

| | Dose | Route, | No. of |
|---|---|---|---|
| Agent | (mg/kg) | Schedule | mice |
| Vehicle control + | 0 + 10 | IV, QWx3 + | 14 |
| IgG isotype control | | IP, BIWx3 | |
| Compound A | 3 | IV, QWx3 | 14 |
| Compound A | 6 | IV, QWx3 | 14 |
| Anti-PD-1 antibody | 10 | IP, BIWx3 | 14 |
| Compound A + | 6 + 10 | IV, QWx3 + | 14 |
| Anti-PD-1 antibody | | IP, BIWx3 | |

BIWx3 = twice a week for 3 weeks with a total of 6 doses;
IP = intraperitoneal;
IV = intravenous;
QWx3 = once a week for a total of 3 doses.

TABLE 21
Study #3: Control and Test Treatment
Groups in CT-26 Tumor-Bearing Mice.

| | Dose | Route, | No. of |
|---|---|---|---|
| Agent | (mg/kg) | Schedule | mice |
| Vehicle control + | 0 + 10 | IV, QWx3 + | 14 |
| IgG isotype control | | IP, BIWx3 | |
| Compound A | 1 | IV, QWx3 | 14 |
| Compound A | 3 | IV, QWx3 | 14 |
| Compound A | 6 | IV, QWx3 | 14 |
| Compound A | 9 | IV, QWx3 | 14 |
| Anti-PD-1 antibody | 10 | IP, BIWx3 | 14 |
| Compound A + | 1 + 10 | IV, QWx3 + | 14 |
| Anti-PD-1 antibody | | IP, BIWx3 | |
| Compound A + | 3 + 10 | IV, QWx3 + | 14 |
| Anti-PD-1 antibody | | IP, BIWx3 | |
| Compound A + | 6 + 10 | IV, QWx3 + | 14 |
| Anti-PD-1 antibody | | IP, BIWx3 | |

BIWx3 = twice a week for 3 weeks with a total of 6 doses;
IP = intraperitoneal;
IV = intravenous;
QWx3 = once a week for a total of 3 doses;
Q2Wx2 = once every 2 weeks for a total of 2 doses.

In Study 1, CT-26 tumor-bearing mice were treated with vehicle IV once a week for a total of 3 doses (QWx3) or Compound A at 0.3, 1, or 3 mg/kg IV, either once a week for a total of three doses (QWx3), or once every 2 weeks for a total of 2 doses (Q2Wx2), starting on Day 4 following tumor cell inoculation when the average tumor volume was ˜80 mm³.

In Study 2, CT-26 tumor-bearing mice were treated on Day 5 following tumor cell inoculation when the average tumor volume was ˜80 mm³. Dosing was with vehicle IV QWx3+ IgG isotype control IP or Compound A at 3 or 6 mg/kg IV, on a QWx3 dosing schedule, or anti-PD-1 antibody at 10 mg/kg IP, or the combination of Compound A at 6 mg/kg IV QWx3+ anti-PD-1 antibody at 10 mg/kg IP. The IP dosing of the antibody in all cases was twice a week for 3 weeks with a total of 6 doses (BIWx3).

In Study 3, CT-26 tumor-bearing mice were treated on Day 7 following tumor cell inoculation when the average tumor volume was ˜70 mm3. Dosing was with vehicle IV QWx3+ IgG isotype control IP BIWx3; or Compound A at 1, 3, 6, or 9 mg/kg IV on a QWx3 dosing schedule, or anti-PD-1 antibody at 10 mg/kg IP BIWx3; or the combination of Compound A at 1, 3, or 6 mg/kg IV QWx3+ anti-PD-1 antibody at 10 mg/kg IP BIWx3.

A summary of all three studies is shown in Table 22. Animals were observed daily for clinical signs. In accordance with IACUC guidelines, animals were humanely euthanized when tumors grew over 2000 mm³ in volume or they were observed to have a continuing deteriorating condition or showing obvious signs of severe distress and/or pain.

The survival of each mouse was monitored for over 100 days, at which time surviving tumor-free animals in Studies 2 and 3 were included in a re-challenge continuation of the study for two cycles, 2 months apart. Specifically, tumor-free animals were re-challenged via inoculation of the same type of tumor cells (CT-26) in the opposite lower flank. Control animals were age-matched naïve mice that were concurrently inoculated with the same number of CT-26 tumor cells in the opposite lower flank.

Tumor growth was monitored using digital caliper measurements every 3 to 4 days until the end of the study. Tumor volume was calculated as Width²×Length/2, where width is the smallest dimension and length is the largest. Raw tumor volume data are presented in the study reports.

Mean tumor volume data for each group was plotted overtime with standard error of the mean (SEM) bars. Additionally, individual tumor volume data for the last day before animal sacrifice was plotted along with mean and SEM bars to examine the distribution of the data.

A statistical analysis of the tumor volume data for the last day before animal sacrifice was performed using the using GraphPad Prism v.7.0. Data was analyzed for significance using a one-way ANOVA. Pairwise comparisons were made using Tukey's test procedures (2-sided). The p-value for each individual comparison was reported.

The percent tumor growth inhibition (% TGI) in each treated group vs. a control group was calculated as:

[(Control-Control baseline)−(Treated-Treated baseline)]/(Control-Control baseline)×100%.

The survival of each mouse was recorded, and a Kaplan-Meir plot was generated to show survival by treatments group and the significance was assessed by log-rank (Mantel-Cox) test. Survival was monitored for over 100 days following treatment initiation in Studies #1, #2, and #3, and over the two re-challenge cycles in surviving tumor-free mice in Studies #2 and #3. Analyses were performed using GraphPad Prism version 7.0.

TABLE 22
Tumor Growth Inhibition in Mice with Compound A as Monotherapy
and in Combination with Anti-Mouse PD-1 Antibody.

| | Compound A | | | | |
|---|---|---|---|---|---|
| | Dose | % TGI (Relative to Vehicle Control) | | | |
| | [Antibody | Study #1 | | Study #2 | Study #3 |
| | Dose^{a}] (mg/kg) | QWx3 | Q2Wx2 | QWx3 | QWx3 |
| Compound A | 0.3 | 19 | 20 | — | — |
| Monotherapy | 1 | 31 | 27 | — | 30 |
| | 3 | 51* | 45* | 56 | 59*** |
| | 6 | — | — | 36* | 86*** |
| | 9 | — | — | — | 85*** |
| | Antibody [10] | — | — | 44^{#} | 44^{#} |
| Combination | 6 + [10] | — | — | 75** | 84** |
| Therapy^{b} | | | | | |

^{a}Dosed BIW for 3 weeks (6 total doses);
^{b}Data for the 1 and 3 mg/kg Compound A combination groups not shown.
% TGI was calculated on Day 15 (Study 1) and Day 17 (Studies 2 and 3).
Results are mean ± SEM.

QWx3=once a week for a total of 3 doses; Q2Wx2=once every 2 weeks for a total of 2 doses; TGI=tumor growth inhibition. *p<0.05 vs. vehicle control; **p<0.05 vs. monotherapies (Compound A or antibody); ***p<0.001 vs. vehicle or antibody isotype control; ^{#}p<0.01 vs. antibody isotype control.

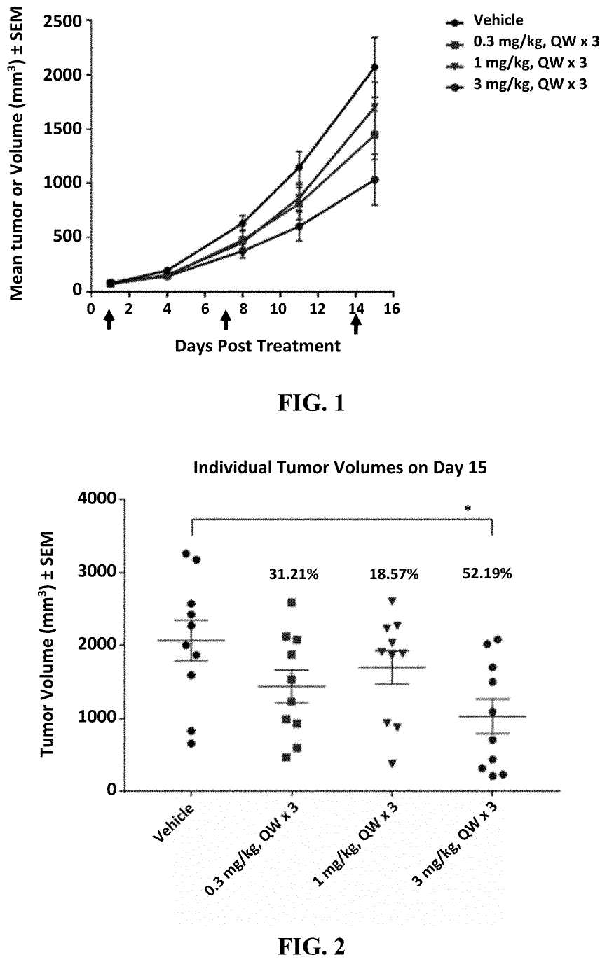

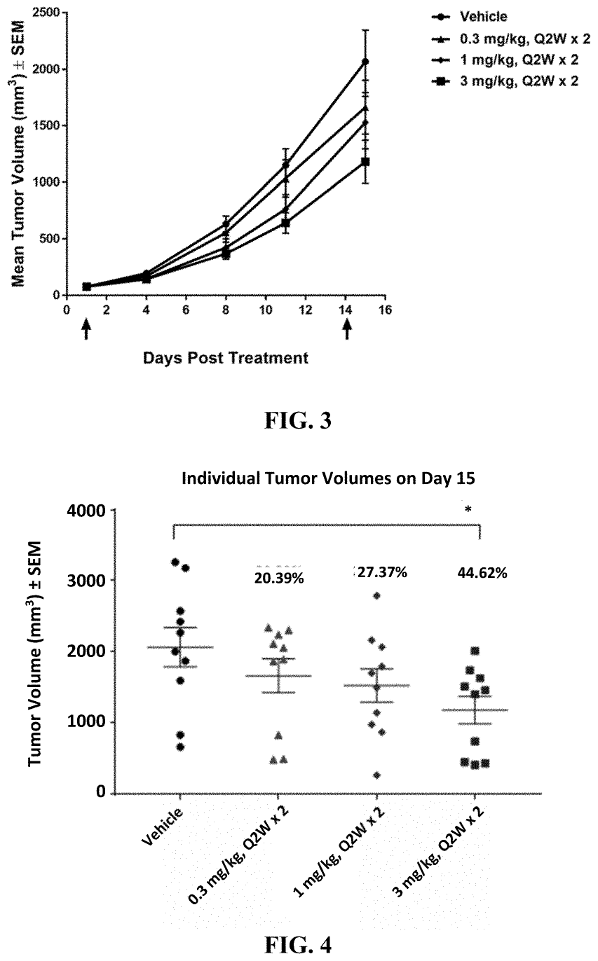

In Study 1, Compound A was evaluated for dose-dependent efficacy as a single agent in female Balb/c mice bearing subcutaneously established CT-26 colon tumors. The study formally ended on Day 15 after treatment initiation according to the humane endpoint set forth by the IACUC when several tumors in the control group reached over 2000 mm³ in volume. FIG. 1 shows mean tumor volume over time for groups treated QWx3 dosing with Compound A. FIG. 2 shows tumor volumes on Day 15 post treatment for each animal treated QWx3 dosing with Compound A. FIG. 3 shows mean tumor volume over time for groups treated Q2Wx2 dosing with Compound A. FIG. 4 shows tumor volumes on Day 15 post treatment for each animal with Q2Wx2 dosing with Compound A.

On the QWx3 dosing schedule, Compound A demonstrated dose-dependent single agent anti-tumor activity resulting in % TGI compared to the vehicle control of 31%, 19%, and 52% for the 0.3, 1, and 3 mg/kg dose groups, respectively. Similarly, on the Q2Wx2 dosing schedule, Compound A demonstrated dose-dependent single agent anti-tumor activity resulting % TGI compared to the vehicle control of 20%, 27%, and 45% for the 0.3, 1, and 3 mg/kg dose groups, respectively. However, on both dosing schedules, only the 3 mg/kg dose was statistically significant (p<0.05) compared to the vehicle control. Both dosing schedules demonstrated comparable anti-tumor activity. Therefore, for the subsequent studies in this mouse model, the QWx3 dosing schedule was chosen.

In FIGS. 1, 3, 5, and 8, black arrows denote days of Compound A dosing. Data in FIGS. 1 and 3 are mean tumor growth curves with QWx3 dosing and Q2Wx2 dosing with Compound A; black arrows denote days of Compound A dosing. Data in FIGS. 2 and 4 represent individual tumor volume and mean tumor volume standard error of the mean (SEM) (10 mice/group) on day 15 post-treatment with QWx3 and Q2Wx2 dosing with Compound A. Data represent individual tumor volumes; the mean SEM and % TGI compared to the vehicle control are also displayed.

Data in FIG. 3 represents mean tumor volume standard error of the mean (SEM) (10 mice/group) in animals with Q2Wx2 dosing with Compound A. Data in FIG. 4 represents individual and mean tumor volume data on Day 15 post treatment with Q2Wx2 dosing with Compound A. *p<0.05 vs. vehicle control on Day 15.

There were two separate studies (Studies 2 and 3) conducted in CT-26 colon tumor-bearing mice to assess Compound A as a single agent and in combination with a murine anti-PD-1 checkpoint inhibitor antibody. The dose ranges for Compound A between the studies overlapped, with Study 3 having a wider dose range. In both studies, Compound A was administered QWx3 and the same dose level of antibody was administered BIWx3.

In Study 2, anti-tumor activity of Compound A was evaluated as a single agent at 3 and 6 mg/kg (QWx3) in female Balb/c mice bearing subcutaneously established CT-26 colon tumors. Additionally, the combination anti-tumor activity was evaluated with IV dosing of Compound A at 6 mg/kg (QWx3) and anti-PD-1 antibody at 10 mg/kg P (BIWx3). The % TGI was calculated on Day 15 after treatment initiation because several tumors in the vehicle control group reached over 2000 mm³ in volume. However, the animals in treatment groups that demonstrated complete tumor regression were followed with tumor measurements at a frequency of once or twice a week.

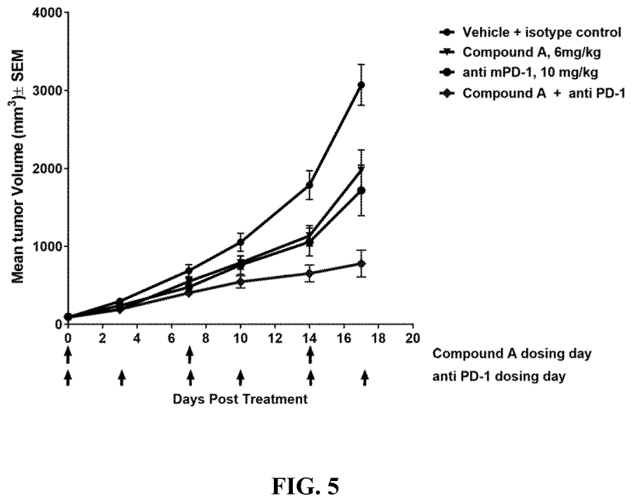

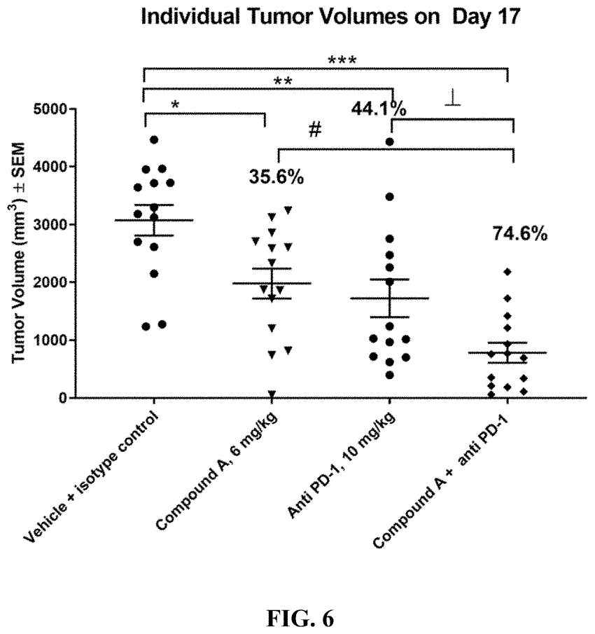

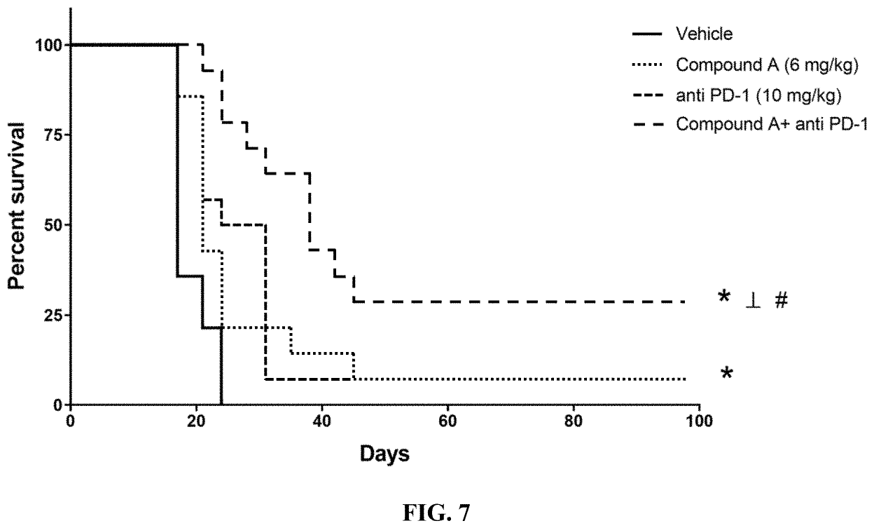

Compound A demonstrated single agent anti-tumor activity resulting in % TGI compared to the vehicle control of 56.3% and 35.6% for the 3 and 6 mg/kg dose groups, respectively. In the combination study, CT-26 tumor-bearing mice were treated IV with Compound A at 6 mg/kg QWx3, or IP with anti-PD-1 antibody BIWx3, or the combination with the same dosing schedules, starting 5 days following tumor cell inoculation when the average tumor volume was ˜80 mm³. Mean tumor growth curves are shown in FIG. 5 for treatment of mice with vehicle, 6 mg/kg Compound A as a single agent, anti-PD-1 antibody as a single agent, and the combination of 6 mg/kg Compound A and anti-PD-1 antibody. Data in FIG. 5 represent mean tumor volume±SEM (14 mice/group). Upper arrows denote days of Compound A dosing and lower arrows denote days of anti-PD-1 antibody dosing. The combination anti-tumor activity was significantly enhanced compared to Compound A or anti-PD-1 antibody alone (p<0.05). The % TGI data is shown in FIG. 6 and shows significant anti-tumor effects on Day 15 post treatment in the group treated with the combination of Compound A and anti-PD-1 antibody, compared to the groups treated with vehicle, Compound A alone or the anti-PD-1 antibody alone (35.6% for the Compound A alone group; 44.1% for the anti-PD-1 antibody alone group; and 74.6% for the group administered the combination of Compound A and anti-PD-1 antibody). Data represent individual tumor volumes; the mean SEM and % TGI compared to the vehicle control are also displayed. *p<0.05, **p<0.01, and ***p<0.01; vs. vehicle control. ^{⊥}p<0.05 vs. anti-PD-1 antibody. #p<0.05 vs. Compound A. The median survival times of the groups are shown in FIG. 7 and were 17, 27, 27.5, and 38 days for the control, Compound A, anti-PD-1 antibody, and Compound A+anti-PD-1 antibody groups, respectively. The median survival time of the combination group was significantly longer than both the Compound A (p<0.05) and anti-PD-1 antibody (p<0.05) single agent treatment groups. At 98 days post treatment, only 1 out of 14 animals (7%) in each of Compound A and anti-PD-1 antibody dose groups survived tumor-free, while 4 of 14 animals (29%) in the combination group survived tumor-free. Data in FIG. 7 represent Kaplan-Meier survival curves for treatment groups. *p<0.05 vs. vehicle control. ^{⊥}p<0.05 vs. anti-PD-1 antibody. #p<0.05 vs. Compound A.

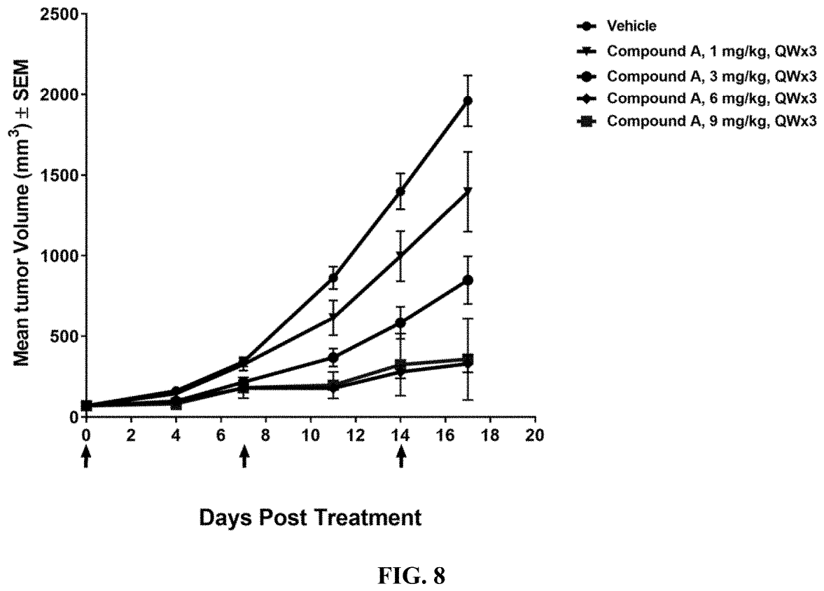

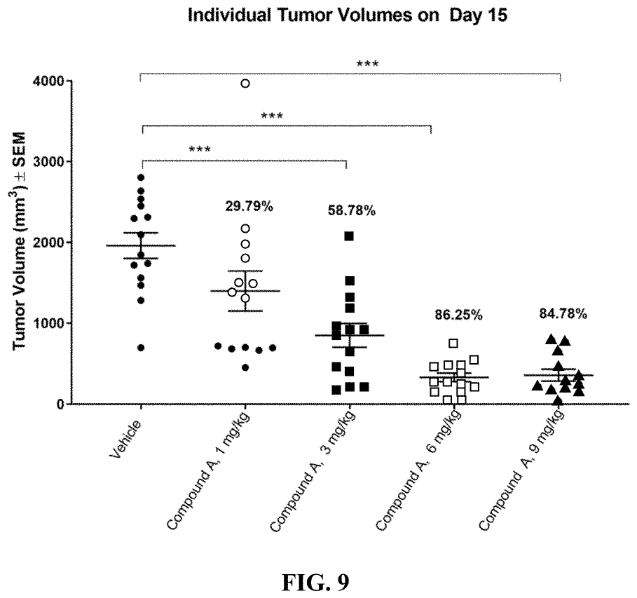

In Study 3, the single agent anti-tumor activity of Compound A was evaluated in female Balb/c mice bearing SC CT-26 colon tumors at a wider dose range (1, 3, 6, and 9 mg/kg) as compared to Study 2 on the same IV QWx3 dosing schedule. Data in FIG. 8 represent mean tumor growth curves when Compound A was dosed a single agent at 1 mg/kg, 3 mg/kg, 6 mg/kg, and 9 mg/kg. Data represent mean tumor volume±SEM (14 mice/group; except 12 mice/group for 9 mg/kg Compound A). Black arrows denote days of Compound A dosing. Compound A dosed alone at 1 mg/kg, 3 mg/kg, 6 mg/kg, and 9 mg/kg also demonstrated dose-dependent anti-tumor activity resulting in % TGI compared to the vehicle control of 29.8%, 58.8%, 86.2%, and 84.8% for 1, 3, 6, and 9 mg/kg dose groups, respectively (FIG. 9). The % TGI was calculated on Day 15 after treatment initiation because several tumors in the vehicle control group reached over 2000 mm³. However, the animals in treatment groups that demonstrated complete tumor regression were followed with tumor measurements at a frequency of once or twice a week. Data in FIG. 9 represent individual tumor volumes on Day 15 post treatment. Data represent individual tumor volumes; the mean±SEM and % TGI compared to the vehicle control are also displayed. ***p<0.01 vs. vehicle control. The lowest dose (1 mg/kg) did not show statistically significant anti-tumor activity while the other 3 dose groups were statistically significant (p<0.001) compared to the vehicle treated group. The data also showed that % TGI for the two high dose groups (6 mg/kg and 9 mg/kg) were similar indicating maximal anti-tumor activity was reached at the 6 mg/kg dose. In the 9 mg/kg dose group, 2 of the 14 animals were found dead following >15% body weight loss due to treatment.

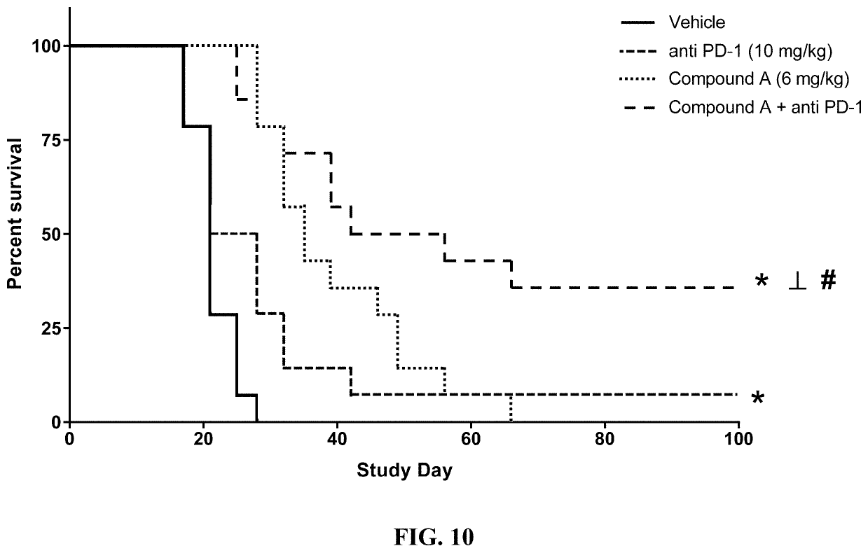

In the combination phase of the study, Compound A at 1, 3, or 6 mg/kg (QWx3) was dosed with anti-PD-1 antibody at 10 mg/kg IP (BIWx3). CT-26 tumor-bearing mice were treated IV with Compound A at 1, 3, 6, or 9 mg/kg QWx3, or IP anti-PD-1 antibody BIWx3, or the combination with the same dosing schedules, starting 7 days following tumor cell inoculation when the average tumor volume was ˜70 mm3. Note that for the 9 mg/kg Compound A single agent group, two animals were found dead after >15% body weight loss and are not included in the analysis. With the combination of 1 mg/kg Compound A+anti-PD-1 antibody, no additive anti-tumor activity was observed based on survival data. At Compound A days post treatment, 1 of 14 animals (7%) in the anti-PD-1 antibody group survived, while 0 of the animals in the 3 mg/kg single agent group survived. However, in the 3 mg/kg Compound A+anti-PD-1 antibody group, 2 of 14 animals (14%) survived up to Compound A days. As shown in FIG. 10, the combination of 6 mg/kg Compound A+anti-PD-1 antibody resulted in prolonged survival compared to each single agent alone. The median survival times were 21, 35, 24.5, and 49 days for the vehicle control, Compound A (6 mg/kg), anti-PD-1 antibody (10 mg/kg), and Compound A+anti-PD-1 antibody groups (6 mg/kg Compound A and 10 mg/kg anti-PD-1 antibody), respectively. The median survival time of the combination group was significantly longer than the Compound A and anti-PD-1 antibody (p<0.05) single agent treatment groups. Specifically, at Compound A days post treatment, 0 of the animals in the 6 mg/kg Compound A group survived while only 1 of 14 animals (7%) in the anti-PD-1 antibody group survived tumor-free. However, in the combination group, 5 of 14 (36%) animals survived tumor-free (p<0.05). Data in FIG. 10 represent Kaplan-Meier survival curves for treatment groups. *p<0.05 vs. vehicle control. ^{⊥}p<0.05 vs. anti-PD-1 antibody. #p<0.05 vs. Compound A.

### Example 12

Whole Blood Cytokine Release Assay

Human whole blood samples from 6 healthy donors were incubated with a serial titration of IL-2_P65[AzK_L1_PEG30kD]-1 (Compound B) or IL-2 alone or in combination with PEM (“Pembro” or pembrolizumab) or NIVO (nivolumab) for 24 h. Cytokines released from the whole blood after treatment into the supernatant were measured using the Meso Scale Discovery (MSD) U-plex kit for six analytes (IFN-γ, IL-4, IL-5, IL-6, IL-8, TNF-α).

### Protocol.

Blood in heparin tubes from 6 healthy donors were obtained from The Scripps Research Institute (TSRI; San Diego, Calif.) blood donor service. Samples were tested on the day of the collection; donors 1-3 on one day and donors 4-6 on another day.

Human whole blood was incubated with various concentrations of IL-2_P65[AzK_L1_PEG30kD]-1, and 90 μg/mL of pembrolizumab or 127 μg/mL of nivolumab (expected Cₘₐₓ values of clinical dose).

The whole blood was first diluted 2-fold with RPMI 1640 Medium. A volume of 180 μL of prediluted whole blood was seeded onto a 96-well tissue culture plate. Then, 20 μL of 10× final test concentration of compounds was added to the assay plate. Test conditions included a serial titration of IL-2_P65[AzK_L1_PEG30kD]-1 (4.5 μg/mL, 1.5 μg/mL, 0.8 μg/mL, 0.45 μg/mL, and 0.2 μg/mL) or IL-2 (0.8 μg/mL, 0.45 μg/mL, 0.2 μg/mL, 0.1 μg/mL, and 0.03 μg/mL) and a serial titration of either IL-2_P65[AzK_L1_PEG30kD]-1 or IL-2 plus 90 μg/mL PEM or 127 μg/mL NIVO. In addition, positive and negative controls were included to show the detection and specification of the assay: 100 μg/mL pre-coated Ultra LEAF mouse anti-human CD3 were used as the positive controls for the assay. 50 μg/mL pre-coated Ultra LEAF mouse IgG1, a are the isotype control for the above anti-human CD3. IL-2_P65[AzK_L1_PEG30kD]-1 formulation buffer only was a negative control for the assay. No treatment also served as a negative control for the assay.

After adding the compound(s) to the well, the blood was incubated at 37° C. After centrifugation of the samples, supernatant was collected 24 h post treatment and stored at −80° C. before analysis. The human cytokines (IFN-7, TNF-α, IL-8, IL-6, IL-5, IL-4) released from the assay were quantified with the MSD U-plex kit. The lowest limit of detection for the assay is listed in the table below. The detection limit of each plate is reported from the MSD software, Discovery workbench. Each plate has a slightly different detection limit.

### Materials

TABLE 23
Materials and sources used.

| Material | Vendor | Catalog # | Formulation | Stock |
|---|---|---|---|---|
| IL-2_P65 [AzK_L1_PEG30kD]-1 | Cytovance | N/A | Formulation | 2.18 |
| Pilot 2 #0590-164 | | | Buffer | mg/mL |
| Formulation Buffer: 10 mM | Cytovance | N/A | N/A | N/A |
| Histidine pH 6.0, 5% sorbitol, | | | | |
| 0.01% pollysorbate 80 | | | | |
| IL-2 | R&D | 202-IL-500 | 0.1M Acetic | 1 |
| | Systems | | Acid | mg/mL |
| Biolegend Ultra-LEAF ™ Purified | Biolegend | 300438 | PBS | 1 |
| anti-human CD3 (UCHT1) | | | | mg/mL |
| Biolegend Ultra-LEAF ™ Purified | Biolegend | 400166 | PBS | 1 |
| Mouse IgG1, κ Isotype Ctrl | | | | mg/mL |
| (MOPC-21) | | | | |
| Pembrolizumab (PEM) | Selleckchem | A200502 | PBS | 9.97 |
| | | | | mg/mL |
| Ultra-LEAF ™ Purified Human | Biolegend | 403702 | PBS | 1 |
| IgG4 Isotype Control | | | | mg/mL |
| Recombinant Antibody | | | | |
| U-plex biomarker Group 1 | Meso Scale | K15067L-2 | N/A | N/A |
| (human IFN-γ, TNF-α, IL-10, | Discovery) | | | |
| IL-6, IL-4, IL-5) | (MSD) | | | |
| Gibco ™ RPMI 1640 Medium, | Gibco ™ | 22400089 | | |
| HEPES | | | | |
| Falcon ™ Polystyrene | Corning | 353077 | | |
| Microplates, 96 wells, TC treated | | | | |

### Results.

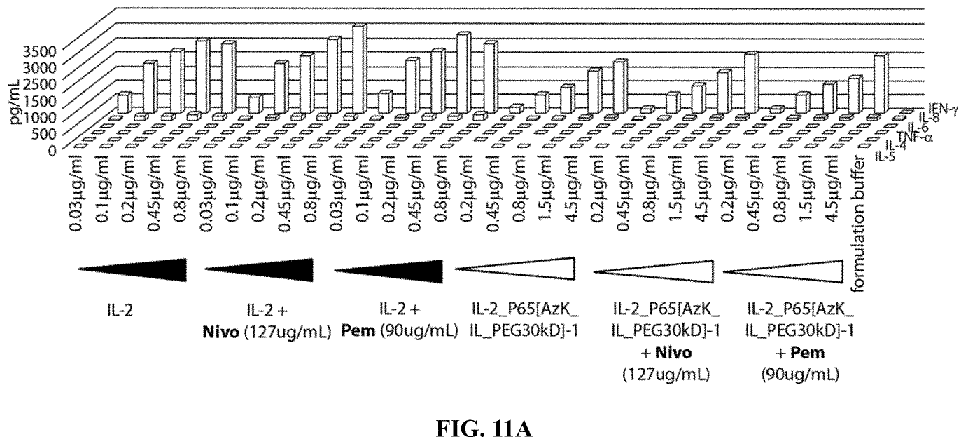

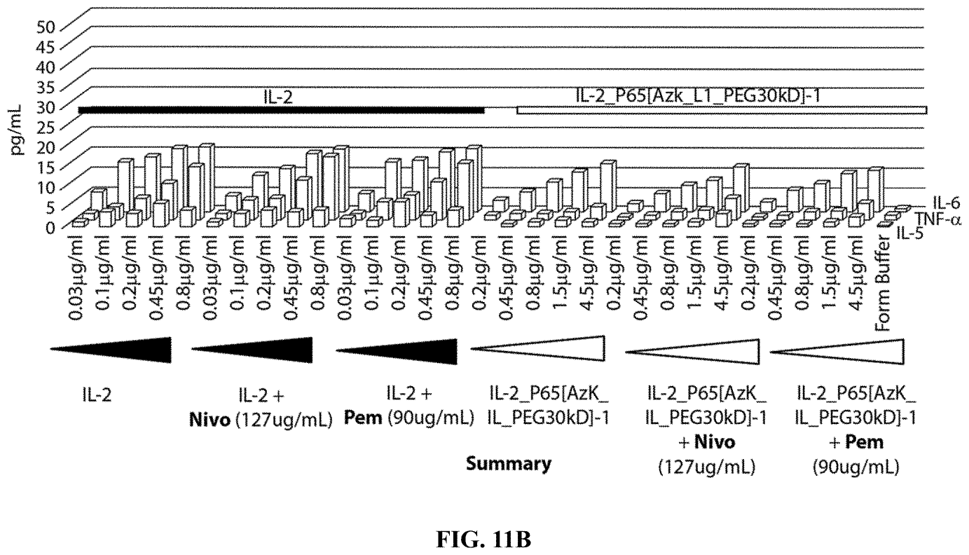

IL-2_P65[AzK_L1_PEG30kD]-1 and IL-2 induced a dose-dependent induction of IFN-γ at 24 hr treatment but did not induce significant release of the other 5 cytokines (IL4, IL5, IL8, TNFa, IL6) tested in the assay. In combination with either Pem or Nivo, both IL-2_P65[AzK_L1_PEG30kD]-1 and IL-2 (R&D) did not cause the significant change to the cytokine profile (IFN-γ, IL-4, IL-5, IL-8, TNF-α, IL-6) tested in the study. Data for individual donors is listed in Tables 24-32. Representative graphs for a single donor are shown in FIGS. 11A-11B.

TABLE 24
Control conditions for IFN-γ and IL-6 levels.

| | Conc. | IFN-γ | | | | | | IL-6 | | | | | |
|---|---|---|---|---|---|---|---|---|---|---|---|---|---|
| Treatment | ug/ml | D1 | D2 | D3 | D4 | D5 | D6 | D1 | D2 | D3 | D4 | D5 | D6 |
| Compound | n/a | 43.91 | 8.77 | 7.75 | 9.31 | 268.77 | 13.03 | 2.77 | 0.68 | 1.31 | 0.49 | 5.60 | 0.73 |
| B | | | | | | | | | | | | | |
| Formulation | | | | | | | | | | | | | |
| buffer | | | | | | | | | | | | | |
| no | n/a | 21.58 | 5.12 | 8.35 | 7.03 | 288.70 | 11.78 | 0.93 | 0.31 | 0.99 | 2.23 | 4.53 | 1.28 |
| treatment | | | | | | | | | | | | | |
| (culture | | | | | | | | | | | | | |
| media) | | | | | | | | | | | | | |
| IgG4 iso | 50 | 33.42 | 29.17 | 7.86 | 12.79 | 262.93 | 10.33 | 4.73 | 0.47 | 1.50 | 0.56 | 8.54 | 21.30 |
| control | | | | | | | | | | | | | |
| mouse | 100 | 21.75 | BDL | 7.19 | 6.44 | 94.74 | 132.02 | 1.64 | 0.88 | 0.89 | 0.71 | 4.27 | 1.85 |
| IgG1,κ | | | | | | | | | | | | | |
| PEM | 90 | 52.78 | 9.62 | 8.80 | 11.71 | 301.71 | 10.08 | 1.09 | 0.39 | 0.92 | 0.65 | 8.95 | 1.06 |
| NIVO | 127 | 29.77 | 9.34 | 6.61 | 7.56 | 256.75 | 9.51 | 1.28 | 0.48 | 1.09 | 0.59 | 3.29 | 0.62 |
| coated anti- | 100 | 13095.85 | 110278.12 | 2890.41 | 12415.49 | 46221.98 | 137695.00 | 279.20 | 106.57 | 20.18 | 66.79 | 452.34 | 1414.77 |
| CD3 | | | | | | | | | | | | | |

BDL = below detection limit.

TABLE 25
Control conditions for IL-5 and IL-8 levels.

| | Conc. | IL-8 | | | | | | TNF-α | | | | | |
|---|---|---|---|---|---|---|---|---|---|---|---|---|---|
| Treatment | ug/ml | D1 | D2 | D3 | D4 | D5 | D6 | D1 | D2 | D3 | D4 | D5 | D6 |
| Compound | n/a | 48.07 | 8.77 | 39.07 | 39.28 | 143.62 | 30.69 | 4.17 | 0.68 | 1.2 | 1 | 5.82 | 1.04 |
| B | | | | | | | | | | | | | |
| Formulation | | | | | | | | | | | | | |
| buffer | | | | | | | | | | | | | |
| no | n/a | 20.56 | 8.77 | 7.84 | 27.93 | 78.91 | 23.45 | 3.03 | 0.68 | 1.24 | 1.11 | 5.61 | 1.25 |
| treatment | | | | | | | | | | | | | |
| (culture | | | | | | | | | | | | | |
| media) | | | | | | | | | | | | | |
| IgG4 iso | 50 | 37.11 | 12.77 | 9.58 | 24.88 | 91.61 | BDL | 2.66 | 1.33 | 1.82 | 0.75 | 6.58 | 0.91 |
| control | | | | | | | | | | | | | |
| mouse | 100 | 20.37 | 7.42 | 7.92 | 22.37 | 57.12 | 24.8 | 1.42 | 0.27 | 0.14 | 0.69 | 3.44 | BDL |
| IgG1,κ | | | | | | | | | | | | | |
| PEM | 90 | 22.01 | 8.48 | 11.36 | 23.29 | 95.5 | 28.32 | 2.33 | 0.68 | 1.67 | 0.97 | 5.88 | 1.43 |
| NIVO | 127 | 20.59 | 7.68 | 7.95 | 22.61 | 67.38 | 19.68 | 2.35 | 0.44 | 1.33 | 1.21 | 4.89 | BDL |
| coated anti- | 100 | 5211.87 | 1780.7 | 1346.96 | 4031.78 | 5306.3 | 10117.33 | 475.92 | 2519.34 | 136.1 | 894.5 | 1187.77 | 3780.63 |
| CD3 | | | | | | | | | | | | | |

BDL = below detection limit.

TABLE 26
Control conditions for TNF-α and IL-4 levels.

| | Conc. | IL-5 | | | | | | IL-4 | | | | | |
|---|---|---|---|---|---|---|---|---|---|---|---|---|---|
| Treatment | ug/ml | D1 | D2 | D3 | D4 | D5 | D6 | D1 | D2 | D3 | D4 | D5 | D6 |
| Compound B | n/a | 0.71 | 0.06 | 0.59 | BDL | BDL | BDL | 0.05 | 0.06 | 0.08 | 0.09 | BDL | BDL |
| Formulation | | | | | | | | | | | | | |
| buffer | | | | | | | | | | | | | |
| no treatment | n/a | 0.81 | 0.14 | 0.87 | BDL | BDL | BDL | 0.11 | 0.06 | 0.11 | 0.05 | BDL | BDL |
| (culture media) | | | | | | | | | | | | | |
| IgG4 iso | 50 | 0.26 | 0.23 | 0.64 | BDL | 0.77 | BDL | 0.05 | 0.12 | BDL | 0.12 | BDL | BDL |
| control | | | | | | | | | | | | | |
| mouse | 100 | 0.66 | 0.09 | 0.56 | BDL | BDL | BDL | 0.04 | 0.14 | 0.16 | BDL | BDL | 0.09 |
| IgG1,κ | | | | | | | | | | | | | |
| PEM | 90 | 1.03 | 0.72 | 0.79 | BDL | BDL | BDL | 0.10 | 0.03 | 0.03 | 0.09 | BDL | BDL |
| NIVO | 127 | 2.65 | 1.50 | 0.91 | BDL | BDL | BDL | 0.11 | 0.11 | 0.05 | 0.10 | BDL | BDL |
| coated anti-CD3 | 100 | 34.49 | 308.57 | 7.64 | 31.81 | 137.92 | 656.77 | 11.33 | 47.81 | 1.33 | 11.65 | 18.59 | 82.57 |

BDL = below detection limit.

TABLE 27
Treatment with IL-2 on IFN-γ and IL-6 levels.

| Treat- | Conc. | IFN-γ | | | | | | IL-6 | | | | | |
|---|---|---|---|---|---|---|---|---|---|---|---|---|---|
| ment | μg/ml | D1 | D2 | D3 | D4 | D5 | D6 | D1 | D2 | D3 | D4 | D5 | D6 |
| IL-2 | 0.8 | 115.31 | 290.42 | 314.96 | 81.72 | 61.31 | 850.98 | 22.76 | 17.26 | 10.13 | 19.67 | 17.12 | 51.15 |
| 1L-2 | 0.45 | 105.72 | 352.81 | 331.98 | 85.34 | 58.16 | 433.00 | 18.47 | 11.07 | 7.20 | 12.82 | 18.02 | 65.04 |
| IL-2 | 0.2 | 104.20 | 228.19 | 278.17 | 104.40 | 51.97 | 437.67 | 15.93 | 5.48 | 4.12 | 6.96 | 12.56 | 49.09 |
| IL-2 | 0.1 | 95.61 | 135.05 | 224.27 | 68.31 | 47.44 | 387.76 | 13.48 | 2.66 | 2.73 | 4.02 | 10.94 | 28.98 |
| IL-2 | 0.03 | 64.49 | 62.13 | 81.74 | 45.09 | 39.50 | 170.85 | 7.89 | 1.07 | 1.35 | 2.31 | 9.18 | 16.60 |
| IL-2 + | 0.8 | 138.79 | 274.43 | 318.86 | 117.65 | 51.99 | 437.11 | 27.72 | 17.63 | 10.91 | 22.39 | 32.49 | 50.58 |
| Pembro | | | | | | | | | | | | | |
| IL-2 + | 0.45 | 107.58 | 247.26 | 354.82 | 111.66 | 55.99 | 430.47 | 23.19 | 11.71 | 7.45 | 31.52 | 15.11 | 55.85 |
| Pembro | | | | | | | | | | | | | |
| IL-2 + | 0.2 | 112.00 | 231.40 | 283.69 | 76.41 | 64.28 | 244.58 | 20.84 | 6.54 | 4.93 | 7.65 | 14.72 | 29.07 |
| Pembro | | | | | | | | | | | | | |
| IL-2 + | 0.1 | 99.87 | 151.13 | 241.42 | 106.24 | 50.17 | 274.30 | 16.91 | 3.10 | 3.44 | 13.06 | 13.48 | 28.87 |
| Pembro | | | | | | | | | | | | | |
| IL-2 + | 0.03 | 76.19 | 44.44 | 87.79 | 40.85 | 38.04 | 131.98 | 11.01 | 1.46 | 1.47 | 2.10 | 9.98 | 15.04 |
| Pembro | | | | | | | | | | | | | |
| IL-2 + | 0.8 | 112.83 | 245.07 | 396.28 | 67.50 | 53.02 | 344.33 | 24.21 | 17.31 | 12.10 | 19.46 | 16.29 | 50.22 |
| Nivo | | | | | | | | | | | | | |
| IL-2 + | 0.45 | 98.79 | 262.60 | 335.91 | 66.15 | 47.95 | 457.22 | 17.85 | 13.65 | 7.85 | 11.72 | 13.01 | 40.96 |
| Nivo | | | | | | | | | | | | | |
| IL-2 + | 0.2 | 101.09 | 204.58 | 260.76 | 59.56 | 46.18 | 238.28 | 17.54 | 6.37 | 4.23 | 6.38 | 13.08 | 30.47 |
| Nivo | | | | | | | | | | | | | |
| IL-2 + | 0.1 | 100.69 | 423.63 | 230.39 | 92.71 | 37.74 | 206.75 | 12.71 | 3.10 | 3.98 | 9.98 | 9.74 | 20.84 |
| Nivo | | | | | | | | | | | | | |
| IL-2 + | 0.03 | 72.59 | 40.13 | 76.36 | nd | 38.22 | 149.74 | 8.23 | 1.22 | 1.44 | 29.61 | 8.51 | 14.21 |
| Nivo | | | | | | | | | | | | | |

Data for each donor (e.g.. D1-D6 was normalized by its specific formulation buffer value.
nd = not determined.

TABLE 28
Treatment with IL-2 on IL-8 and IFN-α levels.

| | Conc. | IL-8 | | | | | | TNF-α | | | | | |
|---|---|---|---|---|---|---|---|---|---|---|---|---|---|
| Treatment | μg/ml | D1 | D2 | D3 | D4 | D5 | D6 | D1 | D2 | D3 | D4 | D5 | D6 |
| IL-2 | 0.8 | 5.88 | 6.00 | 4.53 | 2.28 | 5.80 | 25.94 | 5.97 | 14.38 | 13.84 | 4.88 | 16.45 | 24.31 |
| IL-2 | 0.45 | 4.67 | 5.98 | 4.98 | 2.02 | 5.32 | 36.29 | 5.04 | 14.83 | 13.27 | 4.48 | 16.22 | 37.41 |
| IL-2 | 0.2 | 4.34 | 5.42 | 4.01 | 1.88 | 4.89 | 27.24 | 5.10 | 12.68 | 11.74 | 4.18 | 15.14 | 26.06 |
| IL-2 | 0.1 | 3.80 | 5.00 | 3.76 | 1.60 | 4.44 | 17.45 | 4.60 | 11.07 | 10.68 | 3.16 | 12.89 | 16.94 |
| IL-2 | 0.03 | 2.70 | 2.50 | 1.42 | 1.38 | 3.95 | 11.11 | 3.52 | 3.88 | 4.45 | 2.55 | 10.86 | 12.96 |
| IL-2 + Pembro | 0.8 | 7.00 | 6.95 | 4.83 | 2.97 | 7.84 | 24.43 | 6.02 | 17.35 | 13.48 | 6.42 | 16.66 | 23.88 |
| IL-2 + Pembro | 0.45 | 5.77 | 6.41 | 4.43 | 3.71 | 5.86 | 27.94 | 5.46 | 17.44 | 12.60 | 6.33 | 15.51 | 25.44 |
| IL-2 + Pembro | 0.2 | 5.37 | 6.73 | 3.59 | 2.24 | 6.01 | 15.52 | 5.20 | 15.55 | 11.03 | 3.54 | 15.96 | 17.18 |
| IL-2 + Pembro | 0.1 | 5.39 | 4.85 | 3.08 | 2.73 | 5.47 | 18.57 | 4.85 | 11.49 | 10.45 | 5.06 | 13.80 | 16.99 |
| IL-2 + Pembro | 0.03 | 3.60 | 2.28 | 1.52 | 1.65 | 4.72 | 8.60 | 4.24 | 4.94 | 4.19 | 2.82 | 11.29 | 8.91 |
| IL-2 + Nivo | 0.8 | 6.66 | 6.26 | 4.31 | 2.02 | 6.22 | 26.11 | 5.71 | 17.11 | 13.39 | 4.52 | 14.18 | 26.91 |
| IL-2 + Nivo | 0.45 | 5.06 | 6.42 | 4.04 | 1.80 | 5.28 | 22.75 | 5.35 | 15.39 | 12.10 | 4.61 | 13.60 | 22.81 |
| IL-2 + Nivo | 0.2 | 4.09 | 4.96 | 3.16 | 1.49 | 4.82 | 19.00 | 4.97 | 11.83 | 9.17 | 3.23 | 12.57 | 17.17 |
| IL-2 + Nivo | 0.1 | 3.80 | 4.31 | 2.97 | 3.04 | 5.09 | 12.19 | 4.33 | 10.74 | 7.90 | 5.14 | 11.43 | 11.44 |
| IL-2 + Nivo | 0.03 | 2.28 | 1.88 | 1.03 | 2.86 | 3.65 | 8.55 | 3.35 | 4.04 | 3.60 | 5.41 | 10.15 | 9.42 |

Data for each donor (e.g., D1-D6) was normalized by its specific formulation buffer value.

TABLE 29
Treatment with IL-2 on IL-5 and IL-4 levels.

| | | IL-5 | | | | | | IL-4 | | | | | |
|---|---|---|---|---|---|---|---|---|---|---|---|---|---|
| Treatment | Conc. μg/ml | D1 | D2 | D3 | D4 | D5 | D6 | D1 | D2 | D3 | D4 | D5 | D6 |
| IL-2 | 0.8 | 9.45 | 6.04 | 4.34 | 0.71 | 10.86 | 22.27 | 0.25 | 0.21 | 0.21 | 0.13 | 0.85 | 1.02 |
| IL-2 | 0.45 | 10.57 | 11.00 | 5.85 | BDL | 15.40 | 22.08 | 0.14 | 0.34 | 0.32 | 0.08 | 0.68 | 0.89 |
| IL-2 | 0.2 | 14.53 | 8.79 | 3.46 | 0.93 | 12.29 | 28.22 | 0.30 | 0.42 | 0.28 | 0.10 | 0.77 | 1.05 |
| IL-2 | 0.1 | 5.24 | 6.79 | 3.85 | 0.26 | 9.57 | 23.02 | 0.21 | 0.32 | 0.14 | 0.09 | 0.75 | 0.83 |
| IL-2 | 0.03 | 7.56 | 3.73 | 1.35 | 0.66 | 22.70 | 13.05 | 0.27 | 0.18 | 0.15 | 0.14 | 0.74 | 0.52 |
| IL-2 + Pembro | 0.8 | 5.62 | 8.14 | 4.45 | 1.55 | 14.17 | 24.07 | 0.24 | 0.35 | 0.33 | 0.28 | 0.78 | 1.15 |
| IL-2 + Pembro | 0.45 | 4.54 | 7.16 | 3.00 | 0.93 | 9.82 | 21.83 | 0.16 | 0.39 | 0.27 | 0.26 | 0.70 | 1.20 |
| IL-2 + Pembro | 0.2 | 8.95 | 11.58 | 6.50 | 0.56 | 12.53 | 15.88 | 0.31 | 0.31 | 0.47 | 0.12 | 0.82 | 0.76 |
| IL-2 + Pembro | 0.1 | 7.42 | 7.03 | 1.90 | BDL | 8.53 | 23.41 | 0.21 | 0.27 | 0.36 | 0.12 | 0.64 | 0.76 |
| IL-2 + Pembro | 0.03 | 5.87 | 2.86 | 2.05 | 0.42 | 14.25 | 8.53 | 0.17 | BDL | 0.13 | 0.08 | 0.82 | 0.51 |
| IL-2 + Nivo | 0.8 | 7.16 | 7.98 | 4.18 | 1.38 | 20.46 | 20.83 | 0.22 | 0.25 | 0.44 | 0.14 | 1.00 | 1.21 |
| IL-2 + Nivo | 0.45 | 7.14 | 7.05 | 4.22 | BDL | 11.55 | 18.52 | 0.21 | 0.31 | 0.28 | 0.17 | 0.54 | 0.88 |
| IL-2 + Nivo | 0.2 | 7.32 | 7.45 | 4.18 | 1.97 | 12.97 | 20.73 | 0.21 | 0.34 | 0.30 | 0.25 | 0.74 | 0.75 |
| IL-2 + Nivo | 0.1 | 8.67 | 5.37 | 3.43 | 3.04 | 10.46 | 16.09 | 0.26 | 0.21 | 0.18 | 0.14 | 0.61 | 0.62 |
| IL-2 + Nivo | 0.03 | 3.77 | 2.02 | 1.37 | BDL | 11.82 | 12.10 | 0.17 | BDL | 0.11 | 0.19 | 0.74 | 0.63 |

BDL = below detection limit.

TABLE 30
Treatment with Compound B (IL-2 P65[AzK_L1_PEG30kD]-1) on IFN-γ and IL-6 levels.

| | Conc. | IFN-γ | | | | | | IL-6 | | | | | |
|---|---|---|---|---|---|---|---|---|---|---|---|---|---|
| Treatment | (μg/ml) | D1 | D2 | D3 | D4 | D5 | D6 | D1 | D2 | D3 | D4 | D5 | D6 |
| Compound | 4.5 | 213.24 | 135.44 | 233.77 | 75.99 | 36.04 | 611.22 | 39.77 | 0.56 | 2.49 | 4.94 | 10.61 | 38.95 |
| B | | | | | | | | | | | | | |
| Compound | 1.5 | 93.02 | 96.56 | 195.89 | 49.83 | 31 | 122.98 | 11.82 | 0.84 | 1.82 | 1.87 | 8.33 | 16.01 |
| B | | | | | | | | | | | | | |
| Compound | 0.8 | 66.82 | 53.46 | 119.28 | 44.78 | 26.93 | 92.69 | 9.38 | 0.43 | 1.45 | 1.71 | 7.56 | 11.61 |
| B | | | | | | | | | | | | | |
| Compound | 0.45 | 49.07 | 35.17 | 85.18 | 33.58 | 21.99 | 74.08 | 7.87 | 0.74 | 1.06 | 1.29 | 6.06 | 3.41 |
| B | | | | | | | | | | | | | |
| Compound | 0.2 | 32.02 | 12.82 | 32.15 | 27.63 | 14.39 | 21.95 | 4.50 | BDL | 0.91 | 1.11 | 3.84 | 1.09 |
| B | | | | | | | | | | | | | |
| Compound | 4.5 | 102.42 | 164.69 | 263.89 | 68.16 | 38.09 | 275.34 | 15.12 | 1.79 | 3.26 | 3.03 | 10.86 | 36.39 |
| B + | | | | | | | | | | | | | |
| Pembro | | | | | | | | | | | | | |
| Compound | 1.5 | 80.56 | 82.28 | 161.63 | 49.21 | 35.01 | 98.36 | 11.90 | 1.29 | 1.96 | 3.01 | 11.07 | 9.88 |
| B + | | | | | | | | | | | | | |
| Pembro | | | | | | | | | | | | | |
| Compound | 0.8 | 74.09 | 43.39 | 130.28 | 38.2 | 29.55 | 62.73 | 12.28 | 0.82 | 1.71 | 1.31 | 9.23 | 6..49 |
| B + | | | | | | | | | | | | | |
| Pembro | | | | | | | | | | | | | |
| Compound | 0.45 | 46.77 | 27.81 | 86.44 | 31.51 | 19.78 | 37.93 | 7.53 | 0.46 | 1.17 | 1.52 | 6.70 | 1.78 |
| B + | | | | | | | | | | | | | |
| Pembro | | | | | | | | | | | | | |
| Compound | 0.2 | 29.43 | 8.62 | 25.97 | 20.27 | 14.52 | 17.75 | 5.59 | BDL | 0.74 | 0.93 | 5.04 | 0.86 |
| B + | | | | | | | | | | | | | |
| Pembro | | | | | | | | | | | | | |
| Compound | 4.5 | 91.59 | 126.64 | 269.98 | 59.66 | 38.57 | 901.52 | 11.56 | 1.33 | 4.23 | 2.83 | 9.24 | 34.57 |
| B + Nivo | | | | | | | | | | | | | |
| Compound | 1.5 | 82.94 | 71.26 | 185.79 | 50.31 | 30.37 | 210.47 | 10.75 | 1.04 | 1.97 | 1.40 | 7.14 | 11.44 |
| B + Nivo | | | | | | | | | | | | | |
| Compound | 0.8 | 59.41 | 67.83 | 124.17 | 30.37 | 24.23 | 41.88 | 7.60 | 7.56 | 1.56 | 0.87 | 6.38 | 2.36 |
| B + Nivo | | | | | | | | | | | | | |
| Compound | 0.45 | 51.93 | 21.19 | 84.35 | 26.44 | 18.76 | 30.17 | 7.73 | 0.48 | 1.17 | 1.33 | 4.39 | 2.01 |
| B + Nivo | | | | | | | | | | | | | |
| Compound | 0.2 | 32.55 | 7 | 25.07 | 16.26 | 12.1 | 18.32 | 4.57 | BDL | 0.71 | 1.06 | 2.59 | 0.84 |
| B + Nivo | | | | | | | | | | | | | |

“Compound B” is IL-2 P65[AzK_L1_PEG30kD]-1. Data for each donor (e.g., D1-D6) was normalized by its specific formulation buffer value.
BDL = below detection limit.

TABLE 31
Treatment with Compound B (IL-2_P65[AzK_L1_PEG30kD]-1) on IL-8 and TNF-α levels.

| | Conc. | IL-8 | | | | | | TNF-α | | | | | |
|---|---|---|---|---|---|---|---|---|---|---|---|---|---|
| Treatment | (μg/ml) | D1 | D2 | D3 | D4 | D5 | D6 | D1 | D2 | D3 | D4 | D5 | D6 |
| Compound | 4.5 | 7.67 | 4.09 | 2.99 | 2.18 | 4.24 | 20.12 | 6.23 | 11.39 | 10.42 | 4.96 | 12.33 | 26.03 |
| B | | | | | | | | | | | | | |
| Compound | 1.5 | 2.83 | 3.87 | 3.09 | 1.66 | 3.64 | 11.46 | 4.43 | 8.33 | 8.80 | 3.30 | 10.85 | 13.94 |
| B | | | | | | | | | | | | | |
| Compound | 0.8 | 3.08 | 2.86 | 2.20 | 1.36 | 3.30 | 6.84 | 4.16 | 6.48 | 6.68 | 2.92 | 9.94 | 8.02 |
| B | | | | | | | | | | | | | |
| Compound | 0.45 | 2.21 | 2.61 | 1.43 | 1.42 | 2.69 | 3.50 | 3.56 | 5.28 | 4.56 | 3.12 | 8.20 | 5.30 |
| B | | | | | | | | | | | | | |
| Compound | 0.2 | 1.48 | 1.60 | 0.71 | 1.27 | 2.22 | 1.30 | 2.64 | 3.10 | 2.84 | 2.24 | 6.23 | 2.00 |
| B | | | | | | | | | | | | | |
| Compound | 4.5 | 4.19 | 4.64 | 2.61 | 2.13 | 4.65 | 21.72 | 5.10 | 11.12 | 8.91 | 4.49 | 12.42 | 20.50 |
| B + Pembro | | | | | | | | | | | | | |
| Compound | 1.5 | 4.10 | 3.46 | 2.79 | 2.66 | 4.45 | 15.57 | 4.87 | 8.68 | 8.24 | 3.80 | 12.07 | 15.39 |
| B + Pembro | | | | | | | | | | | | | |
| Compound | 0.8 | 3.36 | 2.80 | 2.10 | 1.53 | 4.13 | 5.51 | 4.41 | 6.88 | 6.21 | 3.15 | 10.56 | 6.04 |
| B + Pembro | | | | | | | | | | | | | |
| Compound | 0.45 | 2.35 | 2.19 | 1.58 | 1.69 | 3.21 | 2.09 | 3.51 | 4.09 | 4.89 | 2.52 | 8.33 | 3.46 |
| B + Pembro | | | | | | | | | | | | | |
| Compound | 0.2 | 1.66 | 1.41 | 0.62 | 1.20 | 2.46 | 1.23 | 2.97 | 2.59 | 2.41 | 2.69 | 6.18 | 2.09 |
| B + Pembro | | | | | | | | | | | | | |
| Compound | 4.5 | 3.97 | 4.16 | 3.22 | 1.63 | 4.05 | 16.97 | 5.36 | 9.71 | 9.57 | 3.83 | 12.26 | 21.81 |
| B + Nivo | | | | | | | | | | | | | |
| Compound | 1.5 | 2.86 | 2.62 | 2.16 | 1.42 | 3.70 | 6.78 | 4.84 | 6.45 | 7.11 | 3.15 | 10.53 | 9.76 |
| B + Nivo | | | | | | | | | | | | | |
| Compound | 0.8 | 2.97 | 2.55 | 1.97 | 1.19 | 3.56 | 1.99 | 3.93 | 5.33 | 5.87 | 2.87 | 8.88 | 3.17 |
| B + Nivo | | | | | | | | | | | | | |
| Compound | 0.45 | 2.15 | 1.72 | 1.19 | 1.10 | 2.53 | 2.05 | 3.66 | 3.88 | 3.94 | 2.86 | 7.22 | 3.38 |
| B + Nivo | | | | | | | | | | | | | |
| Compound | 0.2 | 1.45 | 1.30 | 0.59 | 0.91 | 1.86 | 0.90 | 2.67 | 2.35 | 2.21 | 1.88 | 4.85 | 2.31 |
| B + Nivo | | | | | | | | | | | | | |

“Compound B” is IL-2_P65[AzK_L1_PEG30kD]-1. Data for each donor (e.g., D1-D6) was normalized by its specific formulation buffer value.

TABLE 32
Treatment with Compound B (IL-2_P65[AzK_L1_PEG30kD]-1) on IL-5 and IL-4 levels.

| | Conc. | IL-5 | | | | | | IL-4 | | | | | |
|---|---|---|---|---|---|---|---|---|---|---|---|---|---|
| Treatment | (μg/ml) | D1 | D2 | D3 | D4 | D5 | D6 | D1 | D2 | D3 | D4 | D5 | D6 |
| Compound B | 4.5 | 6.39 | 6.87 | 1.39 | 0.91 | 8.54 | 31.80 | 0.18 | 0.12 | 0.18 | 0.21 | 0.44 | 1.07 |
| Compound B | 1.3 | 3.73 | 5.22 | 1.57 | 1.02 | 8.15 | 12.23 | 0.20 | 0.13 | 0.16 | 0.14 | 0.60 | 0.43 |
| Compound B | 0.8 | 8.67 | 2.03 | 1.51 | BDL | 3.16 | 4.73 | 0.20 | BDL | BDL | 0.06 | 0.20 | 0.25 |
| Compound B | 0.45 | 3.63 | 1.17 | 1.03 | BDL | 1.26 | 5.18 | 0.08 | 0.14 | 0.10 | 0.11 | 0.29 | 0.25 |
| Compound B | 0.2 | 0.66 | BDL | BDL | BDL | 0.31 | 0.27 | 0.10 | 0.08 | 0.12 | 0.07 | 0.04 | BDL |
| Compound B +Pembro | 4.5 | 7.22 | 8.02 | 2.77 | 0.74 | 9.13 | 27.48 | 0.26 | 0.30 | 0.20 | 0.14 | 0.59 | 1.10 |
| Compound B +Pembro | 1.5 | 3.91 | 4.10 | 1.36 | BDL | 7.45 | 5.54 | 0.21 | 0.13 | 0.16 | 0.09 | 0.59 | 0.41 |
| Compound B +Pembro | 0.8 | 4.74 | 6.45 | 0.92 | BDL | 5.71 | 2.69 | 0.18 | 0.10 | 0.11 | 0.11 | 0.48 | 0.15 |
| Compound B +Pembro | 0.45 | 2.54 | 0.59 | 0.82 | BDL | 2.69 | BDL | 0.17 | 0.08 | BDL | 0.07 | 0.30 | 0.08 |
| Compound B +Pembro | 0.2 | 0.84 | BDL | 0.71 | BDL | 0.33 | 0.35 | 0.14 | BDL | BDL | 0.09 | 0.03 | BDL |
| Compound B +Nivo | 4.5 | 4.76 | 5.65 | 3.54 | 0.20 | 9.21 | 20.27 | 0.23 | 0.27 | 0.24 | 0.15 | 0.74 | 1.00 |
| Compound B +Nivo | 1.5 | 3.66 | 2.11 | 1.41 | 0.36 | 9.57 | 5.42 | 0.20 | 0.09 | 0.17 | 0.16 | 0.69 | 0.30 |
| Compound B +Nivo | 0.8 | 3.20 | BDL | 0.90 | BDL | 3.96 | 1.10 | 0.21 | BDL | 0.14 | 0.09 | 0.34 | 0.07 |
| Compound B +Nivo | 0.45 | 5.11 | BDL | 1.03 | 0.11 | 2.08 | 1.06 | 0.16 | BDL | 0.13 | 0.08 | 0.20 | 0.07 |
| Compound B +Nivo | 0.2 | 2.83 | 0.60 | 0.73 | BDL | 1.97 | BDL | 0.10 | 0.14 | BDL | 0.05 | 0.07 | BDL |

“Compound B” is IL-2_P65[AzK_L1_PEG30kD]-1.
BDL = below detection limit.

### Example 13

Allogeneic Human Mixed Lymphocyte Reaction (MLR) Assay

The allogeneic human mixed lymphocyte reaction (MLR) assay was used to evaluate the ability of Compound B (IL-2_P65[AzK_L1_PEG30kD]-1) to enhance TCR activation as a single agent and in combination with a checkpoint inhibitor (nivolumab or pembrolizumab), as a model for cytolytic response to tumor cells presenting specific antigens or neoantigens.

TABLE 33
Materials.

| | | | | Stock |
|---|---|---|---|---|
| Material | Vendor | Catalog # | Formulation | Concentration |
| Compound B (IL- | Synthorx | Lot | Formulation | 2 mg/mL |
| 2_P65 [AzK_L1_PEG30kD]-1) | | #0590- | Buffer | |
| | | 187 | (see below) | |
| Formulation Buffer: 10 mM | Cytovance | N/A | N/A | N/A |
| histidine pH 6.0, 5% sorbitol, | | | | |
| 0.01% polysorbate 80 | | | | |
| Nivolumab (nivo) | Selleckchem | A2002 | PBS | 5 mg/mL |
| Nivolumab | Bristol-Meyers | NDC | N/A | 10 mg/mL |
| | Squibb | 0003- | | |
| | | 3772-11 | | |
| Pembrolizumab | Merck & Co. | NDC | N/A | 25 mg/mL |
| | | 0006- | | |
| | | 3026-01 | | |
| Ultra-LEAF ™ Purified | Biolegend | 403702 | PBS | 1 mg/mL |
| Human IgG4 Isotype Control | | | | |
| Recombinant Antibody | | | | |
| EasySep human CD4 + | Stemcell | 17952 | N/A | N/A |
| T cell isolation kit | | | | |
| EasySep human | Stemcell | 19359 | N/A | N/A |
| monocyte isolation kit | | | | |
| Human IFN-γ ELBA kit | Abcam | Ab46025 | N/A | N/A |

TABLE 34
Materials, cont'd.

| | | | | |
|---|---|---|---|---|
| | | | Stock | Working |
| | | | Concen- | Concen- |
| Ingredient | Vendor | Catalog # | tration | tration |

MonoDC trans-differentiation media

| | | | | |
|---|---|---|---|---|
| BioWhittaker | Lonza | 04418Q | N/A | N/A |
| X-VIVO15 | | | | |
| hematopoietic | | | | |
| serum free | | | | |
| culture medium | | | | |
| FBS | Corning | 35011CV | 100% | 5% |
| Penicillin- | Fisher | 15140122 | 100% | 1% |
| Streptomycin | Scientific | | | |
| MEM Non- | Gibco | 11140-050 | 100x | 1x |
| Essential Amino | | | | |
| Acids Solution | | | | |
| Sodium pyruvate | Gibco | 11360-070 | 100 mM | 1 mM |
| HEPES | Gibco | 15630080 | 1 M | 10 mM |
| Recombinant | R&D | 204-IL-050 | 1.5e + | 1500 |
| human IL-4 | | | 6 IU/mL | IU/mL |
| protein (/CF) | | | | |
| Recombinant | R&D | 215-GM-050 | 1.5e + | 1500 |
| humanGM-CSF | | | 6 IU/mL | IU/mL |
| protein (/CF) | | | | |

MLR co-culture media

| | | | | |
|---|---|---|---|---|
| RPMI 1640 | Gibco | 22400089 | N/A | N/A. |
| medium, | | | | |
| HEPES | | | | |
| FBS | Corning | 35011CV | 100% | 5% |
| Penicillin- | Fisher | 15140122 | 100% | 1% |
| Streptomycin | Scientific | | | |
| MEM Non- | Gibco | 11140-050 | 100x | 1x |
| Essential | | | | |
| amino acids | | | | |
| solution | | | | |
| Sodium pyruvate | Gibco | 11360-070 | 100 mM | 1 mM |
| HEPES | Gibco | 15630080 | 1 M | 10 mM |
| 2-Mercapto- | Gibco | 21985023 | 50 mM | 50 μM |
| ethanol | | | | |

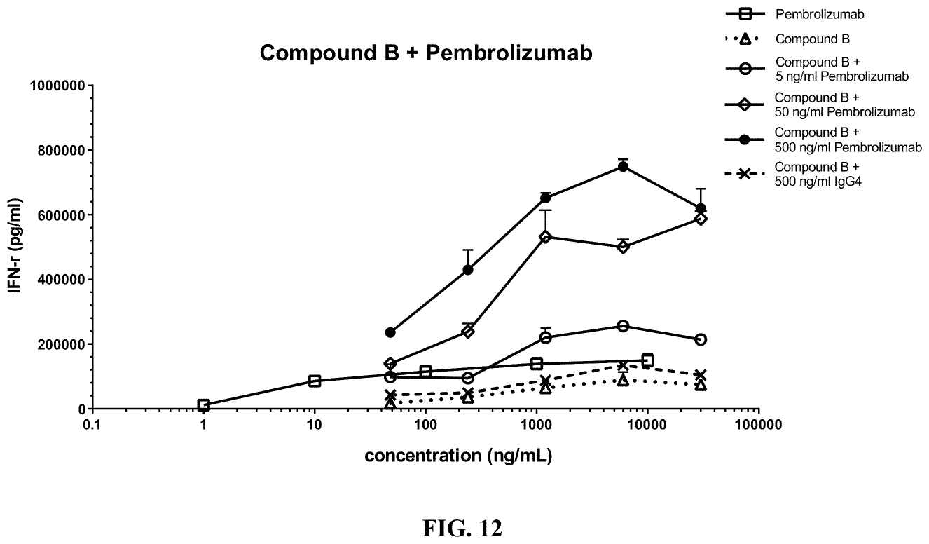

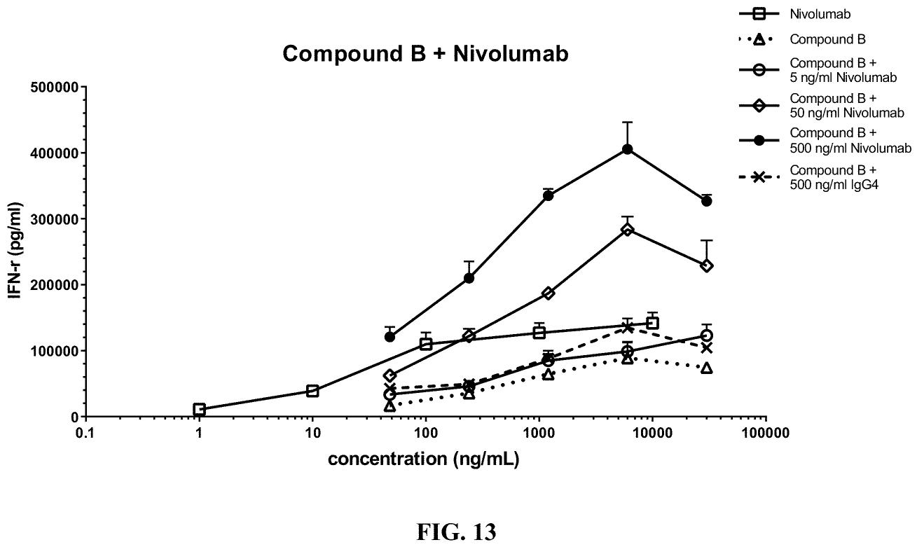

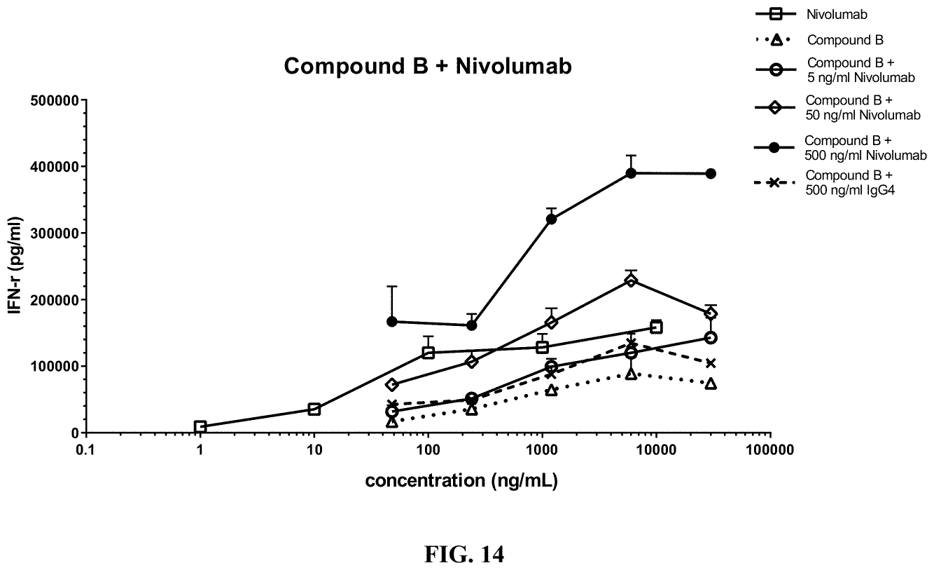

Peripheral blood mononuclear cells (PBMCs) from two normal, healthy donors were used for each assay run. MonoDCs were generated by culturing monocytes isolated from PBMCs using a negative monocyte selection kit (Stemcell) in vitro for 7 days, involving addition of 1,500 IU/mL of IL-4 and 1,500 IU/mL of GM-CSF. The culture medium was replaced on Days 3 and 5. MonoDCs were collected on Day 7. On that day, CD4+ T cells were isolated from a different donor with a negative selection kit (Stemcell). CD4+ T cells (1×105) and allogeneic monoDC (1×104) were co-cultured in 96-well microtiter plates, in the presence of a concentration range of Compound B (0.005-100 μg/mL) alone, or with 5, 50, or 500 ng/mL of nivolumab, pembrolizumab or isotype IgG control. Each run was set up in 3 replicates for each treatment condition. After 5 days of co-culture, IFN-γ secretion in culture supernatants was analyzed employing an enzyme-linked immunosorbent assay (ELISA) kit (Abcam; catalog #Ab46025). IFN-γ levels from the combination of Compound B and pembrolizumab are shown in FIG. 12. IFN-γ levels from the combination of Compound B and nivolumab are shown in FIGS. 13 and 14. Clinical grade nivolumab was used to generate the data shown in FIG. 13, and research grade nivolumab (Selleckchem) was used to generate the data shown in FIG. 14. The results demonstrated that Compound B, nivolumab and pembrolizumab, used as single agents, induced the release of IFN-γ in a concentration-dependent manner. Surprisingly, the combination of (a) Compound B and nivolumab, and (b) Compound B and pembrolizumab demonstrated a synergistic effect in the MLR assay. Data shown in FIGS. 12, 13, and 14 represent the mean of 3 replicates standard error of the mean from one donor pair.

### Example 14

Compound B Induces the Expression of Ki67 in CD8+ T, NK, and Treg Cells but Only Expands CD8+ T and NK Cells and not Tregs in Peripheral Blood and within CT-26 Tumors

To evaluate the pharmacokinetic (PK) and pharmacodynamic (PD) properties of Compound B, Balb/c female mice (6-8 weeks of age with an average weight of 16 to 22 g, Jackson Laboratories or Taconic Biosciences) were inoculated subcutaneously in the flank region with CT-26 tumor cells (ATCC). Tumor growth was monitored by measuring the tumors, three times a week. When the tumor volumes reached approximately 150 mm³, mice were randomized into control and treatment groups. Following IV administration of a single dose of Compound B at 3 mg/kg to CT-26 tumor-bearing mice, terminal blood and tumor samples were collected on day 0 (2 h, 8 h, and 12 h), day 1 (N=3 mice per time point), day 2, 3, 5, 7, 10, and 12 days post-dose (N=4 mice per time point). Plasma and tumor samples were analyzed for Compound B using an ELISA assay.

### Pharmacokinetic Analysis

The tumor was separated into two halves, one half was weighed and frozen down in liquid nitrogen for tumor PK analysis. Frozen tumor samples were homogenized with the lysis buffer (1 tablet of protease inhibitor (SIGMA, catalog #4693159001) in 10 mL 1× PBS). Every 0.1 g tissue was mixed with 0.4 mL of lysis buffer. To each tissue collection tube, a 5 mm stainless steel bead was added (Qiagen, catalog #69989) prior to homogenization with Tissue Lyser II (Qiagen) at 20 Hz for 20 sec. Following homogenization, the tumor lysate was spun down, and the supernatant was collected for Compound B PK analysis.

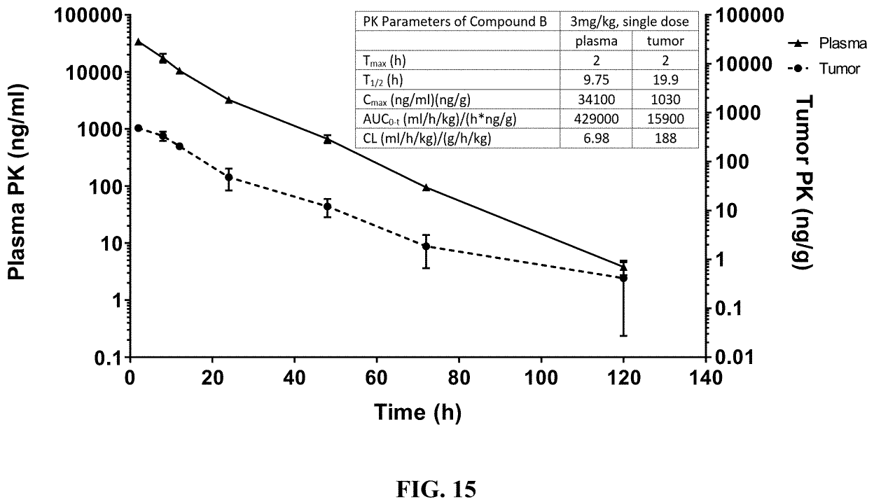

Tumor exposure of Compound B was approximately 4% of plasma exposure (AUC₀₋₄ 429,000 and 15,900 h·ng/mL for plasma and tumor, respectively) as shown in FIG. 15. Tumor t_{1/2} was nearly twice that of plasma t_{1/2}(19.9 h and 9.8 h in tumor and plasma, respectively) indicating that Compound B distributes into the tumor and is retained there for a longer time relative to the blood compartment.

### Pharmacodynamic Analysis

Flow Cytometry. For whole blood immune cell phenotyping, blood samples were lysed and fixed immediately following terminal collection. Briefly, blood samples were treated with 20 volumes of pre-warmed 1× lyse/fix buffer (BD phosflow™, cat #558049) according to the manufacturer's protocol. Cell suspensions were blocked with Fc block (TruStain fc-X anti CD16/32, BioLegend) before antibody staining. After blocking, cells were first stained with the following cell surface markers: Ax488 anti-mouse CD3 (17A2), Bv786 anti-mouse CD4 (RM4-5), Bv711 anti-mouse CD8a (53-6.7), Bv421 anti-mouse CD49b (DX5), biotin anti-mouse CD25 (REA568), Bv605 anti-mouse CD335 (29A1.4). Cells were then permeabilized with 4° C. pre-cooled methanol (Fisher Chemical, A412-4) and stained internally with PE anti-mouse FoxP3 (FJK-16s), PerCP eFluor710 anti-Ki67, and Ax647 anti-Pstat5 (Py694), as well as with PEcy7 anti-CD44 (IM7) and BUV395 Streptavidin for biotin. Samples were read utilizing BD LSRFortessa, with analysis by FlowJo software.

CD8 cells were identified as CD3+CD8a+. Treg cells were identified as CD3+CD4+CD25+FoxP3+. Natural killer cells were gated with CD3−CD335+CD49b+. Memory CD8 cells were assessed with CD3+CD8+CD44^{hi}.

Tumor FACS. A single cell suspension of mouse tumor samples was prepared by mincing tumors into small pieces and digesting with MACS mouse tumor dissociation kit (Miltenyi, 130-096-730) according to the manufacturer's protocol. Live cells were identified by eFluor 780 fixable viability dye (eBioscience, 65-0865-14). Cell suspensions were blocked with anti-mouse CD16/32 antibody (TruStain FcX, BioLegend, cat. 101319), followed by cell surface marker staining. Then cells were fixed and permeabilized with FoxP3/Transcription factor fixation/permeabilization reagent (eBioscience, cat. 00-5521-00), and then stained for intracellular marker. Antibodies used for surface antigens were PEcy7 anti-mouse CD45 (30-F11), BUV395 anti-mouse CD3e (17A2), BV510 anti-mouse CD4 (GK1.5), PE-eF610 CD8a (53-6.7), BV605 anti-mouse CD335 (29A1.4), AF700 anti-mouse CD25 (PC61), APC anti-mouse CD49b (DX5). Antibodies for intracellular antigens were PE anti-FoxP3 (FJK-16s) and AF488 anti-Ki67 (11F6). The CD8 cell population was identified as CD3+CD8+, while the NK cell population was defined as CD3−CD335+CD49b+. Treg cells were gated with CD3+CD4+CD25+FoxP3+. Events were acquired with BD LSRFortessa, and analyzed by FlowJo software.

Results. Blood and tumor samples were analyzed for PD readouts in cell subpopulations (CD8+T, NK, and Treg cells) including intracellular phosphorylated STAT5 (pSTAT5, a marker of receptor occupancy and early signaling), Ki67 (a cell proliferation marker), and CD8+T, NK, and Treg cell counts. pSTAT5 on the various cell types was measured only in blood.

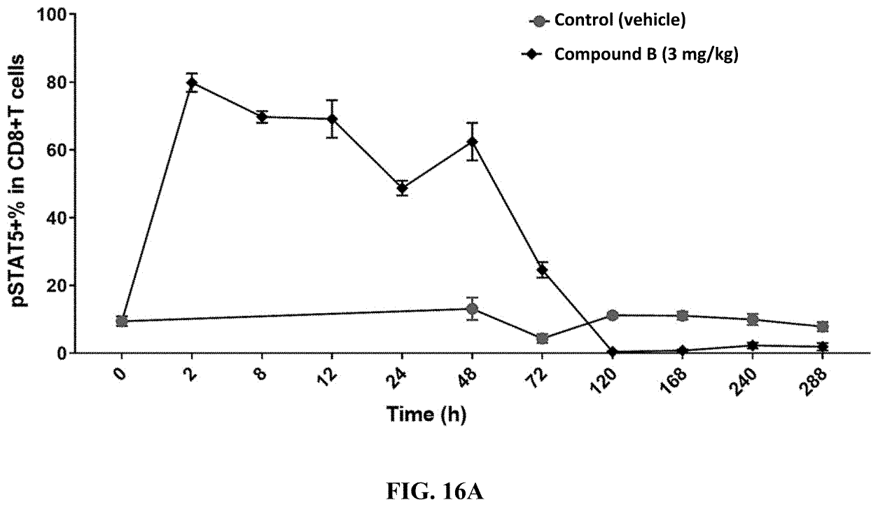

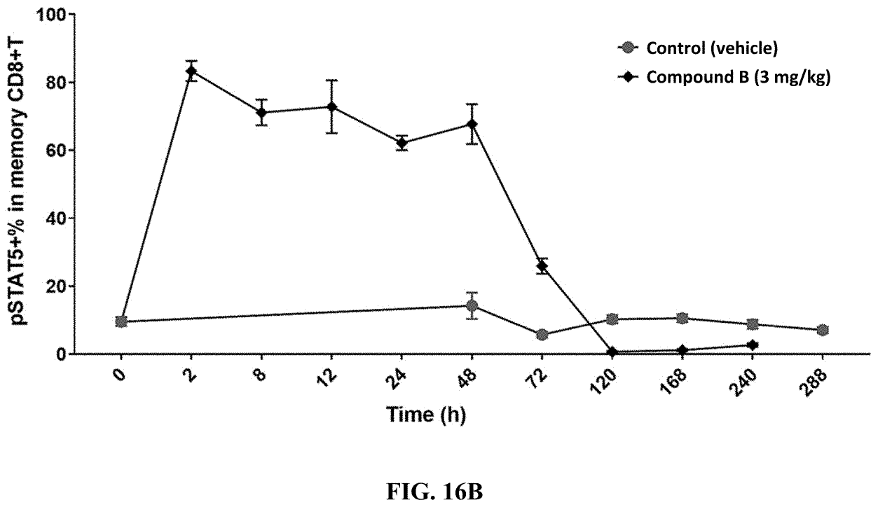

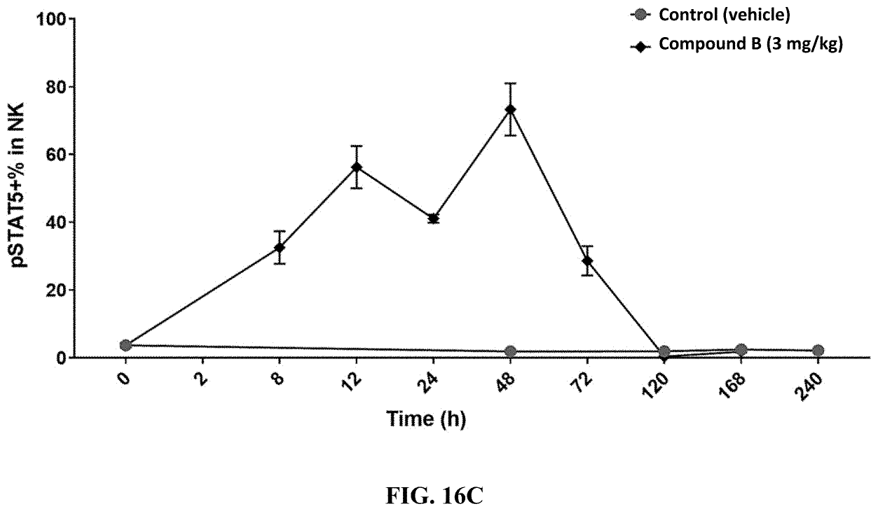

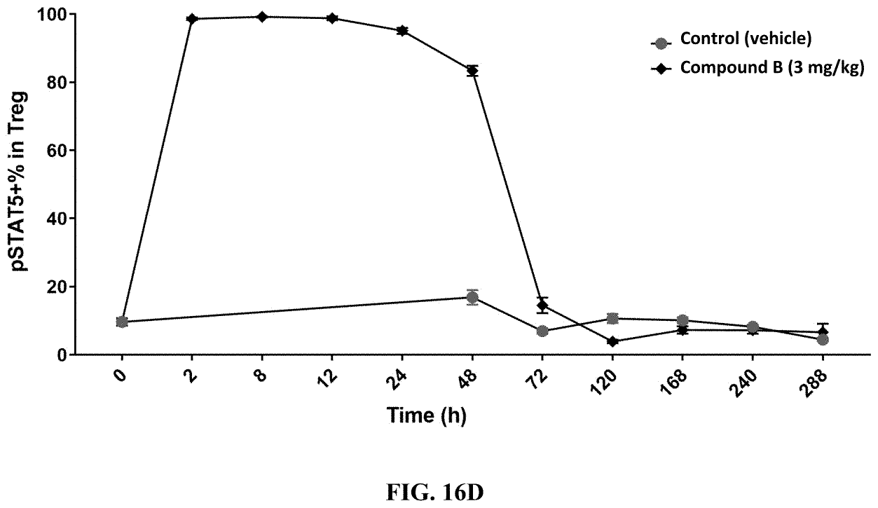

CT-26 tumor-bearing mice dosed with 3 mg/kg of Compound B showed persistent induction of pSTAT5 in peripheral blood in CD8+T, CD8+ memory T, NK and Treg cell populations. Percentage of pSTAT5+ cells in peripheral blood CD8+ T cells (FIG. 16A) and CD8+ memory T cells (FIG. 16B) peaked at 2 h post-dosing and remained elevated to approximately 48 h, and returned to baseline by 72 h. With the NK cells (FIG. 16C), the pSTAT5+ cells gradually increased post-dose to peak at 48 h and returned to baseline by 120 h. The induction of pSTAT5+ in Treg cells (FIG. 16D) followed a similar pattern to that of CD8+ T cells.

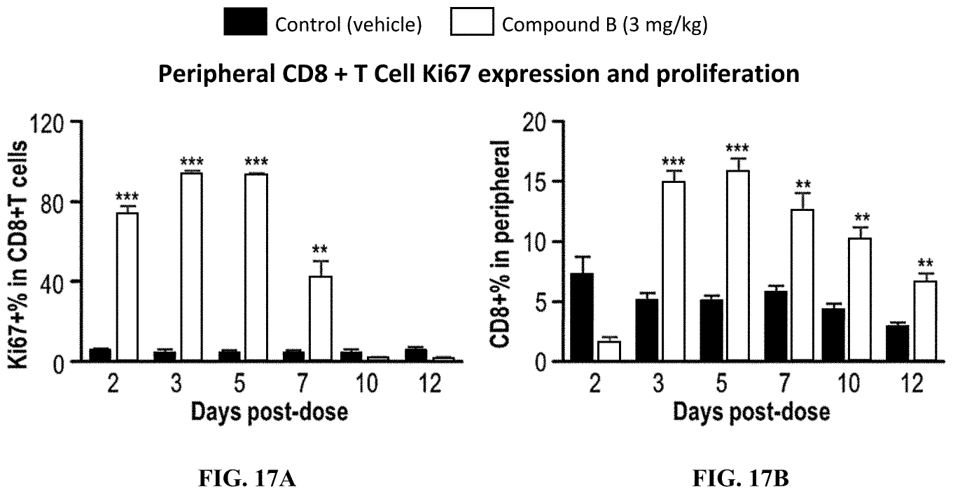

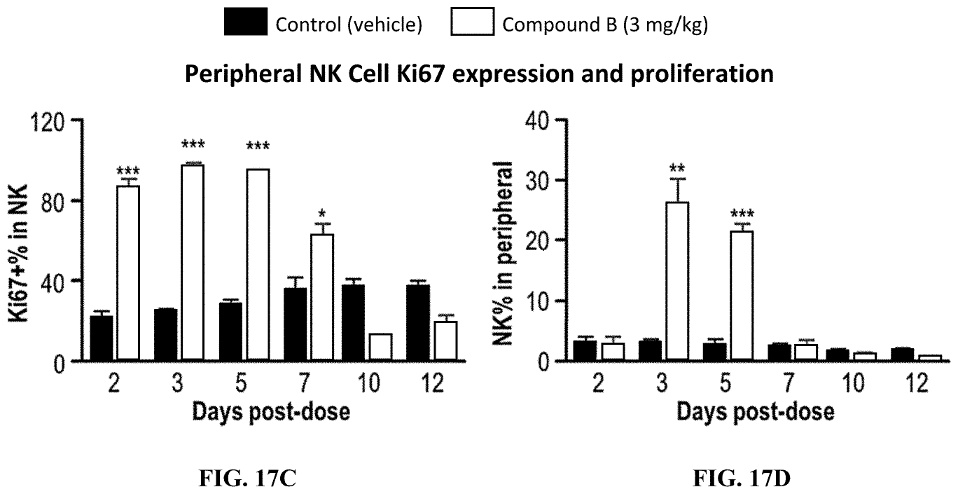

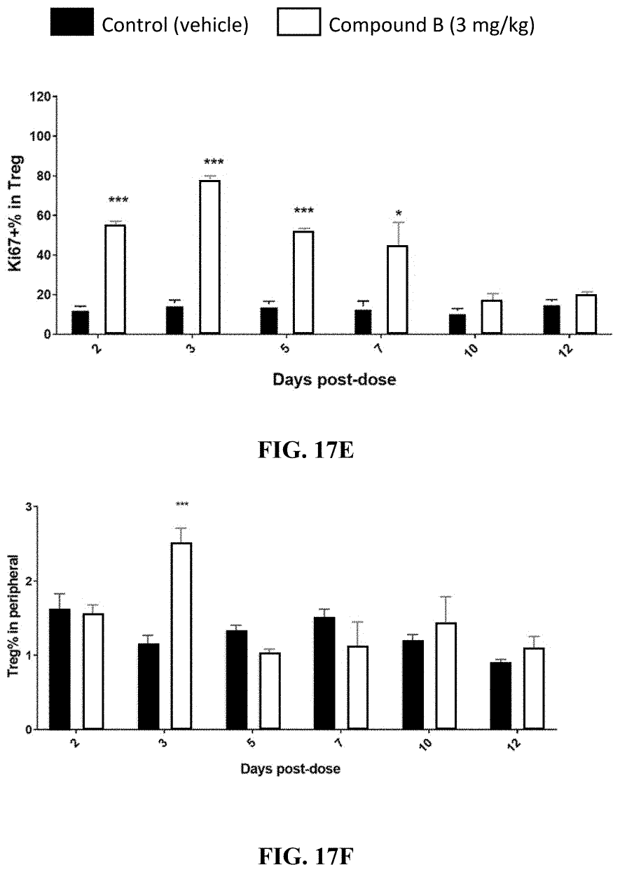

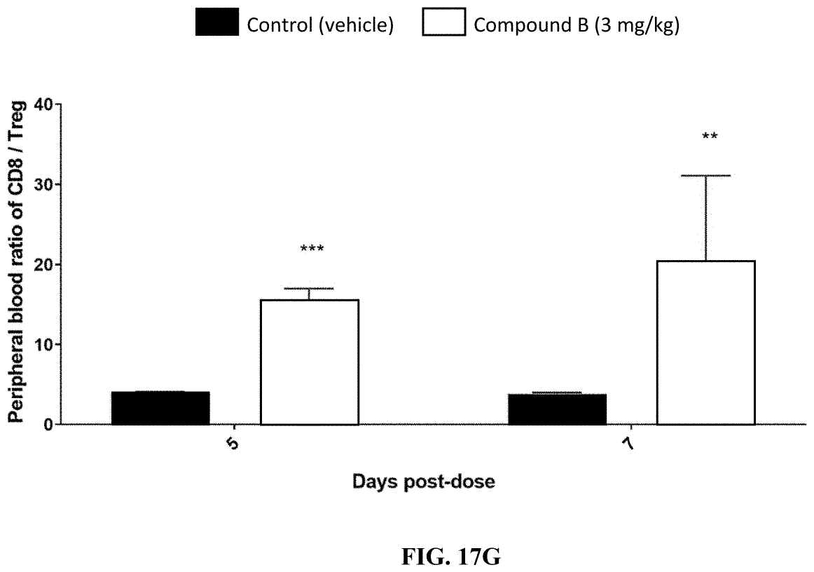

Post-pSTAT5 induction, Compound B induced significant activation of Ki67 in all three cell populations (CD8+ T, NK, and Treg cells) to the same degree from day 1 through 7 (p<0.05), before returning to vehicle control levels by day 10 (FIG. 17A-17F). As shown in FIG. 17A and FIG. 17B, activation of Ki67 by Compound B translated into significant proliferative responses of CD8+ T cells from 3 to 12 days (p<0.05 vs. control). Phenotypic analysis of CD8+ T cells revealed substantial expansion of CD44+ memory cells within this population over the same time course. In contrast to CD8+ T cells, Compound B induced maximal NK cell expansion at 3 days post dose and remained elevated on day 5 (p<0.05 vs. control) before returning to vehicle treated control levels by day 7 (FIG. 17C and FIG. 17D). In contrast to both CD8+T and NK cells, Compound B only caused very transient and a greatly reduced level (only 2.5% compared to 15-25% of CD8+T and NK cells) of Treg cell expansion on day 3 post-dose (FIGS. 17E and 17F). The expansion of CD8+ T cells and the lack of a significant expansion of CD4+ Treg cell subpopulation resulted in a progressive increase in the CD8+T/Treg ratio, peaking at Day 7 for the 3 mg/kg dose group (FIG. 17G).

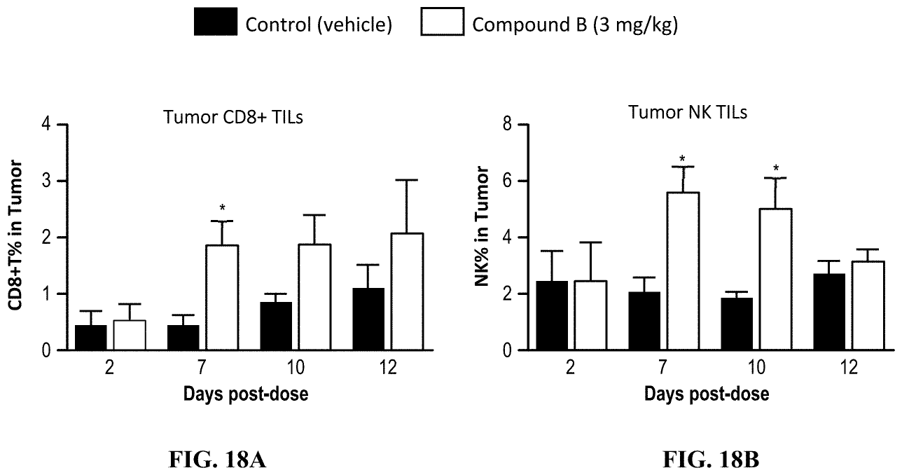

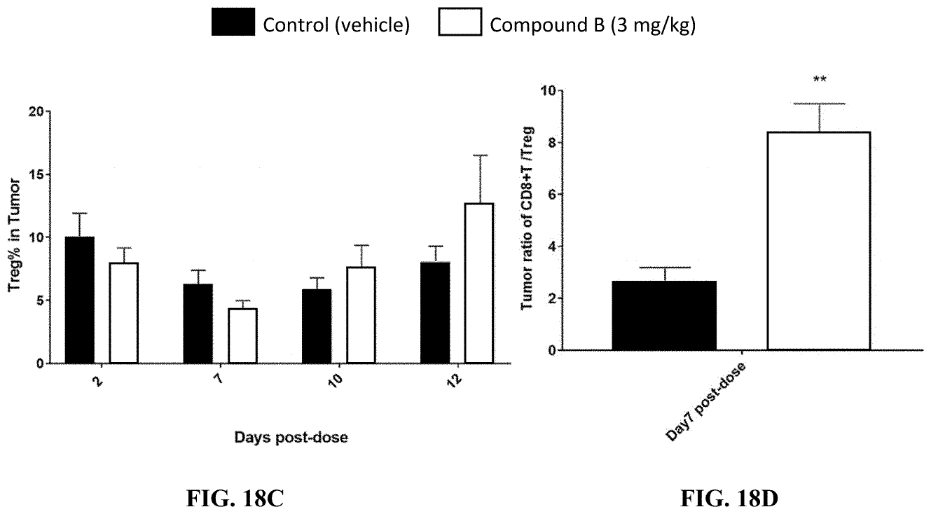

Analysis of tumor samples revealed that 7 days following treatment with Compound B, both CD8+ T cell and NK cells significantly expanded within the tumor (p<0.05 vs. control) and remained elevated through day 10 (FIG. 18A-18B). However, in response to Compound B, the Treg cell population within the tumor did not show significant expansion relative to the vehicle over time (FIG. 18C). The expansion of CD8+ T cells and the lack of an expansion of CD4+ Treg cell subpopulation resulted in a progressive increase in the CD8+T/Treg ratio, peaking at Day 7 for the 3 mg/kg dose group (FIG. 18D).

Summary. In CT-26 tumor-bearing mice, Compound B at 3 mg/kg induced peripheral pSTAT5 activation in all immune cell types including CD8+T, CD8+ memory, NK, and Treg cells, indicating engagement of the IL-2Rβ/γ receptor complex. Additionally, Compound B induced the proliferation marker Ki-67 in all these cell types at this dose, but proliferation was observed only in CD8+T and NK cells. This resulted in a CD8/Treg ratio of approximately 20 at this dose in the peripheral blood. Although the tumor exposure of Compound B was approximately 4% of plasma exposure, it was retained in the tumor for twice as long, resulting in CD8T/Treg ratios that was sufficient to show tumor growth inhibition. At higher doses of 6 and 9 mg/kg, greater tumor growth inhibition resulting in tumor regression was observed.

### Example 15

Compound B Increases Intra-Tumoral T Cell Fraction and TCR Diversity in Mouse CT-26 Tumors

The effect of Compound B as a single agent and in combination with an anti-PD-1 antibody on T cell repertoire was examined using CT-26 tumor-bearing mice. Balb/c female mice (6-8 weeks of age with an average weight of 16 to 22 g, Jackson Laboratories or Taconic Biosciences) were inoculated subcutaneously in the flank region with CT-26 tumor cells (ATCC), and the tumor growth was monitored by measuring the tumors, three times a week. When the tumors reached approximately 180 mm³, mice (N=4 for each group) were randomized into the following groups: control (Compound B vehicle+isotype control), Compound B (6 mg/kg single IV dose on day 0), mouse anti-PD-1 antibody (10 mg/kg, two doses on day 0 and 3, IP) or the combination of Compound B+anti-PD-1 antibody. Blood and tumor samples were collected pre-dose and at day 5, 8, 12, and 16 post-dose, and stored at −80° C. until analysis. The samples were analyzed for intra-tumoral T cell fraction and TCR diversity (Adaptive Biotechnologies, Seattle, Wash.). TCR sequencing was performed on infiltrating T cell via immunoSEQ™.

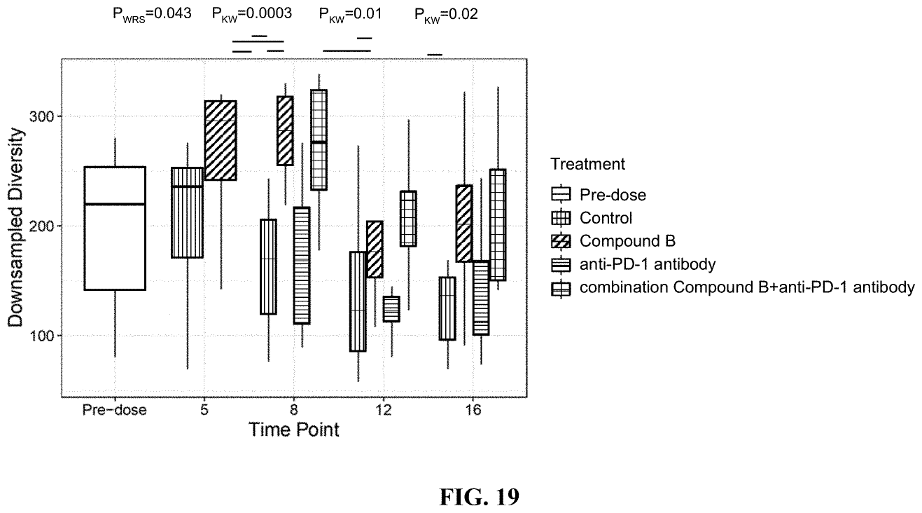

By day 8, CT-26 tumors treated with 6 mg/kg of Compound B alone, or in combination with mouse anti-PD-1 antibody, showed significantly lower TCR repertoire clonality as determined by post-hoc Dunn's test compared to either the vehicle or the anti-PD-1 antibody groups alone (p=0.005). Clonality was quantitated by the extent of mono- or oligoclonal dominance within a repertoire by measuring the shape of the clone frequency distribution. Clonal diversity was determined by downsampling to the minimum number of templates. Consistent with clonality, at days 5 and 8, TCR diversity showed the opposite trend and was higher in groups treated with Compound B or the combination of Compound+anti-PD-1 antibody (p<0.05) (FIG. 19). No significant difference was observed with either Compound B or the anti-PD-1 antibody combination treatment in T cell repertoire metrics at day 12 or 16.

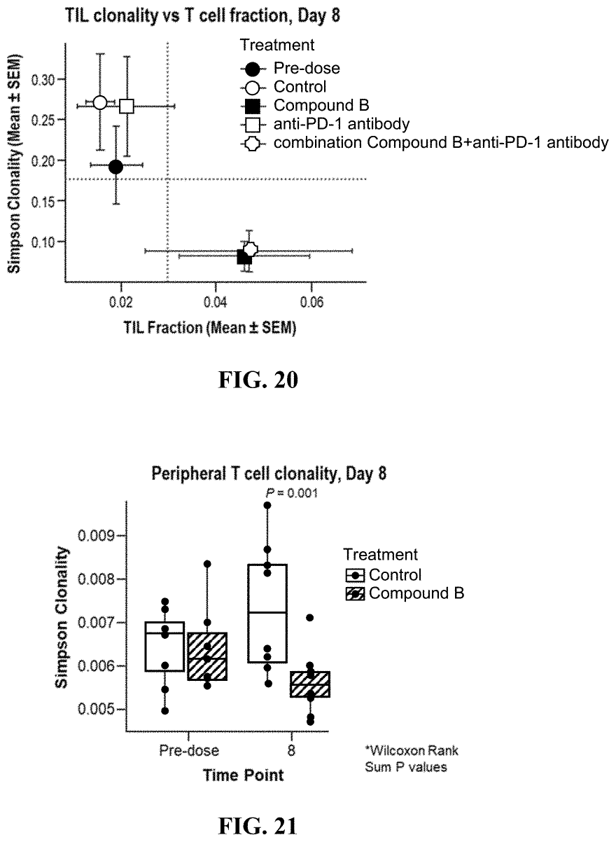

As shown in FIG. 20, TCR sequencing also demonstrated that Compound B elevates the tumor infiltrating lymphocytes (TIL) fraction alone or in combination with anti-PD-1 antibody (p<0.05). Analysis of day 8 peripheral blood samples revealed that Compound B also significantly decreased (p=0.001) T cell clonality compared to vehicle control consistent with the observations made in the tumor (FIG. 21).

### Example 16

Compound B Reprograms the CT-26 Tumor Microenvironment for High T_{eff} Activity, IFN-γ Induction, and Checkpoint Ligand Expression

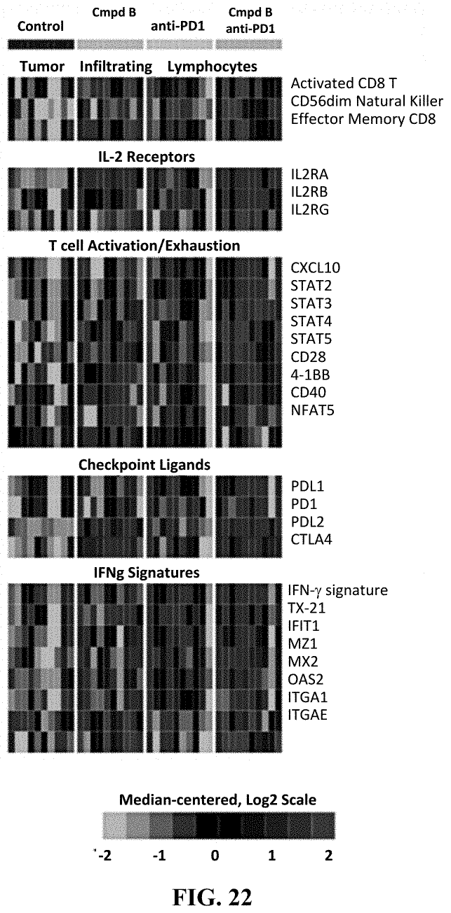

The CT-26 tumor samples from the study described above in Example 15 were also profiled via mRNAseq (Ominiseq, Buffalo, N.Y.) and analyzed by GeneCentric (Research Triangle Park, NC) to identify cell and molecular signatures of lymphocyte infiltration and activation. The data is presented as an expression heatmap from Day 8 CT26 tumor samples following treatment with control (vehicle), Compound B (6 mg/kg), mouse anti-PD-1 (10 mg/kg), or combination of Compound B and mouse anti-PD-1 (N=10 mice per group). All signatures and individual genes are shown with K-W p-values<0.05 (except PD-L1, p=0.23). Compound B and anti-PD1 treatment-induced immune activation is presented as heatmaps based on values generated using log 2 median-centered expression values of genes making up different immune signatures and individual genes. Boxplots showing individual immune activation signatures or individual gene expression levels were also created. Within these box plots, pairwise comparisons across control and treatment groups for days 8 and 12 were conducted and p-values were displayed when Wilcoxon Rank Sum Test p-values were <0.05). Heatmaps and box plots were generated using R program version 3.5.3. Box plots show lower quartile, median and upper quartile expression data. Plot whiskers show the full distribution of the expression data. The nomenclature provided in FIG. 22 corresponds to human ortholog genes.

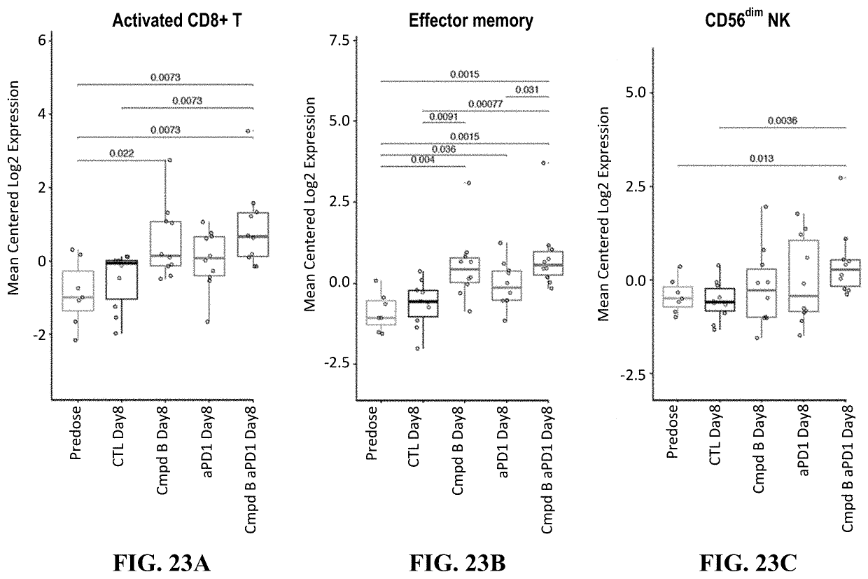

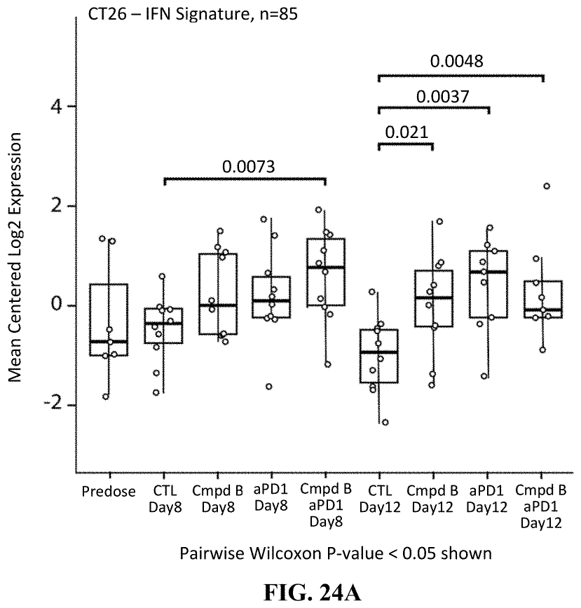

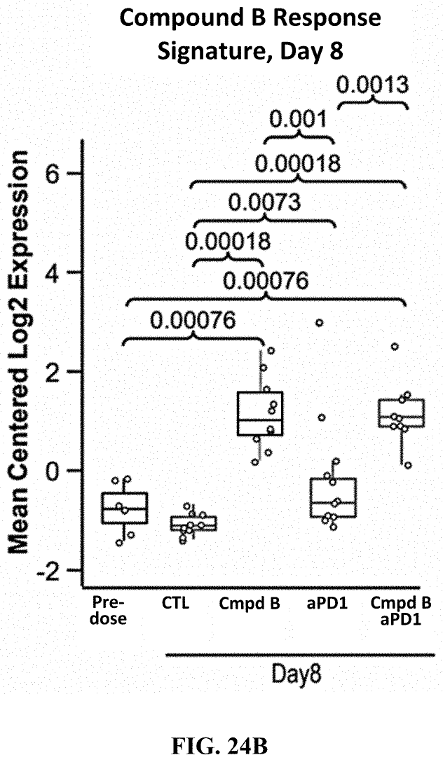

The top row of the heatmap of FIG. 22 shows that 8 days following a single dose of Compound B, the CT26 tumors were infiltrated with activated CD8+ effector and memory T cells and CD56^{dim} (cytolytic phenotype) NK cells. These cell populations were further enhanced by the combination with anti-PD-1 antibody. The mean centered log 2 expression levels (FIG. 23) show that Compound B significantly (p<0.05) elevated activated and memory CD8+ T cells in these tumors relative to pre-dose levels, whereas the combination of Compound B and anti-PD-1 antibody significantly increased CD56^{dim} NK cells compared to the pre-dose level or control (p<0.01). As shown in FIG. 22, Compound B induced multiple markers of IL-2 response and T cell activation, including the three IL-2 receptor chains CD28, 4-1BB, and CD40. In addition, Compound B treatment resulted in elevated expression of the checkpoint inhibitory receptor PD-1 and CTLA4, and the PD-1 ligands PD-L1 and PD-L2. Compound B also induced genes reporting on IFN-γ release and activation of IFN-γ signaling pathways (FIG. 24A).

In order to construct a signature response to Compound B in the CT-26 tumor-bearing mice, supervised analysis was conducted to ID differentially expressed genes (FDR<0/01) between vehicle control versus Compound B treated animals at day 8 post treatment. Gene expression profiles were contrasted between day 8 Compound B treated mice and day 8 vehicle control samples using significance analyses. Multiple comparisons were adjusted using false discovery rate (FDR=0.01). Genes were ranked by FDR-adjusted p-value, with corresponding fold change are listed in Table 35. A fold change greater than one indicates gene expression is higher in the Compound B treated arm; a fold change less than one indicates gene expression is higher in the control arm.

TABLE 35
Supervised analysis of expression changes to identify
new gene markers for development of a Compound
B redietive response signature (PRS).

| | Raw p- | P- | |
|---|---|---|---|
| Gene | value | value | Fold |
| F10 | 1.12E−6 | 0.000 | 3.2 |
| IL2RA | 1.12E−6 | 0.008 | 3.8 |
| CDH1 | 2.24E−6 | 0.008 | 3.0 |
| GZMK | 2.24E−6 | 0.009 | 4.0 |
| SEPT3 | 5.88E−6 | 0.009 | 5.3 |
| F7 | 5.88E−6 | 0.011 | 5.4 |
| PDCD1LG2 | 5.88E−6 | 0.011 | 4.6 |
| ASGR2 | 5.88E−6 | 0.011 | 4.6 |
| RAMP3 | 5.88E−6 | 0.011 | 3.1 |
| IL12B | 5.88E−6 | 0.011 | 6.5 |
| FLT3 | 8.12E−6 | 0.011 | 3.9 |
| CCL17 | 8.96E−6 | 0.011 | 7.3 |
| NR4A3 | 9.24E−6 | 0.011 | 2.5 |
| BCL2L14 | 9.24E−6 | 0.011 | 6.1 |
| DCSTAMP | 9.24E−6 | 0.011 | 4.1 |
| CTLA4 | 1.20E−5 | 0.012 | 3.7 |
| ARHGEF37 | 1.20E−5 | 0.012 | 3.0 |
| STRIP2 | 1.57E−5 | 0.015 | 4.6 |
| FAM169A | 2.24E−5 | 0.015 | 1.7 |
| CCL22 | 2.41E−5 | 0.015 | 3.8 |
| MEX3B | 2.55E−5 | 0.015 | 1.6 |
| HR | 2.58E−5 | 0.015 | 4.3 |
| TACR1 | 2.69E−5 | 0.015 | 2.7 |

Forty-two out of 43 of the most differentially expressed genes were used for a prototype Compound B PRS. Gene GT(ROSA)26 was excluded from the prototype PRS as this gene is used as a knock-in locus in mouse. Prototype Compound B calls were made and compared across the control, Compound B, and anti-PD1 treatment arms. Pairwise comparisons of the PRS were made across control and treatment groups for days 8 and 12, and are presented as box plots (FIG. 24A). P-values generated by pairwise Wilcoxon Rank Sum Test (p-value<0.05) are shown. Box plots were generated using the R program version 3.5.3 showing lower quartile, median and upperquartile expression data. Plot whiskers show median plus or minus 1.5 times the inter quartile range (IQR), or the minimum/maximum expression data when min/max fall within 1.5 times IQR. within 1.5 times IQR.

Forty-two genes were upregulated by Compound B on day 8 relative to the control. Twenty-three human orthologs shown in Table 35 were used to build a prototype signature of response. As shown by underlined text in Table 35, several genes with known IL-2 related biology were detected. Significant signature calls were observed with Compound B single agent and in combination with mouse anti-PD-1 antibody relative to the vehicle treated mice (FIG. 24B).

### Example 17

Compound B Promotes the Establishment of Persistent Memory T Cell Responses, Preventing CT-26 Tumor Growth in Surviving Animals Challenged by Re-Injection of CT-26 Cells

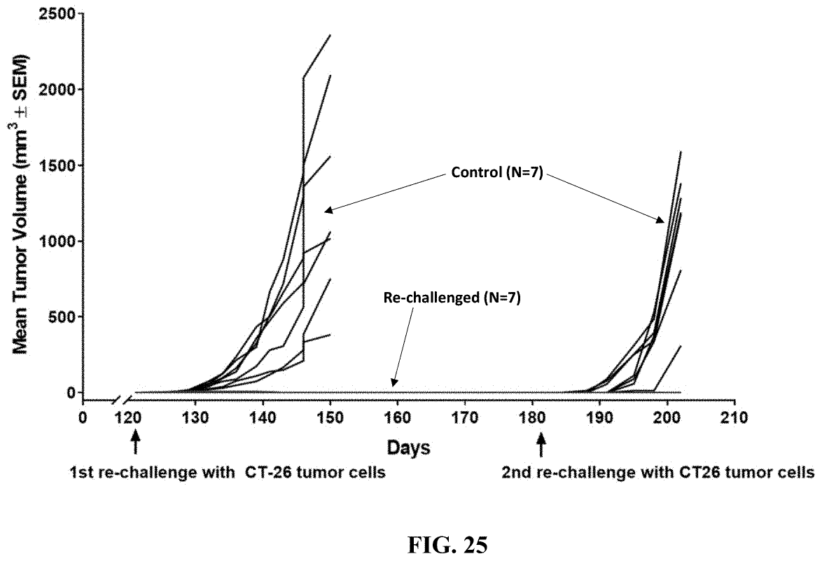

In Example 15, 7 mice remained tumor-free at Day 100 and included 1 animal each from the Compound B at 3 mg/kg, Compound B at 6 mg/kg, and the anti-PD-1 antibody groups, and 4 animals from the combination of Compound B at 6 mg/kg+anti-PD-1 antibody group. These 7 mice and 7 nave control mice were inoculated concurrently with the same number of CT-26 tumor cells on Day 121 (Study #2). As shown in FIG. 25, none of the 7 mice previously treated with Compound B, anti-PD-1 antibody, or the combination developed tumors, whereas all control animals grew tumors, indicating the establishment of durable memory T cell populations in response to the initial treatments. Two months later, on Day 181, the 7 treated tumor-free animals that survived the first re-challenge were inoculated again with CT-26 tumor cells, together with 7 control mice. Again, the 7 treated tumor-free animals did not develop tumors whereas tumors grew in the control animals (FIG. 25). The 7 tumor-free animals survived to Day 202 when the study was terminated.

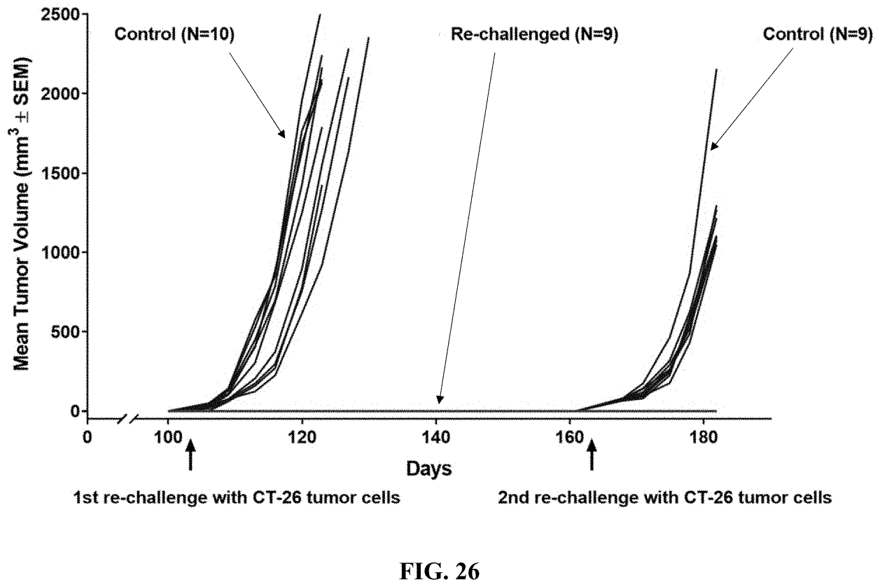

The re-challenge experiment was repeated with surviving animals in a further study (Study #3). A total of 9 surviving tumor-free mice (1 animal from each of the Compound B at 9 mg/kg and anti-PD-1 antibody groups, 2 animals from the combination Compound B at 3 mg/kg+anti-PD-1 antibody group, and 5 animals from the combination Compound B at 6 mg/kg+anti-PD-1 antibody group) with complete tumor regression at Day 102 were re-challenged by subcutaneous inoculation of CT-26 tumor cells. An additional 10 naïve control mice were also inoculated concurrently. As shown in FIG. 26, none of the 9 animals developed tumors indicating the establishment of durable memory T cell populations in response to the initial treatment. In contrast, all 10 control animals developed tumors. Two months later, on Day 163, a second re-challenge was performed in the same 9 animals that survived the first one; an additional 9 naïve animals served as controls. As was the case with the first re-challenge, the 9 surviving animals did not develop tumors, confirming the durability of memory T cell responses following initial treatments, whereas tumors grew in the control animals (FIG. 26). The 9 tumor-free animals survived to Day 184 when the study was terminated.

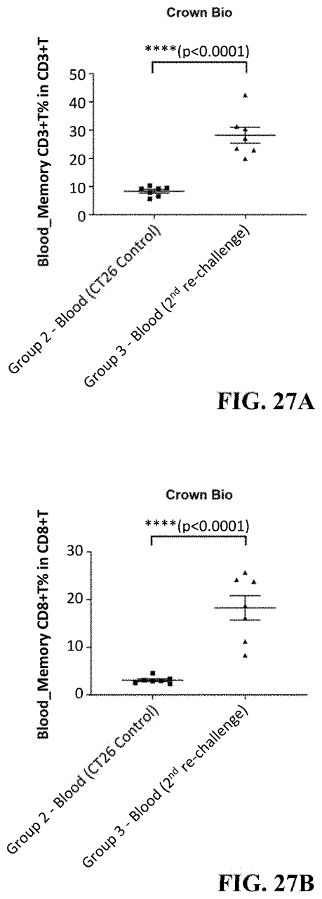

To determine the ability of Compound B to promote persistent T cell, memory response, 60 days following the second re-challenge blood samples were collected from the 7 surviving mice from Study #2. FACS analysis of blood samples for expression of memory cells revealed that Compound B promotes the establishment of durable immunological memory against CT-26 tumors, observed as an overall increase in peripheral memory T cells (CD3+), including memory CD8+ T cells (FIG. 27A-27B).

Summary Analysis of TILs in the Compound B 6 mg/kg treated tumors revealed an increase in T cell repertoire and activated CD8+ T cells (including effector memory) and NK cells and IFNγ signatures that induce check point ligands. Immune checkpoint therapy has been widely used and shown efficacy in a growing number of cancers, including metastatic melanoma and renal cell carcinoma (Hodi F. S. et al., N. Engl. J. Med. (2010) 363(8):711-723; Topalian S. L. et al., N. Engl. J. Med. (2012) 366(26):2443-2454; Wolchok J. D. et al., N. Engl. J. Med. (2013) 369(2):122-133; Sharma P. et al. Cell (2015) 161(2):205-214; Alsaab H. O. et al., Frontiers in Pharmacology (2017) 8:561, 1-15; Pardoll D. M. Nat. Rev. Cancer (2016) 12(4): 252-264). However, complete response rate is still quite low. Checkpoint inhibitors release the breaks on the dysfunctional cytotoxic T lymphocyte and prime them, while cytokine therapies such as IL-2 can activate and proliferate them. In addition, IL-2 based therapies can expand and activate Fc+ lymphocytes such as NK cells. Thus, combination of check point inhibitors with IL-2 complement each other, mediating effects on immune responses for improved anti-tumor responses. In the current studies, combination treatment of Compound B and anti-PD-1 resulted in a survival advantage compared to each single agent due to enhancement of both diversity and clonality of activated cytotoxic CD8+T and NK cells. Moreover, the tumor free mice treated with either Compound B or the combination had a persistent memory T cell population. This resulted in rejection of tumors cells in the surviving mice following re-challenges with the same tumor cells on two occasions, 2 months apart, with the first re-challenge 100 days after the last Compound B, anti-PD-1 antibody, or the combination treatment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby. The disclosures of all patent and scientific literature cited herein are expressly incorporated herein in their entirety by reference.

What is claimed is:

1. A method of treating cancer in a subject in need thereof, comprising administering to the subject a therapeutically effective amount of (a) an IL-2 conjugate, and (b) one or more immune checkpoint inhibitors, wherein the IL-2 conjugate comprises the amino acid sequence of SEQ ID NO: 3 in which at least one amino acid residue in the IL-2 conjugate is replaced by the structure of Formula (I): 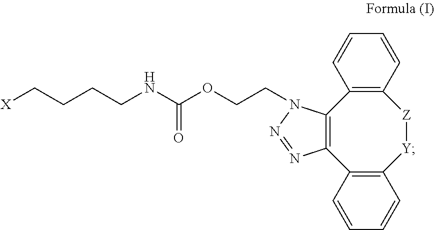
wherein:
Z is CH₂ and Y is 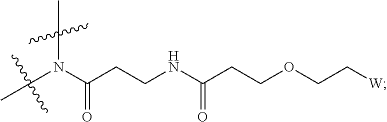
Y is CH₂ and Z is 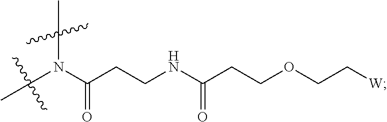
Z is CH₂ and Y is 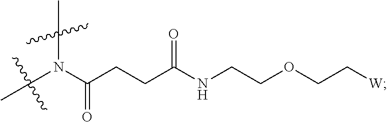
or
Y is CH₂ and Z is 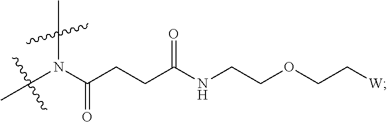
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
X has the structure: 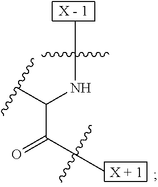 [SMILES: *NC(*)C(*)=O]
X−1 indicates the point of attachment to the preceding amino acid residue; and
X+1 indicates the point of attachment to the following amino acid residue;
wherein the position of the structure of Formula (I) in SEQ ID NO: 3 is selected from K34, F41, F43, K42, E61, P64, R37, T40, E67, Y44, V68, and L71.

2. The method according to claim 1, wherein in the IL-2 conjugate Z is CH₂ and Y is

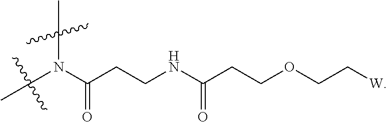

3. The method according to claim 1, wherein in the IL-2 conjugate Y is CH₂ and Z is

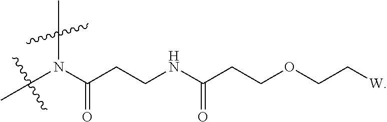

4. The method according to claim 1, wherein in the IL-2 conjugate Z is CH₂ and Y is

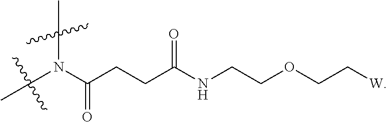

5. The method according to claim 1, wherein in the IL-2 conjugate Y is CH₂ and Z is

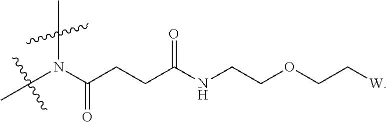

6. The method according to any one of claims 1-5, wherein in the IL-2 conjugate the PEG group has an average molecular weight of 25 kDa, 30 kDa, or 35 kDa.

7. The method according to claim 6, wherein in the IL-2 conjugate the PEG group has an average molecular weight of 30 kDa.

8. The method according to any one of claims 1-7, wherein in the IL-2 conjugate the position of the structure of Formula (I) in SEQ ID NO: 3 is P64.

9. The method of claim 1, wherein the structure of Formula (I) has the structure of Formula (X) or Formula (XI), or is a mixture of Formula (X) and Formula (XI): 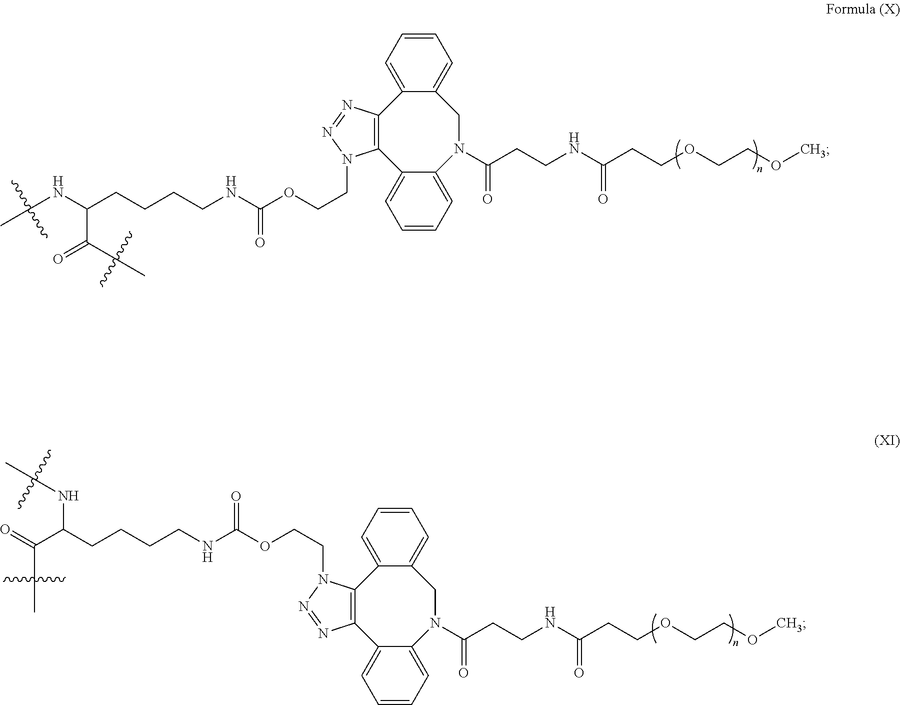
wherein:
n is an integer in the range from about 2 to about 5000; and
the wavy lines indicate covalent bonds to amino acid residues within SEQ ID NO: 3 that are not replaced.

10. The method of claim 9, wherein in the IL-2 conjugate the position of the structure of Formula (X) or Formula (XI) in SEQ ID NO: 3 is P64.

11. The method of claim 9 or 10, wherein in the IL-2 conjugate n is an integer such that —(OCH₂CH₂)ₙ—OCH₃ has a molecular weight of about 25 kDa, 30 kDa, or 35 kDa.

12. The method of claim 11, wherein in the IL-2 conjugate n is an integer such that —(OCH₂CH₂)ₙ—OCH₃ has a molecular weight of about 30 kDa.

13. The method of claim 1, wherein the structure of Formula (I) has the structure of Formula (XII) or Formula (XIII), or is a mixture of Formula (XII) and Formula (XIII): 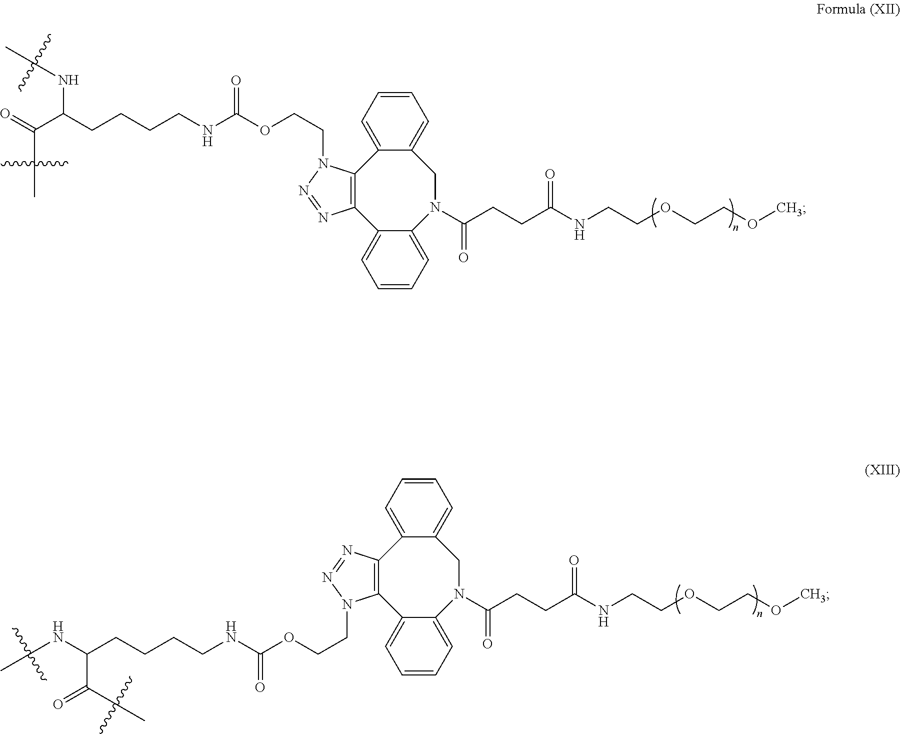
wherein:
n is an integer in the range from about 2 to about 5000; and
the wavy lines indicate covalent bonds to amino acid residues within SEQ ID NO: 3 that are not replaced.

14. The method of claim 13, wherein in the IL-2 conjugate the position of the structure of Formula (XII) or Formula (XIII) in SEQ ID NO: 3 is P64.

15. The method of claim 13 or 14, wherein in the IL-2 conjugate n is an integer such that —(OCH₂CH₂)ₙ—OCH₃ has a molecular weight of about 25 kDa, 30 kDa, or 35 kDa.

16. The method of claim 15, wherein in the IL-2 conjugate n is an integer such that —(OCH₂CH₂)ₙ—OCH₃ has a molecular weight of about 30 kDa.

17. A method of treating a cancer in a subject in need thereof, comprising administering to the subject a therapeutically effective amount of (a) an IL-2 conjugate, and (b) one or more immune checkpoint inhibitors, wherein the IL-2 conjugate comprises the amino acid sequence of SEQ ID NO: 50, wherein [AzK_L1_PEG30kD] has the structure of Formula (IV) or Formula (V), or is a mixture of the structures of Formula (IV) and Formula (V): 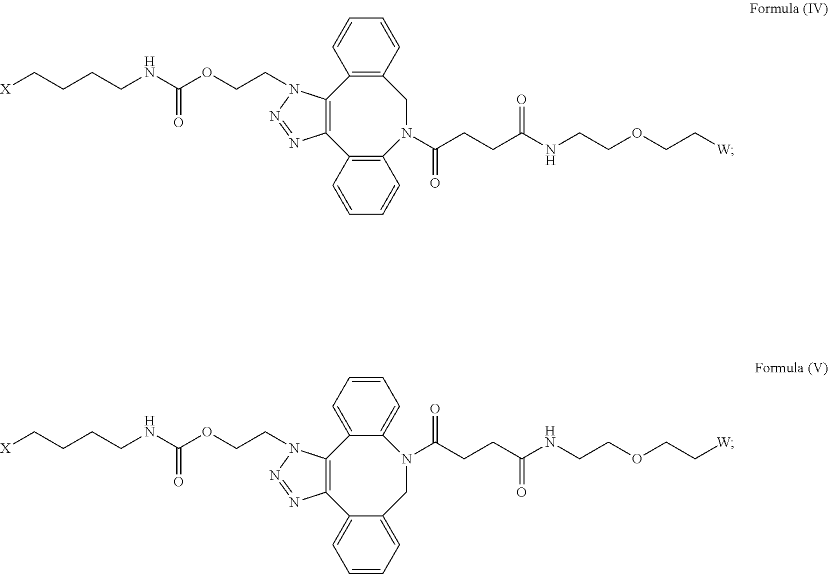
wherein:
W is a PEG group having an average molecular weight selected from 5 kDa, 10 kDa, 15 kDa, 20 kDa, 25 kDa, 30 kDa, 35 kDa, 40 kDa, 45 kDa, 50 kDa, and 60 kDa;
X has the structure: 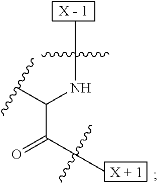 [SMILES: *NC(*)C(*)=O]
X−1 indicates the point of attachment to the preceding amino acid residue; and
X+1 indicates the point of attachment to the following amino acid residue.

18. The method according to claim 17, wherein W is a PEG group having an average molecular weight selected from 25 kDa, 30 kDa, or 35 kDa.

19. The method according to claim 18, wherein W is a PEG group having an average molecular weight of 30 kDa.

20. A method of treating a cancer in a subject in need thereof, comprising administering to the subject a therapeutically effective amount of (a) an IL-2 conjugate, and (b) one or more immune checkpoint inhibitors, wherein the IL-2 conjugate comprises the amino acid sequence of SEQ ID NO: 50, wherein [AzK_L1_PEG30kD] has the structure of Formula (XII) or Formula (XIII), or is a mixture of the structures of Formula (XII) and Formula (XIII): 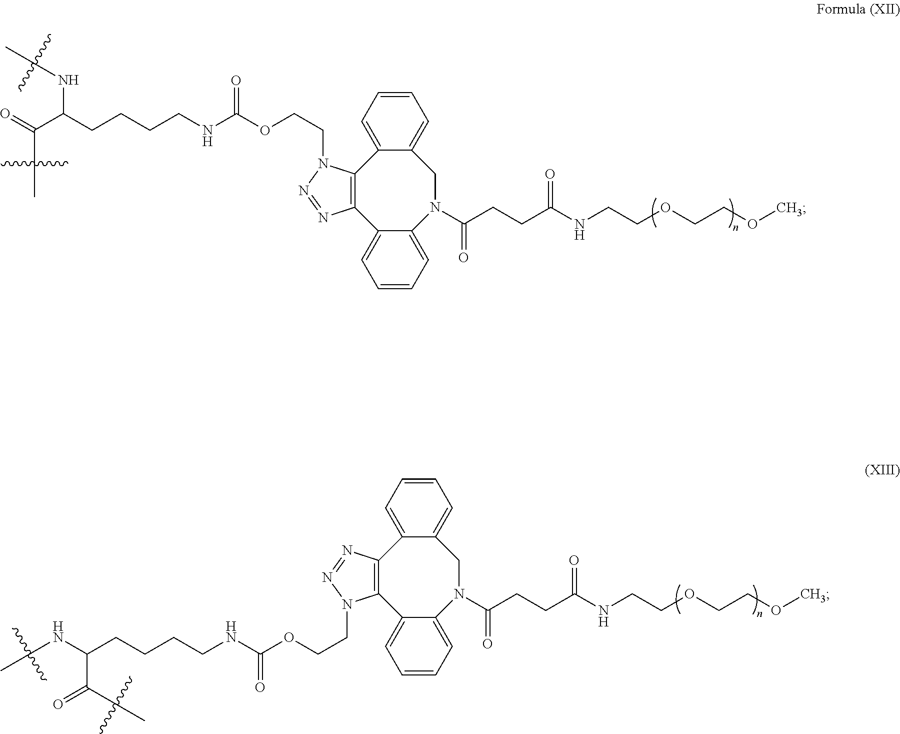
wherein:
n is an integer such that —(OCH₂CH₂)ₙ—OCH₃ has a molecular weight of about 30 kDa; and
the wavy lines indicate covalent bonds to amino acid residues within SEQ ID NO: 50 that are not replaced.

21. The method according to any one of claims 1-20, wherein the one or more immune checkpoint inhibitors is one or more PD-1 inhibitors.

22. The method according to claim 21, wherein the one or more PD-1 inhibitors is selected from pembrolizumab, nivolumab, and cemiplimab.

23. The method according to claim 22, wherein the one or more PD-1 inhibitors is pembrolizumab.

24. The method according to claim 22, wherein the one or more PD-1 inhibitors is nivolumab.

25. The method according to any one of claims 1-24, wherein the cancer is selected from renal cell carcinoma (RCC), non-small cell lung cancer (NSCLC), head and neck squamous cell cancer (HNSCC), classical Hodgkin lymphoma (cHL), primary mediastinal large B-cell lymphoma (PMBCL), urothelial carcinoma, microsatellite unstable cancer, microsatellite stable cancer, gastric cancer, colon cancer, colorectal cancer (CRC), cervical cancer, hepatocellular carcinoma (HCC), Merkel cell carcinoma (MCC), melanoma, small cell lung cancer (SCLC), esophageal, esophageal squamous cell carcinoma (ESCC), glioblastoma, mesothelioma, breast cancer, triple-negative breast cancer, prostate cancer, castrate-resistant prostate cancer, metastatic castrate-resistant prostate cancer, or metastatic castrate-resistant prostate cancer having DNA damage response (DDR) defects, bladder cancer, ovarian cancer, tumors of moderate to low mutational burden, cutaneous squamous cell carcinoma (CSCC), squamous cell skin cancer (SCSC), tumors of low- to non-expressing PD-L1, tumors disseminated systemically to the liver and CNS beyond their primary anatomic originating site, and diffuse large B-cell lymphoma.

26. The method according to any one of claims 1-25, wherein the IL-2 conjugate is administered to the subject once per week, once every two weeks, once every three weeks, once every 4 weeks, once every 5 weeks, once every 6 weeks, once every 7 weeks, or once every 8 weeks.

27. The method according to any one of claims 1-26, wherein the IL-2 conjugate is administered to a subject by intravenous administration.

28. The method according to any one of claims 1-27, wherein the IL-2 conjugate is a pharmaceutically acceptable salt, solvate, or hydrate.


SEQUENCE LISTING (98 sequences)

SEQ ID NO: 1 - protein, 133 aa, Homo sapiens [sequence omitted]

SEQ ID NO: 2 - protein, 153 aa, Homo sapiens
  misc_feature: IL-2 (homo sapiens) (precursor)
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 132 aa, Artificial Sequence
  Synthetic aldesleukin
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 4 - protein, 133 aa, Artificial Sequence
  Synthetic IL-2_C125S
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, Artificial Sequence
  Synthetic IL-2_P65X
  misc_feature (65)..(65): Xaa is any unnatural amino acid
APTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 6 - protein, 133 aa, Artificial Sequence
  Synthetic IL-2_E62X
  misc_feature (62)..(62): Xaa is any unnatural amino acid
APTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFSQSIISTLT

SEQ ID NO: 7 - protein, 133 aa, Artificial Sequence
  Synthetic IL-2_F42X
  misc_feature (42)..(42): Xaa is any unnatural amino acid
APTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 133 aa, Artificial Sequence
  Synthetic IL-2_K43X
  misc_feature (43)..(43): Xaa is any unnatural amino acid
APTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFSQSIISTLT

SEQ ID NO: 9 - protein, 133 aa, Artificial Sequence
  Synthetic IL-2_K35X
  misc_feature (35)..(35): Xaa is any unnatural amino acid
APTSSSTKKTQLQLEHLLLDLQMILNGINNYKNPXLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 10 - protein, 133 aa, Artificial Sequence; identical to SEQ ID NO: 5
  Synthetic IL-2_P65[AzK]
  misc_feature (65)..(65): Xaa is [AzK]

SEQ ID NO: 11 - protein, 133 aa, Artificial Sequence; identical to SEQ ID NO: 6
  Synthetic IL-2_E62[AzK]
  misc_feature (62)..(62): Xaa is [AzK]

SEQ ID NO: 12 - protein, 133 aa, Artificial Sequence; identical to SEQ ID NO: 7
  Synthetic IL-2_F42[AzK]
  misc_feature (42)..(42): Xaa is [AzK]

SEQ ID NO: 13 - protein, 133 aa, Artificial Sequence; identical to SEQ ID NO: 8
  Synthetic IL-2_K43[AzK]
  misc_feature (43)..(43): Xaa is [AzK]

SEQ ID NO: 14 - protein, 133 aa, Artificial Sequence; identical to SEQ ID NO: 9
  Synthetic IL-2_K35[AzK]
  misc_feature (35)..(35): Xaa is [AzK]

SEQ ID NO: 15 - protein, 133 aa, Artificial Sequence; identical to SEQ ID NO: 5
  Synthetic IL-2_P65[AzK_PEG]
  misc_feature (65)..(65): Xaa is [AzK_PEG]

SEQ ID NO: 16 - protein, 133 aa, Artificial Sequence; identical to SEQ ID NO: 6
  Synthetic IL-2_E62[AzK_PEG]
  misc_feature (62)..(62): Xaa is [AzK_PEG]

SEQ ID NO: 17 - protein, 133 aa, Artificial Sequence; identical to SEQ ID NO: 7
  Synthetic IL-2_F42[AzK_PEG]
  misc_feature (42)..(42): Xaa is [AzK_PEG]

SEQ ID NO: 18 - protein, 133 aa, Artificial Sequence; identical to SEQ ID NO: 8
  Synthetic IL-2_K43[AzK_PEG]
  misc_feature (43)..(43): Xaa is [AzK_PEG]

SEQ ID NO: 19 - protein, 133 aa, Artificial Sequence; identical to SEQ ID NO: 9
  Synthetic IL-2_K35[AzK_PEG]
  misc_feature (35)..(35): Xaa is [AzK_PEG]

SEQ ID NO: 20 - protein, 133 aa, Artificial Sequence; identical to SEQ ID NO: 5
  Synthetic IL-2_P65[AzK_PEG5kD]
  misc_feature (65)..(65): Xaa is [AzK_PEG5kD]

SEQ ID NO: 21 - protein, 133 aa, Artificial Sequence; identical to SEQ ID NO: 6
  Synthetic IL-2_E62[AzK_PEG5kD]
  misc_feature (62)..(62): Xaa is [AzK_PEG5kD]

SEQ ID NO: 22 - protein, 133 aa, Artificial Sequence; identical to SEQ ID NO: 7
  Synthetic IL-2_F42[AzK_PEG5kD]
  misc_feature (42)..(42): Xaa is [AzK_PEG5kD]

SEQ ID NO: 23 - protein, 133 aa, Artificial Sequence; identical to SEQ ID NO: 8
  Synthetic IL-2_K43[AzK_PEG5kD]
  misc_feature (43)..(43): Xaa is [AzK_PEG5kD]

SEQ ID NO: 24 - protein, 133 aa, Artificial Sequence; identical to SEQ ID NO: 9
  Synthetic IL-2_K35[AzK_PEG5kD]
  misc_feature (35)..(35): Xaa is [AzK_PEG5kD]

SEQ ID NO: 25 - protein, 133 aa, Artificial Sequence; identical to SEQ ID NO: 5
  Synthetic IL-2_P65[AzK_PEG30kD]
  misc_feature (65)..(65): Xaa is [AzK_PEG30kD]

SEQ ID NO: 26 - protein, 133 aa, Artificial Sequence; identical to SEQ ID NO: 6
  Synthetic IL-2_E62[AzK_PEG30kD]
  misc_feature (62)..(62): Xaa is [AzK_PEG30kD]

SEQ ID NO: 27 - protein, 133 aa, Artificial Sequence; identical to SEQ ID NO: 7
  Synthetic IL-2_F42[AzK_PEG30kD]
  misc_feature (42)..(42): Xaa is [AzK_PEG30kD]

SEQ ID NO: 28 - protein, 133 aa, Artificial Sequence; identical to SEQ ID NO: 8
  Synthetic IL-2_K43[AzK_PEG30kD]
  misc_feature (43)..(43): Xaa is [AzK_PEG30kD]

SEQ ID NO: 29 - protein, 133 aa, Artificial Sequence; identical to SEQ ID NO: 9
  Synthetic IL-2_K35[AzK_PEG30kD]
  misc_feature (35)..(35): Xaa is [AzK_PEG30kD]

SEQ ID NO: 30 - protein, 132 aa, Artificial Sequence
  Synthetic IL-2_P65X-1
  misc_feature (64)..(64): Xaa is any unnatural amino acid
PTSSSTKKTQLQLEHLLLDLQMILNGINNYKNPKLTRMLTFKFYMPKKATELKHLQCLEEELKXLEEVLNLAQSKNFHLRPRDLISNINVIVLELKGSETTFMCEYADETATIVEFLNRWITFSQSIISTLT

SEQ ID NO: 31 - protein, 132 aa, Artificial Sequence
  Synthetic IL-2_E62X-1
  misc_feature (61)..(61): Xaa is any unnatural amino acid
PTSSSTKKTQLQLEHLLLDLQMILNGINNYKNPKLTRMLTFKFYMPKKATELKHLQCLEEXLKPLEEVLNLAQSKNFHLRPRDLISNINVIVLELKGSETTFMCEYADETATIVEFLNRWITFSQSIISTLT

SEQ ID NO: 32 - protein, 132 aa, Artificial Sequence
  Synthetic IL-2_F42X-1
  misc_feature (41)..(41): Xaa is any unnatural amino acid
PTSSSTKKTQLQLEHLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 33 - protein, 132 aa, Artificial Sequence
  Synthetic IL-2_K43X-1
  misc_feature (42)..(42): Xaa is any unnatural amino acid
PTSSSTKKTQLQLEHLLLDLQMILNGINNYKNPKLTRMLTFXFYMPKKATELKHLQCLEEELKPLEEVLNLAQSKNFHLRPRDLISNINVIVLELKGSETTFMCEYADETATIVEFLNRWITFSQSIISTLT

SEQ ID NO: 34 - protein, 132 aa, Artificial Sequence
  Synthetic IL-2_K35X-1
  misc_feature (34)..(34): Xaa is any unnatural amino acid
PTSSSTKKTQLQLEHLLLDLQMILNGINNYKNPXLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 35 - protein, 132 aa, Artificial Sequence; identical to SEQ ID NO: 30
  Synthetic IL-2_P65[AzK]-1
  misc_feature (64)..(64): Xaa is [AzK]

SEQ ID NO: 36 - protein, 132 aa, Artificial Sequence; identical to SEQ ID NO: 31
  Synthetic IL-2_E62[AzK]-1
  misc_feature (61)..(61): Xaa is [AzK]

SEQ ID NO: 37 - protein, 132 aa, Artificial Sequence; identical to SEQ ID NO: 32
  Synthetic IL-2_F42[AzK]-1
  misc_feature (41)..(41): Xaa is [AzK]

SEQ ID NO: 38 - protein, 132 aa, Artificial Sequence; identical to SEQ ID NO: 33
  Synthetic IL-2_K43[AzK]-1
  misc_feature (42)..(42): Xaa is [AzK]

SEQ ID NO: 39 - protein, 132 aa, Artificial Sequence; identical to SEQ ID NO: 34
  Synthetic IL-2_K35[AzK]-1
  misc_feature (34)..(34): Xaa is [AzK]

SEQ ID NO: 40 - protein, 132 aa, Artificial Sequence; identical to SEQ ID NO: 30
  Synthetic IL-2_P65[AzK_L1_PEG]-1
  misc_feature (64)..(64): Xaa is [AzK_L1_PEG]

SEQ ID NO: 41 - protein, 132 aa, Artificial Sequence; identical to SEQ ID NO: 31
  Synthetic IL-2_E62[AzK_ L1_PEG]-1
  misc_feature (61)..(61): Xaa is [AzK_L1_PEG]

SEQ ID NO: 42 - protein, 132 aa, Artificial Sequence; identical to SEQ ID NO: 32
  Synthetic IL-2_F42[AzK_ L1_PEG]-1
  misc_feature (41)..(41): Xaa is [AzK_L1_PEG]

SEQ ID NO: 43 - protein, 132 aa, Artificial Sequence; identical to SEQ ID NO: 33
  Synthetic IL-2_K43[AzK_ L1_PEG]-1
  misc_feature (42)..(42): Xaa is [AzK_L1_PEG]

SEQ ID NO: 44 - protein, 132 aa, Artificial Sequence; identical to SEQ ID NO: 34
  Synthetic IL-2_K35[AzK_ L1_PEG]-1
  misc_feature (34)..(34): Xaa is [AzK_L1_PEG]

SEQ ID NO: 45 - protein, 132 aa, Artificial Sequence; identical to SEQ ID NO: 30
  Synthetic IL-2_P65[AzK_ L1_PEG5kD]-1
  misc_feature (64)..(64): Xaa is [AzK_L1_PEG5kD]

SEQ ID NO: 46 - protein, 132 aa, Artificial Sequence; identical to SEQ ID NO: 31
  Synthetic IL-2_E62[AzK_ L1_PEG5kD]-1
  misc_feature (61)..(61): Xaa is [AzK_L1_PEG5kD]

SEQ ID NO: 47 - protein, 132 aa, Artificial Sequence; identical to SEQ ID NO: 32
  Synthetic IL-2_F42[AzK_ L1_PEG5kD]-1
  misc_feature (41)..(41): Xaa is [AzK_L1_PEG5kD]

SEQ ID NO: 48 - protein, 132 aa, Artificial Sequence; identical to SEQ ID NO: 33
  Synthetic IL-2_K43[AzK_ L1_PEG5kD]-1
  misc_feature (42)..(42): Xaa is [AzK_L1_PEG5kD]

SEQ ID NO: 49 - protein, 132 aa, Artificial Sequence; identical to SEQ ID NO: 34
  Synthetic IL-2_K35[AzK_ L1_PEG5kD]-1
  misc_feature (34)..(34): Xaa is [AzK_L1_PEG5kD]

SEQ ID NO: 50 - protein, 132 aa, Artificial Sequence; identical to SEQ ID NO: 30
  Synthetic IL-2_P65[AzK_ L1_PEG30kD]-1
  misc_feature (64)..(64): Xaa is [AzK_L1_PEG30kD]

SEQ ID NO: 51 - protein, 132 aa, Artificial Sequence; identical to SEQ ID NO: 31
  Synthetic IL-2_E62[AzK_ L1_PEG30kD]-1
  misc_feature (61)..(61): Xaa is [AzK_L1_PEG30kD]

SEQ ID NO: 52 - protein, 132 aa, Artificial Sequence; identical to SEQ ID NO: 32
  Synthetic IL-2_F42[AzK_ L1_PEG30kD]-1
  misc_feature (41)..(41): Xaa is [AzK_L1_PEG30kD]

SEQ ID NO: 53 - protein, 132 aa, Artificial Sequence; identical to SEQ ID NO: 33
  Synthetic IL-2_K43[AzK_ L1_PEG30kD]-1
  misc_feature (42)..(42): Xaa is [AzK_L1_PEG30kD]

SEQ ID NO: 54 - protein, 132 aa, Artificial Sequence; identical to SEQ ID NO: 34
  Synthetic IL-2_K35[AzK_ L1_PEG30kD]-1
  misc_feature (34)..(34): Xaa is [AzK_L1_PEG30kD]

SEQ ID NO: 55 - protein, 133 aa, Artificial Sequence; identical to SEQ ID NO: 5
  Synthetic IL-2_P65[AzK_L1_PEG]-2
  misc_feature (65)..(65): Xaa is [AzK_L1_PEG]

SEQ ID NO: 56 - protein, 133 aa, Artificial Sequence; identical to SEQ ID NO: 6
  Synthetic IL-2_E62[AzK_ L1_PEG]-2
  misc_feature (62)..(62): Xaa is [AzK_L1_PEG]

SEQ ID NO: 57 - protein, 133 aa, Artificial Sequence; identical to SEQ ID NO: 7
  Synthetic IL-2_F42[AzK_ L1_PEG]-2
  misc_feature (42)..(42): Xaa is [AzK_L1_PEG]

SEQ ID NO: 58 - protein, 133 aa, Artificial Sequence; identical to SEQ ID NO: 8
  Synthetic IL-2_K43[AzK_ L1_PEG]-2
  misc_feature (43)..(43): Xaa is [AzK_L1_PEG]

SEQ ID NO: 59 - protein, 133 aa, Artificial Sequence; identical to SEQ ID NO: 9
  Synthetic IL-2_K35[AzK_ L1_PEG]-2
  misc_feature (35)..(35): Xaa is [AzK_L1_PEG]

SEQ ID NO: 60 - protein, 133 aa, Artificial Sequence; identical to SEQ ID NO: 5
  Synthetic IL-2_P65[AzK_ L1_PEG5kD]-2
  misc_feature (65)..(65): Xaa is [AzK_L1_PEG5kD]

SEQ ID NO: 61 - protein, 133 aa, Artificial Sequence; identical to SEQ ID NO: 6
  Synthetic IL-2_E62[AzK_ L1_PEG5kD]-2
  misc_feature (62)..(62): Xaa is [AzK_L1_PEG5kD]

SEQ ID NO: 62 - protein, 133 aa, Artificial Sequence; identical to SEQ ID NO: 7
  Synthetic IL-2_F42[AzK_ L1_PEG5kD]-2
  misc_feature (42)..(42): Xaa is [AzK_L1_PEG5kD]

SEQ ID NO: 63 - protein, 133 aa, Artificial Sequence; identical to SEQ ID NO: 8
  Synthetic IL-2_K43[AzK_ L1_PEG5kD]-2
  misc_feature (43)..(43): Xaa is [AzK_L1_PEG5kD]

SEQ ID NO: 64 - protein, 133 aa, Artificial Sequence; identical to SEQ ID NO: 9
  Synthetic IL-2_K35[AzK_ L1_PEG5kD]-2
  misc_feature (35)..(35): Xaa is [AzK_L1_PEG5kD]

SEQ ID NO: 65 - protein, 133 aa, Artificial Sequence; identical to SEQ ID NO: 5
  Synthetic IL-2_P65[AzK_ L1_PEG30kD]-2
  misc_feature (65)..(65): Xaa is [AzK_L1_PEG30kD]

SEQ ID NO: 66 - protein, 133 aa, Artificial Sequence; identical to SEQ ID NO: 6
  Synthetic IL-2_E62[AzK_ L1_PEG30kD]-2
  misc_feature (62)..(62): Xaa is [AzK_L1_PEG30kD]

SEQ ID NO: 67 - protein, 133 aa, Artificial Sequence; identical to SEQ ID NO: 7
  Synthetic IL-2_F42[AzK_ L1_PEG30kD]-2
  misc_feature (42)..(42): Xaa is [AzK_L1_PEG30kD]

SEQ ID NO: 68 - protein, 133 aa, Artificial Sequence; identical to SEQ ID NO: 8
  Synthetic IL-2_K43[AzK_ L1_PEG30kD]-2
  misc_feature (43)..(43): Xaa is [AzK_L1_PEG30kD]

SEQ ID NO: 69 - protein, 133 aa, Artificial Sequence; identical to SEQ ID NO: 9
  Synthetic IL-2_K35[AzK_ L1_PEG30kD]-2
  misc_feature (35)..(35): Xaa is [AzK_L1_PEG30kD]

SEQ ID NO: 70 - protein, 132 aa, Artificial Sequence; identical to SEQ ID NO: 30
  Synthetic IL-2_P65[AzK_PEG]-1
  misc_feature (64)..(64): Xaa is [AzK_PEG]

SEQ ID NO: 71 - protein, 132 aa, Artificial Sequence; identical to SEQ ID NO: 31
  Synthetic IL-2_E62[AzK_ PEG]-1
  misc_feature (61)..(61): Xaa is [AzK_PEG]

SEQ ID NO: 72 - protein, 132 aa, Artificial Sequence; identical to SEQ ID NO: 32
  Synthetic IL-2_F42[AzK_ PEG]-1
  misc_feature (41)..(41): Xaa is [AzK_PEG]

SEQ ID NO: 73 - protein, 132 aa, Artificial Sequence; identical to SEQ ID NO: 33
  Synthetic IL-2_K43[AzK_ PEG]-1
  misc_feature (42)..(42): Xaa is [AzK_PEG]

SEQ ID NO: 74 - protein, 132 aa, Artificial Sequence; identical to SEQ ID NO: 34
  Synthetic IL-2_K35[AzK_ PEG]-1
  misc_feature (34)..(34): Xaa is [AzK_PEG]

SEQ ID NO: 75 - protein, 132 aa, Artificial Sequence; identical to SEQ ID NO: 30
  Synthetic IL-2_P65[AzK_ PEG5kD]-1
  misc_feature (64)..(64): Xaa is [AzK_PEG5kD]

SEQ ID NO: 76 - protein, 132 aa, Artificial Sequence; identical to SEQ ID NO: 31
  Synthetic IL-2_E62[AzK_PEG5kD]-1
  misc_feature (61)..(61): Xaa is [AzK_PEG5kD]

SEQ ID NO: 77 - protein, 132 aa, Artificial Sequence; identical to SEQ ID NO: 32
  Synthetic IL-2_F42[AzK_PEG5kD]-1
  misc_feature (41)..(41): Xaa is [AzK_PEG5kD]

SEQ ID NO: 78 - protein, 132 aa, Artificial Sequence; identical to SEQ ID NO: 33
  Synthetic IL-2_K43[AzK_PEG5kD]-1
  misc_feature (42)..(42): Xaa is [AzK_PEG5kD]

SEQ ID NO: 79 - protein, 132 aa, Artificial Sequence; identical to SEQ ID NO: 34
  Synthetic IL-2_K35[AzK_PEG5kD]-1
  misc_feature (34)..(34): Xaa is [AzK_PEG5kD]

SEQ ID NO: 80 - protein, 132 aa, Artificial Sequence; identical to SEQ ID NO: 30
  Synthetic IL-2_P65[AzK_PEG30kD]-1
  misc_feature (64)..(64): Xaa is [AzK_PEG30kD]

SEQ ID NO: 81 - protein, 132 aa, Artificial Sequence; identical to SEQ ID NO: 31
  Synthetic IL-2_E62[AzK_PEG30kD]-1
  misc_feature (61)..(61): Xaa is [AzK_PEG30kD]

SEQ ID NO: 82 - protein, 132 aa, Artificial Sequence; identical to SEQ ID NO: 32
  Synthetic IL-2_F42[AzK_PEG30kD]-1
  misc_feature (41)..(41): Xaa is [AzK_PEG30kD]

SEQ ID NO: 83 - protein, 132 aa, Artificial Sequence; identical to SEQ ID NO: 33
  Synthetic IL-2_K43[AzK_PEG30kD]-1
  misc_feature (42)..(42): Xaa is [AzK_PEG30kD]

SEQ ID NO: 84 - protein, 132 aa, Artificial Sequence; identical to SEQ ID NO: 34
  Synthetic IL-2_K35[AzK_PEG30kD]-1
  misc_feature (34)..(34): Xaa is [AzK_PEG30kD]

SEQ ID NO: 85 - protein, 133 aa, Artificial Sequence
  Synthetic IL-2_F44X
  misc_feature (44)..(44): Xaa is any unnatural amino acid
APTSSSTKKTQLQLEHLLLDLQMILNGINNYKNPKLTRMLTFKXYMPKKATELKHLQCLEEELKPLEEVLNLAQSKNFHLRPRDLISNINVIVLELKGSETTFMCEYADETATIVEFLNRWITFCQSIISTLT

SEQ ID NO: 86 - protein, 132 aa, Artificial Sequence
  Synthetic IL-2_F44X-1
  misc_feature (43)..(43): Xaa is any unnatural amino acid
PTSSSTKKTQLQLEHLLLDLQMILNGINNYKNPKLTRMLTFKXYMPKKATELKHLQCLEEELKPLEEVLNLAQSKNFHLRPRDLISNINVIVLELKGSETTFMCEYADETATIVEFLNRWITFCQSIISTLT

SEQ ID NO: 87 - protein, 133 aa, Artificial Sequence
  Synthetic IL-2_R38X
  misc_feature (38)..(38): Xaa is any unnatural amino acid
APTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 88 - protein, 132 aa, Artificial Sequence
  Synthetic IL-2_R38X-1
  misc_feature (37)..(37): Xaa is any unnatural amino acid
PTSSSTKKTQLQLEHLLLDLQMILNGINNYKNPKLTXMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 89 - protein, 133 aa, Artificial Sequence
  Synthetic IL-2_T41X
  misc_feature (41)..(41): Xaa is any unnatural amino acid
APTSSSTKKTQLQLEHLLLDLQMILNGINNYKNPKLTRMLXFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 90 - protein, 132 aa, Artificial Sequence
  Synthetic IL-2_T41X-1
  misc_feature (40)..(40): Xaa is any unnatural amino acid
PTSSSTKKTQLQLEHLLLDLQMILNGINNYKNPKLTRMLXFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 91 - protein, 133 aa, Artificial Sequence
  Synthetic IL-2_E68X
  misc_feature (68)..(68): Xaa is any unnatural amino acid
APTSSSTKKTQLQLEHLLLDLQMILNGINNYKNPKLTRMLTFKFYMPKKATELKHLQCLEEELKPLEXVLNLAQSKNFHLRPRDLISNINVIVLELKGSETTFMCEYADETATIVEFLNRWITFCQSIISTLT

SEQ ID NO: 92 - protein, 132 aa, Artificial Sequence
  Synthetic IL-2_E68X-1
  misc_feature (67)..(67): Xaa is any unnatural amino acid
PTSSSTKKTQLQLEHLLLDLQMILNGINNYKNPKLTRMLTFKFYMPKKATELKHLQCLEEELKPLEXVLNLAQSKNFHLRPRDLISNINVIVLELKGSETTFMCEYADETATIVEFLNRWITFCQSIISTLT

SEQ ID NO: 93 - protein, 133 aa, Artificial Sequence
  Synthetic IL-2_Y45X
  misc_feature (45)..(45): Xaa is any unnatural amino acid
APTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 94 - protein, 132 aa, Artificial Sequence
  Synthetic IL-2_Y45X-1
  misc_feature (44)..(44): Xaa is any unnatural amino acid
PTSSSTKKTQLQLEHLLLDLQMILNGINNYKNPKLTRMLTFKFXMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, Artificial Sequence
  Synthetic IL-2_V69X
  misc_feature (69)..(69): Xaa is any unnatural amino acid
APTSSSTKKTQLQLEHLLLDLQMILNGINNYKNPKLTRMLTFKFYMPKKATELKHLQCLEEELKPLEEXLNLAQSKNFHLRPRDLISNINVIVLELKGSETTFMCEYADETATIVEFLNRWITFCQSIISTLT

SEQ ID NO: 96 - protein, 132 aa, Artificial Sequence
  Synthetic IL-2_V69X-1
  misc_feature (68)..(68): Xaa is any unnatural amino acid
PTSSSTKKTQLQLEHLLLDLQMILNGINNYKNPKLTRMLTFKFYMPKKATELKHLQCLEEELKPLEEXLNLAQSKNFHLRPRDLISNINVIVLELKGSETTFMCEYADETATIVEFLNRWITFCQSIISTLT

SEQ ID NO: 97 - protein, 133 aa, Artificial Sequence
  Synthetic IL-2_L72X
  misc_feature (72)..(72): Xaa is any unnatural amino acid
APTSSSTKKTQLQLEHLLLDLQMILNGINNYKNPKLTRMLTFKFYMPKKATELKHLQCLEEELKPLEEVLNXAQSKNFHLRPRDLISNINVIVLELKGSETTFMCEYADETATIVEFLNRWITFCQSIISTLT

SEQ ID NO: 98 - protein, 132 aa, Artificial Sequence
  Synthetic IL-2_L72X-1
  misc_feature (71)..(71): Xaa is any unnatural amino acid
PTSSSTKKTQLQLEHLLLDLQMILNGINNYKNPKLTRMLTFKFYMPKKATELKHLQCLEEELKPLEEVLNXAQSKNFHLRPRDLISNINVIVLELKGSETTFMCEYADETATIVEFLNRWITFCQSIISTLT